(19) United States
(12) Patent Application Publication
(10) Pub. No.: US 2017/0189443 A1
(43) Pub. Date: Jul. 6, 2017

(54) COMPOSITIONS AND METHODS FOR TREATMENT OF DISEASES AND CONDITIONS EMPLOYING ORAL ADMINISTRATION OF SODIUM PENTOSAN POLYSULFATE AND OTHER PENTOSAN POLYSULFATE SALTS

(71) Applicants: C. Lowell PARSONS, Henderson, NV (US); Michael GOLDBERG, San Diego, CA (US); Christopher P. MEENAN, Kinnelon, NJ (US); URIGEN PHARMACEUTICALS, INC., Wilmington, DE (US)

(72) Inventors: C. Lowell PARSONS, Henderson, NE (US); Michael GOLDBERG, San Diego, CA (US); Christopher P. MEENAN, Kinnelon, NJ (US)

(21) Appl. No.: 15/121,312

(22) PCT Filed: Feb. 24, 2015
(86) PCT No.: PCT/US2015/017205

§ 371 Date: Aug. 24, 2016

Related U.S. Application Data
(60) Provisional application No. 61/943,824, filed on Feb. 24, 2014.

(51) Int. Cl. A61K 31/737; A61K 47/00; A61K 45/06

(52) U.S. Cl. CPC A61K 31/737; A61K 45/06; A61K 47/00

(57) ABSTRACT

The present invention is directed to a pharmaceutical composition comprising: (1) a therapeutically effective quantity of sodium pentosan polysulfate; (2) a quantity of a penetration enhancer sufficient to improve the bioavailability of the sodium pentosan polysulfate; and (3) optionally, a pharmaceutically acceptable carrier and to methods for the oral administration of sodium pentosan polysulfate with improved bioavailability for the treatment of interstitial cystitis and other urinary tract diseases and conditions. Such compositions and methods allow the administration of sodium pentosan polysulfate at lower dosages to reduce the frequency and severity of side effects.

### CROSS-REFERENCES TO RELATED APPLICATIONS

This application claims the benefit of U.S. Provisional Application Ser. No. 61/943,824, filed Feb. 24, 2014, by Dr. C. Lowell Parsons, Dr. Michael Goldberg, and Christopher P. Meenan, and entitled “Compositions and Methods for Treatment of Diseases and Conditions Employing Oral Administration of Sodium Pentosan Polysulfate and Other Polysulfate Salts,” the contents of which are incorporated herein in their entirety by this reference.

### FIELD OF THE INVENTION

This invention is directed to compositions and methods for oral administration of sodium pentosan polysulfate for the treatment of a number of diseases and conditions, including interstitial cystitis and other urinary tract diseases and conditions, such as, but not limited to, renal calculi, radiation cystitis, prostatitis, overactive bladder, and urinary infections, as well as other diseases and conditions, including, but not limited to, HIV infection, prostate cancer, osteoarthritis, rheumatoid arthritis, juvenile rheumatoid arthritis, psoriatic arthritis, ankylosing spondylitis, lupus erythematosus, multiple sclerosis, asthma, prion disease, including variant Creutzfeldt-Jakob disease, inflammatory myocardial injury, osteonecrosis, intervertebral disc degeneration, amyloid-β-induced toxicity in Alzheimer's disease, atherosclerosis, and abnormal coagulation. The diseases and conditions can be treated in humans and in animals.

### BACKGROUND OF THE INVENTION

A large number of diseases and conditions occur in the lower urinary tract and are associated with one or more pelvic symptoms of pain, urge, frequency, or incontinence. In gynecologic patients, pelvic pain is referred to as chronic pelvic pain and may be of unknown origin or may be related to bacterial cystitis, fungal/yeast cystitis, vulvar vestibulitis, vulvodynia, dyspareunia, or endometriosis. Regardless of the perceived source of pelvic pain, in many cases the actual source of pain may be the bladder and/or the lower urinary tract. Frequency and urge together encompass the symptoms of overactive bladder. Overactive bladder may also be associated with incontinence, particularly urge incontinence.

In both male and female patients that are treated with cytotoxic therapies for cancer, this may result in any one or more lower urinary tract symptoms of pelvic pain, urge, frequency or incontinence. Localized radiation therapy to the pelvis due to bladder, cervical, ovarian, rectum, colon, vagina/vulva or prostate cancer therapy, may result in damaging the epithelium of the bladder wall leading to one or more of lower urinary tract symptoms of pain, urge, and/or frequency. Cytotoxic cancer chemotherapy, most notably cyclophosphamide and ifosfamide treatment for breast cancer patients (male and female) may also lead to the same series of symptoms.

In male patients, any one or more lower urinary tract pelvic symptoms of pelvic pain, urge, frequency or incontinence is observed in patients with prostatitis, chronic pelvic pain syndrome, urethral syndrome, or overactive bladder.

There are no specific treatments for lower urinary tract pelvic pain and instead patients are prescribed oral NSAIDs such as aspirin or acetaminophen. For severe chronic pain, some subjects rely on oral and/or transdermal narcotics which typically results in an irreversible worsening of symptoms.

For the symptoms of urinary urge and frequency, also termed overactive bladder, oral anticholinergic drugs such as detroloxybutynin chloride (Ditropan XL®) and tolterodine (Detrusitol®, Detrol LA®) reduce the contraction of the smooth muscle of the bladder wall. However, these drugs do not treat the underlying cause of the problem. Additionally, these drugs may result in side effects such as dry mouth, constipation, headache, blurred vision, hypertension, drowsiness, and urinary retention in approximately 50% of patients receiving them. The benefits of these drugs do not appear to overcome their risks/detriments since only 20% of patients refill their prescriptions.

There is one agent, Mesnex® (mesna) that is used for the prevention of hemorrhagic cystitis due to ifosfamide treatment in cancer patients. This agent is a detoxifying agent and binds and detoxifies the cancer drug. The drug does not treat acute pain and actually results in very high frequency of adverse events (all AEs for IV=85%, for oral=89%), most notable adverse events are nausea, vomiting, and constipation.

Although heparinoid-based therapy (heparin or the oral agent pentosan polysulfate sodium [PPS]) is an effective treatment for interstitial cystitis (IC), patients may require several months of therapy or more before they experience relief of pain and urgency/frequency (P. M. Hanno, “Analysis of Long-Term Elmiron Therapy for Interstitial Cystitis,” Uroloqy 49(Suppl 5A): 93-99 (1997)). Heparinoids, which are believed to augment the dysfunctional epithelium that is present in many cases of the disease, take time to reach full effectiveness in reversing the disease process and thereby reducing symptoms (C. L. Parsons, “Epithelial Coating Techniques in the Treatment of Interstitial Cystitis. Uroloqy 49(Suppl 5A): 100-104 (1997)). In addition, particularly in severe or long-standing cases of IC, there is significant upregulation of the sensory nerves in the bladder. (T J. Christmas et al., “Nerve Fibre Proliferation in Interstitial Cystitis,” Virchows Archiv. A Pathol. Anat. 416: 447-451 (1990); X. Pang et al., “Increased Number of Substance P Positive Nerve Fibres in Interstitial Cystitis,” Br. J. Urol. 75:744-750 (1995); C. A. Buffington & S. A. Wolfe, Jr., “High Affinity Binding Sites for [³H]Substance P in Urinary Bladders of Cats with Interstitial Cystitis,” J. Urol. 160:605-611 (1998)). Heparinoids allow natural downregulation of the nerves over time by gradually restoring the barrier function of the mucus and thus preventing further irritation by urinary constituents such as potassium (J. C. Nickel et al., “Randomized, Double-Blind, Dose-Ranging Study of Pentosan Polysulfate Sodium (PPS) for Interstitial Cystitis (IC),” J. Urol. 165(5 Suppl): 67 (2001); C. L. Parsons et al., “Effect of Pentosan Polysulfate Therapy on Intravesical Potassium Sensitivity,” Uroloqy 59: 329-333 (2002)) The use of heparinoids does not provide immediate symptom relief without destroying the nerve endings (T. W. Cannon & M. B. Chancellor, “Pharmacotherapy of the Overactive Bladder and Advances in Drug Delivery,” Clin. Obstet. Gynecol. 45: 205-17 (2002); M. B. Chancellor & N. Yoshimura, “Treatment of Interstitial Cystitis,” Uroloqy 63(3 Suppl 1): 85-89 (2004); M. Lazzeri et al., “Intravesical Infusion of Resiniferatoxin by a Temporary in Situ Drug Delivery System to Treat Interstitial Cystitis: A Pilot Study,” Eur. Urol. 45: 98-102 (2004)) or employing narcotics.

Although heparinoids have proven effective for the treatment of IC and similar conditions, as described above, heparin itself has not been available for oral administration and has not been used to treat IC or similar conditions by oral administration to patients suffering from these diseases or conditions.

Intravesical agents have been used for many years as adjuncts to oral treatment regimens or as second-line therapies for IC. One of the most widely used is heparin, which is effective in approximately 50% of patients treated. Heparin is a sulfated polysaccharide that is believed to augment the protective effect of the natural bladder surface mucus. Intravesical heparinoid agents alone, however, do not produce immediate and sustained relief of IC symptoms. Like the oral heparinoids, they take several months to produce symptom relief. Also, as indicated above, heparin has not been available for oral administration.

Other treatments have also been tried, with limited success. For example, treatments with dimethylsulfoxide (DMSO), approved for IC in 1977 on the basis of data from uncontrolled trials, can be useful with weekly intravesical instillations for 6 to 8 weeks then every two weeks for 3-12 months for maintenance. However DMSO therapy results in benefit for approximately only 50% of IC patients treated and the treatment takes a long time to reduce symptoms. Furthermore, this therapy causes pain that is unrelieved by local anesthetics by themselves due to their lack of absorption into the bladder wall. Narcotics are given for immediate relief of symptoms; however, they are only minimally effective. The use of narcotics, of course, carries a significant risk of tolerance and addiction. Some patients benefit from formal 8- to 12-week, one-on-one course of behavior modification. Patients are also advised to avoid potassium-rich foods, particularly citrus fruits, tomatoes, chocolate, and coffee.

Many urologists treat interstitial cystitis patients with their own “homebrew” of drugs by administering the drug(s) or mixtures thereof into the lumen of the bladder. As these procedures are typically done in the office without any quantitative assessment of severity of initial symptoms prior to or subsequent to treatment, there is no scientific rigor in assessing the benefit of these treatments. Consequently, patients are treated with drugs in their non-approved indications with no real scientific guidance as to whether the patient will benefit from the treatment or not.

Consequently, there is a tremendous need for scientifically-validated and improved treatments that provide immediate relief for treating lower urinary tract symptoms and disorders, particularly those with severe interstitial cystitis. Additionally, these treatments should be based on validated quantitative assessment of benefit, not on wishful thinking which has been the basis of urologists “homebrew” treatments that are not assessed quantitatively. There is a particular need for improved treatments and compositions for use in those treatments that provide immediate relief and do not require several months until the patients experience relief. In particular, there is a need for improved oral treatments for IC and other related conditions because oral treatments are far easier for the patient to administer and tolerate than intravesical treatments and do not carry the risk that invasive intravesical treatments may carry, such as the risk of injury or infection from catheterization.

Previously, Parsons (Parsons, C. L., “Evidence-based strategies for recognizing and managing IC,” Contemporary Uroloqy, February 2003, pp. 22-35), published a recipe of three FDA-approved drug components for the treatment of interstitial cystitis, which is a painful bladder disorder of unknown etiology. The components were 80 mg lidocaine (8 ml 1% lidocaine), 40,000 units heparin (4 ml of 10,000 units/ml heparin sodium), and 252 mg bicarbonate (3 ml of 8.4% sodium bicarbonate) in a total aqueous volume of 15 ml.

An additional limitation of the Parsons approach, as described in the 2003 Contemporary Urology article, is that components have to be measured out immediately before use from three separate solutions. In many treatment settings such as clinics or doctor's offices there are neither the pharmaceutical personnel resources qualified to measure out these components from stock solutions or the possibility exists of accidental mismeasurement leading to the potential for incorrect treatment or lidocaine overdose. Additionally, this mixing in a non-sterile environment may result in contamination with an infectious agent or other detrimental component that would be directly instilled in a compromised bladder.

One agent that has proven of value in treatment of interstitial cystitis and related conditions is sodium pentosan polysulfate, marketed as Elmiron®. Sodium pentosan polysulfate is described in U.S. Pat. No. 5,180,715 to Parsons, incorporated herein by this reference. In U.S. Pat. No. 5,180,715, the administration of sodium pentosan polysulfate by instillation into the bladder as well as by the oral route is described. (E. L. Davis et al., “Safety and Efficacy of the Use of Intravesical and Oral Pentosan Polysulfate Sodium for Interstitial Cystitis: A Randomized Double-Blind Trial,” J. Urol. 179: 177-185 (2008); J. C. Nickel et al., “Safety and Efficacy of up to 900 mg/Day Polysulfate Sodium (Elmiron) in Patients with Interstitial Cystitis,” Uroloqy 57 (Suppl. 1): 122-123 (2001)). However, the oral availability of sodium pentosan polysulfate is extremely limited, about 2-6%.

The structure of sodium pentosan polysulfate is shown below as Formula (I):

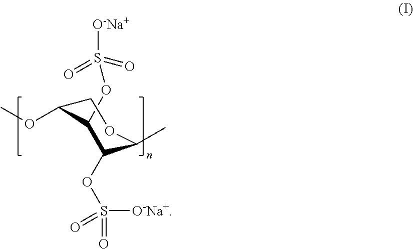

The reasons for the poor bioavailability of sodium pentosan polysulfate include the presence of charged groups (sulfate moieties). It is well known that charged groups have great difficulty penetrating the lipid bilayer of the cell membrane, because such lipid bilayers are extremely hydrophobic and the passage of a compound with multiple charged groups such as sulfate moieties through a lipid bilayer is energetically unfavorable. The relatively large size of the molecule also contributes to its poor bioavailability.

One study (M. Simon et al., “Metabolism of [³H]pentosan Polysulfate Sodium (PPS) in Healthy Human Volunteers,” Xenobiotica 35: 775-784 (2005), incorporated herein by this reference), reported that more than 94% of sodium pentosan polysulfate is excreted unchanged in feces without providing any beneficial effect and only 6% is excreted through urine. However, this study used tritiated pentosan polysulfate and did not directly measure pentosan polysulfate; the tritiated pentosan polysulfate spontaneously releases ³H. The key point from this study is that oral absorption of pentosan polysulfate is low.

Therefore, because of the relatively poor bioavailability of sodium pentosan polysulfate, relatively large doses are required when sodium pentosan polysulfate is administered orally to treat interstitial cystitis and other diseases or conditions affecting the urinary tract. The requirement for such large doses may lead to side effects. Patients who have taken sodium pentosan polysulfate orally have reported a variety of side effects, primarily gastrointestinal complaints such as diarrhea, heartburn, and stomach pain. Hair loss, headache, rash, and insomnia have also been reported.

The importance of providing compositions and methods possessing improved bioavailability of sodium pentosan polysulfate is increased with the discovery that sodium pentosan polysulfate has utility for the treatment of a substantial number of diseases and conditions other than urinary tract diseases and conditions. These diseases and conditions include: HIV infection (M. Wityrouw et al., “Establishment of a Bioassay to Determine Serum Levels of Dextran Sulfate and Pentosan Polysulfate, Two Potent Inhibitors of Human Immunodeficiency Virus,” J. Acquir. Immune Defic. Syndr. 3: 343-347 (1990); M. Peters et al., “Pharmacokinetics of Intravenous Pentosan Polysulfate (HOE/BAY 946) in HIV-Positive Patients,” AIDS 5: 1534-1535 (1991); M. Rusnati et al., “Pentosan Polysulfate as an Inhibitor of Extracellular HIV-1 Tat,” J. Biol. Chem. 276: 22420-22425 (2001)); prostatic carcinoma (S. Zaslau et al., “Pentosan Polysulfate (Elmiron): In Vitro Effects on Prostate Cancer Cells Regarding Cell Growth and Vascular Endothelial Growth Factor Production,” Am. J. Surq. 192: 640-643 (2006)); osteoarthritis (K. Kumagai et al., “Sodium Pentosan Polysulfate Resulted in Cartilage Improvement in Knee Osteoarthritis—An Open Clinical Trial,” BMC Clin. Pharmacol. 10: 7 (2010); P. Ghosh et al., “Pentosan Polysulfate, a Rational Therapy for the Treatment of Osteoarthritis. Results of a Double Blind Placebo Controlled Clinical Trial,” Ann. Rheum. Dis. 64: 1578 (2005); P. Ghosh, “The Pathobiology of Osteoarthritis and the Rationale for the Use of Pentosan Polysulfate for Its Treatment,” Semin. Arthritis Rheum. 28: 211-267 (1999)); rheumatoid arthritis and other rheumatoid disorders; prion-induced disease, including variant Creutzfeldt-Jakob disease (A. Parry et al., “Long Term Survival in a Patient with Variant Creutzfeldt-Jakob Disease Treated with Intraventricular Pentosan Polysulphate,” J. Neurol. Neurosurq. Psychiatry 78: 733-734 (2007); S. Dealler & N. G. Rainov, “Pentosan Polysulfate as a Prophylactic and Therapeutic Agent Against Prion Disease, IDrugs 6: 470-478 (2003)); osteonecrosis (N. Miyata et al. “Pentosan Reduces Osteonecrosis of Femoral Head in SHRSP,” Clin. Exp. Hypertens. 32: 511-516 (2010)); complement-mediated myocardial injury (K. S. Kilgore et al., “The Semisynthetic Polysaccharide Pentosan Polysulfate Prevents Complement-Mediated Myocardial Injury in the Rabbit Perfused Heart,” J. Pharmacol. Exp. Ther. 285: 987 (1998)); intervertebral disc degeneration (J. G. Zhao et al., “Calcium Pentosan Polysulfate and Sodium Pentosan Polysulfate May Be Used to Treat Intervertebral Disc Degeneration,” Med. Hypotheses 76: 610-613 (2011)); amyloid-β-induced toxicity in Alzheimer's disease (M. A. Deli et al., “Protection of the Blood-Brain Barrier by Pentosan Against Amyloid-β-Induced Toxicity,” J. Alzheimers Dis. 22: 777-794 (2010)); and atherosclerosis (E. Lupia et al., “Pentosan Polysulfate Inhibits Atherosclerosis in Watanabe Heritable Hyperlipidemic Rabbits: Differential Modulation of Metalloproteinase-2 and -9,” Lab. Invest. 92: 236-245 (2012); A. Klegeris et al., “Effect of C-Reactive Protein and Pentosan Polysulphate on Human Complement Activation,” Immunoloqy 106: 381-388 (2002)). Additionally, pentosan polysulfate can be and is used as an anticoagulant to control or prevent abnormal coagulation, such as in thrombotic conditions such as deep vein thrombosis, pulmonary embolism, or other diseases or conditions where inhibition or control of coagulation is required.

Therefore, there is an unfulfilled need to develop oral preparations containing sodium pentosan polysulfate with improved bioavailability for the treatment of diseases and conditions such as interstitial cystitis and other urinary tract diseases and conditions, described generally as lower urinary dysfunctional epithelium (LUDE), which can include, in addition to IC, overactive bladder (OAB), prostatitis (CP/CPPS), urethral syndrome (US) and gynecologic chronic pelvic pain (CPP), renal calculi, radiation cystitis, and urinary infections in addition to other diseases and conditions. Additionally, there is an unfulfilled need to develop oral preparations containing sodium pentosan polysulfate or other pentosan polysulfate salts, such as calcium pentosan polysulfate and potassium pentosan polysulfate, for the treatment of other diseases and conditions, such as, but not necessarily limited to, HIV infection, prostate cancer, osteoarthritis, rheumatoid arthritis, other rheumatoid conditions, prion disease, inflammatory myocardial injury, osteonecrosis, intervertebral disc degeneration, amyloid-β-induced toxicity in Alzheimer's disease, atherosclerosis, and abnormal coagulation. Preferably, such oral preparations allow the administration of lower dosages of sodium pentosan polysulfate or other salts of pentosan polysulfate that retain therapeutic effectiveness while reducing the frequency and severity of possible side effects.

### SUMMARY OF THE INVENTION

Accordingly, we have developed compositions and methods for the oral administration of sodium pentosan polysulfate with improved bioavailability for the treatment of interstitial cystitis and other urinary tract diseases and conditions. Such compositions and methods allow the administration of sodium pentosan polysulfate at lower dosages to reduce the frequency and severity of side effects.

One aspect of the invention is a pharmaceutical composition comprising:
(1) a therapeutically effective quantity of sodium pentosan polysulfate;
(2) a quantity of a penetration enhancer sufficient to improve the bioavailability of the sodium pentosan polysulfate; and
(3) optionally, a pharmaceutically acceptable carrier.

Typically, the therapeutically effective quantity of sodium pentosan polysulfate originally present in the composition is from about 50 mg to about 300 mg per unit dose of the composition. Preferably, the therapeutically effective quantity of sodium pentosan polysulfate originally present in the composition is from about 100 mg to about 200 mg per unit dose of the composition. These dosages are stated in terms of the dosage that is present and actually administered in the composition to a patient, not in terms of the dosage that is absorbed and bioavailable.

Typically, the quantity of penetration enhancer is from about 50 mg to about 800 mg per unit dose of the composition. Preferably, the quantity of penetration enhancer is from about 100 mg to about 500 mg per unit dose of the composition. More preferably, the quantity of penetration enhancer is from about 150 mg to about 400 mg per unit dose of the composition.

Typically, the ratio, by weight, of the penetration enhancer to the sodium pentosan polysulfate is from about 0.167:1 to about 8:1. Preferably, the ratio, by weight, of the penetration enhancer to the sodium pentosan polysulfate is from about 0.50:1 to about 3:1. More preferably, the ratio, by weight, of the penetration enhancer to the sodium pentosan polysulfate is from about 0.75:1 to about 2:1.

Typically, the quantity of penetration enhancer used is sufficient to increase the bioavailability of sodium pentosan polysulfate to at least 5%. Preferably, the quantity of penetration enhancer used is sufficient to increase the bioavailability of sodium pentosan polysulfate to at least 10%. More preferably, the quantity of penetration enhancer used is sufficient to increase the bioavailability of sodium pentosan polysulfate to at least 20%. Most preferably, the quantity of penetration enhancer used is sufficient to increase the bioavailability of sodium pentosan polysulfate to at least 30%.

A large number of transcellular and paracellular penetration enhancers are known in the art and can be used. These include, but are not limited to: (1) penetration enhancers selected from the group consisting of N-benzoyl-α-amino acids of Formula (II) and salts, analogues, or bioisosteres thereof:

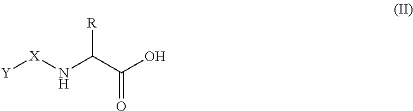

wherein the α-amino acid is selected from the group consisting of glycine, alanine, valine, leucine, phenylalanine, tyrosine, aspartic acid, glutamic acid, lysine, ornithine, arginine, and serine, wherein X is selected from the group consisting of C(O) and SO₂, and wherein Y is selected from the group consisting of phenyl and cyclohexyl; (2) penetration enhancers selected from the group consisting of derivatized leucines of Formula (III) and salts, analogues, or bioisosteres thereof:

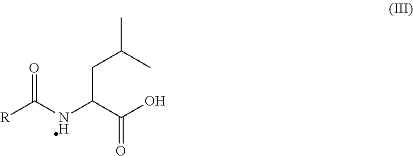

wherein R is selected from the group consisting of cyclohexyl, 2-methylcyclohexyl, 3-methylcyclohexyl, 4-methylcyclohexyl, cycloheptyl, cyclopentyl, cyclopropyl, 2-carboxycyclohexyl, benzoyl, 3-methoxyphenyl, 2-nitrophenyl, 3-nitrophenyl, 4-nitrophenyl, and (CH₂)₂cyclohexyl; (3) penetration enhancers selected from the group consisting of derivatives of 4-aminobenzoic acid, 2-(4-aminophenyl)acetic acid, 3-(4-aminophenyl)propionic acid, or 4-(4-aminophenyl)butyric acid of Formula (VI) and salts, analogues, or bioisosteres thereof:

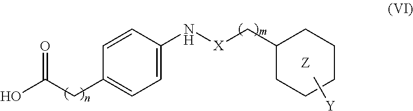

wherein: (a) Y is selected from the group consisting of H, F, 2-OH, 2,3-Ph, 4-Ph, 3,4-Ph, 4-OCH₃, 4-F, 2-Cl, 2-F, 2,4-(OH)₂, 3-CF₃, 3-Cl, 2-CH₃, 2,6-(OH)₂, 3-N(CH₃), 3,4-OCH₂O, 2,6-diCH₃, 2-COOH, 2-NO₂, 2-OCH₃, 3-NO₂, 2-OCF₃, 4-CH₃, and 4-i-Bu; (b) n is 0, 1, 2, 3, 4, or a vinyl group; (c) m is 0, 1, or 2, a vinyl group, a CHMe group, a CHEt group; a (CH₂)₂O group, a (CH₂)₂C═O group, or a (CH₂OH)₂ group; (d) X is C═O, SO₂, or CH₂; and (e) Z is phenyl, cyclohexyl, or cycloheptyl; and (4) penetration enhancers selected from the group consisting of compounds of Formula (VII):

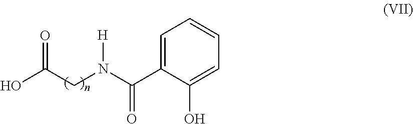

wherein n is 1, 2, 3, 4, 5, 6, 7, 8, 9, 10, or 11, and salts, analogues, or bioisosteres thereof, for which preferred penetration enhancers have n as 7, 8, or 9, including sodium N-[8-(2-hydroxybenzoyl)amino]caprylate. Other penetration enhancers have been described and are known in the art.

In a composition according to the present invention, the penetration enhancer can be a salt of a compound whose non-ionized form is a penetration enhancer. In another alternative, the penetration enhancer can be a compound possessing both at least one hydrophobic group and at least one hydrophilic group. The at least one hydrophobic group can be selected from the group consisting of carboxylic acid groups, carboxylic acid ester groups, amide groups, amino groups, and carbonyl groups.

When the pharmaceutical composition comprises the pharmaceutically acceptable carrier, the pharmaceutically acceptable carrier can be selected from the group consisting of an acidifying agent, an aerosol propellant, an air displacement, an alcohol denaturant, an alkalizing agent, an antifoaming agent, an antimicrobial preservative, an antioxidant, a buffering agent, a chelating agent, a coating agent, a colorant, a complexing agent, a desiccant, an emulsifying and/or solubilizing agent, a filtering aid, a flavor or perfume, a glidant and/or anticaking agent, a humectant, a plasticizer, a polymer, a solvent, a sorbent, a carbon dioxide sorbent, a stiffening agent, a suspending and/or viscosity-increasing agent, a sweetening agent, a tablet binder, a tablet and/or capsule diluent, a tablet disintegrant, a tonicity agent, a flavored and/or sweetened vehicle, an oleaginous vehicle, a solid carrier vehicle, a sterile vehicle, a water-repelling agent, and a wetting and/or solubilizing agent.

Another aspect of the invention is a method of treating lower urinary dysfunctional epithelium (LUDE) or a disease, condition, or syndrome associated with LUDE comprising the step of administering orally: (1) a pharmaceutically effective quantity of sodium pentosan polysulfate; and (2) a quantity of a penetration enhancer effective to improve the bioavailability of sodium pentosan polysulfate to a patient in need of treatment for LUDE or a disease, condition, or syndrome associated with LUDE in order to treat LUDE or a disease, condition, or syndrome associated with LUDE.

In this method, in one alternative, the sodium pentosan polysulfate and the penetration enhancer are administered in a pharmaceutical composition as described above. In another alternative, the sodium pentosan polysulfate and the penetration enhancer are administered separately. In this alternative, either the sodium pentosan polysulfate, the penetration enhancer, or both can be administered together with at least one filler, excipient, or carrier.

Typically, the disease, condition, or syndrome associated with LUDE and treated by the method is interstitial cystitis.

In another alternative, pentosan polysulfate, including sodium pentosan polysulfate, and, in some applications, potassium pentosan polysulfate and calcium pentosan polysulfate, can be used to treat other diseases and conditions, including, but not limited to, HIV infection, prostate cancer, osteoarthritis, prion disease, including variant Creutzfeldt-Jakob disease, inflammatory myocardial injury, osteonecrosis, intervertebral disc degeneration, amyloid-β-induced toxicity in Alzheimer's disease, and atherosclerosis. For treatment of these diseases and conditions, the sodium pentosan polysulfate, or, in some cases potassium pentosan polysulfate or calcium pentosan polysulfate, and the penetration enhancer can be administered in a pharmaceutical composition. In another alternative for treatment of these diseases and conditions, the sodium pentosan polysulfate, or, in some cases the potassium pentosan polysulfate or calcium pentosan polysulfate, and the penetration enhancer are administered separately. In this alternative, either the sodium pentosan polysulfate (or, in some cases, the potassium pentosan polysulfate or calcium pentosan polysulfate), the penetration enhancer, or both can be administered together with at least one filler, excipient, or carrier.

Pharmaceutical compositions suitable for use in methods according to the present invention are described above.

Yet another aspect of the invention is a method of treating a disease or condition associated with inflammation comprising the step of administering:
(1) a therapeutically effective quantity of a pentosan polysulfate salt; and
(2) a quantity of a penetration enhancer sufficient to improve the bioavailability of the pentosan polysulfate salt.

Typically, the disease or condition associated with inflammation is selected from the group consisting of rheumatoid arthritis, juvenile rheumatoid arthritis, osteoarthritis, psoriasis, psoriatic arthritis, ankylosing spondylitis, lupus erythematosus, multiple sclerosis, or asthma. Preferably, the disease or condition associated with inflammation is selected from the group consisting of osteoarthritis and rheumatoid arthritis. For these methods, the subject can be a human subject or can be a socially or economically important animal selected from the group consisting of a dog, a cat, a horse, a mule, a cow, a pig, a goat, and a sheep. Typically, the pentosan polysulfate salt is selected from the group consisting of sodium pentosan polysulfate, calcium pentosan polysulfate, and potassium pentosan polysulfate. Preferably, the pentosan polysulfate salt is sodium pentosan polysulfate. Typically, the pentosan polysulfate salt is administered in a pharmaceutical composition wherein the pharmaceutical composition further comprises at least one pharmaceutically acceptable carrier, excipient, or filler. In one alternative, the pharmaceutical composition comprises the penetration enhancer.

The method can further comprise administering a therapeutically effective quantity of at least one additional agent that is effective in treating inflammation. In one alternative, when the method further comprises administering the at least one additional agent, the penetration enhancer is effective in increasing the bioavailability of the at least one additional agent that is administered. Various combinations of the pentosan polysulfate salt and the at least one additional agent can be included in one or more pharmaceutical compositions. The at least one additional agent can be selected from the group consisting of:
(1) calcitonin selected from the group consisting of salmon calcitonin, eel calcitonin, and human calcitonin;
(2) a calcitonin derivative selected from including (Asu^{1,7})eel calcitonin, variants of calcitonin, fragments of calcitonin including amino acid residues 17-21 of calcitonin, and truncated derivatives of calcitonin lacking amino acid residues 1-9;
(3) a bisphosphonate selected from the group consisting of zoledronic acid, etidronate, clodronate, tiludronate, pamidronate, neridronate, olpadronate, alendronate, ibandronate, minodronate, incadronate, and risedronate;
(4) strontium ranelate;
(5) bone morphogenetic protein 7 (BMP-7), and homologs thereof including one or more conservative amino acid substitutions;
(6) a selective iNOS (inducible nitric oxide synthase) inhibitors including cindunistat; aminoguanidine hydrochloride; 2-amino-5,6-dihydro-6-methyl-4H-1,3-thiazine hydrochloride; AR-C 102222 (5-[(4′-amino-5′,8′-difluorospiro[piperidine-4,2′(1′H)-quinaxolin]-1-yl)carbonyl]-2-pyridinecarbonitrile hydrochloride); BYK 191023 dihydrochloride (2-[2-(4-methoxy-2-pyridinyl)ethyl]-1H-imidazo[4,5-b]pyridine dihydrochloride); (S)-ethylisothiourea hydrobromide; 2-iminopiperidine hydrochloride; (S)-isopropylisothiourea hydrobromide; (S)-methylisothiourea sulfate; N⁶-(1-iminoethyl)-L-lysine hydrochloride; N⁵-(1-iminoethyl)-L-ornithine dihydrochloride; and N-[[3-(Aminomethyl)phenyl]methyl]-ethanimidamide dihydrochloride);
(7) matrix metalloproteinase (MMP) inhibitors, wherein the MMP is selected from the group consisting of aggrecanase, MMP-1, MMP-13, MMP-3, cathepsin K, or another protease that participates in the catabolic process of tissue destruction, including batimastat, marimastat, ilomastat, prinomastat, cipemastat, MMI-166 (N-α-[4-(2-phenyl-2H-tetrazole-5-yl) phenyl sulfonyl]-D-tryptophan), MMI-270 ((2R)—N-hydroxy-2-[(4-methoxyphenyl)sulfonyl-(pyridin-3-ylmethyl)amino]-3-methylbutanamide), ABT-770 ((S)—N-[1-[[4′-trifluoromethoxy-[1,1′-biphenyl]-4-yl]oxy]methyl-2-(4,4-dimethyl-2,5-dioxo-1-imidazolidinyl)ethyl]-N-hydroxyformamide), RS-130830 (4-(((3-(4-chlorophenoxy)phenyl)sulfonyl)methyl)-N-hydroxytetrahydro-2H-pyran-4-carboxamide), CAS Reg. No. 239796-97-5 (1-benzyl-(4-(4-chlorophenoxy)phenyl)sulfonyl)-N-hydroxypiperidine-4-carboxamide), solimastat, KB-R-7785, GI-129471, rebimastat, tanomastat, Ro-28-2653, 544678-85-5, pyridine dicarboxamides, 868-68-30-3, CAS Reg. No. 582311-81-7, doxycycline, and metastat;
(8) endogenous inhibitors of metalloproteinases, including TIMP3;
(9) inhibitors of cathepsin K, including odanacatib;
(10) a COX-2 inhibitor such as rofecoxib, valdecoxib, celecoxib, etoricoxib, lumiracoxib, parecoxib, deracoxib, tiracoxib, meloxicam, nimesulide, (1,1-dimethylheptyl)-6a,7,10,10a-tetrahydro-1-hydroxy-6,6-dimethyl-6H-dibenzo[b,d]pyran carboxylic acid (CT-3), 5,5-dimethyl-3-(2-propoxy)-4-methanesulfonylphenyl)-2(5H)-furanone; carprofen; 2-(acetyloxy)benzoic acid 3-[(nitrooxy)methyl]phenyl ester (NCX4016), P54 (a turmeric derivative); 2,6-bis(1,1-dimethylethyl)[(E)-(2-ethyl-1,1-dioxo isothiazolidinylidene)methyl]phenol (S-2474), 5(R)-thiosulfonamide-3(2H)-benzofuranone (SVT-2016) and N-[3-(formyl-amino)oxophenoxy-4H benzopyranyl]methanesulfonamide (T-614); or a pharmaceutically acceptable salt thereof;
(11) a mixed COX-1/COX-2 inhibitor such as diclofenac;
(12) an inhibitor of TNFα such as etanercept, aldalimumab, or infliximab;
(13) a non-steroidal anti-inflammatory drug (NSAID) painkiller such as: an enolic acid selected from the group consisting of piroxicam, tenoxicam and meloxicam; a heteroaryl acetic acid selected from the group consisting of tolmetin, ketorolac, misoprostol, and zomepirac; an indole or indene acetic acid selected from the group consisting of indomethacin, mefenamic acid, sulindac and etodolac; a p-aminophenol derivative selected from the group consisting of phenacetin and acetaminophen; a propionic acid selected from the group consisting of naproxen, flurbiprofen, fenoprofen, oxaprozin, carprofen, ketoprofen and ibuprofen; a sulfonanilide selected from the group consisting of nimesulide; a fenamate selected from the group consisting of mefenamic acid, meclofenamate and flufenamic acid; an alkanone; a pyrazolone selected from the group consisting of phenylbutazone, oxyphenbutazone, antipyrine, aminopyrine, and kebuzone; and a salicylate selected from the group consisting of acetylsalicylic acid (aspirin), salicylate, salsalate, diflunisal, olsalazine, fendosal, sulfasalazine and thiosalicylate;
(14) a bone forming agent selected from the group consisting of an anti-Dkk1 antibody and an activin antagonist;
(15) a bone antiresorbing agent;
(16) a steroid hormone that is an estrogen, a partial estrogen agonist or estrogen-gestagen combination, wherein the hormone is selected from the group consisting of prednisolone, prednisone, methylprednisolone, betamethasone, hydrocortisone, cortisone, triamcinolone, dexamethasone, beclomethasone, budesonide, deoxycortone and fludrocortisone;
(17) a SERM (Selective Estrogen Receptor Modulator) selected from the group consisting of bazedoxifene acetate, ospemifene, raloxifene, arzoxifene, droloxifene, tamoxifen, 4-hydroxy-tamoxifen, 4′-iodotamoxifen, toremifene, (deaminohydroxy)-toremifene, chlomiphene, levormeloxifene, ormeloxifene, chroman derivatives, coumarin derivatives, idoxifene, nafoxidine, miproxifen phosphate (TAT-59), arzoxifene, lasofoxifene, (E)-1-butanamine, 4-(4-(2-chloro-1,2-diphenylethenyl)phenoxy)-N,N-diethyl-dihydrogen citrate (MDL-103323), acolbifene, (EM-652), EM-800, fulvestrant, N-(n-butyl)-1-[3,17β-dihydroxyestra-1,3,5(10)-trien-7α-yl]N-methylundecanamide (ICI 164,384), diethylstilbestrol, genistein, nafoxidine, nitromifene, moxesterol, diphenol hydrochrysene, erythro-MEA, allenolic acid, equilin-3-sulfate, cyclophenyl, chlorotrianisene, ethamoxytriphetol, lasofoxifene, bazedoxifene, genistein, tibolone, ospemifene, tesmilifene, droloxifene, panomifene, zindoxifene, meproxifene and faslodex;
(18) vitamin D or an analog thereof;
(19) parathyroid hormone (PTH), a PTH fragment or a PTH derivative selected from the group consisting of PTH (1-84), PTH (1-34), PTH (1-36), PTH (1-38), PTH (1-31)NH2 and PTS 893;
(20) a PTH releaser selected from the group consisting of 2-chloro-N-[(1R)-1-(3-methoxyphenyl)ethyl]-benzenepropanamine hydrochloride and cinacalcet;
(21) a strontium-containing compound that is an organic strontium salt selected from the group consisting of strontium malonate, strontium succinate, strontium fumarate, strontium ascorbate, strontium aspartate in either L- and/or D-form, strontium glutamate in either L- and/or D-form, strontium pyruvate, strontium tartrate, strontium glutarate, strontium maleate, strontium methanesulfonate, strontium benzenesulfonate, strontium acetyl salicylate, strontium salicylate, strontium citrate, strontium alendronate, strontium risedronate, strontium chlodronate, strontium ethidronate and strontium L-threonate, strontium ibandronate, strontium ibuprofenate, strontium flubiprofenate, strontium ketoprofenate, strontium phorbol 12,13-didecanoate 20-homovanillate, strontium indomethacinate, strontium carprofenate, strontium naproxenate, strontium acetyloxy-benzoate, strontium 2-iminopiperidine, strontium methotrexate, strontium salsalate and strontium sulfasalazinate;
(22) glucosamine;
(23) a disease modifying anti-rheumatic compound (DMARD) selected from the group consisting of doxycycline, chondroitin sulfate, methotrexate, leflunomide, dimethylnitrosamine, azatriopine, hydroxychloroquine, cyclosporine, minocycline, salazopyrine, penicillamine, aurothiomalate (gold salt), cyclophosphamide, azathioprine and pharmacologically active metabolites thereof;
(24) an aromatase inhibitor selected from the group consisting of aminoglutethimide, testolactone, anastrozole, letrozole, exemestane, vorozole, formestane, fadrozole, 4-hydroxyandrostenedione, 1,4,6-androstatrien-3,17-dione, and 4-androstene-3,6,17-trione;
(25) a COX-3 inhibitor selected from the group consisting of acetaminophen, dipyrone, antipyrine, and dimethylaminopyrene;
(26) an opioid selected from the group consisting of fentanyl, morphine, oxycodone, hydrocodone, methadone, buprenorphine, pentazocine, butorphanol, dezocine, nalbuphine, meperidine, normeperidine, hydromorphone, codeine, levorphanol, tramadol, endorphin, nociceptin, endomorphin, and active metabolites thereof;
(27) an inhibitor/antagonist of IL-1 that is a monoclonal antibody specifically binding IL-1 or a soluble IL-1 receptor derivative;
(28) an inhibitor/antagonist of interleukin-I converting enzyme;
(29) an inhibitor of RANK-ligand selected from the group consisting of OPG and monoclonal antibody 162;
(30) an anabolic growth factor selected from the group consisting of: (i) an anabolic growth factor derived from a bone or cartilage matrix protein selected from the group consisting of segments of or fragments from collagen type I, collagen type II, collagen type IX, collagen type XI, bone sialo protein (BSP), osteonectin, osteopontin, osteocalcin (also known as bone GLA protein), cartilage oligomeric matrix protein (COMP), cartilage intermediate layer protein (CILP) and aggrecan; (ii) human growth hormone (hGH); (iii) glucagon like peptide-2 (GLP-2); and (iv) insulin like growth factor-1 (IGF-1) with or without IGF binding protein 3 (IGFBP-3);
(31) a statin selected from the group consisting of nystatin, pravastatin, fluvostatin, atorvastatin, and cerivastatin and therapeutically active derivatives thereof;
(32) an endothelin-1 antagonist/inhibitor selected from the group consisting of bosentan, sitaxentan, ambrisentan, atrasentan, BQ-123 (2-[(3R,6R,9S,12R,15S)-6-(1H-indol-3-ylmethyl)-9-(2-methylpropyl)-2,5,8,11,14-pentaoxo-12-propan-2-yl-1,4,7,10,13-pentazabicyclo[13.3.0]octadecan-3-yl]acetic acid), zibotentan, macitentan, tenosentan, BQ-788 (N-[(cis-2,6-Dimethyl-1-piperidinyl)carbonyl]-4-methyl-L-leucyl-1-(methoxycarbonyl)-D-tryptophyl-D-norleucine sodium salt), and A192621 ((2R,3R,4S)-4-(1,3-benzodioxol-5-yl)-1-[2-(2,6-diethylanilino)-2-oxoethyl]-2-(4-propoxyphenyl)pyrrolidine-3-carboxylic acid);
(33) an NMDA receptor antagonist selected from the group consisting of R-2-amino-5-phosphonopentanoate, 2-amino-7-phosphonoheptanoic acid, 3-[(R)-2-carboxypiperazin-4-yl]-prop-2-enyl-1-phosphonic acid, selfotel, amantidine, atomoxetine, lanicemine, dextrallorphan, dizocilpine, gacyclidine, memantine, nitromemantine, neramexane, eliprodil, WMS-259 ((2S,4S)-2-[(4S)-2,2-Diphenyl-1,3-dioxolan-4-yl]-4-fluoropiperidine) remacemide, delucemine, aptiganel, rapastinel, NRX-1074 1-aminocyclopropane-1-carboxylic acid, and 5,7-dichlorokynurenic acid;
(34) a calcitonin gene related peptide-α antagonist selected from the group including olcegepant, telcagepant, ubrogepant, and an antibody or fragment thereof specifically binding calcitonin gene related peptide-α;
(35) chondroitin sulfate;
(36) keratin sulfate;
(37) a glycine antagonist selected from the group consisting of bicuculline, brucine, and tutin;
(38) a vanilloid receptor antagonist selected from the group consisting of AMG 517 (N-(4-((6-(4-trifluoromethyl)phenyl)pyrimidin-4-yl)oxy)benzo[d]thiazole-2-yl)acetamide), SB-705498 ((R)-1-(2-bromophenyl)-3-(1-(5-(trifluoromethyl)pyridin-2-yl)pyrrolidin-3-yl)urea), GRC 6211, AZD1386 and NGD 8243;
(39) a N-acetylcholine receptor antagonist selected from the group consisting of hexamethonium, mecamylamine, trimethaphan, atracurium, doxacurium, mivacurium, pancuronium, vecuronium, and 18-methoxycoronaridine;
(40) a neurokinin antagonist selected from the group consisting of RPR-100893 ((2S)-1-[(3aS,4S,7aS)-4-hydroxy-4-(2-methoxyphenyl)-7,7-diphenyl-1,3,3a,5,6,7a-hexahydroisoindol-2-yl]-2-(2-methoxyphenyl)propan-1-one), CP-99994 ((2S,3S)—N-[(2-methoxyphenyl)methyl]-2-phenyl-3-piperidinamine dihydrochloride), L-733,060 ((2S,3S)-3-{[3,5-bis(trifluoromethyl)benzyl]oxy}-2-phenylpiperidine), aprepitant, fosaprepitant, vofopitant, lanepitant, and TAK-637 (R)-7-(3,5-bis(trifluoromethyl)benzyl)-9-methyl-5-(p-tolyl)8,9,10,11-tetrahydro-7H-[1,4]diazocino[2,1-g][1,7]naphthyridine-6,13-dione);
(41) a neuroleptic agent selected from the group consisting of benperidol, bromperidol, droperidol, haloperidol, moperone, pipamperone, timiperone, fluspirilene, penfluridol, pimozide, acepromazine, chlorpromazine, cyamemazine, dixyrazine, fluphenazine, levomepromazine, mesoridazine, perazine, pericyazine, perphenazine, pipotiazine, prochlorperazine, promazine, promethazine, prothipendyl, thioproperazine, thioridazine, trifluoperazine, triflupromazine, chlorprothixene, clopenthixol, flupentixol, tiotixene, zuclopenthixol, clotiapine, loxapin, prothipendyl, carpipramine, clocapramine, molindone, mosapramine, sulpiride, sultopiride, veralipride, amisulpride, amoxapine, aripiprazole, asenapine, clozapine, blonanserin, iloperidone, lurasidone, melperone, nemonapride, olanzapine, paliperidone, perosperone, quetiapine, remoxipride, risperidone, sertindole, trimipramine, ziprasidone, and zotepine;
(42) a PAR2 receptor antagonist selected from the group consisting of AC-55541 (N-[[1-(3-bromo-phenyl)-eth-(E)-ylidene-hydrazinocarbonyl]-(4-oxo-3,4-dihydro-phthalazin-1-yl)-methyl]-benzamide) and AC-264613 (2-oxo-4-phenylpyrrolidine-3-carboxylic acid [1-(3-bromo-phenyl)-(E/Z)-ethylidene]-hydrazide; and
(43) a sulfated cyclodextrin.

Suitable penetration enhancers are as described above.

Yet another aspect of the invention is a pharmaceutical composition formulated for treatment or prevention of a disease or condition associated with inflammation comprising:
(1) a therapeutically effective quantity of a pentosan polysulfate salt;
(2) a quantity of a penetration enhancer sufficient to improve the bioavailability of the pentosan polysulfate salt; and
(3) optionally, a pharmaceutically acceptable carrier.

The disease or condition associated with inflammation is as described above. Suitable penetration enhancers are as described above. In one alternative, the pharmaceutical composition further comprises at least one additional agent that is effective in treating inflammation. Suitable additional agents are as described above.

### DETAILED DESCRIPTION OF THE INVENTION

One aspect of the present invention is a pharmaceutical composition comprising:
(1) a therapeutically effective quantity of a pentosan polysulfate salt;
(2) a quantity of a penetration enhancer sufficient to improve the bioavailability of the pentosan polysulfate salt; and
(3) optionally, at least one filler, excipient, or carrier.

Pentosan polysulfate (PPS) is a semi-synthetic, polysulfated oligosaccharide comprising a mixture of multiply charged anionic polysaccharides. PPS is produced by chemical sulfation of polysaccharides such as xylan obtained from woody plants such as beechwood trees. The resulting product typically contains approximately 15-17% sulfur in the form of approximately 1.5-1.9 covalently bound sulfate groups per sugar residue in a mixture of polydisperse polymeric molecules estimated to have a molecular weight of from about 4,000 to about 10,000 daltons. PPS consists of sulfated, linear polysaccharides of about 12 to 30 1-4 conjugated P3-D-xylopyranose units (Mᵣ=˜4000-˜10000) which has a D-gluconic acid at approximately every tenth unit. PPS can also be obtained from microorganisms.

United States Patent Application Publication No. 2011/0212914 by Ellinghuysen et al., incorporated herein by this reference, discloses stabilized pentosan polysulfate formulations, including formulations that are stable without refrigeration in a solution with a pH from about 4 to about 8, or with a pH of from about 7 to about 8. In another alternative, the formulation is stable without refrigeration after terminal sterilization. The formulation may comprise pentosan polysulfate in a concentration of from about 25 mg/mL to about 500 mg/mL, preferably from about 100 mg/mL to about 250 mg/mL. The formulation can undergo terminal sterilization by moist heat with or without rapid cooling fluids, ethylene oxide, or radiation. Formulations can comprise additional components such as one or more buffers, such as sodium bisulfite, sodium citrate, or citric acid; one or more chelating agents, such as EDTA; one or more preservatives; one or more antimicrobial agents, such as methylparaben; one or more antioxidants; or other suitable excipients. Pentosan polysulfate preparations can also include an amino sugar and hyaluronic acid.

Because pentosan polysulfate is a polyanion, a counterion is required. As described below, typically, when the pentosan polysulfate is used to treat a disease or condition of the lower urinary tract, such as interstitial cystitis, the counterion is sodium, leading to sodium pentosan polysulfate. However, in other therapeutic applications, particularly therapeutic applications not directed to diseases and conditions affecting the lower urinary tract, other counterions, such as potassium or calcium, can be used alternatively, leading to potassium pentosan polysulfate or calcium pentosan polysulfate. Accordingly, the pentosan polysulfate salt is typically selected from the group consisting of sodium pentosan polysulfate, potassium pentosan polysulfate, and calcium pentosan polysulfate.

In describing the dosages of sodium pentosan polysulfate (or, alternatively, other salts such as potassium pentosan polysulfate or calcium pentosan polysulfate as described below), it is important to distinguish the administered dose from the therapeutically effective quantity actually absorbed. When the reference is to the quantity of sodium pentosan polysulfate (or other salts) in a unit dose of the composition, it refers to the administered dose and not to the therapeutically effective quantity actually absorbed by the subject to whom the dose is administered; the therapeutically effective quantity actually absorbed is specifically referenced.

Typically, the quantity of sodium pentosan polysulfate originally present in the pharmaceutical composition is from about 50 mg to about 300 mg per unit dose in the composition. Preferably, the quantity of sodium pentosan polysulfate originally present in the pharmaceutical composition is from about 100 mg to about 200 mg per unit dose in the composition.

Typically, without the use of a penetration enhancer according to the present invention, the therapeutically effective quantity of sodium pentosan polysulfate actually absorbed is from about 2.5 mg to about 5.0 mg (by giving 200 mg of two capsules of 100 mg which is the only strength available) mg per unit dose of the composition. Preferably, the therapeutically effective quantity of sodium pentosan polysulfate actually absorbed, with the use of a penetration enhancer according to the present invention, is from about 2.5 mg to about 20 mg per unit dose of the composition. More preferably, the therapeutically effective quantity of sodium pentosan polysulfate actually absorbed, with the use of a penetration enhancer according to the present invention, is from about 10 mg to about 15 mg per unit dose of the composition.

Typically, the quantity of penetration enhancer is from about 50 mg to about 800 mg per unit dose of the composition. Preferably, the quantity of penetration enhancer is from about 100 mg to about 500 mg per unit dose of the composition. More preferably, the quantity of penetration enhancer is from about 150 mg to about 400 mg per unit dose of the composition.

Typically, the ratio, by weight, of the penetration enhancer to the sodium pentosan polysulfate is from about 0.167:1 to about 8:1. Preferably, the ratio, by weight, of the penetration enhancer to the sodium pentosan polysulfate is from about 0.50:1 to about 3:1. More preferably, the ratio, by weight, of the penetration enhancer to the sodium pentosan polysulfate is from about 0.75:1 to about 2:1.

Typically, the quantity of penetration enhancer used is sufficient to increase the bioavailability of sodium pentosan polysulfate to at least 5%. Preferably, the quantity of penetration enhancer used is sufficient to increase the bioavailability of sodium pentosan polysulfate to at least 10%. More preferably, the quantity of penetration enhancer used is sufficient to increase the bioavailability of sodium pentosan polysulfate to at least 20%. Still more preferably, the quantity of penetration enhancer used is sufficient to increase the bioavailability of sodium pentosan polysulfate to at least 30%.

Suitable penetration enhancers and, where employed, pharmaceutically acceptable carriers, are described below.

Penetration enhancers suitable for use in methods according to the present invention can include, but are not limited to the enhancers described below as (1)-(10). Additional penetration enhancers are described below and can be used in methods according to the present invention.

One group of penetration enhancers is: (1) N-benzoyl-α-amino acids of Formula (II) and salts, analogues, or bioisosteres thereof:

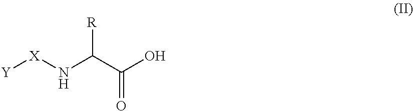

wherein the α-amino acid is selected from the group consisting of glycine, alanine, valine, leucine, phenylalanine, tyrosine, aspartic acid, glutamic acid, lysine, ornithine, arginine, and serine, wherein X is selected from the group consisting of C(O) and SO₂, and wherein Y is selected from the group consisting of phenyl and cyclohexyl.

Another group of penetration enhancers is: (2) derivatized leucines of Formula (III) and salts, analogues, or bioisosteres thereof:

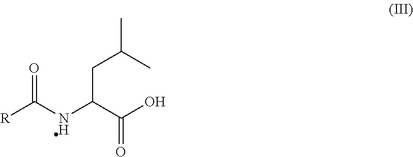

wherein R is selected from the group consisting of cyclohexyl, 2-methylcyclohexyl, 3-methylcyclohexyl, 4-methylcyclohexyl, cycloheptyl, cyclopentyl, cyclopropyl, 2-carboxycyclohexyl, benzoyl, 3-methoxyphenyl, 2-nitrophenyl, 3-nitrophenyl, 4-nitrophenyl, and (CH₂)₂cyclohexyl.

Yet another group of penetration enhancers is: (3) N-cyclohexanoylamino acids of Formula (IV) and salts, analogues, or bioisosteres thereof:

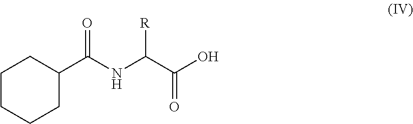

wherein R is selected from the group consisting of CH₂Ph, (CH₂)₃NHC(NH)NH₂, i-butyl, s-butyl, (CH₂)₄NH, CH₂(4-C₆H₄OH), (CH₂)₃NHC(O)NH₂, CH₂(imidazole), and phenyl.

Yet another group of penetration enhancers is: (4) derivatized phenylglycines of Formula (V) and salts, analogues, or bioisosteres thereof:

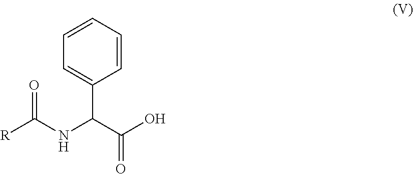

wherein R is selected from the group consisting of cyclohexyl, cyclopentyl, cycloheptyl, methylcyclohexyl, (CH₂)₂cyclohexyl, phenyl, and 2-hydroxyphenyl.

Still another group of penetration enhancers is (5): derivatives of 4-aminobenzoic acid, 2-(4-aminophenyl)acetic acid, 3-(4-aminophenyl)propionic acid, or 4-(4-aminophenyl)butyric acid of Formula (VI) and salts, analogues, or bioisosteres thereof:

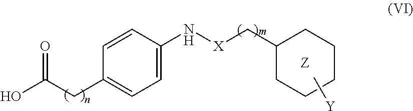

wherein: (a) Y is selected from the group consisting of H, F, 2-OH, 2,3-Ph, 4-Ph, 3,4-Ph, 4-OCH₃, 4-F, 2-Cl, 2-F, 2,4-(OH)₂, 3-CF₃, 3-Cl, 2-CH₃, 2,6-(OH)₂, 3-N(CH₃), 3,4-OCH₂O, 2,6-diCH₃, 2-COOH, 2-NO₂, 2-OCH₃, 3-NO₂, 2-OCF₃, 4-CH₃, and 4-i-Bu; (b) n is 0, 1, 2, 3, 4, or a vinyl group; (c) m is 0, 1, or 2, a vinyl group, a CHMe group, a CHEt group; a (CH₂)₂O group, a (CH₂)₂C═O group, or a (CH₂OH)₂ group; (d) X is C═O, SO₂, or CH₂; and (e) Z is phenyl, cyclohexyl, or cycloheptyl.

Yet another group of penetration enhancers is: (6) compounds of Formula (VII):

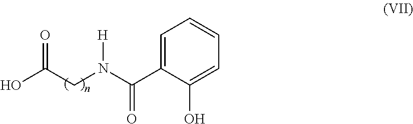

wherein n is 1, 2, 3, 4, 5, 6, 7, 8, 9, 10, or 11, and salts, analogues, or bioisosteres thereof. Preferred enhancers are compounds or salts of Formula (VII) that have n as 7, 8, or 9.

A particularly preferred penetration enhancer is sodium N-[8-(2-hydroxybenzoyl)amino]caprylate, also known as salcaprozate sodium or SNAC (S. A. Mousa et al., “Pharmacokinetics and Pharmacodynamics of Oral Heparin Solid Dosage Form in Healthy Human Subjects,” J. Clin. Pharmacol. 47: 1508-1520 (2007), incorporated herein by this reference). This is the sodium salt of a compound of Formula (VI) with n equal to 7.

Other penetration enhancers are known in the art. U.S. Pat. No. 8,410,309 to Leone-Bay et al., incorporated by this reference, describes phenoxycarboxylic acid compounds as penetration enhancers, specifically phenoxycarboxylic acid compounds of Formula (VIII):

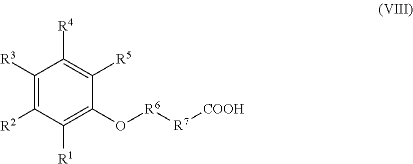

wherein: (i) R¹, R², R³, and R⁴ are each independently hydrogen, hydroxyl, halo, C₁-C₄ alkyl, C₂-C₄ alkenyl, C₁-C₄ alkoxy, —C(O)R⁸, —NO₂, —NR⁹R¹⁰, or —N⁺R⁹R¹⁰R¹¹(R¹²)⁻; (ii) R⁵ is hydrogen, hydroxyl, —NO₂, halo, trifluoromethyl, —NR¹⁴R¹⁵, —N⁺R¹⁴R¹⁵R¹⁶(R¹³)⁻, amide, C₁-C₁₂ alkoxy, C₁-C₁₂ alkyl, C₂-C₁₂ alkenyl, carbamate, carbonate, urea, or —C(O)R¹⁸; (iii) R⁵ is optionally substituted with halo, hydroxyl, sulfhydryl, or carboxyl; (iv) R⁵ is optionally interrupted by O, N, S, or —C(O)—; (v) R⁶ is a C₁-C₁₂ alkylene, C₂-C₁₂ alkenylene, or arylene; (vi) C⁶ is optionally substituted with a C₁-C₄ alkyl, C₂-C₄ alkenyl, C₁-C₄ alkoxy, hydroxyl, sulfhydryl, halo, amino, or —CO₂R⁸; (vii) R⁶ is optionally interrupted by O or N; (viii) R⁷ is a bond or arylene; (ix) R⁷ is optionally substituted with hydroxyl, halogen, —C(O)CH₃, —NR¹⁰R¹¹, or —N⁺R¹⁰R¹¹R¹²(R¹³)⁻; (X) R⁸ is hydrogen, C₁-C₄ alkyl, C₂-C₄ alkenyl, or amino; (xi) R⁹, R¹⁰, R¹¹, and R¹² are each independently hydrogen or C₁-C₁₀ alkyl; (xii) R¹³ is a halide, hydroxide, sulfate, tetrafluoroborate, or phosphate; (xiv) R¹⁴, R¹⁵, and R¹⁶ are each independently hydrogen, C₁-C₁₀ alkyl, C₁-C₁₀ alkyl substituted with carboxyl, C₂-C₁₂ alkenyl, C₂-C₁₂ alkenyl substituted with carboxyl, or C(O)R¹⁷; (xv) R¹⁷ is hydroxyl, C₁-C₁₀ alkyl, or C₂-C₁₂ alkenyl; (xvi) R¹⁸ is hydrogen, C₁-C₆ alkyl, hydroxyl, —NR¹⁴R¹⁵, or —N⁺R¹⁴R¹⁵R¹⁶(R¹³)⁻; with the proviso that: (a) when R¹, R², R³, R⁴, and R⁵ are hydrogen and R⁷ is a bond, then R⁶ is not a C₁-C₆, C₉, or C₁₀ alkyl; (b) when R¹, R², R³, and R⁴ are hydrogen, R⁵ is hydroxyl, and R⁷ is a bond, then R⁶ is not a C₁-C₃ alkyl; (c) when at least one of R¹, R², R³, and R⁴ is not hydrogen, R⁵ is hydroxyl, and R⁷ is a bond, then R⁶ is not a C₁-C₄ alkyl; (d) when R¹, R², and R³ are hydrogen, R⁴ is —OCH₃, R⁵ is C(O)CH₃, and R⁶ is a bond, then R⁷ is not a C₃ alkyl; and (e) when R¹, R², R⁴, and R⁵ are hydrogen, R³ is hydroxyl, and R⁷ is a bond, then R⁶ is not a methyl group.

U.S. Pat. No. 8,383,852 to Tang et al., incorporated herein by this reference, describes compounds having a cyclic moiety as penetration enhancers, specifically compounds of Formula (IX):

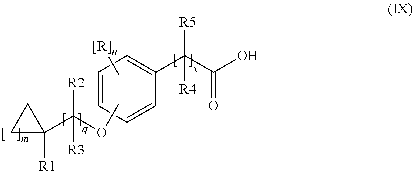

wherein: m is 1, 2, 3, 4, 5, or 6; n is 0, 2, 3, or 4, q and x are independently chosen from 0, 1, 2, 3, 4, 5, 6, 7, 8, 9, or 10; R may be the same or different and is selected from hydrogen, halogen, a substituted or non-substituted alkyl, substituted or non-substituted alkyloxyl, substituted or non-substituted alkenyloxyl, substituted or non-substituted alkynyloxyl and substituted or non-substituted aryloxyl; and R1, R2, R3, R4 and R5 are independently selected from hydrogen, halogen, substituted or non-substituted alkyl, substituted or non-substituted alkenyl, substituted or non-substituted alkynyl, substituted or non-substituted alkyloxyl, substituted or non-substituted aryloxyl, substituted or non-substituted aryl groups, substituted or non-substituted heteroaryl, substituted or non-substituted cycloalkyl, and substituted or non-substituted heterocycloalkyl groups.

U.S. Pat. No. 8,273,794 to Gomez-Orellana et al., incorporated herein by this reference, discloses penetration enhancers with an aromatic nucleus of Formula (X):

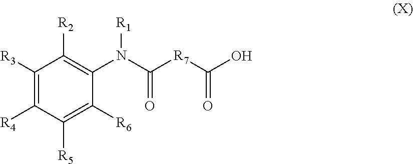

wherein: (i) R₁ is —(CH₂)ₘ—R₈, wherein m is 0 or 1; (ii) R₂, R₃, R₄, R₅, and R₆ are each independently selected from hydrogen, hydroxyl, halo, C₁-C₄ alkyl, C₂-C₄ alkenyl, C₂-C₄ alkynyl, C₁-C₄ alkoxy, and cyano; (iii) R₇ is selected from C₁-C₁₀ alkyl, C₂-C₁₀ alkenyl, and C₂-C₁₀ alkynyl; (iv) R₈ is selected from cyclopentyl, cyclohexyl, and phenyl, wherein, when R₈ is phenyl, m is 1; and (v) R₈ is optionally substituted with C₁-C₄ alkyl, C₁-C₄ alkoxy, halo, hydroxyl, or a combination thereof.

U.S. Pat. No. 8,207,227 to Bay et al., incorporated herein by this reference, discloses disodium salts, monohydrates, and ethanol solvates as penetration enhancers. Specifically, the penetration enhancers are: (1) disodium salts of Formula (XI); (2) monohydrates of disodium salts of Formula (XI); and (3) alcohol solvates of disodium salts of Formula (XI), wherein the alcohol is methanol, ethanol, propanol, propylene glycol, or other monohydroxylic or dihydroxylic alcohols:

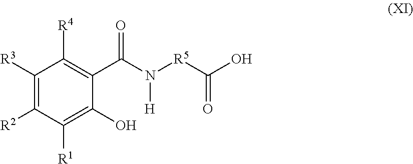

wherein: (i) R¹, R², R³, and R⁴ are each independently hydrogen, hydroxyl, —NR⁶R⁷, halo, C₁-C₄ alkyl, or C₁-C₄ alkoxy; (ii) R⁵ is a substituted or unsubstituted C₂-C₁₆ alkylene, substituted or unsubstituted C₁-C₁₂ alkyl(arylene), or substituted or unsubstituted aryl(C₁-C₁₂ alkylene); and (iii) R⁶ and R⁷ are each independently hydrogen, oxygen, or C₁-C₄ alkyl. Preferred compounds of Formula (XI) include N-(5-chlorosalicyloyl)-8-aminocaprylic acid (5-CNAC), N-(10-[2-hydroxybenzoyl]amino)decanoic acid (SNAD), N-(8-[2-hydroxybenzoyl]amino)caprylic acid (SNAC), 8-(N-2-hydroxy-4-methoxybenzoyl)aminocaprylic, and N-(9-(2-hydroxybenzoyl)aminononanoic acid.

U.S. Pat. No. 8,110,547 by Lee et al., incorporated herein by this reference, discloses several penetration enhancers, including, but not limited to, 8-(N-2-hydroxy-4-methoxybenzoyl)-aminocaprylic acid (“4-MOAC”), N-(8-[2-hydroxybenzoyl]-amino) caprylic acid (“NAC”), N-(8-[2-hydroxybenzoyl]-amino)decanoic acid (“NAD”), N-(8-[2-hydroxy-5-chlorobenzoyl]-amino)octanoic acid (“5-CNAC”), and 4-[(2-hydroxy-4-chlorobenzoyl)amino]butanoate (“4-CNAB”).

U.S. Pat. No. 8,026,392 to Dhoot et al., incorporated herein by this reference, discloses the disodium salt of N-(5-chlorosalicyloyl)-8-aminocaprylic acid as a penetration enhancer.

U.S. Pat. No. 7,977,506 to Boyd et al., incorporated herein by this reference, discloses penetration enhancers of Formula (XII):

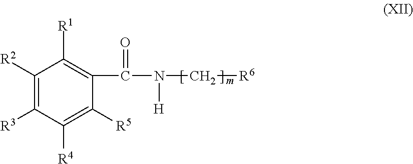

wherein: (i) R¹, R², R³, R⁴, and R⁵ are each independently selected from hydrogen, halo, hydroxyl, —OCH₃, C₁-C₄ alkyl, amino, methylamino, dimethylamino, or nitro; (ii) m is 0, 1, 2, 3, or 4; (iii) R⁶ is phenyl substituted with —O—R⁷—COOH at the ortho, meta, or para position; (iv) R⁶ is optionally substituted with one or more substituents selected from hydrogen, halo, hydroxyl, —OCH₃, C₁-C₄ alkyl, amino, methylamino, dimethylamino, or nitro; and (iv) R⁷ is C₁-C₁₂ alkyl.

U.S. Pat. No. 7,947,841 to Jungheim et al., incorporated herein by this reference, discloses penetration enhancers of Formula (XIII):

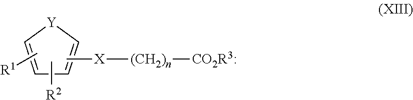

wherein: (i) R¹ and R² are each independently hydrogen, hydroxyl, cyano, C₁-C₆ alkyl, C₁-C₆ alkoxy, CF₃, halo, or NR⁴R^{4′}; (ii) R³ is H or C₁-C₆ alkyl; (iii) X is a 5-membered aromatic heterocycle that is optionally substituted with C₁-C₄ alkyl; wherein the heterocycle contains at least two or three heteroatoms selected from N, S, and O wherein at least one heteroatom is N; (iv) Y is S, CR⁵═N or N═CR⁵; (v) n is 2, 3, 4, 5, 6, or 7; (vi) R⁴ is H, COR⁶, SO₂R⁷, or C₁-C₆ alkyl; (vii) R^{4′} is H or C₁-C₆ alkyl; (viii) R⁵ is H or forms a bond with X; (ix) R⁶ is H or C₁-C₆ alkyl; and (x) R⁷ is H or C₁-C₆ alkyl.

U.S. Pat. No. 7,939,494 to Khan et al., incorporated herein by this reference, discloses a penetration enhancer of Formula (XIV):

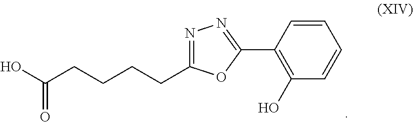 [SMILES: O=C(O)CCCCc1nnc(-c2ccccc2O)o1]

U.S. Pat. No. 7,893,297 to Bhandarkar et al., incorporated herein by this reference, discloses a penetration enhancer that is sodium 4-[(4-chloro-2-hydroxybenzoyl)amino]butanoate.

U.S. Pat. No. 7,744,910 to Gschneidner et al., incorporated herein by this reference, discloses penetration enhancers, including a penetration enhancer of Formula (XV):

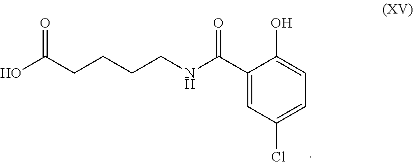 [SMILES: O=C(O)CCCCNC(=O)c1cc(Cl)ccc1O]

U.S. Pat. No. 7,727,558 to Milstein et al., incorporated herein by this reference, discloses polymeric penetration enhancers of Formula (XVa):

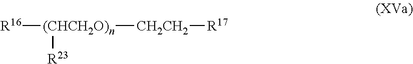

wherein: (i) R¹⁶ is R³-R⁴; (ii) R³
is —NHC(O)NH—, —C(O)NH—, —NHC(O)—, —OOC—, —COO, —NHC(O)O—, —OC(O)NH—, —CH₂NH—, —NHCH₂—, —CH₂NHC(O)O—, —OC(O)NHCH₂—, —CH₂NHCOCH₂O—, —OCH₂C(O)NHCH2-, —NHC(O)CH₂O—, —OCH₂C(O)NH—, —NH—, —O—, or a carbon-carbon bond; R⁴ is Formula (XVIa(1)):

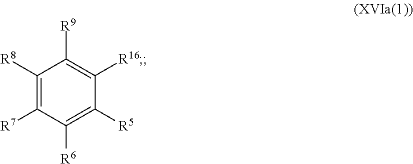

R⁵, R⁶, R⁷, R⁸, and R⁹ are each independently a bond to R³, or hydrogen, chloro, bromo, fluoro, hydroxyl, methyl, methoxy, or —(CH₂)ₘCH₃; R10 is a bond to R³, carboxyl, or —C(O)NHR¹¹R¹²; R¹¹ is a substituted or unsubstituted, linear or branched alkylene having a chain length of 1 to 11 carbon atoms or —R¹³R¹⁴—; R¹² is a bond to R³, carboxyl, amino, hydroxyl, —C(O)—R¹⁵, —COO—R¹⁵, —NHR¹⁵, —OR¹⁵, chloro, or bromo; R¹³ is a substituted or unsubstituted phenylene; R¹⁴ is a substituted or unsubstituted, linear or branched alkylene having a chain length of 1 to 5 carbon atoms; R¹⁵ is a bond to R³; m is 1, 2, 3, or 4; R¹⁷ is hydroxyl or methoxy; R²³ is hydrogen or methyl; and n is an integer from 3 to 200.

U.S. Pat. No. 7,662,771 to Herr et al., incorporated herein by this reference, discloses penetration enhancers of Formula (XVI):

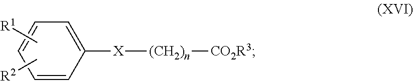

wherein: (i) R¹ and R² are each independently hydrogen, hydroxyl, cyano, C₁-C₆ alkyl, C₁-C₆ alkoxy, CF₃, halo, or NR⁴R^{4′}; (ii) R³ is H or C₁-C₆ alkyl; (iii) R⁴ is H, COR⁵, SO₂R⁶, or C₁-C₆ alkyl; (iv) R^{4′} is H or C₁-C₆ alkyl; (v) R⁵ is H or C₁-C₆ alkyl; (vi) R⁶ is H or C₁-C₆ alkyl; (vii) X is a 5-membered aromatic heterocycle that is optionally substituted with C₁-C₄ alkyl, wherein the heterocycle contains at least two or three heteroatoms selected from N, S, and O, wherein at least one heteroatom is N, and wherein the heterocycle is not 1,3,4-oxadiazole; and (ix) n is 2, 3, 4, 5, 6, or 7.

U.S. Pat. No. 7,553,872 to Sarubbi et al., incorporated herein by this reference, discloses a penetration enhancer of Formula (XVII):

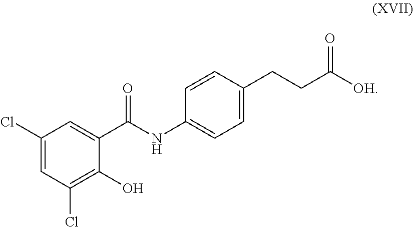 [SMILES: O=C(O)CCc1ccc(NC(=O)c2cc(Cl)cc(Cl)c2O)cc1]

U.S. Pat. No. 7,495,030 to Gschneidner, incorporated herein by this reference, discloses (5-(2-hydroxy-4-chlorobenzoyl) aminovaleric acid as a penetration enhancer.

U.S. Pat. No. 7,390,834 to Moye-Sherman et al., incorporated herein by this reference, discloses penetration enhancers that are cyanophenoxy carboxylic acid compounds of Formula (XVIII):

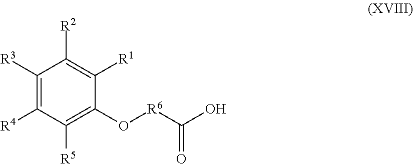

wherein: (i) R¹, R², R³, R⁴, and R⁵ are each independently hydrogen, cyano, hydroxyl, —OCH₃ or halogen, where at least one of R¹, R², R³, R⁴, and R⁵ is cyano; (ii) R⁶ is C₁-C₁₂ linear or branched alkylene, alkenylene, arylene, alkyl(arylene), or aryl(alkylene); with the proviso that where R¹ is cyano, R⁴ is hydrogen or cyano, and R², R³, and R⁵ is not methylene.

U.S. Pat. No. 7,351,741 to Weidner et al., incorporated herein by this reference, discloses a penetration enhancer of Formula (XIX):

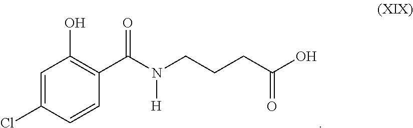 [SMILES: O=C(O)CCCNC(=O)c1ccc(Cl)cc1O]

U.S. Pat. No. 7,297,794 to Gschneidner et al., incorporated herein by this reference, discloses phenoxyamine compounds as penetration enhancers, including 4-(8-(2-hydroxyphenoxy)octyl)morpholine, 8-(2-hydroxyphenoxy)octyldiethanolamine, 7-(4-2-hydroxyphenoxy)heptylmorpholine, 4-(6-(4-hydroxyphenoxy)hexyl)morpholine, 4-(6-(2-hydroxyphenoxy)hexyl)morpholine, 8-(4-hydroxyphenoxy)octanamine, 6-(2-acetylphenoxy)-1-dimethylaminohexane, 7-(2-hydroxyphenoxy)heptyl-2-isopropylimidazole, 6-(2-hydroxyphenoxy)hexyl-2-methylimidazole, and 5-chloro-4-methyl-2-(8-morpholin-4-yloctyloxy)acetophenone.

U.S. Pat. No. 7,279,597 to Leone-Bay et al., incorporated herein by this reference, discloses penetration enhancers of Formula (XX):

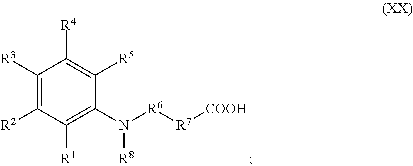

including compounds with the following combinations of substituents: (1) R¹, R², R³, and R⁴ are each hydrogen, R⁵ is carboxyl, R⁶ is (CH₂)₇, R⁷ is a bond, and R⁸ is hydrogen; (2) R¹, R², R³, and R⁴ are each hydrogen, R⁵ is C(O)NH₂, R⁶ is (CH₂)₇, R⁷ is a bond, and R⁸ is hydrogen; (3) R¹, R², R³, and R⁴ are each hydrogen, R⁵ is C(O)CH₃, R⁶ is (CH₂)₇, R⁷ is a bond, and R⁸ is hydrogen; (4) R¹, R², R³, and R⁴ are each hydrogen, R⁵ is C(O)NH₂, R⁶ is (CH₂), R⁷ is p-phenyl, and R⁸ is hydrogen; and (5) R¹, R², R³, and R⁴ are each hydrogen, R⁵ is nitro, R⁶ is (CH₂)₇, R⁷ is a bond, and R⁸ is hydrogen.

U.S. Pat. No. 7,276,534 to Milstein, incorporated herein by this reference, discloses carbon-substituted diketopiperazine penetration enhancers of Formula (XXI):

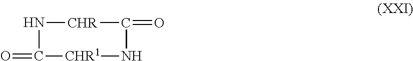

wherein: (i) R and R¹ are C₁-C₂₄ alkyl having a functional group selected from halogen, oxygen, sulfur or nitrogen; (ii) R and R¹ are optionally interrupted with O, N, or S; (iii) R and R¹ are optionally substituted with C₁-C₄ alkyl, C₁-C₄ alkenyl, or CO₂R² or any combination thereof; and (iv) R² is hydrogen, C₁-C₄ alkyl, or C₁-C₄ alkenyl.

U.S. Pat. No. 7,186,414 to Gschneidner et al., incorporated herein by this reference, discloses penetration enhancers, including the compound of Formula (XXII):

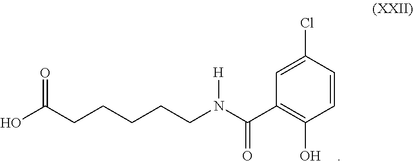 [SMILES: O=C(O)CCCCCNC(=O)c1cc(Cl)ccc1O]

U.S. Pat. No. 7,138,546 to Tang, incorporated herein by this reference, discloses penetration enhancers of Formula (XXIII):

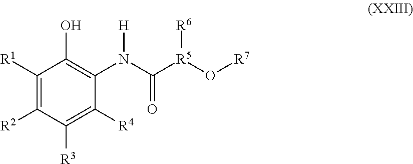

wherein: (i) R¹, R², R³, and R⁴ are each independently hydrogen, hydroxy, halo, C₁-C₄ alkoxy, C₁-C₄ alkyl, C₂-C₄ alkenyl, C₂-C₄ alkynyl, and aryl; (ii) R¹, R², R³, and R⁴ are optionally substituted with halo, hydroxyl, C₁-C₄ alkoxy, or C₁-C₄ alkyl; (iii) R⁵ is C₁-C₄ alkyl; (iv) R⁶ is hydrogen or C₁-C₄ alkyl; (v) R⁷ is hydrogen, C₁-C₄ alkyl, or aryl; and R⁷ is optionally substituted with halogen or hydroxyl.

U.S. Pat. No. 7,125,910 to Leone-Bay et al., incorporated herein by this reference, discloses amino-substituted carboxylic acids including one or more aromatic moieties therein as penetration enhancers; the aromatic moieties can include phenyl, pyrazinyl, pyrimidyl, chromonyl, or other aromatic groups.

U.S. Pat. No. 7,084,279 to Gschneidner, incorporated herein by this reference, discloses oxadiazoles as penetration enhancers.

U.S. Pat. No. 7,067,119 to Leone-Bay et al., incorporated herein by this reference, discloses modified amino acid compounds as penetration enhancers. The modified amino acid compounds may be peptides. A preferred penetration enhancer has the structure of Formula (XXIV):

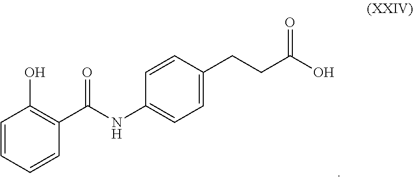 [SMILES: O=C(O)CCc1ccc(NC(=O)c2ccccc2O)cc1]

U.S. Pat. No. 6,991,798 to Gschneidner et al., incorporated herein by this reference, discloses penetration enhancers, including the compound of Formula (XXV):

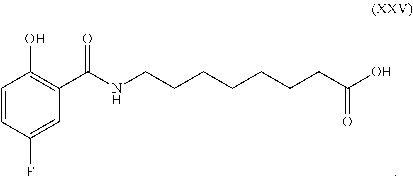 [SMILES: O=C(O)CCCCCCCNC(=O)c1cc(F)ccc1O]

U.S. Pat. No. 6,972,300 to Leone-Bay et al., incorporated herein by this reference, discloses penetration enhancers, including the compound of Formula (XXVI):

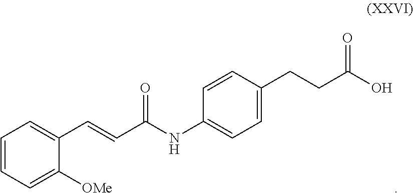 [SMILES: COc1ccccc1/C=C/C(=O)Nc1ccc(CCC(=O)O)cc1]

U.S. Pat. No. 6,960,355 to Leone-Bay et al., incorporated herein by this reference, discloses penetration enhancers of Formula (XXVII)

2-HO—Ar—CONR⁸—R⁷—COOH (XXVII)

wherein: (i) Ar is a phenyl or naphthyl substituted with at least one of C₁-C₅ alkyl, C₂-C₄ alkenyl, fluoro, chloro, hydroxyl, —SO₂, carboxyl, or —SO₃H; (ii) R⁷ is selected from the group consisting of C₄-C₂₀ alkyl, C₄-C₂₀ alkenyl, phenyl, naphthyl, (C₁-C₁₀ alkyl)phenyl, (C₁-C₁₀ alkenyl)phenyl, C₁-C₁₀ alkyl)naphthyl, (C₁-C₁₀ alkenyl)naphthyl, phenyl(C₁-C₁₀ alkyl), phenyl(C₁-C₁₀ alkenyl), naphthyl(C₁-C₁₀ alkyl), and phenyl(C₁-C₁₀ alkenyl); (iii) R⁷ is optionally substituted with C₁-C₄ alkyl, C₁-C₅ alkenyl, C₁-C₅ alkoxy, hydroxyl, sulfhydryl, and —CO₂R⁹ or any combination thereof; (iv) R⁷ is optionally interrupted by oxygen, nitrogen, sulfur, or any combination thereof; (v) R⁸ is selected from the group consisting of hydrogen, C₁-C₄ alkyl, C₁-C₄ alkenyl, hydroxyl, and C₁-C₄ alkoxy; (vi) R⁸ is optionally substituted with C₁-C₄ alkyl, C₁-C₅ alkenyl, C₁-C₅ alkoxy, hydroxyl, sulfhydryl, and —CO₂R⁹ or any combination thereof; and (vii) R⁹ is hydrogen, C₁-C₄ alkyl, or C₁-C₄ alkenyl, with the proviso that the compounds are not substituted with an amino group in the position α to the acid group.

U.S. Pat. No. 6,846,844 to Tang, incorporated herein by this reference, discloses penetration enhancers of Formula (XXVIII):

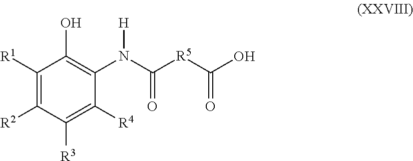

wherein: (i) R¹, R², R³, and R⁴ are independently hydrogen, hydroxyl, halo, C₁-C₄ alkoxy, C₁-C₄ alkyl, C₂-C₄ alkenyl, C₂-C₄ alkynyl, or aryl; (ii) R¹, R², R³, and R⁴ are optionally substituted with halo, hydroxyl, C₁-C₄ alkoxy, or C₁-C₄ alkyl; and (iii) R⁵ is a C₂-C₁₆ branched alkylene, optionally substituted with halogen.

U.S. Pat. No. 6,699,467 to Leone-Bay et al., incorporated herein by this reference, discloses a penetration enhancer of Formula (XXIX):

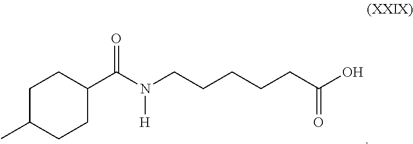 [SMILES: CC1CCC(C(=O)NCCCCCC(=O)O)CC1]

U.S. Pat. No. 6,693,208 to Gschneidner et al., incorporated herein by this reference, discloses penetration enhancers of Formulas (XXX), (XXXI), (XXXII), (XXXIII), (XXXIV), (XXXV), (XXXVI), (XXXVII), (XXXVIII), (XXXIX), (XL), and (XLI):

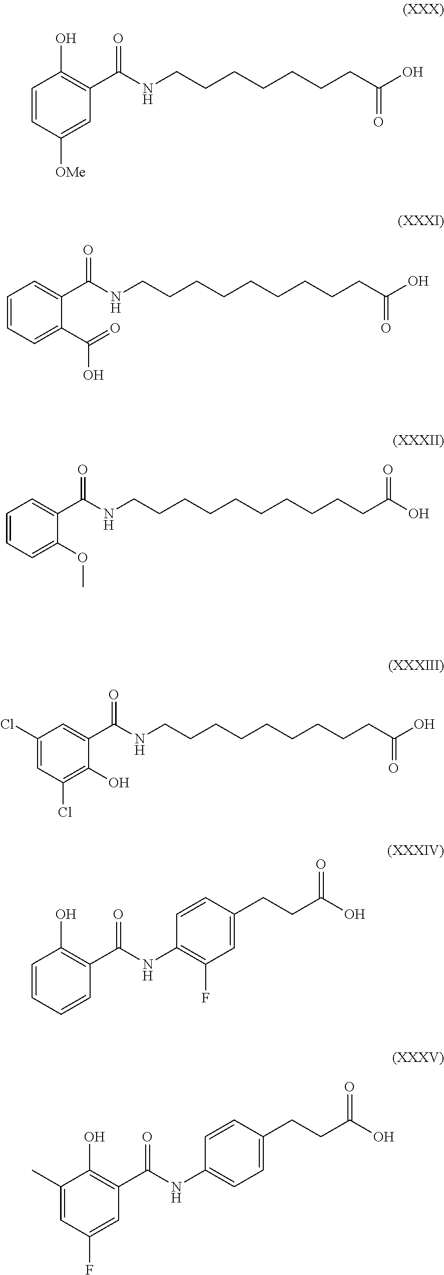 [SMILES: COc1ccc(O)c(C(=O)NCCCCCCCC(=O)O)c1 ; O=C(O)CCCCCCCCCNC(=O)c1ccccc1C(=O)O ; COc1ccccc1C(=O)NCCCCCCCCCCC(=O)O ; O=C(O)CCCCCCCCCNC(=O)c1cc(Cl)cc(Cl)c1O ; O=C(O)CCc1ccc(NC(=O)c2ccccc2O)c(F)c1 ; Cc1cc(F)cc(C(=O)Nc2ccc(CCC(=O)O)cc2)c1O]

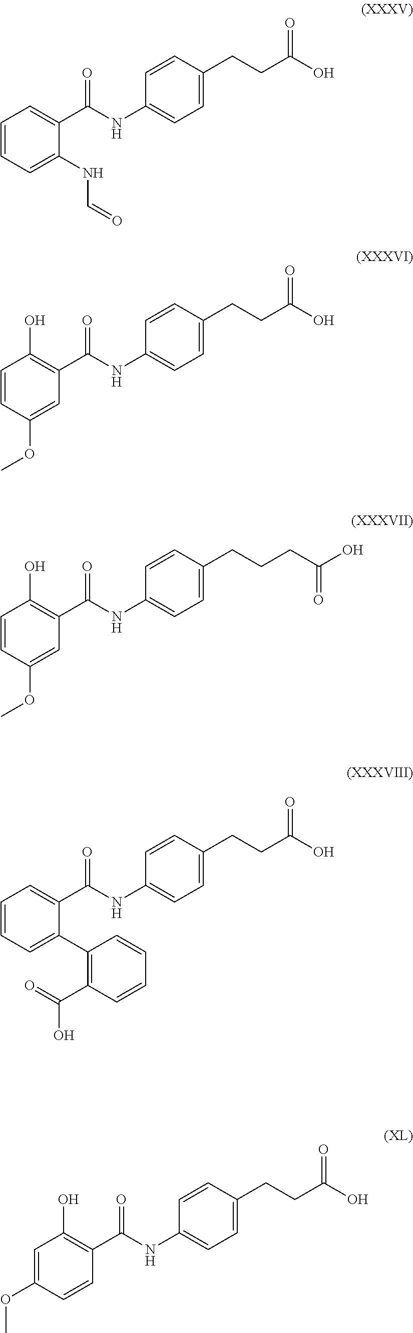 [SMILES: O=CNc1ccccc1C(=O)Nc1ccc(CCC(=O)O)cc1 ; COc1ccc(O)c(C(=O)Nc2ccc(CCC(=O)O)cc2)c1 ; COc1ccc(O)c(C(=O)Nc2ccc(CCCC(=O)O)cc2)c1 ; O=C(O)CCc1ccc(NC(=O)c2ccccc2-c2ccccc2C(=O)O)cc1 ; COc1ccc(C(=O)Nc2ccc(CCC(=O)O)cc2)c(O)c1]

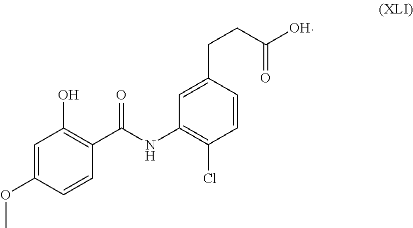 [SMILES: COc1ccc(C(=O)Nc2cc(CCC(=O)O)ccc2Cl)c(O)c1]

U.S. Pat. No. 6,663,887 to Leone-Bay et al., incorporated herein by this reference, discloses a penetration enhancer of Formula (XLII):

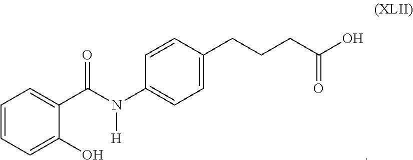 [SMILES: O=C(O)CCCc1ccc(NC(=O)c2ccccc2O)cc1]

U.S. Pat. No. 6,646,162 to Tang et al., incorporated herein by this reference, discloses penetration enhancers of Formula (XLIII):

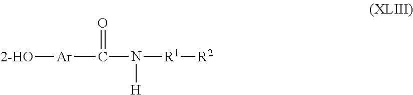

wherein: (i) Ar is phenyl or naphthyl; (ii) Ar is optionally substituted with C₁-C₄ alkyl, C₁-C₄ alkoxy, C₂-C₄ alkenyl, C₂-C₄ alkynyl, aryl, aryloxy, a heterocyclic ring, a C₅-C₇ carbocyclic ring, halo, hydroxyl, sulfhydryl, CO₂R⁶, NR⁷R⁸, or N⁺R⁷R⁸R⁹Y; (iii) (a) R¹ is C₁-C₁₆ alkylene, C₂-C₁₆ alkenylene, C₂-C₁₆ alkynylene, C₆-C₁₆ arylene, (C₁-C₁₆ alkyl)arylene, or aryl(C₁-C₁₆ alkylene); R² is —NR³R⁴, —N⁺R³R⁴, or —N⁺R³R⁴R⁵Y; R³ and R⁴ are each independently hydrogen, oxygen, hydroxyl, substituted or unsubstituted C₁-C₁₆ alkyl, substituted or unsubstituted C₂-C₁₆ alkenyl, substituted or unsubstituted C₂-C₁₆ alkynyl, substituted or unsubstituted aryl, substituted or unsubstituted alkylcarbonyl, substituted or unsubstituted arylcarbonyl, substituted or unsubstituted alkylsulfinyl, substituted or unsubstituted arylsulfinyl, substituted or unsubstituted alkylsulfonyl, substituted or unsubstituted arylsulfonyl, substituted or unsubstituted alkoxycarbonyl, or substituted or unsubstituted aryloxycarbonyl; R⁵ is hydrogen, substituted or unsubstituted C₁-C₁₆ alkyl, substituted or unsubstituted C₂-C₁₆ alkenyl, substituted or unsubstituted C₂-C₁₆ alkynyl, substituted or unsubstituted aryl, substituted or unsubstituted alkylcarbonyl, substituted or unsubstituted arylcarbonyl, substituted or unsubstituted alkylsulfinyl, substituted or unsubstituted arylsulfinyl, substituted or unsubstituted alkylsulfonyl, substituted or unsubstituted arylsulfonyl, substituted or unsubstituted alkoxycarbonyl, or substituted or unsubstituted aryloxycarbonyl; (b) R¹, R², and R⁵ are as above under (a), and R³ and R⁴ are combined to form a 5-, 6-, or 7-membered heterocyclic ring or a aryloxycarbonyl; (b) R¹, R², and R⁵ are as above under (a), and R³ and R⁴ are combined to form a 5-, 6-, or 7-membered heterocyclic ring or a 5-, 6-, or 7-membered heterocyclic ring substituted with C₁-C₆ alkyl, C₁-C₆ alkoxy, aryl, aryloxy, oxo, or carbocyclic ring; or (c) R² and R⁵ are as defined above under (a), and R¹ and R³ are combined to form a 5-, 6-, or 7-membered heterocyclic ring or a 5-, 6-, or 7-membered heterocyclic ring substituted with C₁-C₆ alkyl, C₁-C₆ alkoxy, aryl, aryloxy, oxo, or carbocyclic ring; (iv) R⁴ is hydrogen, oxygen, hydroxyl, substituted or unsubstituted C₁-C₁₆ alkyl, substituted or unsubstituted C₂-C₁₆ alkenyl, substituted or unsubstituted C₂-C₁₆ alkynyl, substituted or unsubstituted aryl, substituted or unsubstituted alkylcarbonyl, substituted or unsubstituted arylcarbonyl, substituted or unsubstituted alkylsulfinyl, substituted or unsubstituted arylsulfinyl, substituted or unsubstituted alkylsulfonyl, substituted or unsubstituted arylsulfonyl, substituted or unsubstituted alkoxycarbonyl, or substituted or unsubstituted aryloxycarbonyl; (v) R⁶ is hydrogen, C₁-C₄ alkyl, C₁-C₄ alkyl substituted with halogen or with hydroxyl, C₂-C₄ alkenyl, or C₂-C₄ alkenyl substituted with halogen or with hydroxyl; (vi) R⁷, R⁸, and R⁹ are each independently hydrogen, oxygen, C₁-C₄ alkyl, C₁-C₄ alkyl substituted with halogen or with hydroxyl, C₂-C₄ alkenyl, or C₂-C₄ alkenyl substituted with halogen or with hydroxyl; and (vii) Y is halogen, hydroxide, sulfate, nitrate, phosphate, alkoxy, perchlorate, tetrafluoroborate, or carboxylate.

U.S. Pat. No. 6,642,411 to Leone-Bay et al., incorporated herein by this reference, discloses a penetration enhancer of Formula (XLIV):

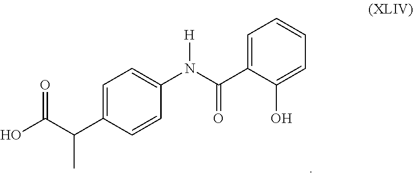 [SMILES: CC(C(=O)O)c1ccc(NC(=O)c2ccccc2O)cc1]

U.S. Pat. No. 6,627,228 to Milstein et al., incorporated herein by this reference, discloses polymeric delivery agents. The polymeric delivery agent comprises a polymer conjugated to modified amino acid or derivative thereof via a linkage group selected from the group consisting of —NHC(O)NH—, —C(O)NH—, —NHC(O)−, —OOC—, —COO—, —NHC(O)O—, —OC(O)NH—, —CH₂NH—, —NHCH₂—, —CH₂NHC(O)O—, —OC(O)NH₂—, CH₂NHCOCH₂O—, —OCH₂C(O)NHCH₂—, —NHC(O)CH₂O—, —OCH₂C(O)NH—, —NH—, —O—, and a carbon-carbon bond, with the proviso that the polymeric delivery agent is not a polypeptide or polyamino acid, wherein the modified amino acids are acylated or sulfonated amino acids, ketones or aldehydes of acylated or sulfonated amino acids, salts thereof, or polyamino acids or polypeptides of any of the foregoing, and the polymer is selected from the group consisting of polyethylene; polyacrylates; polymethacrylates; poly(oxyethylene); poly(propylene); polypropylene glycol; polyethylene glycol (PEG); PEG-maleic anhydride copolymers; and derivatives and combinations thereof.

U.S. Pat. No. 6,623,731 to Leone-Bay et al., incorporated herein by this reference, discloses penetration enhancers, including a penetration enhancer of Formula (XLV):

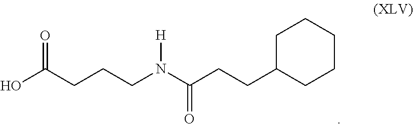 [SMILES: O=C(O)CCCNC(=O)CCC1CCCCC1]

U.S. Pat. No. 6,525,020 to Leone-Bay et al., incorporated herein by this reference, discloses penetration enhancers, including a penetration enhancer of Formula (XLVI):

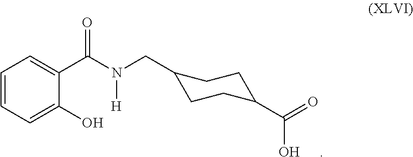 [SMILES: O=C(NCC1CCC(C(=O)O)CC1)c1ccccc1O]

U.S. Pat. No. 6,428,780 to Leone-Bay et al., incorporated herein by this reference, discloses penetration enhancers, including a penetration enhancer of Formula (XLVII):

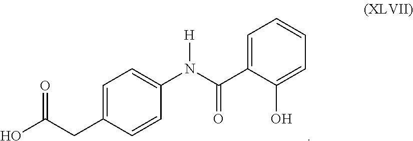 [SMILES: O=C(O)Cc1ccc(NC(=O)c2ccccc2O)cc1]

U.S. Pat. No. 6,358,504 to Leone-Bay et al., incorporated herein by this reference, discloses a number of penetration enhancers, including 6-N-(3,5-dichloro-2-hydroxybenzoyl)aminocaproic acid, 8-(2-aminobenzoylamino)caprylic acid, 8(2-trifluoromethoxy)benzoylaminocaprylic acid, N-(2-hydroxybenzoyl)isonipecotic acid, 4-[4-(2-aminobenzoylamino)phenyl]butyrylhydroxamic acid, 4-(4-(pentafluorobenzoyl)aminophenyl)butyric acid, 4-(4-(3-anisoyl)aminophenyl)butyric acid, 8-(3-anisoyl)aminocaprylic acid, 4-(4-(phenoxyacetyl)aminophenyl)butyric acid, 4-(4-(2-nitrobenzenesulfonyl)aminophenyl)butyric acid, 8-(2-nitrobenzenesulfonyl)aminocaprylic acid, 6-(4-(salicyloyl)aminophenyl)hexanoic acid, 8-(2-methoxybenzoyl)aminocaprylic acid, 2-[4-salicyloylaminophenyl]ethyl methyl sulfone, 1-salicyloy1-2-succinyl-hydrazide, 3-(4-(2,5-dimethoxycinnamoyl)aminophenyl)propionic acid, 4-(4-(2,5-dimethoxycinnamoyl)aminophenyl)butyric acid, 1-salicyloyl-2-glutaryl hydrazide, succinyl-4-aminosalicylic acid, 8-(phenoxyacetylamino)caprylic acid, 8-(2-pyrazinecarbonyl)aminocaprylic acid, 4-(4-(2 pyrazinecarbonyl)aminophenylbutyric acid, 6-(4-(N-2-nitrobenzoyl)aminophenyl)hexanoic acid, 6-(4-(N-2-aminobenzoyl)aminophenyl)hexanoic acid, 4-(4-(2-(3-carbonyl)pyrazinecarboxyl)aminophenyl)butyric acid, 4(2-nitrobenzoyl)aminophenylsuccinic acid, 8-(2-(trifluoromethoxy)benzoyl)aminocaprylic acid, 8-(benzylcarbonylamino)caprylic acid, 8-(phenylcarbonylamino)caprylic acid, 2-[4-(2-methoxybenzoylamino)phenyl]ethyl H₂PO₄, 1-salicyloyl-2-suberyl hydrazide, 4-(4-benzyloxycarbonylaminophenyl)butyric acid, 4-(4-)₂-hydroxyn icotinoyl)aminophenyl)butyric acid, 9-salicyloylaminonanoic acid, 4-(4-phenyloxycarbonylaminophenyl)butyric acid, 3-(2-methoxybenzoylamino)-1-propanol, 8-(2-hydroxynicotinoyl)aminocaprylic acid, 6-(2-methoxybenzoyl)amino nicotinic acid, salicyloylglycine, 4-(1-(2-pyrimidyl)piperazinoyl)butyric acid, 8-(chromone-3-carbonyl)aminocaprylic acid, 8-(vinylbenzoyl)aminocaprylic acid, 4-(4-(chromone-3-carbonyl)aminophenyl)butyric acid, 8-cinnamoylaminocaprylic acid, 5-(N-salicyloylamino)valeric acid, N-(4-salicyloylamino)-6-caproic acid, 4′-flavonic acid, 11-cinnamoylaminoundecanoic acid, 4-octanoylamino-3-hydroxybenzoic acid, (3-phenyl-2,3-dihydroxypropanoyl)-8-aminocaprylic acid, 8-[N-(3-coumarincarbonyl)]aminocaprylic acid, 8-[N-(4-chlorobenzyl)]aminocaprylic acid, 8-[N-(3-fluorobenzyl)]aminocaprylic acid, 8-(N-2,5-dihydroxybenzoyl)aminocaprylic acid, 8-(N-3,5-diacetyloxybenzoyl)aminocaprylic acid, 8-(N-4-hydroxybenzoyl)aminocaprylic acid (dimer), 8-(N-2,4-dihydroxybenzoyl)aminocaprylic acid, 1-(1-(N-2-methoxyanalino)sebacic acid, 10-(N-2-methoxyanilino)sebacic acid, 8-(N-benzoyl)aminocaprylic acid, 2-methoxybenzenaminodecanoic acid, 8-(N-benzoyl)aminocaprylic acid, 8-(N-2-hydroxy-4-methoxybenzoyl)aminocaprylic acid, 8-(N-4-fluorobenzoyl)aminocaprylic acid, 8-(N-3-bromobenzoyl)aminocaprylic acid, 8-(4-(1,2-dihydroxyethyl)benzoyl)aminocaprylic acid, 8-(N-4-bromobenzoyl)aminocaprylic acid, 8-(N-4-iodobenzoyl)aminocaprylic acid, 4-{4-[N-(2-iodobenzoyl)aminophenyl]}butyric acid, 4-{4-[N-(1-hydroxy-2-naphthoyl)aminophenyl]}butyric acid, 4-(4-(2,4-dimethoxybenzoyl)aminophenyl)butyric acid, 4-(o-anisoyl)aminophenylacetic acid, 3-[4-(2,4-dimethoxybenzoyl)aminophenyl]propionic acid, 4-{4-[N-(4-iodobenzoyl)]aminophenyl}butyric acid, 3-[4-(2,3-dimethoxybenzoyl)aminophenyl]propionic acid, 4{4 [N 2 bromobenzoyl)] aminophenyl} butyric acid, 4{4-[N-3[bromobenzoyl) aminophenyl]} butyric acid, 8-(N-3,5-dihydroxybenzoyl)aminocaprylic acid, 8-(N-3,5-dimethoxy 4-hydroxybenzoyl)aminocaprylic acid, 8-(N-2,6-dimethoxybenzoyl)aminocaprylic acid, 4-{4[N-(4 bromobenzoyl)aminophenyl]butyric acid, 8-(2-hydroxy-4-chlorobenzoyl)aminocaprylic acid, 8-(N-2,6-dihydroxybenzoyl)aminocaprylic acid, 8-(N-2-hydroxy-6-methoxybenzoyl)aminocaprylic acid, 8-(5-chloro-o-anisoyl)aminocaprylic acid, 4-(4-(2,3-dimethoxybenzoyl)aminophenyl)butyric acid, 4-(4-(5 chloro-o-anisoyl)aminophenyl)butyric acid, 4-(4-(4-chloro-o-anisoyl)aminophenyl)butyric acid, 8-(4-chloro-o-anisoyl)aminocaprylic acid, 3-(4-(2,5-dimethoxybenzoyl)aminophenyl)propionic acid, 4-{N-[4-(3 iodobenzoyl)aminophenyl]butyric acid, 7-cinnamoylaminoheptanoic acid, 8-N-(3 iodobenzoyl)aminocaprylic acid, 8-N-(4 methoxy-3-nitrobenzoyl)aminocaprylic acid, 8-N-(2 methoxy 4 nitrobenzoyl)aminocaprylic acid, 4-{N-[4-(2-methoxy-4-nitrobenzoyl)aminophenyl]}butyric acid, 4-(4-(2,5-dimethoxybenzoyl)aminophenyl)butyric acid, 8-(N-2-hydroxy-5-bromobenzoyl)aminocaprylic acid, 3-indolebutryic acid, 4-(4-(2,6-dimethoxybenzoyl)aminophenylbutyric acid, 4-[4-N-(4 methoxy-3-nitrobenzoyl)aminophenyl]butyric acid, 8-(N-2-hydroxy-5 chlorobenzoyl)aminocaprylic acid, 8-(N-2-hydroxy-5-iodobenzoyl)aminocaprylic acid, 8-(3-hydroxy-2-naphthoyl)aminocaprylic acid, 8-(N-2-hydroxy-2-nitrobenzoyl)aminocaprylic acid, 8-(N-3-methylsalicyloyl)aminocaprylic acid, 8-(N-5-methylsalicyloyl)aminocaprylic acid, 4-[-N-(2 hydroxy-4-bromobenzoyl)aminophenyl]butyric acid, 8-(N-2,3-dihydroxybenzoyl)aminocaprylic acid, 9-(cinnamoylamino)nonanoic acid, 4-(4-(2-chloro-5-nitrobenzoyl)aminophenyl)butyric acid, 4-[N-(2-hydroxy-5-iodobenzoyl)]aminophenylbutyric acid, N-2-nitrophenyl-N′-(8 octanoic acid) urea, 8-[N-(2-acetoxy-3,5-dibromobenzoyl)aminocaprylic acid, 8-N-(2-chloro-6-fluorobenzoyl)aminocaprylic acid, 8-N-(4-hydroxy-3-nitrobenzoyl)caprylic acid, 4-(4-salicyloylaminophenyl)-4-oxobutyric acid, 12-cinnamoyldodecanoic acid, 4-{4-[N-(3-hydroxy-2-naphthoyl)aminophenyl]}butyric acid, 8-(4-chloro-3-nitrobenzoyl)aminocaprylic acid, 8-(2-chloronicotinoyl)aminocaprylic acid, 8-(2-chloro-5-nitrobenzoyl)aminocaprylic acid, 4-(4-phthalimidophenyl)butyric acid, 4-{4-[N-(3-hydroxy-2-napthoyl)aminophenyl]}propanoic acid, 3-(4-(2,6-dimethoxybenzoyl)aminophenyl)propionic acid, 8-(N-2-hydroxy-3,5-diiodobenzoyl)aminocaprylic acid, 8-(N-2-chloro-4-fluorobenzoyl)aminocaprylic acid, 8 (N 1 hydroxy-2-naphthoyl)aminocaprylic acid, 8-(phthalimido)caprylic acid, 10-(4-chloro-2-hydroxyanilino)sebacic acid monoamide, 6-(anisoyl)aminocaproic acid, 4-(4-(4-chloro-3-nitrobenzoyl)aminophenyl)butyric acid, 11-N-(1-hydroxy-2-naphthoyl)aminoundecanoic acid, bis(N-2-carboxylphenyl-N—(N′-8-octanoic acid)ureal)oxalyl diamide, 2-[2-N-(2-chlorobenzoyl)aminoethoxy]ethanol), 2-[2-N-(4 chlorobenzoyl)aminoethoxy]ethanol, 4-(2-methybenzoyl)amino-3-carboxysulfoxide, 4-(2-methoxybenzoyl)amino 3-carboxypropylsulfone, 4-(4-(3-hydroxyphthalimido)phenyl)butyric acid, 2-[2-N-(2 methoxybenzoyl)aminoethoxyl]ethanol, 2-[2-N-(3 chlorobenzoyl)aminoethoxy]ethanol, bis(N-2-carboxyphenyl)-N—(N′-3(4-aminophenyl)propionic acid)ureal)oxalyl diamide, trans 4 (2 aminobenzamidomethyl)cyclohexamecarboxylic acid, 11-N-(3,5-dichloro-2-hydroxybenzoyl)aminoundecanoic acid, 2-[N-(2-bromobenzoyl)aminoethoxy]ethanol, 7-N-(3,5-dichloro-2-hydroxybenzoyl)aminoheptanoic acid, N-[3,5-dichloro-2-hydroxybenzoyl-4(4-aminophenyl)]butyric acid, trans-4-(N salicyloylaminomethyl)cyclohexane carboxylic acid, N-[3,5-dichloro-2-hydroxybenzoyl-3-(4-aminophenyl)]propionic acid, 12-N-(3,5-dichloro-2-hydroxybenzoyl)aminodecanoic acid, N-(2-hydroxy-4-carboxy)-6-heptenamide, N-(2-bromobenzoyl)morpholine, 8-N-cyclohexanoylaminocaprylic acid, 2-[N-(2-iodobenzoyl)aminoethoxy]ethanol, 5-(4-chloro-2-hydroxyanilinocarbonyl)valeric acid, 8-(2-hydroxyphenoxy)-aminocaprylic acid, N-salicyloyl-5-(3-aminophenyl-valeric acid, 4-(4-(2-ethoxylbenzoyl)aminophenyl)butyric acid, 9-[2-(3-hydroxy)pyridylaminocarbonyl]nonanic acid, 7-(2-hydroxyphenoxyacetyl)aminocaprylic acid, 2-[N-2-hydroxybenzoylamino)ethoxy]ethanol. 4-[N-(3,5-chloro-2-hydroxybenzoyl)]aminophenylacetic acid 8-(2-hydroxy-5-chloroanilinocarbonyl)octanoic acid, N-salicyloyl-5-(4-aminophenyl)valeric acid, 9-(2-hydroxy-5-methylanilinocarbonyl)nonanoic acid, 5-(2-hydroxy-5-methylanilinocarbonyl)valeric acid, 8-(pentafluorobenzoyl)aminocaprylic acid, 3-(3-(salicyloyl)aminophenyl)propionic acid, 8-(2-ethoxybenzoyl)aminocaprylic acid, 4-(4-(2-dimethylamino benzoic)aminophenyl)butyric acid, 8-(3-phenoxylpropionylamino)caprylic acid, 4-(salicyloyl)aminophenylethyltetrazole, 4-(4-(N-(2-fluorocinnamoyl))aminophenyl)butyric acid, 4-(4-(N-8-salicyloyl)aminocaprylic)aminophenyl)butyric acid, 8-(p-anisoyl)aminocaprylic acid, 8-(4-hydroxybenzoyl)aminocaprylic acid, 8-(3-hydroxybenzoyl)aminocaprylic acid, 8-(3,4,5-trimethoxybenzoyl)aminocaprylic acid, 8-(N-4-methylsalicyloyl)aminocaprylic acid, N-10-(2-hydroxy-5-nitroanilino)decanoic acid, and 4-(4-(2-chloronicotinoyl)aminophenyl)butyric acid.

U.S. Pat. No. 6,344,213 to Leone-Bay et al., incorporated herein by this reference, discloses penetration enhancers, including a penetration enhancer of Formula (XLVIII):

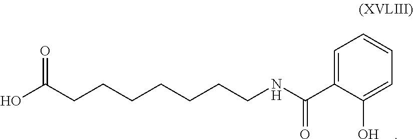 [SMILES: O=C(O)CCCCCCCNC(=O)c1ccccc1O]

U.S. Pat. No. 6,313,088 to Leone-Bay et al., incorporated herein by this reference, discloses 8-[(2-hydroxy-4-methoxy-benzoyl) amino]-octanoic acid as a penetration enhancer.

U.S. Pat. No. 6,180,140 to Leone-Bay et al., incorporated herein by this reference, discloses modified amino acids as penetration enhancers. The penetration enhancer comprises: (i) at least one acylated amino acid; (ii) at least one peptide comprising one acylated amino acid; or (iii) a combination of (i) and (ii), wherein the acylated amino acid is acylated by: (1) a C₃-C₁₀ cycloalkyl acylating agent, the agent being optionally substituted with C₁-C₇ alkyl, C₂-C₇ alkenyl, C₁-C₇ alkoxy, hydroxyl, phenyl, phenoxy, or —CO₂R, wherein R is hydrogen, C₁-C₄ alkyl, or C₂-C₄ alkenyl; or (2) a C₃-C₁₀ cycloalkyl substituted C₁-C₆ alkyl acylating agent. Amino acids suitable for use in these penetration enhancers are typically of formula (XLIX):

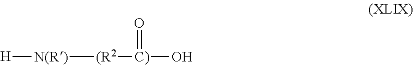

wherein: R¹ is hydrogen, C₁-C₄ alkyl, or C₂-C₄ alkenyl; R² is 01-C₂₄ alkyl, C₂-C₂₄ alkenyl, C₃-C₁₀ cycloalkyl, C₃-C₁₀ cycloalkenyl, phenyl, naphthyl, (C₁-C₁₀ alkyl) phenyl (C₂-C₁₀ alkenyl) phenyl, (C₁-C₁₀ alkyl) naphthyl (C₂-C₁₀ alkenyl) naphthyl, phenyl (C₁-C₁₀ alkyl), phenyl (C₂-C₁₀ alkenyl), naphthyl (C₁-C₁₀ alkyl) naphthyl (C₂-C₁₀ alkenyl); R² can be optionally substituted with C₁-C₄ alkyl, C₂-C₄ alkenyl, C₁-C₄ alkoxy, hydroxyl, sulfhydryl, —CO₂R³, C₃-C₁₀ cycloalkyl, C₃-C₁₀ cycloalkenyl, heterocycle having 3-10 ring atoms wherein the heteroatom is one or more of N, O. or S, or any combination thereof, aryl, C₁-C₁₀ alkaryl, aryl(C₁-C₁₀ alkyl), or any combination thereof; R² can be optionally interrupted by oxygen, nitrogen, sulfur, or any combination thereof; and R³ is hydrogen, C₁-C₄ alkyl, or C₂-C₄ alkenyl. The amino acid can be one of the following naturally-occurring amino acids: alanine, arginine, asparagine, aspartic acid, citrulline, cysteine, cystine, glutamine, glycine, histidine, isoleucine, leucine, lysine, methionine, ornithine, phenylalanine, proline, serine, threonine, tryptophan, tyrosine, valine, hydroxyproline, γ-carboxyglutamate, phenylglycine, or O-phosphoserine. Alternatively, the amino acid can be one of the following non-naturally-occurring amino acids: β-alanine, α-amino butyric acid, γ-amino butyric acid, γ-(aminophenyl) butyric acid, .alpha.-amino isobutyric acid, s-amino caproic acid, 7-amino heptanoic acid, β-aspartic acid, aminobenzoic acid, aminophenyl acetic acid, aminophenyl butyric acid, γ-glutamic acid, S-acetamidomethyl-L-cysteine, ε-lysine, ε-lysine (A-Fmoc), methionine sulfone, norleucine, norvaline, ornithine, D-ornithine, p-nitro-phenylalanine, 1,2,3,4,-tetrahydroisoquinoline-3-carboxylic acid and thioproline.

U.S. Pat. No. 6,071,510 to Leone-Bay et al., incorporated herein by this reference, discloses modified amino acids as penetration enhancers. The modified amino acids can be prepared by acylation or sulfonation of amino acids such as aminobutyric acid, aminocaproic acid, or aminocaprylic acid.

U.S. Pat. No. 6,001,347 to Leone-Bay et al., incorporated herein by this reference, discloses penetration enhancers, including the compound of Formula (L):

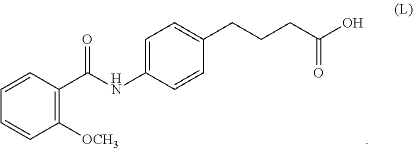 [SMILES: COc1ccccc1C(=O)Nc1ccc(CCCC(=O)O)cc1]

U.S. Pat. No. 5,989,539 to Leone-Bay et al., incorporated herein by this reference, discloses penetration enhancers, including the compound of Formula (LI):

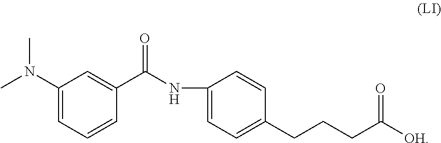 [SMILES: CN(C)c1cccc(C(=O)Nc2ccc(CCCC(=O)O)cc2)c1]

U.S. Pat. No. 5,965,121 to Leone-Bay et al., incorporated herein by this reference, discloses penetration enhancers, including the compound of Formula (LII):

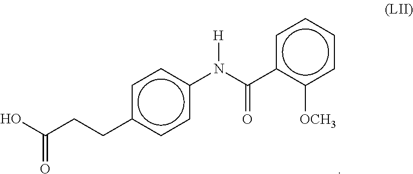

U.S. Pat. No. 5,955,103 to Leone-Bay et al., incorporated herein by this reference, discloses penetration enhancers that are modified amino acids and are of either Formula (LIII) or (LIV):

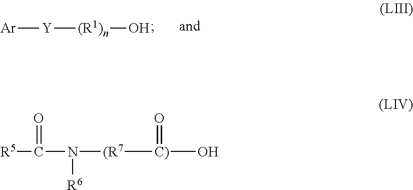

wherein: (i) Ar is an unsubstituted or substituted phenyl or naphthyl; (ii) Y is —C(O)— or —S(O₂)—; (iii) R¹ has the formula —N(R³)—R²—C(O)—; (iv) R² is C₁-C₂₄ alkyl, C₁-C₂₄ alkenyl, phenyl, naphthyl, (C₁-C₁₀ alkyl)phenyl, (C₁-C₁₀ alkenyl)phenyl, (C₁-C₁₀ alkyl)naphthyl, (C₁-C₁₀ alkenyl)naphthyl, phenyl(C₁-C₁₀ alkyl), phenyl(C₁-C₁₀ alkenyl), naphthyl(C₁-C₁₀ alkyl), or naphthyl(C₁-C₁₀ alkenyl); (v) R² is optionally substituted with C₁-C₄ alkyl, C₁-C₄ alkenyl, C₁-C₄ alkoxy, hydroxyl, sulfhydryl, CO₂R⁴, or any combination thereof; (vi) R⁴ is hydrogen, C₁-C₄ alkyl, or C₁-C₄ alkenyl; (vii) R² is optionally interrupted by oxygen, nitrogen, sulfur, or any combination thereof; (viii) R³ is hydrogen, C₁-C₄ alkyl, or C₁-C₄ alkenyl; (ix) R⁵ is either: (A) C₃-C₁₀ cycloalkyl, optionally substituted with C₁-C₇ alkyl, C₂-C₇ alkenyl, C₁-C₇ alkoxy, hydroxyl, phenyl, phenoxy, or —CO₂R⁸, wherein R⁸ is hydrogen, C₁-C₄ alkyl, or C₂-C₄ alkenyl; or (B) C₁-C₆ alkyl substituted with C₃-C₁₀ cycloalkyl; (x) R⁶ is C₃-C₁₀ cycloalkyl; R⁷ is C₁-C₂₄ alkyl, C₂-C₂₄ alkenyl, C₃-C₁₀ cycloalkyl, phenyl, naphthyl, (C₁-C₁₀ alkyl)phenyl, (C₂-C₁₀ alkenyl)phenyl, (C₁-C₁₀ alkyl)naphthyl, (C₂-C₁₀ alkenyl)naphthyl, phenyl(C₁-C₁₀ alkyl), phenyl(C₂-C₁₀ alkenyl), naphthyl(C₁-C₁₀ alkyl), or naphthyl(C₂-C₁₀ alkenyl); (xi) R⁷ is optionally substituted with C₁-C₄ alkyl, C₂-C₄ alkyl, C₁-C₄ alkoxy, hydroxyl, sulfhydryl, —CO₂R⁹, C₃-C₁₀ cycloalkyl, C₃-C₁₀ cycloalkenyl, a heterocycle having 3-10 ring atoms wherein the heteroatom is one or more of N, O, or S or any combination thereof, aryl, (C₁-C₁₀)alkaryl, aryl(C₁-C₁₀ alkyl), or any combination thereof; (xii) R⁷ is optionally interrupted by oxygen, nitrogen, sulfur, or any combination thereof; and (xiii) R⁹ is hydrogen, C₁-C₄ alkyl, or C₂-C₄ alkenyl.

U.S. Pat. No. 5,939,381 to Leone-Bay et al., incorporated herein by this reference, discloses penetration enhancers, including the penetration enhancer of Formula (LV):

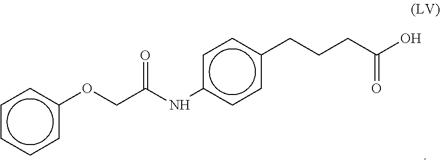

U.S. Pat. No. 5,879,681 to Leone-Bay et al., incorporated herein by this reference, discloses penetration enhancers, including a penetration enhancer of Formula (LVI):

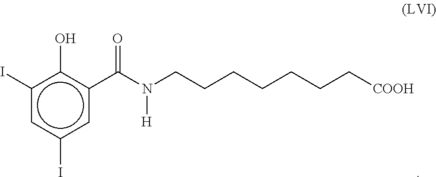

U.S. Pat. No. 5,876,710 to Leone-Bay et al., incorporated herein by this reference, discloses penetration enhancers, including the penetration enhancer of Formula (LVII):

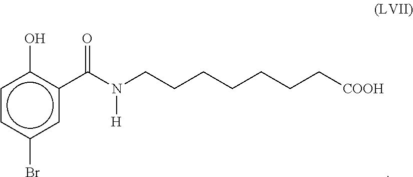

U.S. Pat. No. 5,866,536 to Leone-Bay et al., incorporated herein by this reference, discloses penetration enhancers, including the penetration enhancer of Formula (LVIII):

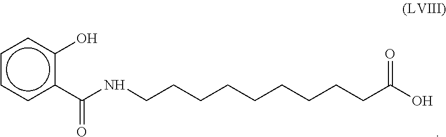

U.S. Pat. No. 5,863,944 to Leone-Bay et al., incorporated herein by this reference, discloses penetration enhancers, including penetration enhancers of Formulas (LVIX), (LX), and (LXI):

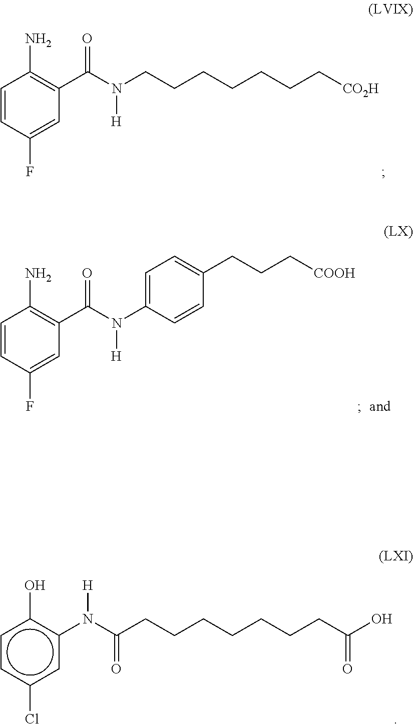

U.S. Pat. No. 5,804,688 to Leone-Bay et al., incorporated herein by this reference, discloses penetration enhancers, including the penetration enhancer of Formula (LXII):

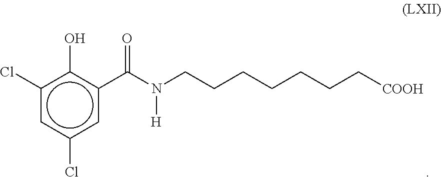

U.S. Pat. No. 5,792,451 to Sarubbi et al., incorporated herein by this reference, discloses penetration enhancers suitable for oral administration, including: (1) (a) at least one acylated aldehyde of an amino acid, (b) at least one acylated ketone of an amino acid, (c) at least one acylated aldehyde of a peptide, (d) at least one acylated ketone of a peptide, (e) any combination of (1)(a), (1)(b), (1)(c), and (1)(d); (2) (a) carboxymethyl-phenylalanylleucine; (b) 2-carboxy-3-phenylpropionylleucine; (c) 2-benzylsuccinic acid; (d) (phenylsulfonamide)phenylbutyric acid; and (e) any combination of (2)(a), (2)(b), (2)(c) and (2)(d); or (3) a combination of (1) and (2).

U.S. Pat. No. 5,776,888 to Leone-Bay et al., incorporated herein by this reference, discloses penetration enhancers, including the penetration enhancer of Formula (LXIII):

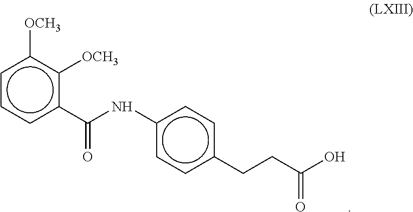

U.S. Pat. No. 5,773,647 to Leone-Bay et al., incorporated herein by this reference, discloses penetration enhancers, including the penetration enhancer of Formula (LXIV):

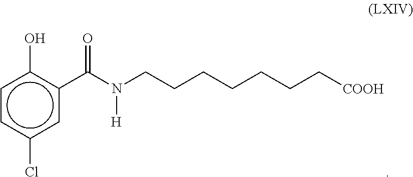

U.S. Pat. No. 5,766,633 to Milstein et al., incorporated herein by this reference, discloses penetration enhancers suitable for oral administration, including: (1) (a) at least one acetylated aldehyde of an amino acid; (b) at least one acetylated ketone of an amino acid; (c) at least one acetylated aldehyde of a peptide; (d) at least one acetylated ketone of a peptide; or (e) any combination of (1)(a), (1)(b), (1)(c), and (1)(d); (2) (a) carboxymethyl-phenylalanylleucine; (b) 2-carboxy-3-phenylpropionylleucine; (c) 2-benzylsuccinic acid; (d) an actinonin; (e) a compound having the formula Ar—Y—(R¹)ₙ—OH, wherein: (i) Ar is a substituted or unsubstituted phenyl or naphthyl; (ii) Y is —C(O)— or —SO₂—; (iii) R¹ is —N(R⁴)—R³—C(O)—, wherein: (A) R³ is C₁-C₂₄ alkyl, C₁-C₂₄ alkenyl, phenyl, naphthyl, (C₁-C₁₀ alkyl)phenyl, (C₁-C₁₀ alkyl)naphthyl, (C₁-C₁₀ alkenyl)phenyl, C₁-C₁₀ alkenyl(naphthyl), phenyl(C₁-C₁₀ alkyl), phenyl(C₁-C₁₀ alkenyl), naphthyl(C₁-C₁₀ alkyl), or phenyl(C₁-C₁₀ alkenyl); (B) R³ is optionally substituted with C₁-C₄ alkyl, C₁-C₄ alkenyl, C₁-C₄ alkoxy, hydroxyl, sulfhydryl, —CO₂R⁵, cycloalkyl, cycloalkenyl, heterocyclyl, aryl, alkaryl, heteroaryl, or heteroalkaryl or any combination thereof; (C) R⁵ is hydrogen, C₁-C₄ alkyl, or C₁-C₄ alkenyl; (D) R³ is optionally interrupted by oxygen, nitrogen, sulfur, or any combination thereof; (E) R⁴ is hydrogen, C₁-C₄ alkyl, or C₁-C₄ alkenyl; and (F) n is an integer from 1 to 5; or (f) any combination of (2)(a), (2)(b), (2)(c), (2)(d), and (2)(e); or (3) a combination of (1) and (2).

U.S. Pat. No. 5,541,155 to Leone-Bay et al., incorporated herein by this reference, discloses penetration enhancers that are acids or acid salts having the general formula RCO₂H, wherein R is C₁-C₂₄ alkyl, C₂-C₂₄ alkenyl, C₃-C₁₀ cycloalkyl, C₃-C₄ cycloalkenyl, phenyl, naphthyl, (C₁-C₁₀ alkyl)phenyl, (C₂-C₁₀ alkenyl)phenyl, (C₁-C₁₀ alkyl)naphthyl, (C₂-C₁₀ alkenyl)naphthyl, phenyl(C₁-C₁₀ alkyl), phenyl(C₂-C₁₀ alkenyl), naphthyl(C₁-C₁₀ alkyl), or naphthyl(C₂-C₁₀ alkenyl), with R being optionally substituted with C₁-C₁₀ alkyl, C₂-C₁₀ alkenyl, C₁-C₄ alkoxy, hydroxyl, sulfhydryl, CO₂R¹, C₃-C₁₀ cycloalkyl, C₃-C₁₀ cycloalkenyl, heterocyclyl having 3-10 ring atoms wherein the heteroatom is one or more atoms of N, O, S or any combination thereof, aryl, (C₁-C₁₀ alk)aryl, aryl(C₁-C₁₀ alkyl), or any combination thereof, R being optionally interrupted by oxygen, nitrogen, sulfur, or any combination thereof; and R¹ is hydrogen, C₁-C₄ alkyl, or C₂-C₄ alkenyl. The penetration enhancer can comprise: (i) an acid as described above; (ii) a salt of the acid; or (iii) a combination of (i) and (ii). The preferred carboxylic acids are cyclohexanecarboxylic acid, cyclopentanecarboxylic acid, cycloheptanecarboxylic acid, hexanoic acid, 3-cyclohexanepropanoic acid, methylcyclohexanecarboxylic acid, 1,2-cyclohexanedicarboxylic acid, 1,3-cyclohexanedicarboxylic acid, 1,4-cyclohexanedicarboxylic acid, 1-adamantanecarboxylic acid, phenylpropanoic acid, adipic acid, cyclohexanepentanoic acid, cyclohexanebutanoic acid, pentylcyclohexanoic acid, 2-cyclopentanehexanoic acid, cyclohexanebutanoic acid, and (4-methylphenyl) cyclohexane acetic acid.

U.S. Pat. No. 7,429,564 to Arbit et al., incorporated by this reference, discloses the use of 4-[(4-chloro-2-hydroxybenzoyl)amino]butanoic acid and its sodium salt, monosodium 4-[(4-chloro-2-hydroxybenzoyl)amino]butanoate (“4-CNAB”). Additional penetration enhancers are disclosed, namely penetration enhancers of Formulas (LXV) and (LXVI):

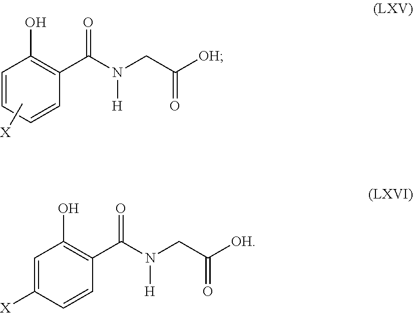

In Formula (LXV), X is one or more of hydrogen, halo, hydroxyl, or C₁-C₃ alkoxy. In Formula (LXVI), X is halo and R is substituted or unsubstituted C₁-C₃ alkylene or substituted or unsubstituted C₁-C₃ alkenylene.

United States Patent Application Publication No. 2012/0258911, by Gschneidner et al., incorporated herein by this reference, discloses phenylalkylcarboxylic acids as penetration enhancers, including, but not limited to, 4-(4-methoxyphenyl)butanoic acid, 5-(2-methoxyphenyl)pentanoic acid, 5-(3-fluorophenyl)pentanoic acid, 5-(3-methoxyphenyl)pentanoic acid, 6-(3-fluorophenyl)hexanoic acid, 3-(4-t-butylphenyl)propanoic acid, 3-(4-n-butylphenyl)propanoic acid, 3-(4-n-propylphenyl)propanoic acid, 3-(4-n-propoxyphenyl)propanoic acid, 3-(4-isopropoxyphenyl)propanoic acid, 3-(4-n-butoxyphenyl)propanoic acid, 3-(3-phenoxyphenyl)propanoic acid, 3-(3-ethoxyphenyl)propanoic acid, 3-(3-isopropoxyphenyl)propanoic acid, 3-(3-n-butoxyphenyl)propanoic acid, 3-(3-n-propoxyphenyl)propanoic acid, 3-(3-isobutoxyphenyl)propanoic acid, 3-(4-isobutoxyphenyl)propanoic acid, 4-(4-ethylphenyl)butanoic acid, 4-(4-isopropylphenyl)butanoic acid, and 5-(4-ethylphenyl)pentanoic acid.

United States Patent Application Publication No. 2011/0183898 by Dinh, incorporated herein by this reference, discloses penetration enhancers including penetration enhancers of Formulas (LXVII), (LXVIII), and (LXIX):

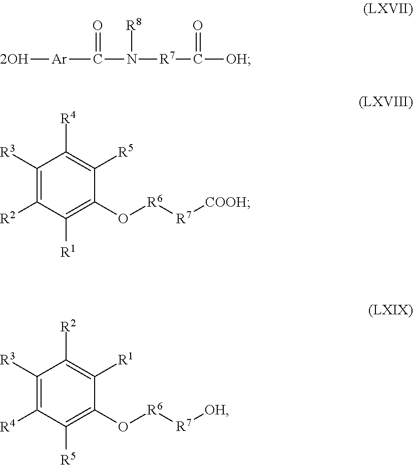

wherein: in Formula (LXVII): (i) Ar is phenyl or naphthyl; (ii) Ar is optionally substituted with one or more of hydroxyl, halo, C₁-C₄ alkyl, C₁-C₄ alkenyl, C₁-C₄ alkoxy, or C₁-C₄ haloalkoxy; (iii) R⁷ is selected from C₄-C₂₀ alkyl, C₄-C₂₀ alkenyl, phenyl, naphthyl, (C₁-C₁₀ alkyl)phenyl, (C₁-C₁₀ alkenyl)phenyl, C₁-C₁₀ alkyl)napthyl, (C₁-C₁₀ alkenyl)naphthyl, phenyl(C₁-C₁₀ alkyl), phenyl(C₁-C₁₀ alkenyl), naphthyl(C₁-C₁₀ alkyl), or naphthyl(C₁-C₁₀ alkenyl); (iv) R⁷ is optionally interrupted by oxygen, nitrogen, sulfur, or any combination thereof; (v) R⁷ optionally substituted with C₁-C₄ alkyl, C₂-C₄ alkenyl, C₁-C₄ alkoxy, C₁-C₄ haloalkoxy, hydroxyl, sulfhydryl, —CO₂R⁹, and combinations thereof; (vi) R⁸ is selected from hydrogen, C₁-C₄ alkyl, C₂-C₄ alkenyl, C₁-C₄ alkoxy, and C₁-C₄ haloalkoxy; and (vii) R⁹ is hydrogen, C₁-C₄ alkyl, or C₂-C₄ alkenyl; in Formula (LXVIII): (i) R¹, R², R³, and R⁴ are each independently hydrogen, hydroxy, halo, C₁-C₄ alkyl, C₂-C₄ alkenyl, C₁-C₄ alkoxy, —C(O)R⁸, —NO₂, —N⁺R⁹R¹⁰, and —NR⁹R¹⁰R¹¹(R¹²)⁻; (ii) R⁵ is hydrogen, hydroxyl, nitro, halo, trifluoromethyl, —NR¹⁴R¹⁵, —N⁺R¹⁴R¹⁵R¹⁶(R¹³)⁻, amide, C₁-C₁₂ alkyl, C₂-C₁₂ alkenyl, carbamate, carbonate, urea, or —C(O)R¹⁸; (iii) R⁵ is optionally substituted with halo, hydroxyl, sulfhydryl, or —COOH; (iv) R⁵ is optionally interrupted by oxygen, nitrogen, sulfur, or —C(O)—; (v) R⁶ is a C₁-C₁₂ alkylene, C₁-C₁₂ alkenylene, or arylene; (vi) R⁶ is optionally substituted with C₁-C₄ alkyl, C₂-C₄ alkenyl, C₁-C₄ alkoxy, hydroxyl, sulfhydryl, halo, amino, or —CO₂R⁸; (vii) R⁶ is optionally substituted with C₁-C₄ alkyl, C₂-C₄ alkenyl, C₁-C₄ alkoxy, hydroxyl, sulfhydryl, amino, or —CO₂R⁸; (viii) R⁶ is optionally interrupted by oxygen or nitrogen; (ix) R⁷ is a bond or arylene; (x) R⁷ is optionally substituted with hydroxyl, halogen, —C(O)CH₃, —NR¹⁰R¹¹, —N⁺R¹⁰R¹¹R¹²(R¹³)⁻; (xi) R⁸ is hydrogen, C₁-C₄ alkyl, C₂-C₄ alkenyl, or amino; (xii) R⁹, R¹⁰, R¹¹, and R¹² are each independently hydrogen or C₁-C₁₀ alkyl; (xiii) R¹³ is a halide, hydroxide, sulfate, tetrafluoroborate, or phosphate; (xiv) R¹⁴, R¹⁵, and R¹⁶ are each independently hydrogen, C₁-C₁₀ alkyl, C₁-C₁₀ alkyl substituted with —COOH, C₂-C₁₂ alkenyl, C₂-C₁₂ alkenyl substituted with —COOH, or —OC(O)R¹⁷; (xv) R¹⁷ is hydroxyl, C₁-C₁₀ alkyl, or C₂-C₁₂ alkenyl; and (xvi) R¹⁸ is hydrogen, C₁-C₆ alkyl, hydroxyl, —NR¹⁴R¹⁵, or N⁺R¹⁴R¹⁵R¹⁶(R¹³); and in Formula (LXIX): (i) R¹, R², R³, R⁴, and R⁵ are independently hydrogen, cyano, hydroxyl, —OCH₃, or halo, provided that at least one of R¹, R², R³, R⁴, and R⁵ is cyano; and (ii) R⁶ is C₁-C₁₂ linear or branched alkylene, alkenylene, arylene, alkyl(arylene), or aryl(alkylene).

United States Patent Application Publication No. 2010/0105604 by Song, incorporated herein by this reference, discloses allyloxy and alkoxy benzoic acid penetration enhancers, including penetration enhancers of Formulas (LXX), (LXXI), and (LXXII):

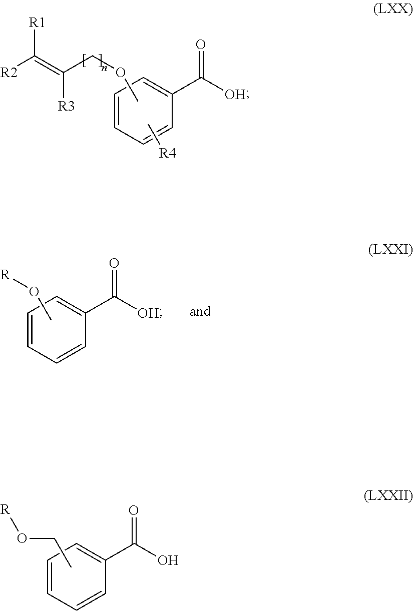

wherein: in Formula (LXX): (i) R1, R2, and R3 are independently hydrogen, methyl, or halo; (ii) R4 is hydrogen, methyl, methoxy, hydroxyl, halo, acetyl, or 2-hydroxy-ethoxy; and (iii) n is 1, 2, 3, or 4; in Formula (LXXI): R is C₁-C₆ straight-chain or branched alkyl; and in Formula (LXXII): R is methyl, ethyl, isopropyl, propyl, butyl, allyl, 1-methylallyl, 2-methylallyl, or butenyl.

United States Patent Application Publication No. 20100074861 by Tang et al., incorporated herein by this reference, discloses penetration enhancers with a cyclic moiety of Formula (LXXIII):

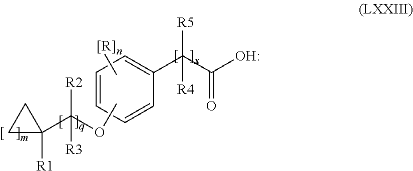

wherein: (i) m is 1, 2, 3, 4, 5, or 6; (ii) n is 0, 1, 2, 3, or 4; (iii) q and x are each independently chosen from 0, 1, 2, 3, 4, 5, 6, 7, 8, 9, or 10; (iv) R in [R]ₙ (where n may be 0, 1, 2, 3, or 4 as set forth above) may be the same or different (if n is 2, 3, or 4) and is hydrogen, halo, a substituted or non-substituted alkyl, a substituted or non-substituted alkoxy, a substituted or non-substituted alkenyloxy, or a substituted or non-substituted aryloxy; and (v) R1, R2, R3, R4, and R5 are each independently selected from hydrogen, halogen, substituted or non-substituted alkyl, substituted or non-substituted alkenyl, substituted or non-substituted alkynyl, substituted or non-substituted alkoxy, substituted or non-substituted aryloxy, substituted or non-substituted aryl, substituted or non-substituted heteroaryl, substituted or non-substituted cycloalkyl, and substituted or non-substituted heterocycloaryl.

United States Patent Application Publication No. 20100062970 by Song, incorporated herein by this reference, discloses propylphenoxy ethers as penetration enhancers, including compounds of Formula (LXXIV):

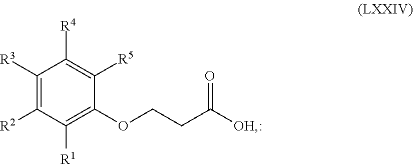

wherein: (i) R¹, R², R³. R⁴, and R⁵ are independently selected from hydrogen, halo, unsubstituted or substituted alkyl, unsubstituted or substituted alkenyl, unsubstituted or substituted alkoxy, unsubstituted or substituted haloalkoxy, hydroxy, —C(O)R⁸, nitro, —NR⁹R¹⁰, —N⁺R⁹R¹⁰R¹¹(R¹²), carbonate, ureido, CX₃, and cyano; (ii) R⁸ is hydrogen, C₁-C₄ alkyl, C₂-C₄ alkenyl, or amino; (iii) R⁹, R¹⁰, R¹¹, and R¹² are each independently hydrogen or C₁-C₁₀ alkyl; and (iv) X is halo.

United States Patent Application Publication No. 2009/0092580 by Song, incorporated herein by this reference, discloses dialkyl ether penetration enhancers, including dialkyl ether penetration enhancers of Formula (LXXV):

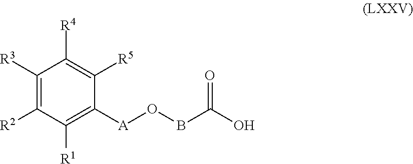

wherein: (i) A is a C₁-C₆ alkylene group that is straight-chain or branched-chain or substituted or unsubstituted; (ii) B is a C₁-C₂ alkylene group that is straight-chain or branched-chain or substituted or unsubstituted; (iii) R₁, R₂, R₃, R₄, and R₅ are each independently hydrogen, halo, unsubstituted or substituted alkyl, unsubstituted or substituted alkenyl, unsubstituted or substituted alkoxy, unsubstituted or substituted haloalkoxy, hydroxy, —C(O)R⁸, nitro, —NR⁹R¹⁰, —N⁺R⁹R¹⁰R¹¹ (R¹²), carbonate, ureido, —CX₃, or cyano, optionally interrupted by an O, N, S, or —C(O)— group, wherein A and R₁ may together form a cycloalkyl group; (iii) R⁸ is hydrogen, C₁-C₄ alkyl, C₂-C₄ alkenyl, or amino; (iv) R⁹, R¹⁰, R¹¹, and R¹² are each independently hydrogen or C₁-C₁₀ alkyl; and X is halo.

United States Patent Application Publication No. 2008/0167217 by Rath et al., incorporated herein by this reference, discloses aryl ketone penetration enhancers, including aryl ketone penetration enhancers of Formula (LXXVI):

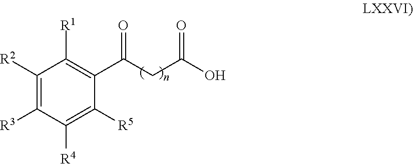

wherein: (i) n is 1, 2, 3, 4, 5, 6, 7, 8, or 9; and (ii) R1, R2, R3, R4, and R5 are each independently hydrogen, C₁-C₆ alkyl, C₁-C₆ alkoxy, C₂-C₆ alkenyl, halo, hydroxyl, —NH—C(O)—CH₃, or —O—C₆H₅. Preferred compounds include 4-oxo-4-phenyl-butyric acid; 10-(4-hydroxy-phenyl)-10-oxodecanoic acid; 10-(2-hydroxy-phenyl)-10-oxo-decanoic acid; 4-(4-methoxy-phenyl)-4-oxo-butyric acid; 5-(4-methoxy-phenyl)-5-oxo-pentanoic acid; 4-(3,5-difluoro-phenyl)-4-oxo-butyric acid; 5-oxo-5-phenyl-pentanoic acid; 4-(2,4-dimethyl-phenyl)-4-oxo-butyric acid; 6-(4-methoxy-3,5-dimethyl-phenyl)-6-oxo-hexanoic acid; 5-(4-isopropyl-phenyl)-5-oxo-pentanoic acid; 4-(2-methoxy-phenyl)-4-oxo-butyric acid; 4-(4-fluoro-phenyl)-4-oxo-butyric acid; 6-(4-methoxy-phenyl)-6-oxo-hexanoic acid; 4-(3,5-dimethyl-phenyl)-4-oxo-butyric acid; 6-(3,4-dimethyl-phenyl)-6-oxo-hexanoic acid; 4-(3,4-dimethyl-phenyl)-4-oxo-butyric acid; 4-oxo-4-(4-phenoxy-phenyl)-butyric acid; 4-(2,5-dimethyl-phenyl)-4-oxo-butyric acid; 8-(3,5-dimethyl-phenyl)-8-oxo-octanoic acid; 6-(2,5-dichloro-phenyl)-6-oxo-hexanoic acid; 4-(2,5-dichloro-phenyl)-4-oxo-butyric acid; 6-(3,5-dimethyl-phenyl)-6-oxo-hexanoic acid; 10-(2,5-dihydroxy-phenyl)-10-oxo-decanoic acid; 8-oxo-8-phenyl-octanoic acid; 6-(2,5-difluoro-phenyl)-6-oxo-hexanoic acid; 7-oxo-7-phenyl-heptanoic acid; 4-(4-ethyl-phenyl)-4-oxo-butyric acid; 4-(2,4-difluoro-phenyl)-4-oxo-butyric acid; 4-(4-butoxy-phenyl)-4-oxo-butyric acid; 4-oxo-4-(4-propyl-phenyl)-butyric acid; 4-oxo-4-(4-pentyl-phenyl)-butyric acid; 4-(4-hexyloxy-phenyl)-4-oxo-butyric acid; 4-(2,5-difluoro-phenyl)-4-oxo-butyric acid; 5-(4-chloro-phenyl)-5-oxo-pentanoic acid; 6-(3,5-difluoro-phenyl)-6-oxo-hexanoic acid; 4-oxo-4-p-tolyl-butyric acid; 6-oxo-6-phenyl-hexanoic acid; 5-oxo-5-(4-phenoxy-phenyl)-pentanoic acid; 5-oxo-5-(3-phenoxy-phenyl)-pentanoic acid; and 7-oxo-7-(3-phenoxy-phenyl)-heptanoic acid.

U.S. Pat. No. 8,575,123 to Manoharan et al., incorporated herein by this reference, describes a range of penetration enhancers such as arachidonic acid, lauric acid, caprylic acid, capric acid, myristic acid, palmitic acid, stearic acid, linoleic acid, linolenic acid, dicaprate, tricaprate, monolein, dilaurin, glyceryl 1-monocaprate, 1-dodecylazacycloheptan-2-one, an acylcarnitine, an acylcholine, or a C₁₋₁₀ alkyl ester, monoglyceride, diglyceride, or a pharmaceutically acceptable salt thereof; also bile salts such as cholic acid, dehydrocholic acid, deoxycholic acid, glucholic acid, glycholic acid, glycodeoxycholic acid, taurocholic acid, taurodeoxycholic acid, chenodeoxycholic acid, ursodeoxycholic acid, sodium tauro-24,25-dihydro-fusidate, or sodium glycodihydrofusidate; polyoxyethylene-9-lauryl ether; also chelating agents such as EDTA or citric acid; a salicylate, an N-acyl derivative of collagen, an N-amino acyl derivative of a beta-diketone; a surfactant, including an ionic or nonionic surfactant, such as sodium lauryl sulfate, polyoxyethylene-20-cetyl ether, a perfluorochemical emulsion; or other compounds that can act as penetration enhancers, such as unsaturated cyclic ureas, 1-alkyl-alkones, 1-alkenylazacyclo-alakanones, glycols, pyrroles, azones, and terpenes.

U.S. Pat. No. 8,569,320 to Melzer et al., incorporated herein by this reference, discloses a range of penetration agents, including polyvalent aliphatic C₂-C₁₀ alcohols, polyalkylene glycols having C₂-C₄ alkylene groups, nonalkoxylated ethers of polyvalent aliphatic C₂-C₁₀ alcohols and polyalkylene glycols having C₂-C₄ alkylene groups, azones, terpenes, terpenoids, pyrrolidones, and sulfoxides.

United States Patent Application Publication No. 2012/0231069 by Nowotnik et al., incorporated herein by this reference, discloses nanoparticles and micelles as penetration agents. The nanoparticles and micelles can be constructed from a polymer, such as, but not necessarily limited to, dextran, carboxymethyl dextran, chitosan, trimethylchitosan, poly(lactic-co-glycolic acid) (PLGA), polylactic acid (PLA), polyglycolic acid (PGA), polyvinylalcohol (PVA), polyanhydrides, polyacylates, polymethacrylates, polyacylamides, polymethacrylate, dextran, chitosan, cellulose, hypromellose, starch, dendrimers, peptides, proteins, polyethyleneglycols and poly(ethyleneglycol-co-propyleneglycol), and synthetic derivatives of the aforementioned polymers. The therapeutically active agent (a pentosan polysulfate salt in this application) can be covalently attached to the polymer, possibly through a linker. The linker can be, for example, a short peptide chain (H—[NHCHR—CO]ₙ—OH) where n is 1-20 and R is the same or different for each of the n amino acids, and is one of the 22 side groups known to be present in natural amino acids; a short alkyl chain (CH₂)ₙ where n=2-10, terminated by two amino groups or two carboxyl groups or one amino group and one carboxyl group; an oligoethyleneoxy chain (CH₂CH₂O)ₙ where n=2-100, terminated by two amino groups or two carboxyl groups or one amino group and one carboxyl group; a poly(lactic-co-glycolic acid) (PLGA), polylactic acid (PLA), polyglycolic acid (PGA) chain of average molecular weight of 2 kDa to 70 kDa terminated by two amino groups or two carboxyl groups or one amino group and one carboxyl group; and any combination of two or more of any of the aforementioned linkers. Micelles and liposomes are also described. The polymer can be a linear, branched, or cross-linked polysaccharide. When the carrier is a micelle or a liposome, the lipid can include, but is not limited to, straight or branched alkanes or alkene functionalized at one end by hydrophilic groups that may be charged or neutral. For the purpose of fulfilling the requirements of this invention, the lipids may be optionally modified by covalent linkage of one or more therapeutically active agent molecules, either directly or via a suitable linker. Suitable lipids include, but are not limited to, both single chain amphiphiles and double chain amphiphiles, such as phospholipids (e.g. phosphatidylcholine). Other components such as cholesterol, fatty acids and other lipid soluble molecules which are known in the art to modify the properties of liposomes and micelles can also be used in the formation of nanocapsules. The nanocarriers can be modified, such as by the introduction of charged or ionizable groups, covalent attachment of the therapeutically active agent, and the introduction of functional groups (for example, hydrophobic or hydrophilic) which either enhance the nanocarrier formation and/or the pharmaceutical qualities of the resultant nanocarriers. Similarly, the lipids can be modified, including, but not limited to, the introduction of charged or ionizable groups, attachment of the therapeutically active agent, and the introduction of functional groups (for example, hydrophobic or hydrophilic) which either enhance the nanocarrier formation and/or the pharmaceutical qualities of the resultant nanocarriers. The carriers can include additional components before, during or after nanocarrier formation in order to control the size of nanoparticles, control stability and/or the drug release profile. Possible additional components include, but are not limited to, polyethylene glycol (PEG) and PEG block copolymers, polyacrylic, polymethacrylic, and other synthetic polymers, starch, cellulose, and other polysaccharides, fatty acids and other surfactants, and metal ions, especially di- and trivalent ions such as zinc, magnesium, and calcium. Additional components might also include a crosslinking agent, for example epoxy compounds, dialdehyde starch, glutaraldehyde, formaldehyde, dimethyl suberimidate, carbodiimides, succinimidyls, diisocyanates, acyl azide, reuterin, and crosslinking effected by ultraviolet irradiation.

U.S. Pat. No. 6,916,789 to O'Mahoney et al., incorporated herein by this reference, discloses synthetic peptide ligands as penetration enhancers.

U.S. Pat. No. 7,087,236 to Brayden, incorporated herein by this reference, discloses biodegradable polymers that are copolymers of lactic acid and glycolic acid or enantiomers thereof as penetration enhancers.

U.S. Pat. No. 7,268,214 to O'Mahoney et al., incorporated herein by this reference, discloses membrane translocating full-length peptide sequences, as well as fragments thereof, motifs derived therefrom, derivatives thereof, analogs thereof, and peptidomimetics based on the peptide sequences, as penetration enhancers.

U.S. Pat. No. 7,491,796 to O'Mahoney et al., incorporated herein by this reference, discloses D-form retro-inverted peptides as penetration enhancers; these peptides have the reverse sequence so that the original amino-terminus of the peptide comprising L-amino acids becomes the carboxyl-terminus of the peptide comprising D-amino acids; amino acid substitutions can be made and the peptides can be modified, such as by glycosylation, acetylation, phosphorylation, amidation, derivation by known protecting/blocking groups, proteolytic cleavage, linkage to an antibody molecule or other cellular ligand, or other methods, as penetration enhancers.

U.S. Pat. No. 7,658,938 to Cumming et al., incorporated herein by this reference, discloses penetration enhancers each of which: (i) is a solid at room temperature; and (ii) is a salt of a medium chain fatty acid having a carbon length of from 8 to 14 carbon atoms in particulate form, such as sodium caprylate, sodium caprate and sodium laurate; rate-controlling polymers such as cellulose such as hydroxypropyl cellulose and hydroxypropyl methyl cellulose; poly(ethylene) oxide; alkyl cellulose such as ethyl cellulose and methyl cellulose; carboxymethyl cellulose, hydrophilic cellulose derivatives; polyethylene glycol; polyvinylpyrrolidone; cellulose acetate; cellulose acetate butyrate; cellulose acetate phthalate; cellulose acetate trimellitate; polyvinyl acetate phthalate; hydroxypropylmethyl cellulose phthalate; hydroxypropylmethyl cellulose acetate succinate; polyvinyl acetaldiethylamino acetate; poly(alkylmethacrylate) and poly (vinyl acetate), or other suitable hydrophobic polymers including polymers and/or copolymers derived from acrylic or methacrylic acid and their respective esters, zein, waxes, shellac and hydrogenated vegetable oils can be used.

U.S. Pat. No. 7,670,626 to Clancy et al., incorporated herein by this reference, discloses penetration enhancers such as mono-, di-, and triglyceride esters of medium-chain (more than about 6 carbon atoms in length) and long-chain (more than about 12 carbon atoms in length) fatty acids, esters of fatty acids and glycols and esters of mixed fatty acids and glycols and mixtures thereof; diesters of propylene glycol having from about 7 to about 55 carbon atoms, propylene glycol esters of capric and caprylic acids, and mixtures thereof, having from 19 to 23 carbon atoms.

U.S. Pat. No. 7,704,977 to Leonard, incorporated herein by this reference, discloses penetration enhancers that are medium chain fatty acids or medium chain fatty acid derivatives having a carbon chain length of from 6 to 20 carbon atoms; with the provisos that (i) where the enhancer is an ester of a medium chain fatty acid, the chain length of from 6 to 20 carbon atoms relates to the chain length of the carboxylate moiety, and (ii) where the enhancer is an ether of a medium chain fatty acid, at least one alkoxy group has a carbon chain length of from 6 to carbon atoms, and wherein the enhancer and the composition are solids at room temperature.

U.S. Pat. No. 7,820,722 to Raoof et al., incorporated herein by this reference, discloses penetration enhancers that are compounds of Formula (LXXVII):

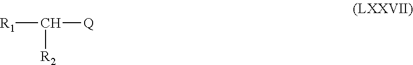

wherein Q is: (1) a partially or completely neutralized —COOH, or (2) a partially or completely neutralized —SO₃H, or (3) a mono- or di-substituted alkyl or alkenyl group having one to about twelve carbon atoms, the substituent(s) thereof being a partially or completely neutralized —COOH or partially or completely neutralized —SO₃H; and R₁ and R₂ are independently: (1) an unsubstituted alkyl or alkenyl group having one to about twelve carbon atoms, or (2) a substituted alkyl or alkenyl group having one to about twelve carbon atoms, the substituent thereof being selected from the group consisting of (i) partially or completely neutralized —COOH, (ii) partially or completely neutralized —SO₃H, (iii) —NH₂, (iv) —CONH₂; and (v) —OH.

U.S. Pat. No. 8,119,159 to Cumming et al., incorporated herein by this reference, discloses penetration enhancers, each of which: (i) is a solid at room temperature; and (ii) is a salt of a medium chain fatty acid having a carbon length of from 8 to 14 carbon atoms in particulate form, such as sodium caprylate, sodium caprate and sodium laurate; rate-controlling polymers such as cellulose such as hydroxypropyl cellulose and hydroxypropyl methyl cellulose; poly(ethylene) oxide; alkyl cellulose such as ethyl cellulose and methyl cellulose; carboxymethyl cellulose, hydrophilic cellulose derivatives; polyethylene glycol; polyvinylpyrrolidone; cellulose acetate; cellulose acetate butyrate; cellulose acetate phthalate; cellulose acetate trimellitate; polyvinyl acetate phthalate; hydroxypropylmethyl cellulose phthalate; hydroxypropylmethyl cellulose acetate succinate; polyvinyl acetaldiethylamino acetate; poly(alkylmethacrylate) and poly (vinyl acetate), or other suitable hydrophobic polymers including polymers and/or copolymers derived from acrylic or methacrylic acid and their respective esters, zein, waxes, shellac and hydrogenated vegetable oils can be used.

United States Patent Application Publication No. 2010/0016549 by O'Mahoney et al., incorporated herein by this reference, discloses penetration enhancers that are purified synthetic polypeptide ligands comprising a 12-mer L-peptide or homologue thereof.

U.S. Pat. No. 7,115,707 to Ben-Sasson et al., incorporated herein by this reference, discloses penetration enhancers that include peptide sequences possessing both hydrophobic amino acids and charged amino acids; optionally, the peptide sequences can be modified by hydrophobic moieties.

U.S. Pat. No. 8,535,695 to Salama et al., incorporated herein by this reference, discloses a penetration enhancer that is a medium chain fatty acid salt associated with a substantially hydrophobic medium, preferably castor oil.

U.S. Pat. No. 8,241,670 to Ben-Sasson, incorporated herein by this reference, discloses penetration enhancers comprising octanoate, sodium decanoate, sodium dodecanoate, and combinations thereof. The composition further includes a hydrophobic medium to produce a suspension, wherein the hydrophobic medium is selected from the group consisting of aliphatic molecules, cyclic molecules, aromatic molecules and combinations thereof, as well as a lecithin, a bile salt or a non-ionic detergent.

United States Patent Application Publication No. 2007/0275055 by Ben-Sasson et al. discloses penetration enhancers including counterions; the counter-ion is a liquid-forming counter-ion, such as cationic amphipathic molecules, i.e., imidazolium derivatives, pyridinium derivatives, phosphonium compounds or tetralkylammonium compounds; the action of the cation can be modified by addition of hydrophobic moieties; a hydrophobic agent can be a single molecule or a combination of hydrophobic molecules, like aliphatic or aromatic molecules; examples of aliphatic hydrophobic agents include fatty acids, mono-, di-, or tri-glycerides, ethers, or cholesterol esters of fatty acids.

United States Patent Application Publication No. 2006/0251713 by Ben-Sasson et al. discloses peptides derived from Escherichia coli as penetration enhancers; the peptides can be modified to make them more hydrophobic.

U.S. Pat. No. 7,651,694 to Lee discloses calcium phosphate nanoparticles as penetration enhancers.

United States Patent Application Publication No. 2011/0142889 by Lee et al. discloses penetration enhancers that include fatty acid, a medium chain glyceride, a surfactant, a steroidal detergent, an acyl carnitine, an alkanoyl choline, an N-acetylated amino acid, esters, salts and derivatives thereof, or any combination thereof.

United States Patent Application Publication No. 2012/0301401 by Botti et al. discloses penetration enhancers that are orthoester derivatives of crown ethers. Typically, these penetration enhancers are compounds of Formula (LXXVIII):

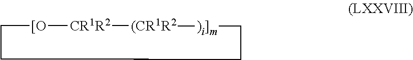

wherein:

(i) m is 4, 5, 6, 7, or 8;

(ii) i is independently for each occurrence, 1 or 2;

(iii) each occurrence of R¹ and R² is independently selected from hydrogen; linear or branched and substituted or unsubstituted C₁-C₁₀ alkyl, alkenyl, or alkynyl; and substituted or unsubstituted aryl with up to 10 ring atoms, or R¹ and R² form an oxo group;

(iv) there is at least one occurrence in the crown ether of R¹, R², and the carbon to which R¹ and R² are bound, the carbon being bound directly to an ether oxygen of Formula (LXXVIII), form together a group of subformula (LXXVIII(a))

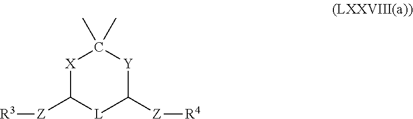

wherein L is a linker that is absent or is selected from a covalent bond and (CR⁵R⁶)ₙ, each occurrence of R⁵ and R⁶ being independently selected from: hydrogen; linear or branched and substituted or unsubstituted C₁-C₁₀ alkyl, alkenyl, or alkynyl; and substituted or unsubstituted aryl with up to 10 ring atoms; n is 1, 2, or 3; X and Y, independently from each other, are selected from O and S; Z, independently for each occurrence, is absent or an electron-withdrawing group; R³ and R⁴, independently for each occurrence, are selected from: hydrogen; linear or branched and substituted or unsubstituted C₁-C₁₀ alkyl, alkenyl, or alkynyl; and substituted or unsubstituted aryl with up to 10 ring atoms; H(OCH₂CH₂)ₖ— H(OCH₂CH₂)ₖO—, wherein k is 1, 2, 3, 4, 5, 6, 7, 8, 9, or 10; and wherein substituents, if present, are selected from hydroxyl, halogens, and O—CH₃.

United States Patent Application Publication No. 2012/0302502 by Botti et al., incorporated herein by this reference, discloses penetration enhancers that are crown compounds employed in a nonaqueous hydrophobic vehicle; the crown compound can be associated with a counterion. The crown compounds include, for example, cyclic polyethers (crown ethers, e.g., 18-crown-6) and cyclic polyesters (crown esters, e.g., polylactones such as nonactine and tetranactine, polyglycolic or lactic esters), and analogues/derivatives thereof. Of particular interest are crown compounds selected from (i) cyclic polyester; (ii) cyclic polyamide; (iii) cyclic polyether; (iv) cyclic polyoxime; (v) polythioester; (vi) polymer of aminoxy acids; (vii) polydisulfide; (viii) cyclic polydioxanones, and (ix) a cyclic compound belonging to more than one of (i) to (ix), where the crown is a cation-binding crown compound capable of forming a charge masking complex with a cation, such as protonated primary amino groups and/or protonated secondary amino groups, and/or protonated guanidinium groups. In some alternatives, the crown compounds are cyclic polyethers, cyclic polyesters, or cyclic depsipeptides. The crown compounds can include a biodegradable linkage. The crown ethers can be oxo-crown ethers comprising 4 to 8 coordinating oxygen ring atoms, 8 to 16 ring carbon atoms, and at least one oxo-substituted side chain, such as oxo-(18-crown-6) compounds and analogs or derivatives thereof, such as those comprising a structure selected from oxo-(18-crown-6), oxo-(18-crown-6) diethyl tartrate, and oxo-(18-crown-6) diglycerol tartrate.

U.S. Pat. Nos. 5,912,014 and 6,086,918 to Stern et al., incorporated herein by this reference, disclose penetration enhancers comprising acylcarnitines, phospholipids, and bile acids.

U.S. Pat. No. 6,673,574 to Stern et al., incorporated herein by this reference, discloses penetration enhancers that have a covalent linkage to a membrane translocator that is a peptide, fatty acid, or bile acid.

U.S. Pat. No. 7,316,819 to Crotts et al., incorporated herein by this reference, discloses lauroyl-L-carnitine as a penetration enhancer. Other acyl-L-carnitines are disclosed in United States Patent Application Publication No. 2004/0197323 by Mehta, incorporated herein by this reference.

U.S. Pat. No. 8,088,734 to Mehta et al., incorporated herein by this reference, discloses penetration enhancers comprising: (i) an anionic agent that is a cholesterol derivative, (ii) a mixture of a negative charge neutralizer and an anionic surface active agent, (iii) non-ionic surface active agents, and (iv) cationic surface active agents. The cationic surfactant and an anionic surfactant can be cholesterol derivatives. The anionic surface active agent can be a bile acid.

U.S. Pat. No. 8,324,156 to Arbit et al., incorporated herein by this reference, discloses (4-[(4-chloro, 2-hydroxybenzoyl)amino] butanoic acid as a penetration enhancer.

U.S. Pat. No. 8,383,852 to Tang et al., incorporated herein by this reference, discloses penetration enhancers that are compounds with a cyclic moiety, including: 3-[4-(cyclopropylmethoxy)phenyl]propanoic acid; 4-(cyclobutylmethoxy)benzoic acid; [4-(cyclobutylmethoxy)-3-methoxyphenyl]acetic acid; 4-(cyclopropylmethoxy)benzoic acid; [4-(cyclopropylmethoxy)phenyl]acetic acid; 2-(cyclobutylmethoxy)benzoic acid; [4-(cyclopentyloxy)-3-methoxyphenyl]acetic acid; [4-(cyclopropylmethoxy)-3-methoxyphenyl]acetic acid; 2-(cyclopropylmethoxy)benzoic acid; 2-(cyclopentyloxy)benzoic acid; 2-(cyclohexylmethoxy)benzoic acid; 3-(cyclopropylmethoxy)benzoic acid; 3-(cyclobutylmethoxy)benzoic acid; 3-(cyclopentyloxy)benzoic acid; 3-(cyclohexylmethoxy)benzoic acid; 4-(cyclopentyloxy)benzoic acid; 4-(cyclopentyloxy)benzoic acid; [4-(cyclobutylmethoxy)phenyl]acetic acid; 3-[4-(cyclobutylmethoxy)phenyl]propanoic acid; [4-(cyclohexylmethoxy)phenyl]acetic acid; 3-[4-(cyclohexylmethoxy)phenyl]propanoic acid; [4-(cyclohexylmethoxy)-3-methoxyphenyl]acetic acid; 3-[2-(cyclopropylmethoxy)phenyl]propanoic acid; [4-(cyclopentyloxy)phenyl]acetic acid; and 3-[4-(cyclopentyloxy)phenyl]propanoic acid.

U.S. Pat. No. 8,207,227 to Bay et al., incorporated herein by this reference, discloses disodium salts, ethanol solvates, and hydrates of a number of penetration enhancers including N-(5-chlorosalicyloyl)-8-aminocaprylic acid, N-(10-[2-hydroxybenzoyl]amino)decanoic acid, and sodium N-(8-[2-hydroxybenzoyl]amino)caprylate.

U.S. Pat. No. 8,431,736 to Dhoot et al., incorporated herein by this reference, discloses crystalline forms of the disodium salt of N-(5-chlorosalicyloyl)-8-aminocaprylic acid as penetration enhancers.

U.S. Pat. No. 8,513,300 to Abbas et al., incorporated herein by this reference, discloses penetration enhancers of Formula (LXXIX):

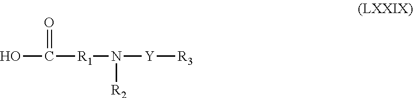

wherein:

(i) Y is carbonyl or SO₂;

(ii) R₁ is C₃-C₂₄ alkyl, C₂-C₂₀ alkenyl, C₂-C₂₀ alkynyl, cycloalkyl, or aromatic;

(iii) R₂ is hydrogen, C₁-C₄ alkyl, or C₂-C₄ alkenyl;

(iv) R₃ is C₁-C₇ alkyl, C₃-C₁₀ cycloalkyl, aryl, thienyl, pyrrolo, or pyridyl, wherein R₃ is optionally substituted with one or more C₁-C₅ alkyl groups, C₂-C₄ alkenyl groups, halogen, SO₂, COOH, or SO₃H.

United States Patent Application Publication No. 2012/0189666 by Dhoot et al., incorporated herein by this reference, discloses penetration enhancers in the form of microparticles or nanoparticles. The particles can comprise an active agent, such as, in this case, a pentosan polysulfate salt, and a penetration enhancer. In one alternative, the particles comprising a delivery agent compound and an active agent have a median particle size less than about 900 or 1000 micrometers. For example, the median particle size can range from about 45 to about 850 micrometers, from about 45 to about 150 micrometers, from about 150 to about 250 micrometers, from about 250 to about 425 micrometers, from about 425 to about 850 micrometers, from about 100 to about 1000 nm, or from about 500 to about 1000 nm. According to another embodiment, the particles have a median particle size less than about 1 micrometer. In some embodiments, particles may be as small as about 1 nanometer and as large as about 999 micrometers. For example, the particles may have a median particle size of less than about 999 micrometers, from about 1 nanometer to about 999 micrometers, about 1 to about 999 micrometers, about 1 to about 999 nanometers, about 45 to about 850 micrometers, about 45 to about 150 micrometers, about 150 to about 250 micrometers, about 250 to about 425 micrometers, about 425 to about 850 micrometers, about 100 to about 1000 nanometers, or about 500 to about 1000 nanometers. Alternatively, the penetration enhancer itself can be in the form of particles. The particles can have a median particle size of less than about 999 micrometers, about 1 nanometer to about 999 micrometers, about 1 to about 999 nanometers, or about 7 to about 16 micrometers. The particles can be in the form of fine granules or micro-beads, and can include a muco-adhesive. The penetration enhancer can be a penetration enhancer of Formula (LXXX), (LXXXI), (LXXXII), (LXXXIII), or (LXXXIV):

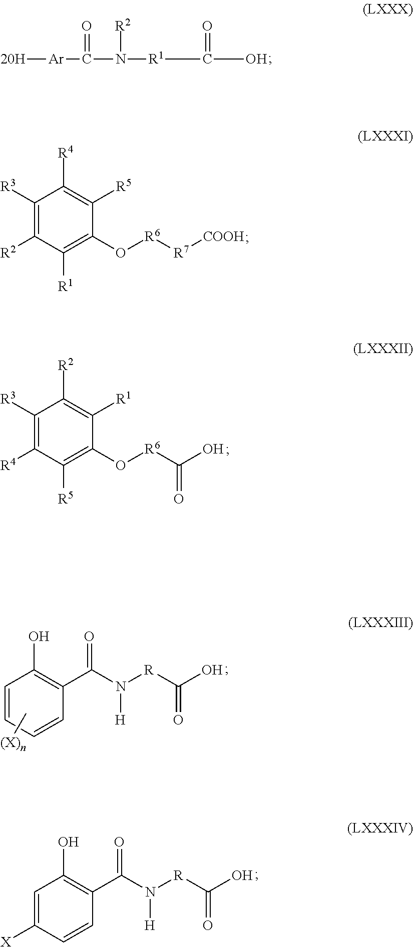

In Formula (LXXX):

(i) Ar is phenyl or naphthyl;

(ii) Ar is optionally substituted with one or more of hydroxyl, halogen, C₁-C₄ alkyl, C₁-C₄ alkenyl, C₁-C₄ alkoxy, or C₁-C₄ haloalkoxy;

(iii) R¹ is C₃-C₂₀ alkyl, C₄-C₂₀ alkenyl, phenyl, naphthyl, (C₁-C₁₀ alkyl)phenyl, (C₁-C₁₀ alkenyl)phenyl, (C₁-C₁₀ alkyl)naphthyl, (C₁-C₁₀ alkenyl)naphthyl, phenyl(C₁-C₁₀ alkyl), phenyl(C₁-C₁₀ alkenyl), naphthyl(C₁-C₁₀ alkyl), or naphthyl(C₁-C₁₀ alkenyl);

(iv) R¹ is optionally substituted with C₁-C₄ alkyl, C₂-C₄ alkenyl, C₁-C₄ alkoxy, C₁-C₄ haloalkoxy, hydroxyl, or sulfhydryl or any combination thereof;

(v) R² is hydrogen, C₁-C₄ alkyl, or C₂-C₄ alkenyl; and

(vi) R¹ is optionally interrupted by oxygen, nitrogen, sulfur, or any combination thereof; wherein the term “2-OH—Ar” refers to a phenyl or naphthyl group having a hydroxyl group at the 2-position.

In Formula (LXXXI):

(i) R¹, R², R³, and R⁴ are each independently hydrogen, hydroxyl, halogen, C₁-C₄ alkyl, C₂-C₄ alkenyl, C₁-C₄ alkoxy, —C(O)R⁸, —NO₂, —NR⁹R¹⁰, or —N⁺R⁹R¹⁰R¹¹(R¹²)⁻;

(ii) R⁵ is hydrogen, hydroxyl, —NO₂, halogen, —CF₃, —NR¹⁴R¹⁵, —N⁺R¹⁴R¹⁵R¹⁶(R¹³)⁻, amide, C₁-C₁₂ alkoxy, C₁-C₁₂ alkyl, C₁-C₁₂ alkenyl, carbamate, carbonate, urea, or —C(O)R¹⁸;

(iii) R⁵ is optionally substituted with halogen, hydroxyl, sulfhydryl, or carboxyl;

(iv) R⁵ is optionally interrupted by O, N, S, or —C(O)—;

(v) R⁶ is a C₁-C₁₂ alkylene, C₁-C₁₂ alkenylene, or arylene;

(vi) R⁶ is optionally substituted with C₁-C₄ alkyl, C₂-C₄ alkenyl, C₁-C₄ alkoxy, hydroxyl, sulfhydryl, halogen, amino, or —CO₂R⁸;

(vii) R⁶ is optionally interrupted by O or N;

(viii) R⁷ is a bond or arylene;

(ix) R⁷ is optionally substituted with hydroxyl, halogen, —C(O)CH₃, —NR¹⁰R¹¹, or —N⁺R¹⁰R¹¹R¹²(R¹³)⁻;

(x) R⁸ is hydrogen, C₁-C₄ alkyl, C₂-C₄ alkenyl, or amino;

(xi) R⁹, R¹⁰, R¹¹, and R¹² are independently hydrogen or C₁-C₁₀ alkyl;

(xii) R¹³ is a halide, hydroxide, sulfate, tetrafluoroborate, or phosphate;

(xiii) R¹⁴, R¹⁵, and R¹⁶ are each independently hydrogen, C₁-C₁₀ alkyl, C₁-C₁₀ alkyl substituted with carboxyl, C₂-C₁₂ alkenyl, C₂-C₁₂ alkenyl substituted with carboxyl, or —C(O)R¹⁷;

(xiv) R¹⁷ is hydroxyl, C₁-C₁₀ alkyl, or C₂-C₁₂ alkenyl;

(xv) R¹⁸ is hydrogen, C₁-C₆ alkyl, hydroxyl, —NR¹⁴R¹⁵, or —N⁺R¹⁴R¹⁵R¹⁶(R¹³)⁻.

In Formula (LXXXII):

(i) R¹, R², R³, R⁴, and R⁵ are each independently hydrogen, —CN, hydroxyl, —OCH₃, or halogen, wherein at least one of R¹, R², R³, R⁴, and R⁵ is —CN; and

(ii) R⁶ is a C₁-C₁₂ linear or branched alkylene, alkenylene, arylene, alkyl(arylene) or aryl(alkylene).

In Formula (LXXXIII):

(i) each occurrence of X is hydrogen, halogen, hydroxyl, or C₁-C₃ alkoxy;

(ii) R is substituted or unsubstituted C₁-C₃ alkylene or substituted or unsubstituted C₂-C₃ alkenylene; and

(iii) n is 1, 2, 3, or 4.

In Formula (LXXXIV):

(i) X is halogen; and

(ii) R is substituted or unsubstituted C₁-C₃ alkylene or substituted or unsubstituted C₂-C₃ alkenylene.

United States Patent Application Publication No. 2012/0064147 by Eibl et al., incorporated herein by this reference, discloses penetration enhancers including 3-(3-hexyloxy-2-hydroxy-propoxy)-propane-1,2-diol and 3-[2-hydroxy-3-(2-hydroxy-2-octyloxy-propoxy)-propoxy]-propane-1,2-diol.

United States Patent Application Publication No. 2009/0143330 by Levchik et al., incorporated herein by this reference, discloses penetration enhancers including polymorphic forms of SNAC.

United States Patent Application Publication No. 2009/0010882 by Bhandarkar et al., incorporated herein by this reference, discloses penetration enhancers including polymorphic forms of sodium 4-[(4-chloro-2-hydroxybenzoyl)amino]butanoate (sodium 4-CNAB).

United States Patent Application Publication No. 2012/0258911 by Gschneidner et al., incorporated herein by this reference, discloses penetration enhancers that are phenylalkylcarboxylic acids, including 4-(4-methoxyphenyl)butanoic acid, 5-(2-methoxyphenyl)pentanoic acid, 5-(3-fluorophenyl)pentanoic acid, 5-(3-methoxyphenyl)pentanoic acid, 6-(3-fluorophenyl)hexanoic acid, 3-(4-t-butylphenyl)propanoic acid, 3-(4-n-butylphenyl)propanoic acid, 3-(4-n-propylphenyl)propanoic acid, 3-(4-n-propoxyphenyl)propanoic acid, 3-(4-isopropoxyphenyl)propanoic acid, 3-(4-n-butoxyphenyl)propanoic acid, 3-(3-phenoxyphenyl)propanoic acid, 3-(3-ethoxyphenyl)propanoic acid, 3-(3-isopropoxyphenyl)propanoic acid, 3-(3-n-butoxyphenyl)propanoic acid, 3-(3-n-propoxyphenyl)propanoic acid, 3-(3-isobutoxyphenyl)propanoic acid, 3-(4-isobutoxyphenyl)propanoic acid, 4-(4-ethylphenyl)butanoic acid, 4-(4-isopropylphenyl)butanoic acid, and 5-(4-ethylphenyl)pentanoic acid or pharmaceutically acceptable salts thereof.

Additional United States patents disclosing penetration enhancers include U.S. Pat. No. 8,466,199 to Song, U.S. Pat. No. 8,492,330 to Khan et al., U.S. Pat. No. 8,541,362 to Tang et al., U.S. Pat. No. 8,546,581 to Jungheim et al., U.S. Pat. No. 8,552,039 to Herr et al., all of which are incorporated herein by this reference.

Additional United States published patent applications disclosing penetration enhancers include United States Patent Application Publication No. 2012/0264834 by Bay et al., United States Patent Application Publication No. 2011/0251125 by Bay et al., United States Patent Application Publication No. 2010/0009621 by Bay et al., United States Patent Application Publication No. 2011/0190205 by Jungheim et al., United States Patent Application Publication No. 2010/0048454 by Arbit et al., United States Patent Application Publication No. 2010/0016229 by Sarubbi, United States Patent Application Publication No. 2009/0286735 by Khan et al., all of which are incorporated herein by this reference.

Other penetration enhancers are known in the art. In general, such penetration enhancers are compounds having both hydrophobic and hydrophilic elements. The hydrophobic elements can be, for example, aromatic moieties or the aliphatic portions of medium and long-chain fatty acids. The hydrophilic elements can be, for example, carboxylic acids or their derivatives. Additionally, derivatives or isosteres of penetration enhancers described above can be used. Isosterism is defined as the presence of substituents or groups that have chemical or physical similarities and that produce broadly similar biological properties. Many isosteric substitutions or replacements are known for moieties such as methyl groups, chloro groups, bromo groups, iodo groups, methylene groups, esters, amides, aromatic moieties, carbonyl groups, carboxylic acid groups, hydroxyl groups, catechol moieties, thioether moieties, thiourea moieties, and spacer groups, and are described in R. B. Silverman, “The Organic Chemistry of Drug Design and Drug Action” (2^{nd} ed., Elsevier, 2004), pp. 29-34, incorporated herein by this reference.

In general, in compositions employing penetration enhancers such as those described above, absorption of the sodium pentosan polysulfate can be detected in subjects treated with the pharmaceutical compositions of the present invention by monitoring the plasma levels of sodium pentosan polysulfate after administration of the composition. The time it takes for an active agent to reach peak concentration in the bloodstream (tₘₐₓ) may depend on many factors such as the following: the nature of the unit dose (i.e., solid, liquid, tablet, capsule, suspension); the possible use of agents that delay absorption of the active agent in the composition being administered; the concentration of active agent and the penetration enhancer in the gastrointestinal (GI) tract, the state of nutrition of the subject, the diet of the subject, the health of the subject, and the ratio of the active agent to the penetration enhancer. In one alternative, using 4-CNAB as the penetration enhancer, the composition provides a peak plasma concentration of sodium pentosan polysulfate from about 0.1 hour to about 3 hours after the administration of the composition. Preferably, the composition provides a peak plasma concentration of sodium pentosan polysulfate from about 0.2 hour to about 0.6 hour after the administration of the composition. More preferably, the composition provides a peak plasma concentration of sodium pentosan polysulfate from about 0.3 hour to about 0.4 hour after the administration of the composition. In another alternative, the composition provides a first peak plasma concentration of sodium pentosan polysulfate at about 0.3 hours after the administration of the composition and a second peak plasma concentration of sodium pentosan polysulfate at about 1.1 hours after the administration of the composition.

The time it takes a penetration enhancer to reach a peak in the bloodstream (tₘₐₓ) may depend on many factors such as the following: the nature of the unit dose (i.e., solid, liquid, tablet, capsule, suspension); the concentration of active agent and the penetration enhancer in the gastrointestinal (GI) tract, the state of nutrition of the subject, the diet of the subject, the health of the subject, and the ratio of the active agent to the penetration enhancer. The penetration enhancers used in compositions according to the present invention are rapidly absorbed from the GI tract when orally administered in an immediate release dosage form, and preferably provide a peak plasma concentration at about 0.1 to about 8 hours after oral administration, and preferably at about 0.1 to about 3 hours after oral administration. In one preferred embodiment, the tₘₐₓ of the penetration enhancer occurs at about 0.3 to about 1.5 hours after oral administration. In certain embodiments, the penetration enhancer achieves a tₘₐₓ of about 2 hours after oral administration, and most preferably, about 1 hour after oral administration.

The amount of penetration enhancer necessary to adequately deliver an active agent into the blood stream of a subject needing the therapeutic effect of that active agent may vary depending on one or more of the following; the chemical nature of the active agent; the chemical structure of the particular penetration enhancer; the nature and extent of interaction between the active agent and penetration enhancer, including noncovalent interactions such as hydrogen bonds, salt links, hydrophobic bonds, and van der Waals interactions; the nature of the unit dose, i.e., solid, liquid, tablet, capsule, suspension; the concentration of penetration enhancer in the GI tract; the feeding state of the subject; the diet of the subject; the health of the subject and the ratio of penetration enhancer to the active agent. In a certain preferred embodiment of the invention, the amount of the penetration enhancer preferred for the pharmaceutical composition is from about 1 mg to about 2000 mg penetration enhancer, more preferably from about 1 mg to about 800 mg of the penetration enhancer, more preferably from about 50 mg to about 700 mg of the penetration enhancer, even more preferably from about 70 mg to about 700 mg of said penetration enhancer, still more preferably from about 100 to about 600 mg.

The optimum ratio of sodium pentosan polysulfate and penetration enhancer can be varied depending on the dose of sodium pentosan polysulfate to be delivered, the presence or absence of other carriers or excipients, and the specific penetration enhancer employed. One of ordinary skill can readily determine this ratio by, for example, making use of pharmacokinetic parameters such as: (1) bioavailability, defined as the degree or ratio (%) to which a drug or agent is absorbed or otherwise available to the treatment site in the body; in the case of sodium pentosan polysulfate, the treatment site is the epithelium of the lower urinary tract; bioavailability is calculated by the formula:

$$Rel . Bioavailability ( \% ) = \frac{Dose SC}{Dose Oral} \times \frac{{AUC}_{INS} Oral}{{AUC}_{INS} SC} \times 100 ;$$

(2) biopotency, defined as the degree or ratio (%) to which the drug or agent is effective to the treatment site in the body, and calculated by the formula:

$$Rel . Biopotency ( \% ) = \frac{Dose SC}{Dose Oral} \times \frac{{AUC}_{GIR} Oral}{{AUC}_{GIR} SC} \times 100 ;$$

(3) Fᵣₑₗ, which means the relative bioavailability of sodium pentosan polysulfate calculated by comparing dose-corrected oral sodium pentosan polysulfate AUC (defined below with the dose-corrected sodium pentosan polysulfate as administered by instillation AUC; (4) Kₑₗ, the terminal elimination rate constant calculated by linear regression of the terminal linear portion of the log concentration versus time curve; (5) AUC₍₀₋ₓ₎, the area under the plasma concentration-time curve using linear trapezoidal summation from time 0 to time x hours post-dose; (6) AUC₍₀₋ₜ₎, the area under the plasma concentration-time curve using linear trapezoidal summation from time 0 to time t hours post-dose, where t is the time of the last measurable concentration (Cₜ); (7) AUC_{(0-∞)}, the area under the plasma concentration-time curve from time 0 to infinity; AUC_{(0-∞)}=AUC₍₀₋ₜ₎+Cₜ/Kₑₗ; (8) AUC_{% Extrap} means the percentage of the total AUC₍₀₋₂₈₎ obtained by extrapolation; (9) AEUC₍₀₋ₜ₎ means the area under the effect-time curve calculated using the linear trapezoidal summation from time 0 to the concentration at time x hours post-dose; (10) AEUC₍₀₋ₜ₎ means the area under the effect-time curve calculated using the linear trapezoidal summation from time 0 to the concentration at time t post-dose, where t is the time of the last measurable effect (E); (11) AURC₍₀₋ₓ₎ means the area under the response-time curve calculated using the linear trapezoidal summation from time zero to the concentration at time x (Baseline Subtracted AUEC); (12) AURC₍₀₋ₓ₎ means the response-time curve calculated using the linear trapezoidal summation from time 0 to the concentration at time t (Baseline Subtracted AUEC), where t is the time of the last measurable response (R); (12) the term CL/F means the apparent total body clearance calculated as Dose/AUC_{(0-∞)}; (13) MRT means the mean residence time calculated as the ratio of the area under the first moment of the plasma concentration-time curve (AUMC) and the area under the plasma concentration-time curve (AUMC)/AUC_{(0-∞)}.

The delivery agent may be used directly by mixing one or more such agents with the active agent (e.g., sodium pentosan polysulfate) prior to administration. The delivery agent and active agent may be mixed in dry powder form or wet granulated together. To this mixture, other pharmaceutically acceptable excipients may be added. The mixture may be then tableted or placed into gelatin capsules containing a unit dose of the active agent and the delivery agent. Alternatively, the delivery agent/active agent mixture may be prepared as an oral solution or suspension. The delivery agent and active agent do not need to be mixed together prior to administration, such that, in certain embodiments, the unit dose of active agent (with or without other pharmaceutically acceptable excipients) is orally administered without the delivery agents of this invention, and the delivery agent is separately orally administered (with or without other pharmaceutically acceptable excipients) before, after, or simultaneously with the active agent.

In certain preferred embodiments, the oral dosage forms of the present invention are solid. The unmodified sodium pentosan polysulfate in dry powder form is stable, and in certain preferred embodiments is simply mixed in a desirable ratio with the delivery agent. The dry powder mixture may then be filled into gelatin capsules, with or without optional pharmaceutical excipients. Alternatively, the unmodified sodium pentosan polysulfate in dry powder form may be mixed with the delivery agent together with optional pharmaceutical excipients, and the mixture may be tableted in accordance with standard tableting procedures known to those having ordinary skill in the art.

The dosage forms of the present invention may be produced by first dissolving the active agent and delivery agents into one solution or separate solutions. The solvent will preferably be an aqueous solution, but organic solvents or aqueous organic solvent mixtures may be used when necessary to solubilize the delivery agent. If two solutions are used, the proportions of each necessary to provide the correct amount of either active agent or delivery agent are combined and the resulting solution may be dried, by lyophilization or equivalent means. In one embodiment of the invention, the oral dosage form may be dried and rehydrated prior to oral administration.

The administration mixtures may be prepared, e.g., by mixing an aqueous solution of the delivery agent with an aqueous solution of the active ingredient, such as sodium pentosan polysulfate, just prior to administration. Alternatively, the delivery agent and the biologically or chemically active ingredient can be admixed during the manufacturing process. The solutions may optionally contain additives such as phosphate buffer salts, citric acid, acetic acid, gelatin, and gum acacia.

Stabilizing additives may be incorporated into the delivery agent solution. With some drugs, the presence of such additives promotes the stability and dispersibility of the agent in solution. The stabilizing additives may be employed at a concentration ranging from about 0.1% to about 5% (w/v), preferably about 0.5% (w/v). Suitable, but non-limiting, examples of stabilizing additives include gum acacia, gelatin, methyl cellulose, polyethylene glycol, carboxylic acids and salts thereof, and polylysine. The preferred stabilizing additives are gum acacia, gelatin and methyl cellulose.

The amount of active agent, e.g., sodium pentosan polysulfate, is an amount effective to accomplish the purpose of the particular active agent. The amount in the composition is a therapeutically effective dose, i.e., a pharmacologically or biologically effective amount. However, the amount can be less than a pharmacologically or biologically effective amount when the composition is used in a dosage unit form, such as a capsule, a tablet or a liquid, because the dosage unit form may contain a multiplicity of delivery agent/biologically or chemically active agent compositions or may contain a divided pharmacologically or biologically effective amount. The total effective amounts can then be administered in cumulative units containing, in total, pharmacologically or biologically or chemically active amounts of biologically or pharmacologically active agent.

The oral dosage forms of the present invention, containing a mixture of the active agent, e.g., sodium pentosan polysulfate, and the delivery agent, e.g., 4-CNAB or another penetration enhancer described herein, or separately containing the active agent and the penetration enhancer, may include additional materials known to those skilled in the art as fillers, excipients, or carriers. These materials are also known generally as pharmaceutically acceptable carriers. Any excipient or ingredient, including pharmaceutical ingredients or excipients, can be used. Such pharmaceutical excipients include, for example, the following: acidifying agents (acetic acid, glacial acetic acid, citric acid, fumaric acid, hydrochloric acid, diluted hydrochloric acid, malic acid, nitric acid, phosphoric acid, diluted phosphoric acid, sulfuric acid, tartaric acid); aerosol propellants (butane, dichlorodifluoromethane, dichlorotetrafluoroethane, isobutane, propane, trichloromonofluoromethane); air displacements (carbon dioxide, nitrogen); alcohol denaturants (denatonium benzoate, methyl isobutyl ketone, sucrose octacetate); alkalizing agents (strong ammonia solution, ammonium carbonate, diethanolamine, diisopropanolamine, potassium hydroxide, sodium bicarbonate, sodium borate, sodium carbonate, sodium hydroxide, trolamine); anticaking agents (see glidants); antifoaming agents (dimethicone, simethicone); antimicrobial preservatives (benzalkonium chloride, benzalkonium chloride solution, benzethonium chloride, benzoic acid, benzyl alcohol, butylparaben, cetylpyridinium chloride, chlorobutanol, chlorocresol, cresol, dehydroacetic acid, ethylparaben, methylparaben, methylparaben sodium, phenol, phenylethyl alcohol, phenylmercuric acetate, phenylmercuric nitrate, potassium benzoate, potassium sorbate, propylparaben, propylparaben sodium, sodium benzoate, sodium dehydroacetate, sodium propionate, sorbic acid, thimerosal, thymol); antioxidants (ascorbic acid, ascorbyl palmitate, butylated hydroxyanisole, butylated hydroxytoluene, hypophosphorous acid, monothioglycerol, propyl gallate, sodium formaldehyde sulfoxylate, sodium metabisulfite, sodium thiosulfate, sulfur dioxide, tocopherol, tocopherols excipient); buffering agents (acetic acid, ammonium carbonate, ammonium phosphate, boric acid, citric acid, lactic acid, phosphoric acid, potassium citrate, potassium metaphosphate, potassium phosphate monobasic, sodium acetate, sodium citrate, sodium lactate solution, dibasic sodium phosphate, monobasic sodium phosphate, bicarbonate, Tris (Tris(hydroxymethyl)aminomethane), MOPS (3-(N-morpholino)propanesulfonic acid), HEPES (N-(2-hydroxyethyl)piperazine-N′-(2-ethanesulfonic acid), ACES (2-[(2-amino-2-oxoethyl)amino]ethanesulfonic acid), ADA (N-(2-acetamido)₂-iminodiacetic acid), AMPSO (3-[(1,1-dimethyl-2-hydroxyethylamino]-2-propanesulfonic acid), BES (N,N-bis(2-hydroxyethyl)-2-aminoethanesulfonic acid, Bicine (N,N-bis(2-hydroxyethylglycine), Bis-Tris (bis-(2-hydroxyethyl)imino-tris(hydroxymethyl)methane, CAPS (3-(cyclohexylamino)-1-propanesulfonic acid), CAPSO (3-(cyclohexylamino)-2-hydroxy-1-propanesulfonic acid), CHES (2-(N-cyclohexylamino)ethanesulfonic acid), DIPSO (3-[N,N-bis(2-hydroxyethylamino]-2-hydroxy-propanesulfonic acid), HEPPS (N-(2-hydroxyethylpiperazine)-N′-(3-propanesulfonic acid), HEPPSO (N-(2-hydroxyethyl)piperazine-N′-(2-hydroxypropanesulfonic acid), MES (2-(N-morpholino)ethanesulfonic acid), triethanolamine, imidazole, glycine, ethanolamine, phosphate, MOPSO (3-(N-morpholino)-2-hydroxypropanesulfonic acid), PIPES (piperazine-N,N′-bis(2-ethanesulfonic acid), POPSO (piperazine-N,N′-bis(2-hydroxypropaneulfonic acid), TAPS (N-tris[hydroxymethyl)methyl-3-aminopropanesulfonic acid), TAPSO (3-[N-tris(hydroxymethyl)methylamino]-2-hydroxy-propanesulfonic acid), TES (N-tris(hydroxymethyl)methyl-2-aminoethanesulfonic acid), tricine (N-tris(hydroxymethyl)methylglycine), 2-amino-2-methyl-1,3-propanediol, and 2-amino-2-methyl-1-propanol); capsule lubricants (see tablet and capsule lubricants); chelating agents (edetate disodium, ethylenediaminetetraacetic acid and salts); coating agents (sodium carboxymethyl-cellulose, cellulose acetate, cellulose acetate phthalate, ethylcellulose, gelatin, pharmaceutical glaze, hydroxypropyl cellulose, hydroxypropyl methylcellulose, hydroxypropyl methylcellulose phthalate, methacrylic acid copolymer, methylcellulose, polyethylene glycol, polyvinyl acetate phthalate, shellac, sucrose, titanium dioxide, carnauba wax, microcystalline wax, zein); colorants (caramel, red, yellow, black or blends, ferric oxide); complexing agents (ethylenediaminetetraacetic acid and salts (EDTA), gentisic acid ethanolamide, oxyquinoline sulfate); desiccants (calcium chloride, calcium sulfate, silicon dioxide); emulsifying and/or solubilizing agents (acacia, cholesterol, diethanolamine (adjunct), glyceryl monostearate, lanolin alcohols, lecithin, mono- and di-glycerides, monoethanolamine (adjunct), oleic acid (adjunct), oleyl alcohol (stabilizer), poloxamer, polyoxyethylene 50 stearate, polyoxyl 35 castor oil, polyoxyl 40 hydrogenated castor oil, polyoxyl 10 oleyl ether, polyoxyl 20 cetostearyl ether, polyoxyl 40 stearate, polysorbate 20, polysorbate 40, polysorbate 60, polysorbate 80, propylene glycol diacetate, propylene glycol monostearate, sodium lauryl sulfate, sodium stearate, sorbitan monolaurate, soritan monooleate, sorbitan monopalmitate, sorbitan monostearate, stearic acid, triethanolamine, emulsifying wax); filtering aids (powdered cellulose, purified siliceous earth); flavors and perfumes (anethole, benzaldehyde, ethyl vanillin, menthol, methyl salicylate, monosodium glutamate, orange flower oil, peppermint, peppermint oil, peppermint spirit, rose oil, stronger rose water, thymol, tolu balsam tincture, vanilla, vanilla tincture, vanillin); glidants and/or anticaking agents (calcium silicate, magnesium silicate, colloidal silicon dioxide, talc); humectants (glycerin, hexylene glycol, propylene glycol, sorbitol); plasticizers (castor oil, diacetylated monoglycerides, diethyl phthalate, glycerin, mono- and di-acetylated monoglycerides, polyethylene glycol, propylene glycol, triacetin, triethyl citrate); polymers (e.g., cellulose acetate, alkyl celluloses, hydroxyalkylcelluloses, acrylic polymers and copolymers); solvents (acetone, alcohol, diluted alcohol, amylene hydrate, benzyl benzoate, butyl alcohol, carbon tetrachloride, chloroform, corn oil, cottonseed oil, ethyl acetate, glycerin, hexylene glycol, isopropyl alcohol, methyl alcohol, methylene chloride, methyl isobutyl ketone, mineral oil, peanut oil, polyethylene glycol, propylene carbonate, propylene glycol, sesame oil, water for injection, sterile water for injection, sterile water for irrigation, purified water); sorbents (powdered cellulose, charcoal, purified siliceous earth); carbon dioxide sorbents (barium hydroxide lime, soda lime); stiffening agents (hydrogenated castor oil, cetostearyl alcohol, cetyl alcohol, cetyl esters wax, hard fat, paraffin, polyethylene excipient, stearyl alcohol, emulsifying wax, white wax, yellow wax); suspending and/or viscosity-increasing agents (acacia, agar, alginic acid, aluminum monostearate, bentonite, purified bentonite, magma bentonite, carbomer 934p, carboxymethylcellulose calcium, carboxymethylcellulose sodium, carboxymethycellulose sodium 12, carrageenan, microcrystalline and carboxymethylcellulose sodium cellulose, dextrin, gelatin, guar gum, hydroxyethyl cellulose, hydroxypropyl cellulose, hydroxypropyl methylcellulose, magnesium aluminum silicate, methylcellulose, pectin, polyethylene oxide, polyvinyl alcohol, povidone, propylene glycol alginate, silicon dioxide, colloidal silicon dioxide, sodium alginate, tragacanth, xanthan gum); sweetening agents (aspartame, dextrates, dextrose, excipient dextrose, fructose, mannitol, saccharin, calcium saccharin, sodium saccharin, sorbitol, solution sorbitol, sucrose, compressible sugar, confectioner's sugar, syrup); tablet binders (acacia, alginic acid, sodium carboxymethylcellulose, microcrystalline cellulose, dextrin, ethylcellulose, gelatin, liquid glucose, guar gum, hydroxypropyl methylcellulose, methycellulose, polyethylene oxide, povidone, pregelatinized starch, syrup); tablet and/or capsule diluents (calcium carbonate, dibasic calcium phosphate, tribasic calcium phosphate, calcium sulfate, microcrystalline cellulose, powdered cellulose, dextrates, dextrin, dextrose excipient, fructose, kaolin, lactose, mannitol, sorbitol, starch, pregelatinized starch, sucrose, compressible sugar, confectioner's sugar); tablet disintegrants (alginic acid, microcrystalline cellulose, croscarmellose sodium, corspovidone, polacrilin potassium, sodium starch glycolate, starch, pregelatinized starch); tablet and/or capsule lubricants (calcium stearate, glyceryl behenate, magnesium stearate, light mineral oil, polyethylene glycol, sodium stearyl fumarate, stearic acid, purified stearic acid, talc, hydrogenated vegetable oil, zinc stearate); tonicity agents (dextrose, glycerin, mannitol, potassium chloride, sodium chloride); vehicle: flavored and/or sweetened (aromatic elixir, compound benzaldehyde elixir, iso-alcoholic elixir, peppermint water, sorbitol solution, syrup, tolu balsam syrup); vehicle: oleaginous (almond oil, corn oil, cottonseed oil, ethyl oleate, isopropyl myristate, isopropyl palmitate, mineral oil, light mineral oil, myristyl alcohol, octyldodecanol, olive oil, peanut oil, persic oil, sesame oil, soybean oil, squalane); vehicle: solid carrier (sugar spheres); vehicle: sterile (bacteriostatic water for injection, bacteriostatic sodium chloride for injection); viscosity-increasing (see suspending agent); water-repelling agent (cyclomethicone, dimethicone, simethicone); and wetting and/or solubilizing agent (benzalkonium chloride, benzethonium chloride, cetylpyridinium chloride, docusate sodium, nonoxynol 9, nonoxynol 10, octoxynol 9, poloxamer, polyoxyl 35 castor oil, polyoxyl 40, hydrogenated castor oil, polyoxyl 50 stearate, polyoxyl 10 oleyl ether, polyoxyl 20, cetostearyl ether, polyoxyl 40 stearate, polysorbate 20, polysorbate 40, polysorbate 60, polysorbate 80, sodium lauryl sulfate, sorbitan monolaureate, sorbitan monooleate, sorbitan monopalmitate, sorbitan monostearate, tyloxapol). This list is not meant to be exclusive, but instead merely representative of the classes of excipients and the particular excipients which may be used in oral dosage forms of the present invention. Other excipients

Other penetration enhancers are known in the art. For example, and not by way of limitation, pharmaceutically acceptable salts of penetration enhancers described above in non-ionized form can be employed. A salt, for example, can be formed between an anion and a positively charged group (e.g., amino) on a penetration enhancer in ionized form. Suitable anions include chloride, bromide, iodide, carbonate, nitrate, sulfate, bisulfate, phosphate, monohydrogen phosphate, dihydrogen phosphate, metaphosphate, pyrophosphate, formate, acetate, adipate, butyrate, propionate, succinate, glycolate, gluconate, lactate, malate, tartrate, citrate, ascorbate, glucuronate, maleate, fumarate, pyruvate, aspartate, glutamate, benzoate, anthranilate, mesylate, 4′-hydroxybenzoate, phenylacetate, mandelate, embonate (pamoate), methanesulfonate, ethanesulfonate, ethanedisulfonate, benzenesulfonate, pantothenate, 2-hydroxyethanesulfonate, p-toluenesulfonate, sulfanilate, cyclohexylaminosulfonate, camphorate, camphorsulfonate, digluconate, cyclopentanepropionate, dodecylsulfonate, glucoheptanoate, glycerophosphonate, heptanoate, hexanoate, 2-hydroxyethanesulfonate, nicotinate, isonicotinate, 1-naphthalenesulfonate, 2-naphthalenesulfonate, oxalate, palmoate, pectinate, persulfurate, 2-phenylpropionate, picrate, pivalate, thiocyanate, mesylate, undecanoate, stearate, algenate, P3-hydroxybutyrate, salicylate, galactarate, galacturonate, caprylate, isobutyrate, malonate, suberate, sebacate, chlorobenzoate, methylbenzoate, dinitrobenzoate, phthalate, phenylacetate, isethionate, lactobionate, p-aminobenzoate, sulfamate, diethylacetate, pimelate, aminosulfonate, acrylate, γ-hydroxybutyrate, and methoxybenzoate. Likewise, a salt can also be formed between a cation and a negatively charged group (e.g., carboxylate) on an agent. Suitable cations include sodium, aluminum, lithium, calcium, magnesium, zinc, ammonium, caffeine, arginine, diethylamine, N-ethylpiperidine, histidine, glucamine, isopropylamine, lysine, morpholine, N-ethylmorpholine, piperazine, piperidine, triethylamine, trimethylamine, ethanolamine, diethanolamine, N-methylglucamine, and tris(hydroxymethyl)aminomethane. Potassium as a counterion is typically avoided because of its association with the etiology of interstitial cystitis and other lower urinary tract dysfunctions, but may be used in applications in which the intention is to treat diseases and conditions not exacerbated by the administration of potassium or in which administration is performed by a route in which the pentosan polysulfate salt does not significantly reach the urinary tract.

Other penetration enhancers are known in the art and can be used. Typically, such penetration enhancers are compounds including both hydrophobic groups, such as phenyl groups, naphthyl groups, cyclohexyl groups, and long-chain aliphatic groups, and hydrophilic groups, such as carboxylic acid groups, carboxylic acid ester groups, amide groups, amino groups, and carbonyl groups. Multiple hydrophobic groups, such as multiple phenyl groups, can be included in the penetration enhancers.

Penetration enhancers such as those described above, and other penetration enhancers known in the art or constructed according to the principles described above, can be optionally substituted with one or more groups that do not substantially affect the ability of the penetration enhancer to enhance the absorption or bioavailability of sodium pentosan polysulfate when administered orally. Definitions for a number of common groups that can be used as optional substituents are provided below; however, the omission of any group from these definitions cannot be taken to mean that such a group cannot be used as an optional substituent as long as the chemical and pharmacological requirements for an optional substituent are satisfied.

As used herein, the term “optionally substituted” indicates that the particular group or groups referred to as optionally substituted may have no non-hydrogen substituents, or the group or groups may have one or more non-hydrogen substituents consistent with the chemistry and pharmacological activity of the resulting molecule. If not otherwise specified, the total number of such substituents that may be present is equal to the total number of hydrogen atoms present on the unsubstituted form of the group being described; fewer than the maximum number of such substituents may be present. Where an optional substituent is attached via a double bond, such as a carbonyl oxygen (C═O), the group takes up two available valences on the carbon atom to which the optional substituent is attached, so the total number of substituents that may be included is reduced according to the number of available valences. As used herein, the term “substituted,” whether used as part of “optionally substituted” or otherwise, when used to modify a specific group, moiety, or radical, means that one or more hydrogen atoms are, each, independently of each other, replaced with the same or different substituent or substituents.

Substituent groups useful for substituting saturated carbon atoms in the specified group, moiety, or radical include, but are not limited to, —Z^{a}, ═O, —OZ^{b}, —SZ^{b}, ═S⁻, —NZ^{c}Z^{c}, ═NZ^{b}, ═N—OZ^{b}, trihalomethyl, —CF₃, —CN, —OCN, —SCN, —NO, —NO₂, ═N₂, —N₃, —S(O)₂Z^{b}, —S(O)₂NZ^{b}, —S(O₂)O⁻, —S(O₂)OZ^{b}, —OS(O₂)OZ^{b}, —OS(O₂)O⁻, —OS(O₂)OZ^{b}, —P(O)(O⁻)₂, —P(O)(OZ^{b})(O⁻), —P(O)(OZ^{b})(OZ^{b}), —C(O)Z^{b}, —C(S)Z^{b}, —C(NZ^{b})Z^{b}, —C(O)O⁻, —C(O)OZ^{b}, —C(S)OZ^{b}, —C(O)NZZ^{c}, —C(NZ^{b})NZ^{c}Z^{c}, —OC(O)Z^{b}, —OC(S)Z^{b}, —OC(O)O—, —OC(O)OZ^{b}, —OC(S)OZ^{b}, —NZ^{b}C(O)Z^{b}, —NZ^{b}C(S)Z^{b}, —NZ^{b}C(O)O⁻, —NZ^{b}C(O)OZ^{b}, —NZ^{b}C(S)OZ^{b}, —NZ^{b}C(O)NZCZ^{c}, —NZ^{b}C(NZ^{b})Z^{b}, —NZ^{b}C(NZ^{b})NZ^{c}Z^{c}, wherein Z^{a} is selected from the group consisting of alkyl, cycloalkyl, heteroalkyl, cycloheteroalkyl, aryl, arylalkyl, heteroaryl and heteroarylalkyl; each Z^{b} is independently hydrogen or Z^{a}; and each Z^{c} is independently Z^{b} or, alternatively, the two Z's may be taken together with the nitrogen atom to which they are bonded to form a 4-, 5-, 6-, or 7-membered cycloheteroalkyl ring structure which may optionally include from 1 to 4 of the same or different heteroatoms selected from the group consisting of N, O, and S. As specific examples, —NZ^{c}Z^{c} is meant to include —NH₂, —NH-alkyl, —N-pyrrolidinyl, and —N-morpholinyl, but is not limited to those specific alternatives and includes other alternatives known in the art. Similarly, as another specific example, a substituted alkyl is meant to include -alkylene-O-alkyl, -alkylene-heteroaryl, -alkylene-cycloheteroaryl, -alkylene-C(O)OZ^{b}, -alkylene-C(O)NZ^{b}Z^{b}, and —CH₂—CH₂—C(O)—CH₃, but is not limited to those specific alternatives and includes other alternatives known in the art. The one or more substituent groups, together with the atoms to which they are bonded, may form a cyclic ring, including, but not limited to, cycloalkyl and cycloheteroalkyl.

Similarly, substituent groups useful for substituting unsaturated carbon atoms in the specified group, moiety, or radical include, but are not limited to, —Z^{a}, halo, —O⁻, —OZ^{b}, —SZ^{b}, —S⁻, —NZ^{c}Z^{c}, trihalomethyl, —CF₃, —CN, —OCN, —SCN, —NO, —NO₂, —N₃, —S(O)₂Z^{b}, —S(O₂)O⁻, —S(O₂)OZ^{b}, —OS(O₂)OZ^{b}, —OS(O₂)O⁻, —P(O)(O⁻)₂, —P(O)(OZ^{b})(O⁻), —P(O)(OZ^{b})(OZ^{b}), —C(O)Z^{b}, —C(S)Z^{b}, —C(NZ^{b})Z^{b}, —C(O)O—, —C(O)OZ^{b}, —C(S)OZ^{b}, —C(O)NZ^{c}Z^{c}, —C(NZ^{b})NZ^{c}Z^{c}, —OC(O)Z^{b}, —OC(S)Z^{b}, —OC(O)O—, —OC(O)OZ^{b}, —OC(S)OZ^{b}, —NZ^{b}C(O)OZ^{b}, —NZ^{b}C(S)OZ^{b}, —NZ^{b}C(O)NZCZ^{c}, —NZ^{b}C(NZ^{b})Z^{b}, and —NZ^{b}C(NZ^{b})NZ^{c}Z^{c}, wherein Z^{a}, Z^{b}, and Z^{c} are as defined above.

Similarly, substituent groups useful for substituting nitrogen atoms in heteroalkyl and cycloheteroalkyl groups include, but are not limited to, —Z^{a}, halo, —O—, —OZ^{b}, —SZ^{b}, —S—, —NZ^{c}Z^{c}, trihalomethyl, —CF₃, —CN, —OCN, —SCN, —NO, —NO₂, —S(O)₂Z^{b}, —S(O₂)O—, —S(O₂)OZ^{b}, —OS(O₂)OZ^{b}, —OS(O₂)O—, —P(O)(O⁻)₂, —P(O)(OZ^{b})(O⁻), —P(O)(OZ^{b})(OZ^{b}), —C(O)Z^{b}, —C(S)Z^{b}, —C(NZ^{b})Z^{b}, —C(O)OZ^{b}, —C(S)OZ^{b}, —C(O)NZ^{c}Z^{c}, —C(NZ^{b})NZ^{c}Z^{c}, —OC(O)Z^{b}, —OC(S)Z^{b}, —OC(O)OZ^{b}, —OC(S)OZ^{b}, —NZ^{b}C(O)Z^{b}, —NZ^{b}C(S)Z^{b}, —NZ^{b}C(O)OZ^{b}, —NZ^{b}C(S)OZ^{b}, —NZ^{b}C(O)NZ^{c}Z^{c}, —NZ^{b}C(NZ^{b})Z^{b}, and —NZ^{b}C(NZ^{b})NZ^{c}Z^{c}, wherein Z^{a}, Z^{b}, and Z^{c} are as defined above.

The compounds described herein may contain one or more chiral centers and/or double bonds and therefore, may exist as stereoisomers, such as double-bond isomers (i.e., geometric isomers such as E and Z), enantiomers or diastereomers. The invention includes each of the isolated stereoisomeric forms (such as the enantiomerically pure isomers, the E and Z isomers, and other stereoisomeric forms) as well as mixtures of stereoisomers in varying degrees of chiral purity or percentage of E and Z, including racemic mixtures, mixtures of diastereomers, and mixtures of E and Z isomers. Accordingly, the chemical structures depicted herein encompass all possible enantiomers and stereoisomers of the illustrated compounds including the stereoisomerically pure form (e.g., geometrically pure, enantiomerically pure or diastereomerically pure) and enantiomeric and stereoisomeric mixtures. Enantiomeric and stereoisomeric mixtures can be resolved into their component enantiomers or stereoisomers using separation techniques or chiral synthesis techniques well known to the skilled artisan. The invention includes each of the isolated stereoisomeric forms as well as mixtures of stereoisomers in varying degrees of chiral purity, including racemic mixtures. It also encompasses the various diastereomers. Other structures may appear to depict a specific isomer, but that is merely for convenience, and is not intended to limit the invention to the depicted olefin isomer. When the chemical name does not specify the isomeric form of the compound, it denotes any one of the possible isomeric forms or mixtures of those isomeric forms of the compound.

The penetration enhancers may also exist in several tautomeric forms, and the depiction herein of one tautomer is for convenience only, and is also understood to encompass other tautomers of the form shown. Accordingly, the chemical structures depicted herein encompass all possible tautomeric forms of the illustrated compounds. The term “tautomer” as used herein refers to isomers that change into one another with great ease so that they can exist together in equilibrium. For example, ketone and enol are two tautomeric forms of one compound.

As used herein, the term “solvate” means a compound formed by solvation (the combination of solvent molecules with molecules or ions of the solute), or an aggregate that consists of a solute ion or molecule, i.e., a compound of the invention, with one or more solvent molecules. When water is the solvent, the corresponding solvate is a “hydrate.” Examples of hydrates include, but are not limited to, hemihydrate, monohydrate, dihydrate, trihydrate, hexahydrate, and other hydrated forms. It should be understood by one of ordinary skill in the art that the pharmaceutically acceptable salt and/or prodrug of the present compound may also exist in a solvate form. The solvate is typically formed via hydration which is either part of the preparation of the present compound or through natural absorption of moisture by the anhydrous compound of the present invention.

As used herein, the term “ester” means any ester of a present compound in which any of the —COOH functions of the molecule is replaced by a —COOR function, in which the R moiety of the ester is any carbon-containing group which forms a stable ester moiety, including but not limited to alkyl, alkenyl, alkynyl, cycloalkyl, cycloalkylalkyl, aryl, arylalkyl, heterocyclyl, heterocyclylalkyl and substituted derivatives thereof. The hydrolyzable esters of the present compounds are the compounds whose carboxyls are present in the form of hydrolyzable ester groups. That is, these esters are pharmaceutically acceptable and can be hydrolyzed to the corresponding carboxyl acid in vivo.

In addition to the substituents described above, alkyl, alkenyl and alkynyl groups can alternatively or in addition be substituted by C₁-C₈ acyl, C₂-C₈ heteroacyl, C₆-C₁₀ aryl, C₃-C₈ cycloalkyl, C₃-C₈ heterocyclyl, or C₅-C₁₀ heteroaryl, each of which can be optionally substituted. Also, in addition, when two groups capable of forming a ring having 5 to 8 ring members are present on the same or adjacent atoms, the two groups can optionally be taken together with the atom or atoms in the substituent groups to which they are attached to form such a ring.

“Heteroalkyl,” “heteroalkenyl,” and “heteroalkynyl” and the like are defined similarly to the corresponding hydrocarbyl (alkyl, alkenyl and alkynyl) groups, but the ‘hetero’ terms refer to groups that contain 1-3 O, S or N heteroatoms or combinations thereof within the backbone residue; thus at least one carbon atom of a corresponding alkyl, alkenyl, or alkynyl group is replaced by one of the specified heteroatoms to form, respectively, a heteroalkyl, heteroalkenyl, or heteroalkynyl group. For reasons of chemical stability, it is also understood that, unless otherwise specified, such groups do not include more than two contiguous heteroatoms except where an oxo group is present on N or S as in a nitro or sulfonyl group.

While “alkyl” as used herein includes cycloalkyl and cycloalkylalkyl groups, the term “cycloalkyl” may be used herein to describe a carbocyclic non-aromatic group that is connected via a ring carbon atom, and “cycloalkylalkyl” may be used to describe a carbocyclic non-aromatic group that is connected to the molecule through an alkyl linker.

Similarly, “heterocyclyl” may be used to describe a non-aromatic cyclic group that contains at least one heteroatom (typically selected from N, O and S) as a ring member and that is connected to the molecule via a ring atom, which may be C (carbon-linked) or N (nitrogen-linked); and “heterocyclylalkyl” may be used to describe such a group that is connected to another molecule through a linker. The heterocyclyl can be fully saturated or partially saturated, but non-aromatic. The sizes and substituents that are suitable for the cycloalkyl, cycloalkylalkyl, heterocyclyl, and heterocyclylalkyl groups are the same as those described above for alkyl groups. The heterocyclyl groups typically contain 1, 2 or 3 heteroatoms, selected from N, O and S as ring members; and the N or S can be substituted with the groups commonly found on these atoms in heterocyclic systems. As used herein, these terms also include rings that contain a double bond or two, as long as the ring that is attached is not aromatic. The substituted cycloalkyl and heterocyclyl groups also include cycloalkyl or heterocyclic rings fused to an aromatic ring or heteroaromatic ring, provided the point of attachment of the group is to the cycloalkyl or heterocyclyl ring rather than to the aromatic/heteroaromatic ring.

As used herein, “acyl” encompasses groups comprising an alkyl, alkenyl, alkynyl, aryl or arylalkyl radical attached at one of the two available valence positions of a carbonyl carbon atom, and heteroacyl refers to the corresponding groups wherein at least one carbon other than the carbonyl carbon has been replaced by a heteroatom chosen from N, O and S.

Acyl and heteroacyl groups are bonded to any group or molecule to which they are attached through the open valence of the carbonyl carbon atom. Typically, they are C₁-C₈ acyl groups, which include formyl, acetyl, pivaloyl, and benzoyl, and C₂-C₈ heteroacyl groups, which include methoxyacetyl, ethoxycarbonyl, and 4-pyridinoyl.

Similarly, “arylalkyl” and “heteroarylalkyl” refer to aromatic and heteroaromatic ring systems which are bonded to their attachment point through a linking group such as an alkylene, including substituted or unsubstituted, saturated or unsaturated, cyclic or acyclic linkers. Typically the linker is C₁-C₈ alkyl. These linkers may also include a carbonyl group, thus making them able to provide substituents as an acyl or heteroacyl moiety. An aryl or heteroaryl ring in an arylalkyl or heteroarylalkyl group may be substituted with the same substituents described above for aryl groups. Preferably, an arylalkyl group includes a phenyl ring optionally substituted with the groups defined above for aryl groups and a C₁-C₄ alkylene that is unsubstituted or is substituted with one or two C₁-C₄ alkyl groups or heteroalkyl groups, where the alkyl or heteroalkyl groups can optionally cyclize to form a ring such as cyclopropane, dioxolane, or oxacyclopentane. Similarly, a heteroarylalkyl group preferably includes a C₅-C₆ monocyclic heteroaryl group that is optionally substituted with the groups described above as substituents typical on aryl groups and a C₁-C₄ alkylene that is unsubstituted or is substituted with one or two C₁-C₄ alkyl groups or heteroalkyl groups, or it includes an optionally substituted phenyl ring or C₅-C₆ monocyclic heteroaryl and a C₁-C₄ heteroalkylene that is unsubstituted or is substituted with one or two C₁-C₄ alkyl or heteroalkyl groups, where the alkyl or heteroalkyl groups can optionally cyclize to form a ring such as cyclopropane, dioxolane, or oxacyclopentane.

Where an arylalkyl or heteroarylalkyl group is described as optionally substituted, the substituents may be on either the alkyl or heteroalkyl portion or on the aryl or heteroaryl portion of the group. The substituents optionally present on the alkyl or heteroalkyl portion are the same as those described above for alkyl groups generally; the substituents optionally present on the aryl or heteroaryl portion are the same as those described above for aryl groups generally.

“Arylalkyl” groups as used herein are hydrocarbyl groups if they are unsubstituted, and are described by the total number of carbon atoms in the ring and alkylene or similar linker. Thus a benzyl group is a C7-arylalkyl group, and phenylethyl is a C8-arylalkyl.

“Heteroarylalkyl” as described above refers to a moiety comprising an aryl group that is attached through a linking group, and differs from “arylalkyl” in that at least one ring atom of the aryl moiety or one atom in the linking group is a heteroatom selected from N, O and S. The heteroarylalkyl groups are described herein according to the total number of atoms in the ring and linker combined, and they include aryl groups linked through a heteroalkyl linker; heteroaryl groups linked through a hydrocarbyl linker such as an alkylene; and heteroaryl groups linked through a heteroalkyl linker. Thus, for example, C7-heteroarylalkyl would include pyridylmethyl, phenoxy, and N-pyrrolylmethoxy.

“Alkylene” as used herein refers to a divalent hydrocarbyl group; because it is divalent, it can link two other groups together. Typically it refers to —(CH₂)ₙ— where n is 1-8 and preferably n is 1-4, though where specified, an alkylene can also be substituted by other groups, and can be of other lengths, and the open valences need not be at opposite ends of a chain. The general term “alkylene” encompasses more specific examples such as “ethylene,” wherein n is 2, “propylene,” wherein n is 3, and “butylene,” wherein n is 4. The hydrocarbyl groups of the alkylene can be optionally substituted as described above.

In general, any alkyl, alkenyl, alkynyl, acyl, or aryl or arylalkyl group that is contained in a substituent may itself optionally be substituted by additional substituents. The nature of these substituents is similar to those recited with regard to the primary substituents themselves if the substituents are not otherwise described.

“Amino” as used herein refers to —NH₂, but where an amino is described as “substituted” or “optionally substituted”, the term includes NR′R″ wherein each R′ and R″ is independently H, or is an alkyl, alkenyl, alkynyl, acyl, aryl, or arylalkyl group, and each of the alkyl, alkenyl, alkynyl, acyl, aryl, or arylalkyl groups is optionally substituted with the substituents described herein as suitable for the corresponding group; the R′ and R″ groups and the nitrogen atom to which they are attached can optionally form a 3- to 8-membered ring which may be saturated, unsaturated or aromatic and which contains 1-3 heteroatoms independently selected from N, O and S as ring members, and which is optionally substituted with the substituents described as suitable for alkyl groups or, if NR′R″ is an aromatic group, it is optionally substituted with the substituents described as typical for heteroaryl groups.

As used herein, the term “carbocycle,” “carbocyclyl,” or “carbocyclic” refers to a cyclic ring containing only carbon atoms in the ring, whereas the term “heterocycle” or “heterocyclic” refers to a ring comprising a heteroatom. The carbocyclyl can be fully saturated or partially saturated, but non-aromatic. For example, the general term “carbocyclyl” encompasses cycloalkyl. The carbocyclic and heterocyclic structures encompass compounds having monocyclic, bicyclic or multiple ring systems; and such systems may mix aromatic, heterocyclic, and carbocyclic rings. Mixed ring systems are described according to the ring that is attached to the rest of the compound being described.

As used herein, the term “heteroatom” refers to any atom that is not carbon or hydrogen, such as nitrogen, oxygen or sulfur. When it is part of the backbone or skeleton of a chain or ring, a heteroatom must be at least divalent, and will typically be selected from N, O, P, and S.

As used herein, the term “alkanoyl” refers to an alkyl group covalently linked to a carbonyl (C═O) group. The term “lower alkanoyl” refers to an alkanoyl group in which the alkyl portion of the alkanoyl group is C₁-C₆. The alkyl portion of the alkanoyl group can be optionally substituted as described above. The term “alkylcarbonyl” can alternatively be used. Similarly, the terms “alkenylcarbonyl” and “alkynylcarbonyl” refer to an alkenyl or alkynyl group, respectively, linked to a carbonyl group.

As used herein, the term “alkoxy” refers to an alkyl group covalently linked to an oxygen atom; the alkyl group can be considered as replacing the hydrogen atom of a hydroxyl group. The term “lower alkoxy” refers to an alkoxy group in which the alkyl portion of the alkoxy group is C₁-C₆. The alkyl portion of the alkoxy group can be optionally substituted as described above. As used herein, the term “haloalkoxy” refers to an alkoxy group in which the alkyl portion is substituted with one or more halo groups.

As used herein, the term “sulfo” refers to a sulfonic acid (—SO₃H) substituent.

As used herein, the term “sulfamoyl” refers to a substituent with the structure —S(O₂)NH₂, wherein the nitrogen of the NH₂ portion of the group can be optionally substituted as described above.

As used herein, the term “carboxyl” refers to a group of the structure —C(O₂)H.

As used herein, the term “carbamyl” refers to a group of the structure —C(O₂)NH₂, wherein the nitrogen of the NH₂ portion of the group can be optionally substituted as described above.

As used herein, the terms “monoalkylaminoalkyl” and “dialkylaminoalkyl” refer to groups of the structure -Alk₁-NH-Alk₂ and -Alk-N(Alk₂)(Alk3), wherein Alk₁, Alk₂, and Alk₃ refer to alkyl groups as described above.

As used herein, the term “alkylsulfonyl” refers to a group of the structure —S(O)₂-Alk wherein Alk refers to an alkyl group as described above. The terms “alkenylsulfonyl” and “alkynylsulfonyl” refer analogously to sulfonyl groups covalently bound to alkenyl and alkynyl groups, respectively. The term “arylsulfonyl” refers to a group of the structure —S(O)₂—Ar wherein Ar refers to an aryl group as described above. The term “aryloxyalkylsulfonyl” refers to a group of the structure —S(O)₂-Alk-O—Ar, where Alk is an alkyl group as described above and Ar is an aryl group as described above. The term “arylalkylsulfonyl” refers to a group of the structure —S(O)₂-AlkAr, where Alk is an alkyl group as described above and Ar is an aryl group as described above.

As used herein, the term “alkyloxycarbonyl” refers to an ester substituent including an alkyl group wherein the carbonyl carbon is the point of attachment to the molecule. An example is ethoxycarbonyl, which is CH₃CH₂OC(O)—. Similarly, the terms “alkenyloxycarbonyl,” “alkynyloxycarbonyl,” and “cycloalkylcarbonyl” refer to similar ester substituents including an alkenyl group, alkenyl group, or cycloalkyl group respectively. Similarly, the term “aryloxycarbonyl” refers to an ester substituent including an aryl group wherein the carbonyl carbon is the point of attachment to the molecule. Similarly, the term “aryloxyalkylcarbonyl” refers to an ester substituent including an alkyl group wherein the alkyl group is itself substituted by an aryloxy group.

Other combinations of substituents are known in the art and, are described, for example, in U.S. Pat. No. 8,344,162 to Jung et al., incorporated herein by this reference. For example, the term “thiocarbonyl” and combinations of substituents including “thiocarbonyl” include a carbonyl group in which a double-bonded sulfur replaces the normal double-bonded oxygen in the group. The term “alkylidene” and similar terminology refer to an alkyl group, alkenyl group, alkynyl group, or cycloalkyl group, as specified, that has two hydrogen atoms removed from a single carbon atom so that the group is double-bonded to the remainder of the structure.

The exact formulation, route of administration and dosage can be chosen by the individual physician in view of the patient's condition. (See e.g., Fingl et al., in The Pharmacological Basis of Therapeutics, 1975, Ch. 1, p. 1). It should be noted that the attending physician would know how to and when to terminate, interrupt, or adjust administration due to toxicity, or to organ dysfunctions. Conversely, the attending physician would also know to adjust treatment to higher levels if the clinical response were not adequate (precluding toxicity). The magnitude of an administered dose in the management of the disorder of interest will vary with the severity of the condition to be treated and to the route of administration. The severity of the condition may, for example, be evaluated, in part, by standard prognostic evaluation methods. Further, the dose and perhaps the dose frequency, will also vary according to the age, body weight, and response of the individual patient, as well as factors such as pharmacokinetic factors such as liver and kidney function.

In methods according to the present invention, when the pentosan polysulfate salt is sodium pentosan polysulfate, the sodium pentosan polysulfate is typically administered in a dosage form that is palatable and acceptable to the patient when administered orally. Many suitable dosage forms are known in the art. Typically, the sodium pentosan polysulfate is administered in the form of a pharmaceutical composition as described above. The pharmaceutical composition includes the sodium pentosan polysulfate, the penetration enhancer, and, optionally, the filler, excipient, or carrier as described above. Similar principles can be applied for the preparation of suitable dosage forms or pharmaceutical compositions when the pentosan polysulfate is other than sodium pentosan polysulfate, such as potassium pentosan polysulfate or calcium pentosan polysulfate.

For example, in general, dosage forms for the oral administration of the sodium pentosan polysulfate can be in the form of tablets, dragees, capsules, or solutions, although, for reasons of palatability and acceptability, solid dosage forms are typically preferred. Suitable solid dosage forms for the oral administration of heparin can be prepared by combining the heparin, the cationic counterion, the penetration enhancer, if present, and any other ingredients such as stabilizers, preservatives, or excipients, adding other inert ingredients as necessary to provide the correct volume of the mixture, and grinding the mixture to homogeneity. The resulting mixture can be pressed into tablets or dragees or incorporated into capsules, as described further below.

Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Other suitable fillers, carriers, and excipients are described above.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses, different penetration enhancers, or different concentrations of penetration enhancers or sodium pentosan polysulfate.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added.

In one particularly preferred dosage form for administration, sodium pentosan polysulfate and the penetration enhancer SNAC are incorporated into soft gelatin capsules.

Other suitable dosage forms are known in the art.

As used herein, terms such as “treating,” “treatment,” and analogous terminology does not imply a complete cure of LUDE or a disease, condition, or syndrome associated with LUDE, such as interstitial cystitis, or, as detailed below, another disease, condition, or syndrome treatable with a pentosan polysulfate salt, such as sodium pentosan polysulfate, potassium pentosan polysulfate, or calcium pentosan polysulfate. The terms “treating,” “treatment,” or analogous terminology as used herein mean that a patient that is treated by a method according to the present invention achieves a detectable result of improvement with respect to LUDE or a disease, condition, or syndrome associated with LUDE, such as interstitial cystitis, or, as detailed below, another disease, condition, or syndrome treatable with a pentosan polysulfate salt, such as sodium pentosan polysulfate, potassium pentosan polysulfate, or calcium pentosan polysulfate. For interstitial cystitis or another disease or condition associated with LUDE, such a detectable result of improvement can be, but is not limited to, a reduction in pain, a reduction in urinary frequency, a reduction of urinary urgency, a reduction of incontinence, an increase in bladder capacity, a reduction of potassium permeability of the bladder epithelium, or any other objective or subjective result experienced by the patient. For other diseases or conditions, such a detectable result of improvement can be a detectable clinical result as recognized by a practitioner skilled in treating the disease or condition; the detectable result of improvement can be objective or subjective.

Methods according to the present invention for the treatment of LUDE or a disease, condition, or syndrome associated with LUDE can be combined with other methods for treatment of LUDE or a disease, condition, or syndrome associated with LUDE, including interstitial cystitis. The pharmaceutical compositions used in these methods can be administered by oral or other routes as appropriate. Such methods include the intravesical administration of a therapeutically effective quantity composition comprising a heparinoid, a local anesthetic, and a buffering compound, as described in U.S. Pat. No. 7,414,039 to Parsons, and other methods described in PCT Patent Application Publication No. WO 2007/073397 by Flashner et al., such as the administration of a therapeutically effective quantity of an oral anticholinergic drug such as detroloxybutynin chloride (Ditropan XL®) or tolterodine (Detrusitol® or Detrol LA), the administration of a therapeutically effective quantity of mesna (Mesnex®), the administration of a therapeutically effective quantity of dimethyl sulfoxide (DMSO), or the administration of a therapeutically effective quantity of an analgesic or a narcotic to control pain.

Another aspect of the present invention is a pharmaceutical composition comprising sodium pentosan polysulfate in a form suitable for oral administration. The pharmaceutical composition is formulated for the treatment of LUDE or a disease, condition, or syndrome associated with LUDE, including interstitial cystitis.

Alternatively, yet another aspect of the present invention is a pharmaceutical composition comprising a salt of pentosan polysulfate selected from the group consisting of sodium pentosan polysulfate, potassium pentosan polysulfate, and calcium pentosan polysulfate in a form suitable for oral administration. The pharmaceutical composition is formulated for the treatment of a disease or condition selected from the group consisting of HIV infection, prostate cancer, osteoarthritis, prion disease, including variant Creutzfeldt-Jakob disease, inflammatory myocardial injury, osteonecrosis, intervertebral disc degeneration, amyloid-β-induced toxicity in Alzheimer's disease, and atherosclerosis.

Therefore, as described above, in general, a pharmaceutical composition suitable for the treatment of LUDE or a disease, condition, or syndrome associated with LUDE according to the present invention comprises:

(1) a quantity of sodium pentosan polysulfate that is pharmaceutically sufficient for the treatment of LUDE or a disease, condition, or syndrome associated with LUDE;

(2) a penetration enhancer as described above in a quantity sufficient to improve the bioavailability of the sodium pentosan polysulfate; and

(3) optionally, at least one filler, excipient, or carrier;

wherein the pharmaceutical composition is formulated for the treatment of LUDE or a disease, condition, or syndrome associated with LUDE.

As another alternative, as described above, in general, a pharmaceutical composition suitable for the treatment for the treatment of a disease or condition selected from the group consisting of HIV infection, prostate cancer, osteoarthritis, prion disease, including variant Creutzfeldt-Jakob disease, inflammatory myocardial injury, osteonecrosis, intervertebral disc degeneration, amyloid-β-induced toxicity in Alzheimer's disease, and atherosclerosis according to the present invention comprises:

(1) a quantity of a salt of pentosan polysulfate selected from the group consisting of sodium pentosan polysulfate, potassium pentosan polysulfate, and calcium potassium phosphate, that is pharmaceutically sufficient for the treatment of a disease or condition selected from the group consisting of HIV infection, prostate cancer, osteoarthritis, prion disease, including variant Creutzfeldt-Jakob disease, inflammatory myocardial injury, osteonecrosis, intervertebral disc degeneration, amyloid-β-induced toxicity in Alzheimer's disease, and atherosclerosis;

(2) a penetration enhancer as described above in a quantity sufficient to improve the bioavailability of the sodium pentosan polysulfate; and

(3) optionally, at least one filler, excipient, or carrier;

wherein the pharmaceutical composition is formulated for the treatment of a disease or condition selected from the group consisting of HIV infection, prostate cancer, osteoarthritis, prion disease, including variant Creutzfeldt-Jakob disease, inflammatory myocardial injury, osteonecrosis, intervertebral disc degeneration, amyloid-β-induced toxicity in Alzheimer's disease, and atherosclerosis.

Typically, a pharmaceutical composition according to the present invention comprises a quantity of sodium pentosan polysulfate from about 10 mg to about 400 mg per unit dose. More typically, a pharmaceutical composition according to the present invention comprises a quantity of sodium pentosan polysulfate from about 50 mg to about 200 mg per unit dose. Preferably, a pharmaceutical composition according to the present invention comprises a quantity of sodium pentosan polysulfate from about 75 mg to about 150 mg per unit dose. The quantities of other pentosan polysulfates used in a pharmaceutical composition according to the present invention, such as potassium pentosan polysulfate or calcium pentosan polysulfate, can be determined by one of skill in the art by calculating the relative equivalents involved taking into account the different formula weights of the counterions (potassium or calcium versus sodium).

As indicated above, a particularly preferred penetration enhancer as used in a pharmaceutical composition according to the present invention is sodium N-[8-(2-hydroxybenzoyl)amino]caprylate (SNAC), as described above. Other penetration agents known in the art are described above.

The dosage form of a pharmaceutical composition according to the present invention is typically a tablet, a dragee, a capsule, or a solution; preferably, the dosage form is a tablet, a dragee, or a capsule. A particularly preferred dosage form is a capsule, such as a soft gelatin capsule. Enteric-coated tablets and capsules can be used. Suitable enteric coatings are known in the art and are described, for example, in United States Patent Application Publication No. 2013/0331361 by Dansereau et al., incorporated herein by this reference. Typically, such enteric coatings are pH dependent and utilize a pH-dependent enteric coating material that is made from a partly methyl-esterified methacrylic acid polymer. Typically, the enteric coating is insoluble or substantially insoluble at a pH below 5.5 (i.e., the pH generally found in the mouth, pharynx, esophagus, and stomach) but is soluble at pH 5.5 or higher (i.e., the pH generally found in the small intestine). A particularly suitable coating is Eudragit L®, particularly Eudragit L 30 D-55® or Eudragit L100-55®; these are manufactured by Rohm Pharma GmbH of Darmstadt, Germany. The coating can, and usually will, contain a plasticizer and possibly other coating excipients such as coloring agents, surfactant, talc, and/or magnesium stearate, many of which are well known in the coating art. In particular, anionic carboxylic acrylic polymers usually will contain 10-25% by weight of a plasticizer, especially triethyl citrate, tributyl citrate, acteyltriethyl citrate, dibutyl phthalate, diethyl phthalate, polyethylene glycol, acetylated monoglycerides propylene glycol, and triacetin. Conventional coating techniques such as fluid-bed or pan coating are employed to apply the coating. Coating thickness must be sufficient to ensure that the oral dosage form remains essentially intact until the desired site of delivery in the lower gastrointestinal tract is reached. Other alternative dosage forms, such as sublingual dosage forms, buccal fast melt tablet dosage forms, and film dosage forms, are known in the art and can be employed. Sublingual dosage forms are described in U.S. Pat. No. 8,846,074 to Bryson et al. and in U.S. Pat. No. 8,778,394 to Palmer et al., both incorporated herein by this reference. Fast melt buccal dosage forms are described in U.S. Pat. No. 8,957,105 to Wasley et al. and in U.S. Pat. No. 8,911,769 to Spencer et al., both incorporated by this reference. Film dosage forms are disclosed in U.S. Pat. No. 8,580,830 to Leichs et al. and in U.S. Pat. No. 8,007,825 to Wynn et al., both incorporated herein by this reference.

Another alternative for dosage forms of a pharmaceutical composition according to the present invention is the use of coated nanoparticles for drug delivery. The use of coated nanoparticles is described in K. S. Soppimath et al., “Biodegradable Polymeric Nanoparticles as Drug Delivery Devices,” J. Controlled Release 70: 1-20 (2001); M. L. Hans & A. M. Lowman, “Biodegradable Nanoparticles for Drug Delivery and Targeting,” Curr. Opin. Solid State & Mater. Sci. 6: 319-327 (2002); and W. H. De Jong & P. J. A. Borm, “Drug Delivery and Nanoparticles: Applications and Hazards,” Int. J. Nanomedicine 3: 133-149 (2008).

Another aspect of the present invention is a method of treating lower urinary dysfunctional epithelium (LUDE) or a disease, condition, or syndrome associated with LUDE comprising the step of administering orally: (1) a pharmaceutically effective quantity of sodium pentosan polysulfate; and (2) a quantity of a penetration enhancer effective to improve the bioavailability of sodium pentosan polysulfate to a patient in need of treatment for LUDE or a disease, condition, or syndrome associated with LUDE in order to treat LUDE or a disease, condition, or syndrome associated with LUDE.

The penetration enhancer is typically one of the penetration enhancers described above. Preferred penetration enhancers are as described above.

Typically, the sodium pentosan polysulfate and the penetration enhancer are administered in a pharmaceutical composition as described above. Alternatively, the sodium pentosan polysulfate and the penetration enhancer can be administered separately. If the sodium pentosan polysulfate and the penetration enhancer are administered separately, either or both can be administered together with at least one filler, excipient, or carrier. Suitable fillers, excipients, and carriers are as described above.

Typically, the quantity of sodium pentosan polysulfate administered is from about 10 mg to about 400 mg per unit dose. Preferably, the quantity of sodium pentosan polysulfate administered is from about 50 mg to about 200 mg per unit dose. More preferably, a pharmaceutical composition according to the present invention comprises a quantity of sodium pentosan polysulfate from about 75 mg to about 150 mg per unit dose.

Typically, the quantity of penetration enhancer administered is from about 50 mg to about 800 mg per unit dose. Preferably, the quantity of penetration enhancer administered is from about 100 mg to about 500 mg per unit dose. More preferably, the quantity of penetration enhancer administered is from about 150 mg to about 400 mg per unit dose.

Typically, the ratio, by weight, of the penetration enhancer to the sodium pentosan polysulfate is from about 0.167:1 to about 8:1. Preferably, the ratio, by weight, of the penetration enhancer to the sodium pentosan polysulfate is from about 0.50:1 to about 3:1. More preferably, the ratio, by weight, of the penetration enhancer to the sodium pentosan polysulfate is from about 0.75:1 to about 2:1.

Typically, the quantity of penetration enhancer used is sufficient to increase the bioavailability of sodium pentosan polysulfate to at least 5%. Preferably, the quantity of penetration enhancer used is sufficient to increase the bioavailability of sodium pentosan polysulfate to at least 10%. More preferably, the quantity of penetration enhancer used is sufficient to increase the bioavailability of sodium pentosan polysulfate to at least 20%. Still more preferably, the quantity of penetration enhancer used is sufficient to increase the bioavailability of sodium pentosan polysulfate to at least 30%.

Typically, the disease, condition, or syndrome associated with LUDE is interstitial cystitis. However, methods according to the present invention can be used to treat another disease, condition, or syndrome associated with LUDE, such as, but not necessarily limited to, renal calculi, radiation cystitis, prostatitis, overactive bladder, and urinary infections.

The term “treating” refers to delaying, halting, alleviating, reversing, or preventing the onset of, the progress of, one or more symptoms of the disease, condition, or syndrome to which the term applies. As used herein, the term “treating” does not imply a cure, permanent or otherwise, for the disease, condition, or syndrome. The effectiveness of treating can be measured by objective or subjective standards. Objective standards include reduction of urinary frequency and reduction of permeability of urinary tract epithelium to potassium. Subjective standards include reduction of pain as reported by the subject being treated by the method, an improvement in mood, an improvement in well-being, or a reduction in disability experienced by the subject.

Another aspect of the present invention is a method of treating a disease or condition selected from the group consisting of HIV infection, prostate cancer, osteoarthritis, prion disease, including variant Creutzfeldt-Jakob disease, inflammatory myocardial injury, osteonecrosis, intervertebral disc degeneration, amyloid-β-induced toxicity in Alzheimer's disease, and atherosclerosis comprising the step of administering orally: (1) a pharmaceutically effective quantity of a salt of pentosan polysulfate selected from the group consisting of sodium pentosan polysulfate, potassium pentosan polysulfate, and calcium pentosan polysulfate; (2) a quantity of a penetration enhancer effective to improve the bioavailability of the salt of pentosan polysulfate; and (3) optionally, a pharmaceutically acceptable carrier, to a patient in need of treatment for a disease or condition selected from the group consisting of HIV infection, prostate cancer, osteoarthritis, prion disease, including variant Creutzfeldt-Jakob disease, inflammatory myocardial injury, osteonecrosis, intervertebral disc degeneration, amyloid-β-induced toxicity in Alzheimer's disease, and atherosclerosis in order to treat a disease or condition selected from the group consisting of HIV infection, prostate cancer, osteoarthritis, prion disease, including variant Creutzfeldt-Jakob disease, inflammatory myocardial injury, osteonecrosis, intervertebral disc degeneration, amyloid-β-induced toxicity in Alzheimer's disease, and atherosclerosis.

In this alternative, the patient can also be administered an additional conventional therapeutic agent intended for the treatment of the disease or condition selected from the group consisting of HIV infection, prostate cancer, osteoarthritis, prion disease, including variant Creutzfeldt-Jakob disease, inflammatory myocardial injury, osteonecrosis, intervertebral disc degeneration, amyloid-β-induced toxicity in Alzheimer's disease, and atherosclerosis. The additional conventional therapeutic agent can be administered together or separately with the pentosan polysulfate salt, and, if administered separately, can be administered in a pharmaceutical composition with a pharmaceutically acceptable carrier.

The penetration enhancer is typically one of the penetration enhancers described above. Preferred penetration enhancers are as described above.

Typically, the pentosan polysulfate salt and the penetration enhancer are administered in a pharmaceutical composition as described above. Alternatively, the pentosan polysulfate salt and the penetration enhancer can be administered separately. If the pentosan polysulfate salt and the penetration enhancer are administered separately, either or both can be administered together with at least one filler, excipient, or carrier. Suitable fillers, excipients, and carriers are as described above.

Typically, when the pentosan polysulfate salt is sodium pentosan polysulfate, the quantity of sodium pentosan polysulfate administered is from about 10 mg to about 400 mg per unit dose. Preferably, the quantity of sodium pentosan polysulfate administered is from about 50 mg to about 200 mg per unit dose. More preferably, a pharmaceutical composition according to the present invention comprises a quantity of sodium pentosan polysulfate from about 75 mg to about 150 mg per unit dose. The quantities of other pentosan polysulfates used in a method according to the present invention, such as potassium pentosan polysulfate or calcium pentosan polysulfate, can be determined by one of skill in the art by calculating the relative equivalents involved taking into account the different formula weights of the counterions (potassium or calcium versus sodium). The quantity of pentosan polysulfate salt to be administered can be varied by one of ordinary skill in the art depending on the condition for which the pentosan polysulfate salt is administered, the target organ, the response to administration, pharmacokinetic factors such as kidney and liver function, the age and weight of the subject, and other variables.

Typically, the quantity of penetration enhancer administered is from about 50 mg to about 800 mg per unit dose. Preferably, the quantity of penetration enhancer administered is from about 100 mg to about 500 mg per unit dose. More preferably, the quantity of penetration enhancer administered is from about 150 mg to about 400 mg per unit dose.

Typically, the ratio, by weight, of the penetration enhancer to the pentosan polysulfate salt is from about 0.167:1 to about 8:1. Preferably, the ratio, by weight, of the penetration enhancer to the pentosan polysulfate salt is from about 0.50:1 to about 3:1. More preferably, the ratio, by weight, of the penetration enhancer to the pentosan polysulfate salt is from about 0.75:1 to about 2:1.

Typically, the quantity of penetration enhancer used is sufficient to increase the bioavailability of the pentosan polysulfate salt to at least 5%. Preferably, the quantity of penetration enhancer used is sufficient to increase the bioavailability of the pentosan polysulfate salt to at least 10%. More preferably, the quantity of penetration enhancer used is sufficient to increase the bioavailability of the pentosan polysulfate salt to at least 20%. Still more preferably, the quantity of penetration enhancer used is sufficient to increase the bioavailability of the pentosan polysulfate salt to at least 30%.

Another aspect of the present invention is the use of pentosan polysulfate salts, including, but not limited to sodium pentosan polysulfate, for the treatment of disease and conditions associated with inflammation. The disease or condition associated with inflammation can be, but is not necessarily limited to, rheumatoid arthritis, juvenile rheumatoid arthritis, osteoarthritis, psoriasis, psoriatic arthritis, ankylosing spondylitis, lupus erythematosus, multiple sclerosis, or asthma. The use of pentosan polysulfate salts is particularly useful for the treatment of osteoarthritis or rheumatoid arthritis. The use of pentosan polysulfate salts to treat arthritic conditions such as osteoarthritis is not limited to use in human patients; pentosan polysulfate salts can also be used to treat arthritic conditions in animals, including socially or economically important animals such as dogs, cats, horses, mules, cows, pigs, goats, or sheep. The use of pentosan polysulfate to treat osteoarthritis in veterinary medicine, particularly in dogs, is described in “Cartrophen Vet: A Disease Modifying Osteoarthritis Drug,” Biopharm Australia Pty. Ltd. (June 2006), incorporated herein by this reference. The pentosan polysulfate salt is administered together with a penetration enhancer as described above. In one alternative, the pentosan polysulfate salt and the penetration enhancer are administered in a single pharmaceutical composition. The pharmaceutical composition can include conventional carriers, fillers, or excipients as described above. In another alternative, the pentosan polysulfate salt is administered separately from the penetration enhancer; in that alternative, the pentosan polysulfate salt can be incorporated into a pharmaceutical composition.

As detailed further below, the pentosan polysulfate salt, such as sodium pentosan polysulfate, can be administered in a therapeutically effective quantity as a single agent, either by itself or part of a pharmaceutical composition, together with a penetration enhancer as described above (the penetration enhancer can be incorporated into a pharmaceutical composition as described above or can be administered separately). Alternatively, the pentosan polysulfate salt can be administered in a therapeutically effective quantity together with a therapeutically effective quantity of at least one additional agent that is effective in treating inflammation; a penetration enhancer as described above is also administered. The penetration enhancer is effective in increasing the bioavailability of the orally administered pentosan polysulfate salt; in some cases, the penetration enhancer may also be effective in increasing the bioavailability of one or more of the additional agents when such agents are administered orally. Either the pentosan polysulfate salt or the at least one additional agent can be included in a pharmaceutical composition; the pentosan polysulfate salt and the at least one additional agent can be included in the same pharmaceutical composition or in different pharmaceutical compositions, depending on the optimal route of administration of the additional agent, which depends on bioavailability and other pharmacokinetic considerations. Various combinations are possible and are within the scope of the invention. For example, in one alternative, when one additional agent is administered, what is administered can be: (i) a pentosan polysulfate salt in a pharmaceutical composition; (ii) a penetration enhancer in the pharmaceutical composition; and (iii) at least one additional agent also in the pharmaceutical composition. In another alternative, what is administered can be: (i) a pentosan polysulfate salt; (ii) a penetration enhancer; and (iii) at least one additional agent. In yet another alternative, what is administered can be: (i) a pentosan polysulfate salt in a pharmaceutical composition; (ii) a penetration enhancer in the pharmaceutical composition; and (iii) at least one additional agent. In yet another alternative, what is administered can be: (i) a pentosan polysulfate salt in a first pharmaceutical composition; (ii) a penetration enhancer also in the first pharmaceutical composition; and (iii) at least one additional agent in a second pharmaceutical composition. In still another alternative, what is administered can be: (i) a pentosan polysulfate salt; (ii) a penetration enhancer; and (iii) at least one additional agent in a pharmaceutical composition. In general, all possible combinations of pentosan polysulfate salts and penetration enhancers as described above can be employed. The pentosan polysulfate salt is administered orally in methods according to the present invention. The at least one additional agent may be administered orally or by other routes depending on the chemical structure of the additional agent or agents and on pharmacokinetic factors known in the art.

Additional agents that are effective in treating inflammation are described below. When at least one additional agent that is effective in treating inflammation is administered together with the pentosan polysulfate salt, the at least one additional agent and the pentosan polysulfate salt can be administered in a single pharmaceutical composition as described above. The pharmaceutical composition can include conventional carriers, fillers, or excipients. Alternatively, the at least one additional agent that is effective in treating inflammation can be administered separately as described above, such as in a pharmaceutical composition including one or more additional agents that are effective in treating inflammation. The pharmaceutical composition can include conventional carriers, fillers, or excipients.

The use of pentosan polysulfate salts in the treatment of arthritis is described in P. Ghosh et al., “Pentosan Polysulfate Promotes Proliferation and Chondrogenic Differentiation of Adult Human Bone Marrow-Derived Mesenchymal Precursor Cells,” Arthritis Res. Ther. 12: R28 (2010); K. Kumagai et al., “Sodium Pentosan Polysulfate Resulted in Cartilage Improvement in Knee Osteoarthritis—An Open Clinical Trial,” BMC Clin. Pharmacol. 10: 7 (2010); P. Ghosh et al., “Pentosan Polysulfate, a Rational Therapy for the Treatment of Osteoarthritis. Results of a Double Blind Placebo Controlled Clinical Trial,” Ann. Rheum. Dis. 64: 1578 (2005); P. Ghosh, “The Pathobiology of Osteoarthritis and the Rationale for the Use of Pentosan Polysulfate for Its Treatment,” Semin. Arthritis Rheum. 28: 211-267 (1999), all of which are incorporated herein by this reference.

Among the additional agents that are effective in treating inflammation that can be administered along with or together with pentosan polysulfate salts such as sodium pentosan polysulfate is calcitonin, including human calcitonin, eel calcitonin, salmon calcitonin, bovine calcitonin, chicken calcitonin, or synthetic calcitonin variants such as (Asu^{1,7})eel calcitonin, wherein “Asu” refers to the non-naturally occurring amino acid aminosuberic acid. As detailed below, other modified variants of naturally occurring calcitonins, including variants of calcitonins including one or more conservative amino acid substitutions, can be used. Commercially available oral calcitonin preparations are described in R. C. Hamdy & D. N. Daley, “Oral Calcitonin,” Int. J. Women's Health 4: 471-479 (2012), incorporated herein by this reference.

As detailed below, other additional agents, besides calcitonin, that are effective in treating inflammation can be used along with or together with pentosan polysulfate salts are known in the art. One or more of these agents can be administered in a therapeutically effective quantity along with or together with a pentosan polysulfate salt.

U.S. Pat. No. 8,835,389 to Azria et al., incorporated herein by this reference, discloses the use of salmon calcitonin in free or salt form for rheumatoid arthritis, including a delivery agent such as N-(5-chlorosalicyloyl)-8-aminocaprylic acid (5-CNAC), N-(10-[2-hydroxybenzoly]amino)decanoic acid (SNAD), N-(8-[2-hydroxybenzoyl]amino)caprylic acid (SNAC), optionally together with another agent such as: a calcitonin analog or derivative, a COX-2 inhibitor such as lumiracoxib, celecoxib, rofecoxib, valdecoxib, etoricoxib, a mixed COX-1/COX-2 inhibitor such as diclofenac, etanercept, a painkiller such as aspirin or acetaminophen, bone formers, and bone antiresorbers. In place of salmon calcitonin, human calcitonin or (Asu^{1,7})eel calcitonin can be used. Still other calcitonins such as pig calcitonin can be used. Calcitonin can also be delivered with a conjugation-stabilized peptide or protein composition.

U.S. Pat. No. 8,765,675 to Azria et al., incorporated herein by this reference, discloses the use of salmon calcitonin in free or salt form for osteoarthritis, including a delivery agent such as N-(5-chlorosalicyloyl)-8-aminocaprylic acid (5-CNAC), N-(10-[2-hydroxybenzoly]amino)decanoic acid (SNAD), N-(8-[2-hydroxybenzoyl]amino)caprylic acid (SNAC), optionally together with another agent such as: a calcitonin analog or derivative, a COX-2 inhibitor such as lumiracoxib, celecoxib, rofecoxib, valdecoxib, etoricoxib, a mixed COX-1/COX-2 inhibitor such as diclofenac, etanercept, a painkiller such as aspirin or acetaminophen, bone formers, and bone antiresorbers. In place of salmon calcitonin, human calcitonin or (Asu^{1,7})eel calcitonin can be used. Still other calcitonins such as pig calcitonin can be used. Calcitonin can also be delivered with a conjugation-stabilized peptide or protein composition. Still other additional agents can be used, including steroid hormones such as an estrogen, a partial estrogen agonist or estrogen-gestagen combination, a SERM (Selective Estrogen Receptor Modulator) e.g. raloxifene, lasofoxifene, TSE-424, FC1271, tibolone, vitamin D or an analogue thereof or parathyroid hormone (PTH), a PTH fragment or a PTH derivative e.g. PTH (1-84), PTH (1-34), PTH (1-36), PTH (1-38), PTH (1-31)NH2 or PTS 893, bisphosphonates (e.g. alendronate, risedronate, zoledronic acid, ibandronate); protease inhibitors, e.g. cathepsin inhibitor, preferably a cathepsin K inhibitor; PTH releasers; SARMs (selective androgen receptor molecules).

United States Patent Application Publication No. 2009/0035315 by Christgau et al., incorporated herein by this reference, discloses the use of a strontium-containing compound for treatment of osteoarthritis and rheumatoid arthritis, alone or in combination with one or more additional agents. The strontium-containing compound can be selected from the group of organic strontium salts consisting of strontium malonate, strontium succinate, strontium fumarate, strontium ascorbate, strontium aspartate in either L and/or D-form, strontium glutamate in either L- and/or D-form, strontium pyruvate, strontium tartrate, strontium glutarate, strontium maleate, strontium methanesulfonate, strontium benzenesulfonate and strontium ranelate, strontium acetyl salicylate, strontium salicylate, strontium citrate, strontium alendronate, strontium risedronate, strontium chlodronate, strontium ethidronate and strontium L-threonate, strontium ibandronate, strontium ibuprofenate, strontium flubiprofenate, strontium ketoprofenate, strontium phorbol 12,13-didecanoate 20-homovanillate, strontium indomethacinate, strontium carprofenate, strontium naproxenate, strontium acetyloxy-benzoate, strontium 2-iminopiperidine, strontium methotrexate, strontium salsalate and strontium sulfasalazinate. The one or more additional agents can be a compound selected from the group consisting of bisphosphonates, glucosamine, palliative agents, analgesic agents, disease modifying anti-rheumatic compounds (DMARDs), selective estrogen receptor modulators (SERMs), aromatase inhibitors, non-steroidal anti-inflammatory agents (NSAIDs), COX-2 inhibitors, COX-3 inhibitors, opioids, inhibitors/antagonists of IL-1, inhibitors/antagonists of TNFα, inhibitors of matrix metalloproteinases (MMPs), cathepsin K inhibitors, inhibitors/antagonists of RANK-ligand, statins, glucocorticoids, chondroitin sulfate, keratin sulfate, statins, an endothelin-1 antagonist or inhibitor, NMDA receptor antagonists, inhibitors of interleukin-I converting enzyme, calcitonin gene related peptide antagonists, glycine antagonists, vanilloid receptor antagonists, inhibitors of inducible nitric oxide synthetase (iNOS), N-acetylcholine receptor agonists, neurokinin antagonists, neuroleptic agents, PAR2 receptor antagonists, sulfated cyclodextrins, and anabolic growth factors acting on joint tissue components. The bisphosphonate can be selected from the group consisting of ibandronate, zoledronate, alendronate, risedronate, ethidronate, chlodronate, tiludronate, minodronate, incadronate, olpadronate and pamidronate. The opioid can be selected from the group consisting of fentanyl, morphine, oxycodone, hydrocodone, methadone, buprenorphine, pentazocine, butorphanol, dezocine, nalbuphine, meperidine, normeperidine, hydromorphone, codeine, levorphanol, tramadol, endorphin, nociceptin, endomorphin, and active metabolites thereof. The NSAID can be selected from the group consisting of: enolic acids such as piroxicam, tenoxicam and meloxicam: heteroaryl acetic acids such as diclofenac, tolmetin, ketorolac, misoprostol and zomepirac; indole and indene acetic acids such as indomethacin, mefenamic acid, sulindac and etodolac; p-aminophenol derivatives such as phenacetin and acetaminophen; propionic acids including naproxen, flurbiprofen, fenoprofen, oxaprozin, carprofen, ketoprofen and ibuprofen; sulfonanilides such as nimesulide; fenamates including mefenamic acid, meclofenamate and flufenamic acid; alkanones such as nabumetone; pyrazolones including phenylbutazone, oxyphenbutazone, antipyrine, aminopyrine and kebuzone; salicylates including acetylsalicylic acid (aspirin), salicylate, salsalate, diflunisal, olsalazine, fendosal, sulfasalazine and thiosalicylate; acetaminophen; or a pharmaceutically acceptable salt thereof. The selective COX-2 inhibitor can be a COX-2 inhibitor that has a 10-fold or greater affinity for the COX-2 isoform as compared with COX-1, and can be selected from the group consisting of rofecoxib, valdecoxib, celecoxib, etoricoxib, lumiracoxib, parecoxib, deracoxib, tiracoxib, meloxicam, nimesulide, (1,1-dimethylheptyl)-6a,7,10,10a-tetrahydro-1-hydroxy-6,6-dimethyl-6H-dibenzo[b,d]pyran carboxylic acid (CT-3), 5,5-dimethyl-3-(2-propoxy)-4-methanesulfonylphenyl)-2(5H)-furanone; carprofen; 2-(acetyloxy)benzoic acid 3-[(nitrooxy)methyl]phenyl ester (NCX4016), P54 (a turmeric derivative); 2,6-bis(1,1-dimethylethyl)[(E)-(2-ethyl-1,1-dioxo isothiazolidinylidene)methyl]phenol (S-2474), 5(R)-thiosulfonamide-3(2H)-benzofuranone (SVT-2016) and N-[3-(formyl-amino)oxophenoxy-4H benzopyranyl]methanesulfonamide (T-614); or a pharmaceutically acceptable salt thereof. The DMARD can be selected from the group consisting of doxycycline, chondroitin sulfate, methotrexate, leflunomide, dimethylnitrosamine, azatriopine, hydroxychloroqine, cyclosporine, minocycline, salazopyrine, penicillamine, aurothiomalate (gold salt), cyclophosphamide, azathioprine and pharmacologically active metabolites thereof. The selective estrogen receptor modulator (SERM) can be selected from the group consisting of raloxifene, arzoxifene, droloxifene, tamoxifen, 4-hydroxy-tamoxifen, 4′-iodotamoxifen, toremifene, (deaminohydroxy)-toremifene, chlomiphene, levormeloxifene, ormeloxifene, chroman derivatives, coumarin derivatives, idoxifene, nafoxidine, miproxifen phosphate (TAT-59), arzoxifene, lasofoxifene, (E)-1-butanamine, 4-(4-(2-chloro-1,2-diphenylethenyl)phenoxy)-N,N-diethyl-dihydrogen citrate (MDL-103323) (R. J. Bauman et al., “Clomiphene Analogs with Activity in Vitro and in Vivo Against Human Breast Cancer Cells,” Biochem. Pharmacol. 15: 841-851 (1998), acolbifene, (EM-652), EM-800, fulvestrant, N-(n-butyl)-1-[3,17β-dihydroxyestra-1,3,5(10)-trien-7α-yl]N-methylundecanamide (ICI 164,384), diethylstilbestrol, genistein, nafoxidine, nitromifene, moxesterol, diphenol hydrochrysene, erythro-MEA, allenolic acid, equilin-3-sulfate, cyclophenyl, chlorotrianisene, ethamoxytriphetol, lasofoxifene, bazedoxifene, genistein, tibolone, ospemifene, tesmilifene, droloxifene, panomifene, zindoxifene, meproxifene and faslodex as well as pharmacologically active metabolites thereof. The inhibitor of IL-1 can be a monoclonal antibody specifically binding IL-1 such as anakinra or a soluble IL-1 receptor derivative, including a derivative modified by attachment to polyethylene glycol. The inhibitor of interleukin-I converting enzyme can be pralnacasan. The inhibitor of TNFα can be etanercept, aldalimumab, and infliximab. The inhibitor of RANK-ligand can be OPG and monoclonal antibody 162. The inhibitor of the MMP can be an inhibitor of aggrecanase, MMP-1, MMP-13, MMP-3, cathepsin K, or another protease that participates in the catabolic process of tissue destruction. The glucocorticoid can be selected from the group consisting of prednisolone, prednisone, methylprednisolone, betamethasone, hydrocortisone, cortisone, triamcinolone, dexamethasone, beclomethasone, budesonide, deoxycortone or fludrocortisone. The anabolic growth factor can be an anabolic growth factor derived from bone or cartilage matrix proteins such as segments of or fragments from collagen type I, collagen type II, collagen type IX, collagen type XI, bone sialo protein (BSP), osteonectin, osteopontin, osteocalcin (also known as bone GLA protein), cartilage oligomeric matrix protein (COMP), cartilage intermediate layer protein (CILP) and aggrecan. Alternatively, the anabolic growth factor can be an anabolic growth factor such as human growth hormone (hGH), parathyroid hormone (PTH), glucagon like peptide-2 (GLP-2), Insulin like growth factor-1 (IGF-1) with or without IGF binding protein 3 (IGFBP-3). The statin can be a statin selected from the group consisting of nystatin, pravastatin, fluvostatin, atorvastatin and cerivastatin and therapeutically active derivatives thereof.

United States Patent Application Publication No. 2008/0160025 by Macintyre et al., incorporated herein by this reference, discloses the use of calcitonin to treat an inflammatory disease or condition. The inflammatory disease or condition can be rheumatoid arthritis, juvenile rheumatoid arthritis, osteoarthritis, psoriasis, psoriatic arthritis, ankylosing spondylitis, lupus erythematosus, multiple sclerosis, or asthma. Alternatively, the calcitonin can be used as immunosuppressive therapy. Other agents can also be administered together with the calcitonin, including a glucocorticoid, an anti-rheumatic drug, and a monoclonal antibody. The glucocorticoid can be prednisolone, dexamethasone, methylprednisolone, budesonide, hydrocortisone, betamethasone, triamcinolone, or fludrocortisone. The anti-rheumatic drug can be methotrexate. The monoclonal antibody can be an antibody or fragment thereof specifically binding the tumor necrosis factor receptor, such as etanercept. The calcitonin can be human, salmon, eel, porcine, bovine, or chicken. The sequences of these polypeptides are as follows: Cys-Ser-Asn-Leu-Ser-Thr-Cys-Val-Leu-Ser-Ala-Tyr-Trp-Arg-Asn-Leu-Asn-Asn-Phe-His-Arg-Phe-Ser-Gly-Met-Gly-Phe-Gly-Pro-Glu-Thr-Pro (Porcine) (SEQ ID NO: 1); Cys-Ser-Asn-Leu-Ser-Thr-Cys-Val-Leu-Ser-Ala-Tyr-Trp-Lys-Asp-Leu-Asn-Asn-Tyr-H is-Arg-Phe-Ser-Gly-Met-Gly-Phe-Gly-Pro-Glu-Thr-Pro (Bovine) (SEQ ID NO: 2); Cys-Ser-Asn-Leu-Ser-Thr-Cys-Val-Leu-Gly-Lys-Leu-Ser-Gln-Glu-Leu-His-Lys-Leu-Gln-Thr-Tyr-Pro-Arg-Thr-Asn-Thr-Gly-Ser-Gly-Thr-Pro (Salmon) (SEQ ID NO: 3); Cys-Ser-Asn-Leu-Ser-Thr-Cys-Val-Leu-Gly-Lys-Leu-Ser-Gln-Glu-Leu-His-Lys-Leu-Gln-Thr-Tyr-Pro-Arg-Thr-Asp-Val-Gly-Ala-Gly-Thr-Pro (Eel) (SEQ ID NO: 4); Cys-Gly-Asn-Leu-Ser-Thr-Cys-Met-Leu-Gly-Thr-Tyr-Thr-Gln-Asp-Phe-Asn-Lys-Phe-His-Thr-Phe-Pro-Gln-Thr-Ala-Leu-Gly-Val-Gly-Ala-Pro (Human) (SEQ ID NO: 5); and Cys-Ala-Ser-Leu-Ser-Thr-Cys-Val-Leu-Gly-Lys-Leu-Ser-Gln-Glu-Leu-His-Lys-Leu-Gln-Thr-Tyr-Pro-Arg-Thr-Asp-Val-Gly-Ala-Gly-Thr-Pro (Chicken) (SEQ ID NO: 6). The calcitonins used can alternatively be variants, fragments, or derivatives, including fusion proteins. Alternatively, the calcitonins can be truncated by deletion of residues 1-9. Other deletions can be made, but it is preferred that residues 17-21, at least, remain in the calcitonin fragment. The truncated sequences are as follows: Ser-Ala-Tyr-Trp-Arg-Asn-Leu-Asn-Asn-Phe-His-Arg-Phe-Ser-Gly-Met-Gly-Phe-Gly-Pro-Glu-Thr-Pro (Porcine (truncated)) (SEQ ID NO: 7); Ser-Ala-Tyr-Trp-Lys-Asp-Leu-Asn-Asn-Tyr-His-Arg-Phe-Ser-Gly-Met-Gly-Phe-Gly-Pro-Glu-Thr-Pro (Bovine (truncated)) (SEQ ID NO: 8); Gly-Lys-Leu-Ser-Gln-Glu-Leu-His-Lys-Leu-Gln-Thr-Tyr-Pro-Arg-Thr-Asn-Thr-Gly-Ser-Gly-Thr-Pro (Salmon (truncated)) (SEQ ID NO: 9); Gly-Lys-Leu-Ser-Gln-Glu-Leu-His-Lys-Leu-Gln-Thr-Tyr-Pro-Arg-Thr-Asp-Val-Gly-Ala-Gly-Thr-Pro (Eel (truncated)) SEQ ID NO: 10); Gly-Thr-Tyr-Thr-Gln-Asp-Phe-Asn-Lys-Phe-His-Thr-Phe-Pro-Gln-Thr-Ala-Leu-Gly-Val-Gly-Ala-Pro (Human (truncated)) (SEQ ID NO: 11); and Gly-Lys-Leu-Ser-Gln-Glu-Leu-His-Lys-Leu-Gln-Thr-Tyr-Pro-Arg-Thr-Asp-Val-Gly-Ala-Gly-Thr-Pro (Chicken (truncated)) (SEQ ID NO: 12).

Conservative amino acid substitutions are well known in the art. More specifically, in a peptide or protein, suitable conservative substitutions of amino acids are known to those of skill in this art and may be made generally without altering the biological activity of the resulting molecule. Those of skill in this art recognize that, in general, single amino acid substitutions in non-essential regions of a polypeptide do not substantially alter biological activity (see, e.g. Watson et al. Molecular Biology of the Gene, 4th Edition, 1987, Benjamin/Cummings, p. 224). In particular, such a conservative variant has a modified amino acid sequence, such that the change(s) do not substantially alter the protein's (the conservative variant's) secondary or tertiary structure and/or activity, specifically binding activity in this context. Conservative amino acid substitution generally involves substitutions of amino acids with residues having similar properties (e.g., acidic, basic, positively or negatively charged, polar or non-polar, etc.) such that the substitutions of even critical amino acids does not substantially alter structure and/or activity. Conservative substitution tables providing functionally similar amino acids are well known in the art. For example, one exemplary guideline to select conservative substitutions includes (original residue followed by exemplary substitution): Ala/Gly or Ser; Arg/Lys; Asn/Gln or His; Asp/Glu; Cys/Ser; Gln/Asn; Gly/Asp; Gly/Ala or Pro; His/Asn or Gin; Ile/Leu or Val; Leu/Ile or Val; Lys/Arg or Gin or Glu; Met/Leu or Tyr or Ile; Phe/Met or Leu or Tyr; Ser/Thr; Thr/Ser; Trp/Tyr; Tyr/Trp or Phe; Val/Ile or Leu. An alternative exemplary guideline uses the following six groups, each containing amino acids that are conservative substitutions for one another: (1) alanine (A or Ala), serine (S or Ser), threonine (T or Thr); (2) aspartic acid (D or Asp), glutamic acid (E or Glu); (3) asparagine (N or Asn), glutamine (Q or Gin); (4) arginine (R or Arg), lysine (K or Lys); (5) isoleucine (I or Ile), leucine (L or Leu), methionine (M or Met), valine (V or Val); and (6) phenylalanine (F or Phe), tyrosine (Y or Tyr), tryptophan (W or Trp); (see also, e.g., Creighton (1984) Proteins, W. H. Freeman and Company; Schulz and Schimer (1979) Principles of Protein Structure, Springer-Verlag). One of skill in the art will appreciate that the above-identified substitutions are not the only possible conservative substitutions. For example, for some purposes, one may regard all charged amino acids as conservative substitutions for each other whether they are positive or negative.

In some cases, non-naturally occurring amino acids can be incorporated into calcitonin variants. The incorporation of non-naturally occurring amino acids is described in U.S. Pat. No. 8,569,233 to Tian et al.; U.S. Pat. No. 8,735,539 to Kraynov et al.; and U.S. Pat. No. 8,791,231 to Miao et al., all incorporated herein by this reference. Methods for accomplishing such incorporation of non-naturally occurring amino acids are known in the art.

United States Patent Application Publication No. 2011/0305711 by Allan et al. is directed to the use of human engineered antibodies specifically binding the polypeptide α-CGRP (alpha-calcitonin gene related peptide). The antibodies or antigen-binding fragments thereof can be used for the treatment of osteoarthritis. The amino acid sequence of human α-CGRP is ACDTATCVTHRLAGLLSRSGGVVKNNFVPT NVGSKAF (SEQ ID NO: 13). Additionally, antagonists of α-CGRP can be used, including, but not limited to, olcegepant, telcagepant, and ubrogepant.

The mechanism of action for calcitonin in treating osteoarthritis or rheumatoid arthritis is described in B. C. Sondergaard et al., “Calcitonin Directly Attenuates Collagen Type II Degradation by Inhibition of Matrix Metalloproteinase Expression and Activity in Articular Chondrocytes,” Osteoarthritis Cartilage 14: 759-768 (2006); M. A. Karsdal et al., “Review: Calcitonin Is Involved in Cartilage Homeostasis: Is Calcitonin a Treatment for OA?,” Osteoarthritis Cartilage 14: 617-624 (2006); and S. Aida, “Effects of Eel Calcitonin on Rheumatoid Arthritis,” Ann. Rheumat. Dis. 50: 202-203 (1991), all incorporated herein by this reference.

The use of matrix metalloproteinase (MMP) inhibitors is described in J. F. Fisher & S. Mobashery, “Recent Advances in MMP Inhibitor Design,” Cancer Metastasis Rev. 25: 115-136 (2006), incorporated herein by this reference. The use of other disease-modifying osteoarthritis drugs (DMOAD) is described in A. J. Barr & P. G. Conaghan, “Disease-Modifying Osteoarthritis Drugs (DMOAD): What Are They and What Can We Expect From Them,” Medicographica 35: 189-196 (2013), incorporated herein by this reference.

Accordingly, the following additional agents can be employed with pentosan polysulfate for treatment of either osteoarthritis or rheumatoid arthritis:

(1) calcitonin, including salmon calcitonin, eel calcitonin, or human calcitonin);

(2) calcitonin derivatives, including (Asu^{1,7})eel calcitonin, variants, fragments, particularly fragments including amino acid residues 17-21 of calcitonin, and truncated derivatives of calcitonin lacking amino acid residues 1-9;

(3) bisphosphonates, including zoledronic acid, etidronate, clodronate, tiludronate, pamidronate, neridronate, olpadronate, alendronate, ibandronate, minodronate, incadronate, and risedronate;

(4) strontium ranelate;

(5) bone morphogenetic protein 7 (BMP-7), and homologs thereof including one or more conservative amino acid substitutions, preferably from 1 to 5 amino acid substitutions;

(6) selective iNOS (inducible nitric oxide synthase) inhibitors selected from the group consisting of: cindunistat; aminoguanidine hydrochloride; 2-amino-5,6-dihydro-6-methyl-4H-1,3-thiazine hydrochloride; AR-C 102222 (5-[(4′-amino-5′,8′-difluorospiro[piperidine-4,2′(1′H)-quinaxolin]-1-yl)carbonyl]-2-pyridinecarbonitrile hydrochloride); BYK 191023 dihydrochloride (2-[2-(4-methoxy-2-pyridinyl)ethyl]-1H-imidazo[4,5-b]pyridine dihydrochloride); (S)-ethylisothiourea hydrobromide; 2-iminopiperidine hydrochloride; (S)-isopropylisothiourea hydrobromide; (S)-methylisothiourea sulfate; N⁶-(1-iminoethyl)-L-lysine hydrochloride; N⁵-(1-iminoethyl)-L-ornithine dihydrochloride; and N-[[3-(Aminomethyl)phenyl]methyl]-ethanimidamide dihydrochloride);

(7) matrix metalloproteinase (MMP) inhibitors, wherein the MMP is selected from the group consisting of aggrecanase, MMP-1, MMP-13, MMP-3, cathepsin K, or another protease that participates in the catabolic process of tissue destruction, and wherein the MMP inhibitor is selected from the group consisting of batimastat, marimastat, ilomastat, prinomastat, cipemastat, MM1-166 (N-α-[4-(2-phenyl-2H-tetrazole-5-yl) phenyl sulfonyl]-D-tryptophan), MMI-270 ((2R)—N-hydroxy-2-[(4-methoxyphenyl)sulfonyl-(pyridin-3-ylmethyl)amino]-3-methylbutanamide), ABT-770 ((S)—N-[1-[[4′-trifluoromethoxy-[1,1′-biphenyl]-4-yl]oxy]methyl-2-(4,4-dimethyl-2,5-dioxo-1-imidazolidinyl)ethyl]-N-hydroxyformamide), RS-130830 (4-(((3-(4-chlorophenoxy)phenyl)sulfonyl)methyl)-N-hydroxytetrahydro-2H-pyran-4-carboxamide), CAS Reg. No. 239796-97-5 (1-benzyl-(4-(4-chlorophenoxy)phenyl)sulfonyl)-N-hydroxypiperidine-4-carboxamide), solimastat, KB-R-7785, GI-129471, rebimastat, tanomastat, Ro-28-2653, 544678-85-5, pyridine dicarboxamides, 868-68-30-3, CAS Reg. No. 582311-81-7, doxycycline, and metastat;

(8) an endogenous inhibitor of metalloproteinases;

(9) an inhibitor of cathepsin K;

(10) a COX-2 inhibitor selected from the group consisting of rofecoxib, valdecoxib, celecoxib, etoricoxib, lumiracoxib, parecoxib, deracoxib, tiracoxib, meloxicam, nimesulide, (1,1-dimethylheptyl)-6a,7,10,10a-tetrahydro-1-hydroxy-6,6-dimethyl-6H-dibenzo[b,d]pyran carboxylic acid (CT-3), 5,5-dimethyl-3-(2-propoxy)-4-methanesulfonylphenyl)-2(5H)-furanone; carprofen; 2-(acetyloxy)benzoic acid 3-[(nitrooxy)methyl]phenyl ester (NCX4016), P54 (a turmeric derivative); 2,6-bis(1,1-dimethylethyl)[(E)-(2-ethyl-1,1-dioxo isothiazolidinylidene)methyl]phenol (S-2474), 5(R)-thiosulfonamide-3(2H)-benzofuranone (SVT-2016) and N-[3-(formyl-amino)oxophenoxy-4H benzopyranyl]methanesulfonamide (T-614); and a pharmaceutically acceptable salt thereof;

(11) a mixed COX-1/COX-2 inhibitor such as diclofenac;

(12) an inhibitor of TNFα such as etanercept, aldalimumab, or infliximab;

(13) a non-steroidal anti-inflammatory drug (NSAID) painkiller such as: an enolic acids such as piroxicam, tenoxicam and meloxicam; heteroaryl acetic acids such as tolmetin, ketorolac, misoprostol and zomepirac; indole and indene acetic acids such as indomethacin, mefenamic acid, sulindac and etodolac; p-aminophenol derivatives such as phenacetin and acetaminophen; propionic acids including naproxen, flurbiprofen, fenoprofen, oxaprozin, carprofen, ketoprofen and ibuprofen; sulfonanilides such as nimesulide; fenamates including mefenamic acid, meclofenamate and flufenamic acid; alkanones such as nabumetone; pyrazolones including phenylbutazone, oxyphenbutazone, antipyrine, aminopyrine and kebuzone; salicylates including acetylsalicylic acid (aspirin), salicylate, salsalate, diflunisal, olsalazine, fendosal, sulfasalazine and thiosalicylate;

(14) a bone forming agent, such as Anti-Dkk1 antibodies or activin antagonists such as RAP-011;

(15) a bone antiresorbing agent;

(16) a steroid hormone such as an estrogen, a partial estrogen agonist or estrogen-gestagen combination, including prednisolone, prednisone, methylprednisolone, betamethasone, hydrocortisone, cortisone, triamcinolone, dexamethasone, beclomethasone, budesonide, deoxycortone or fludrocortisone;

(17) a SERM (Selective Estrogen Receptor Modulator) such as bazedoxifene acetate, ospemifene, raloxifene, arzoxifene, droloxifene, tamoxifen, 4-hydroxy-tamoxifen, 4′-iodotamoxifen, toremifene, (deaminohydroxy)-toremifene, chlomiphene, levormeloxifene, ormeloxifene, chroman derivatives, coumarin derivatives, idoxifene, nafoxidine, miproxifen phosphate (TAT-59), arzoxifene, lasofoxifene, (E)-1-butanamine, 4-(4-(2-chloro-1,2-diphenylethenyl)phenoxy)-N,N-diethyl-dihydrogen citrate (MDL-103323), acolbifene, (EM-652), EM-800, fulvestrant, N-(n-butyl)-11-[3,17β-dihydroxyestra-1,3,5(10)-trien-7α-yl]N-methylundecanamide (ICI 164,384), diethylstilbestrol, genistein, nafoxidine, nitromifene, moxesterol, diphenol hydrochrysene, erythro-MEA, allenolic acid, equilin-3-sulfate, cyclophenyl, chlorotrianisene, ethamoxytriphetol, lasofoxifene, bazedoxifene, genistein, tibolone, ospemifene, tesmilifene, droloxifene, panomifene, zindoxifene, meproxifene and faslodex;

(18) vitamin D or an analog thereof;

(19) parathyroid hormone (PTH), a PTH fragment or a PTH derivative e.g. PTH (1-84), PTH (1-34), PTH (1-36), PTH (1-38), PTH (1-31)NH2 or PTS 893;

(20) PTH releasers, including 2-chloro-N-[(1R)-1-(3-methoxyphenyl)ethyl]-benzenepropanamine hydrochloride and cinacalcet;

(21) strontium-containing compounds, such as organic strontium salts including strontium malonate, strontium succinate, strontium fumarate, strontium ascorbate, strontium aspartate in either L- and/or D-form, strontium glutamate in either L- and/or D-form, strontium pyruvate, strontium tartrate, strontium glutarate, strontium maleate, strontium methanesulfonate, strontium benzenesulfonate, strontium acetyl salicylate, strontium salicylate, strontium citrate, strontium alendronate, strontium risedronate, strontium chlodronate, strontium ethidronate and strontium L-threonate, strontium ibandronate, strontium ibuprofenate, strontium flubiprofenate, strontium ketoprofenate, strontium phorbol 12,13-didecanoate 20-homovanillate, strontium indomethacinate, strontium carprofenate, strontium naproxenate, strontium acetyloxy-benzoate, strontium 2-iminopiperidine, strontium methotrexate, strontium salsalate and strontium sulfasalazinate;

(22) glucosamine;

(23) disease modifying anti-rheumatic compounds (DMARDs) such as doxycycline, chondroitin sulfate, methotrexate, leflunomide, dimethylnitrosamine, azatriopine, hydroxychloroquine, cyclosporine, minocycline, salazopyrine, penicillamine, aurothiomalate (gold salt), cyclophosphamide, azathioprine and pharmacologically active metabolites thereof;

(24) aromatase inhibitors, such as aminoglutethimide, testolactone, anastrozole, letrozole, exemestane, vorozole, formestane, fadrozole, 4-hydroxyandrostenedione, 1,4,6-androstatrien-3,17-dione, and 4-androstene-3,6,17-trione;

(25) COX-3 inhibitors, including acetaminophen, dipyrone, antipyrine, and dimethylaminopyrene;

(26) opioids, including fentanyl, morphine, oxycodone, hydrocodone, methadone, buprenorphine, pentazocine, butorphanol, dezocine, nalbuphine, meperidine, normeperidine, hydromorphone, codeine, levorphanol, tramadol, endorphin, nociceptin, endomorphin, and active metabolites thereof;

(27) inhibitors/antagonists of IL-1, including a monoclonal antibody specifically binding IL-1 such as anakinra or a soluble IL-1 receptor derivative, including a derivative modified by attachment to polyethylene glycol;

(28) inhibitors/antagonists of interleukin-I converting enzyme, including pralnacasan;

(29) an inhibitor of RANK-ligand, including OPG and monoclonal antibody 162;

(30) an anabolic growth factor, such as an anabolic growth factor derived from bone or cartilage matrix proteins such as segments of or fragments from collagen type I, collagen type II, collagen type IX, collagen type XI, bone sialo protein (BSP), osteonectin, osteopontin, osteocalcin (also known as bone GLA protein), cartilage oligomeric matrix protein (COMP), cartilage intermediate layer protein (CILP) and aggrecan, human growth hormone (hGH), glucagon like peptide-2 (GLP-2), Insulin like growth factor-1 (IGF-1) with or without IGF binding protein 3 (IGFBP-3);

(31) statins, including nystatin, pravastatin, fluvostatin, atorvastatin, and cerivastatin and therapeutically active derivatives thereof;

(32) endothelin-1 antagonists/inhibitors, including bosentan, sitaxentan, ambrisentan, atrasentan, BQ-123 (2-[(3R,6R,9S,12R,15S)-6-(1H-indol-3-ylmethyl)-9-(2-methylpropyl)-2,5,8,11,14-pentaoxo-12-propan-2-yl-1,4,7,10,13-pentazabicyclo[13.3.0]octadecan-3-yl]acetic acid), zibotentan, macitentan, tenosentan, BQ-788 (N-[(cis-2,6-Dimethyl-1-piperidinyl)carbonyl]-4-methyl-L-leucyl-1-(methoxycarbonyl)-D-tryptophyl-D-norleucine sodium salt), and A192621 ((2R,3R,4S)-4-(1,3-benzodioxol-5-yl)-1-[2-(2,6-diethylanilino)-2-oxoethyl]-2-(4-propoxyphenyl)pyrrolidine-3-carboxylic acid);

(33) NMDA receptor antagonists, including R-2-amino-5-phosphonopentanoate, 2-amino-7-phosphonoheptanoic acid, 3-[(R)-2-carboxypiperazin-4-yl]-prop-2-enyl-1-phosphonic acid, selfotel, amantidine, atomoxetine, lanicemine, dextrallorphan, dizocilpine, gacyclidine, memantine, nitromemantine, neramexane, eliprodil, WMS-259 ((2S,4S)-2-[(4S)-2,2-Diphenyl-1,3-dioxolan-4-yl]-4-fluoropiperidine) remacemide, delucemine, aptiganel, rapastinel, NRX-1074 1-aminocyclopropane-1-carboxylic acid, and 5,7-dichlorokynurenic acid;

(34) calcitonin gene related peptide-a antagonists, including olcegepant, telcagepant, ubrogepant, and antibodies or fragments thereof specifically binding calcitonin gene related peptide-α, including human or humanized antibodies;

(35) chondroitin sulfate;

(36) keratin sulfate;

(37) glycine antagonists, including bicuculline, brucine, and tutin;

(38) vanilloid receptor antagonists, including AMG 517 (N-(4-((6-(4-trifluoromethyl)phenyl)pyrimidin-4-yl)oxy)benzo[d]thiazole-2-yl)acetamide), SB-705498 ((R)-1-(2-bromophenyl)-3-(1-(5-(trifluoromethyl)pyridin-2-yl)pyrrolidin-3-yl)urea), GRC 6211, AZD1386 and NGD 8243;

(39) N-acetylcholine receptor antagonists, including hexamethonium, mecamylamine, trimethaphan, atracurium, doxacurium, mivacurium, pancuronium, vecuronium, and 18-methoxycoronaridine;

(40) neurokinin antagonists, including RPR-100893 ((2S)-1-[(3aS,4S,7aS)-4-hydroxy-4-(2-methoxyphenyl)-7,7-diphenyl-1,3,3a,5,6,7a-hexahydroisoindol-2-yl]-2-(2-methoxyphenyl)propan-1-one), CP-99994 ((2S,3S)—N-[(2-Methoxyphenyl)methyl]-2-phenyl-3-piperidinamine dihydrochloride), L-733,060 ((2S,3S)-3-{[3,5-bis(trifluoromethyl)benzyl]oxy}-2-phenylpiperidine), aprepitant, fosaprepitant, vofopitant, lanepitant, and TAK-637 (R)-7-(3,5-bis(trifluoromethyl)benzyl)-9-methyl-5-(p-tolyl)8,9,10,11-tetrahydro-7H-[1,4]diazocino[2,1-g][1,7]naphthyridine-6,13-dione);

(41) neuroleptic agents, including benperidol, bromperidol, droperidol, haloperidol, moperone, pipamperone, timiperone, fluspirilene, penfluridol, pimozide, acepromazine, chlorpromazine, cyamemazine, dixyrazine, fluphenazine, levomepromazine, mesoridazine, perazine, pericyazine, perphenazine, pipotiazine, prochlorperazine, promazine, promethazine, prothipendyl, thioproperazine, thioridazine, trifluoperazine, triflupromazine, chlorprothixene, clopenthixol, flupentixol, tiotixene, zuclopenthixol, clotiapine, loxapin, prothipendyl, carpipramine, clocapramine, molindone, mosapramine, sulpiride, sultopiride, veralipride, amisulpride, amoxapine, aripiprazole, asenapine, clozapine, blonanserin, iloperidone, lurasidone, melperone, nemonapride, olanzapine, paliperidone, perosperone, quetiapine, remoxipride, risperidone, sertindole, trimipramine, ziprasidone, and zotepine;

(42) PAR2 receptor antagonists, including AC-55541 (N-[[1-(3-bromo-phenyl)-eth-(E)-yl idene-hydrazinocarbonyl]-(4-oxo-3,4-dihydro-phthalazin-1-yl)-methyl]-benzamide) and AC-264613 (2-oxo-4-phenylpyrrolidine-3-carboxylic acid [1-(3-bromo-phenyl)-(E/Z)-ethylidene]-hydrazide; and

(43) sulfated cyclodextrins (referred to herein as “Additional Agents (1)-(43)”).

Therefore, another aspect of the present invention is a method of treating a disease or condition associated with inflammation. The method comprises the step of administering: (i) a therapeutically effective quantity of a pentosan sulfate salt as described above; and (ii) a quantity of a penetration enhancer sufficient to improve the bioavailability of the pentosan polysulfate salt as described above to a subject with a disease or condition associated with inflammation or at risk of contracting such a disease or condition. The subject with the disease or condition associated with inflammation or at risk of contracting such a disease or condition can be human, or, alternatively, can be a socially or economically important animal such as a dog, a cat, a horse, a mule, a cow, a pig, a goat, or a sheep. The disease or condition can be rheumatoid arthritis, juvenile rheumatoid arthritis, osteoarthritis, psoriasis, psoriatic arthritis, ankylosing spondylitis, lupus erythematosus, multiple sclerosis, or asthma. Typically, the disease or condition is rheumatoid arthritis or osteoarthritis. Suitable pentosan polysulfate salts are described above. Typically, the pentosan polysulfate salt is sodium pentosan polysulfate. Suitable penetration enhancers are described above.

In one alternative, the method further comprises administering a therapeutically effective quantity of at least one additional agent effective to treat a disease or condition associated with inflammation. Typically, the at least one additional agent effective to treat the disease or condition associated with inflammation is at least one of Additional Agents (1)-(43).

The pentosan polysulfate salt can be included in a pharmaceutical composition that further includes at least one carrier, excipient, or filler as described above. In one alternative, the penetration enhancer is included in the pharmaceutical composition together with the pentosan polysulfate salt. If an additional agent is administered, it can be included in the pharmaceutical composition that includes the pentosan polysulfate salt. Alternatively, it can be administered separately, either alone or as part of a second pharmaceutical composition that includes at least one carrier, excipient, or filler; this second pharmaceutical composition does not include the pentosan polysulfate salt. Various combinations of the pentosan polysulfate salt and the additional agent, if used, are described above in terms of the inclusion of either the pentosan polysulfate salt and the additional agent in one or more pharmaceutical compositions; all of these combinations are within the scope of the invention.

Yet another aspect of the present invention is a pharmaceutical composition formulated for treatment or prevention of a disease or condition associated with inflammation comprising:

(1) a therapeutically effective quantity of a pentosan polysulfate salt as described above;

(2) a quantity of a penetration enhancer as described above sufficient to improve the bioavailability of the pentosan polysulfate salt; and

(3) optionally, a pharmaceutically acceptable carrier.

The pharmaceutical composition can be formulated for treatment of rheumatoid arthritis, juvenile rheumatoid arthritis, osteoarthritis, psoriasis, psoriatic arthritis, ankylosing spondylitis, lupus erythematosus, multiple sclerosis, or asthma. Typically, the disease or condition is rheumatoid arthritis or osteoarthritis.

The pharmaceutical composition can further comprise a therapeutically effective quantity of at least one additional agent for treatment of the disease or condition. Typically, the additional agent is at least one of Additional Agents (1)-(43).

The following references may be useful in understanding the invention. These references are not necessarily prior art and are not identified herein as prior art. These references are referred to herein by numerals in parenthesis, e.g., (1).
1. Parsons, C. L., et al., Abnormal sensitivity to intravesical potassium in interstitial cystitis and radiation cystitis. Neurourol Urodyn. 1994; 13(5):515-20.
2. Parsons, C. L., The role of the urinary epithelium in the pathogenesis of interstitial cystitis/prostatitis/urethritis. Urology. 2007; 69(4 Suppl):9-16.
3. Hanno P M, Landis J R, Matthews-Cook Y, Kusek J, Nyberg L Jr The diagnosis of interstitial cystitis revisited: lessons learned from the National Institutes of Health Interstitial Cystitis Database study. J Urol. 1999 February; 161(2):553-7.
4. Parsons, C. L., et al., Abnormal urinary potassium metabolism in patients with interstitial cystitis. J Urol. 2005; 173(4):1182-5.
5. Parsons, C. L., et al., The role of urinary potassium in the pathogenesis and diagnosis of interstitial cystitis. J Urol. 1998; 159(6):1862-6; discussion 1866-7.
6. Hassan A A, Elgamal S A, Sabaa M A, Salem K Evaluation of intravesical potassium sensitivity test and bladder biopsy in patients with chronic prostatitis/chronic pelvic pain syndrome. Int J Urol. 2007 August; 14(8):738-42.
7. Daha, L., et al., Comparative (saline vs. 0.2M potassium chloride) assessment of maximum bladder capacity: a well tolerated alternative to the 0.4M potassium sensitivity test (PST). J Urol. 2001; 165(suppl):68.
8. Philip, J., S. Willmott, and P. Irwin, Interstitial cystitis versus detrusor overactivity: a comparative, randomized, controlled study of cystometry using saline and 0.3 M potassium chloride. J Urol. 2006; 175(2):566-70; discussion 570-1.
9. Abrams P, Hanno P, Wein A Overactive Bladder and Painful Bladder Syndrome: There Need not be Confusion. Neurourology and Urodynamics 24:149-150 (2005)
10. Parsons, C. L., et al., The prevalence of interstitial cystitis in gynecologic patients with pelvic pain, as detected by intravesical potassium sensitivity. Am J Obstet Gynecol. 2002; 187(5):1395-400.
11. Parsons C L, The potassium sensitivity test: a new gold standard for diagnosing and understanding the pathophysiology of interstitial cystitis. J Urol. 2009 August; 182(2):432-4
12. Parsons C L, Successful management of radiation cystitis with sodium pentosanpolysulfate. J Urol. 1986 October; 136(4):813-4
13. Parsons C L, et al Treatment of interstitial cystitis with intravesical heparin Br J Urol. 1994 May; 73(5):504-7).
14. Parsons C L et al, A quantitatively controlled method to study prospectively interstitial cystitis and demonstrate the efficacy of pentosanpolysulfate. J Urol. 1993 September; 150(3):845-8)
15. Parsons C L et al, Inhibition of sodium urate crystal adherence to bladder surface by polysaccharide. J Urol. 1985 September; 134(3):614-6.
16. Mousa S A et al, Pharmacokinetics and pharmacodynamics of oral heparin solid dosage form in healthy human subjects. J Clin Pharmacol. 2007 December; 47(12):1508-20.

### Advantages of the Invention

The present invention provides improved treatment methods and compositions for the oral treatment of LUDE, or a disease, condition, or syndrome associated with LUDE, including interstitial cystitis, overactive bladder (OAB), prostatitis (CP/CPPS), urethral syndrome (US) and gynecologic chronic pelvic pain (CPP), renal calculi, radiation cystitis, and urinary infections as well as other diseases and conditions, such as a disease or condition selected from the group consisting of HIV infection, prostate cancer, osteoarthritis, prion disease, including variant Creutzfeldt-Jakob disease, inflammatory myocardial injury, osteonecrosis, intervertebral disc degeneration, amyloid-β-induced toxicity in Alzheimer's disease, and atherosclerosis, as well as a number of inflammation-related diseases and conditions, including rheumatoid arthritis, juvenile rheumatoid arthritis, psoriatic arthritis, ankylosing spondylitis, lupus erythematosus, multiple sclerosis, and asthma The treatment methods and compositions according to the present invention provide improved oral delivery and bioavailability of the pharmacologically active agent sodium pentosan polysulfate and are well accepted by patients. They are free of side effects and can be used together with other therapies for treatment of LUDE, or, alternatively, a disease or condition selected from the group consisting of HIV infection, prostate cancer, osteoarthritis, prion disease, including variant Creutzfeldt-Jakob disease, inflammatory myocardial injury, osteonecrosis, intervertebral disc degeneration, amyloid-β-induced toxicity in Alzheimer's disease, atherosclerosis, and abnormal coagulation.

Methods according to the present invention possess industrial applicability for the preparation of a medicament for the oral treatment of LUDE, or a disease, condition, or syndrome associated with LUDE, including interstitial cystitis, overactive bladder (OAB), prostatitis (CP/CPPS), urethral syndrome (US) and gynecologic chronic pelvic pain (CPP), renal calculi, radiation cystitis, and urinary infections or, alternatively, a disease or condition selected from the group consisting of HIV infection, prostate cancer, osteoarthritis, prion disease, including variant Creutzfeldt-Jakob disease, inflammatory myocardial injury, osteonecrosis, intervertebral disc degeneration, amyloid-β-induced toxicity in Alzheimer's disease, atherosclerosis, and abnormal coagulation, as well as rheumatoid arthritis, juvenile rheumatoid arthritis, psoriatic arthritis, ankylosing spondylitis, lupus erythematosus, multiple sclerosis, and asthma. Pharmaceutical compositions according to the present invention also possess industrial applicability as compositions of matter.

The method claims of the present invention provide specific method steps that are more than general applications of laws of nature and require that those practicing the method steps employ steps other than those conventionally known in the art, in addition to the specific applications of laws of nature recited or implied in the claims, and thus confine the scope of the claims to the specific applications recited therein. In some contexts, these claims are directed to new ways of using an existing drug.

With respect to ranges of values, the invention encompasses each intervening value between the upper and lower limits of the range to at least a tenth of the lower limit's unit, unless the context clearly indicates otherwise. Moreover, the invention encompasses any other stated intervening values and ranges including either or both of the upper and lower limits of the range, unless specifically excluded from the stated range.

Unless defined otherwise, the meanings of all technical and scientific terms used herein are those commonly understood by one of ordinary skill in the art to which this invention belongs. One of ordinary skill in the art will also appreciate that any methods and materials similar or equivalent to those described herein can also be used to practice or test this invention.

The publications and patents discussed herein are provided solely for their disclosure prior to the filing date of the present application. Nothing herein is to be construed as an admission that the present invention is not entitled to antedate such publication by virtue of prior invention. Further the dates of publication provided may be different from the actual publication dates which may need to be independently confirmed.

All the publications cited are incorporated herein by reference in their entireties, including all published patents, patent applications, and literature references, as well as those publications that have been incorporated in those published documents. However, to the extent that any publication incorporated herein by reference refers to information to be published, applicants do not admit that any such information published after the filing date of this application to be prior art.

As used in this specification and in the appended claims, the singular forms include the plural forms. For example the terms “a,” “an,” and “the” include plural references unless the content clearly dictates otherwise. Additionally, the term “at least” preceding a series of elements is to be understood as referring to every element in the series. The inventions illustratively described herein can suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the future shown and described or any portion thereof, and it is recognized that various modifications are possible within the scope of the invention claimed. Thus, it should be understood that although the present invention has been specifically disclosed by preferred embodiments and optional features, modification and variation of the inventions herein disclosed can be resorted by those skilled in the art, and that such modifications and variations are considered to be within the scope of the inventions disclosed herein. The inventions have been described broadly and generically herein. Each of the narrower species and subgeneric groupings falling within the scope of the generic disclosure also form part of these inventions. This includes the generic description of each invention with a proviso or negative limitation removing any subject matter from the genus, regardless of whether or not the excised materials specifically resided therein. In addition, where features or aspects of an invention are described in terms of the Markush group, those schooled in the art will recognize that the invention is also thereby described in terms of any individual member or subgroup of members of the Markush group. It is also to be understood that the above description is intended to be illustrative and not restrictive. Many embodiments will be apparent to those of in the art upon reviewing the above description. The scope of the invention should therefore, be determined not with reference to the above description, but should instead be determined with reference to the appended claims, along with the full scope of equivalents to which such claims are entitled. Those skilled in the art will recognize, or will be able to ascertain using no more than routine experimentation, many equivalents to the specific embodiments of the invention described. Such equivalents are intended to be encompassed by the following claims.

1. A pharmaceutical composition comprising:
(a) a therapeutically effective quantity of a pentosan polysulfate salt;
(b) a quantity of a penetration enhancer sufficient to improve the bioavailability of the pentosan polysulfate salt; and
(c) optionally, a pharmaceutically acceptable carrier.

2. The pharmaceutical composition of claim 1 wherein the pentosan polysulfate salt is selected from the group consisting of sodium pentosan polysulfate, potassium pentosan polysulfate, and calcium pentosan polysulfate.

3. The pharmaceutical composition of claim 2 wherein the pentosan polysulfate salt is sodium pentosan polysulfate.

4. The pharmaceutical composition of claim 3 wherein the quantity of sodium pentosan polysulfate originally present in the composition is from about 50 mg to about 300 mg per unit dose of the composition.

5.-7. (canceled)

8. The pharmaceutical composition of claim 1 wherein the quantity of penetration enhancer is from about 50 mg to about 800 mg per unit dose of the composition.

9.-13. (canceled)

14. The composition of claim 1 wherein the ratio, by weight, of the penetration enhancer to the pentosan polysulfate salt is from about 0.167:1 to about 8:1.

15.-19. (canceled)

20. The composition of claim 3 wherein the quantity of penetration enhancer used is sufficient to increase the bioavailability of sodium pentosan polysulfate to at least 5%.

21.-23. (canceled)

24. The composition of claim 3 wherein the composition provides a peak plasma concentration of sodium pentosan polysulfate from about 0.1 hour to about 3 hours after the administration of the composition.

25.-27. (canceled)

28. The composition of claim 1 wherein the penetration enhancer is selected from the group consisting of N-benzoyl-α-amino acids of Formula (II) and salts thereof: 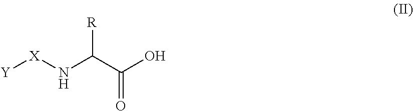
wherein the α-amino acid is selected from the group consisting of glycine, alanine, valine, leucine, phenylalanine, tyrosine, aspartic acid, glutamic acid, lysine, ornithine, arginine, and serine, wherein X is selected from the group consisting of C(O) and SO₂, and wherein Y is selected from the group consisting of phenyl and cyclohexyl.

29. The composition of claim 1 wherein the penetration enhancer is selected from the group consisting of derivatized leucines of Formula (III) and salts; thereof: 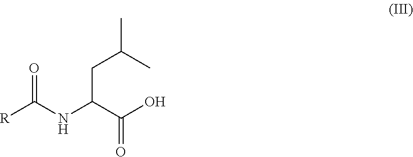
wherein R is selected from the group consisting of cyclohexyl, 2-methylcyclohexyl, 3-methylcyclohexyl, 4-methylcyclohexyl, cycloheptyl, cyclopentyl, cyclopropyl, 2-carboxycyclohexyl, benzoyl, 3-methoxyphenyl, 2-nitrophenyl, 3-nitrophenyl, 4-nitrophenyl, and (CH₂)₂cyclohexyl.

30. The composition of claim 1 wherein the penetration enhancer is selected from the group consisting of derivatives of 4-aminobenzoic acid, 2-(4-aminophenyl)acetic acid, 3-(4-aminophenyl)propionic acid, or 4-(4-aminophenyl)butyric acid of Formula (VI) and salts thereof: 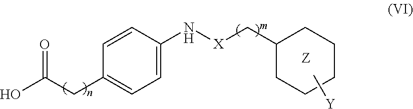
wherein: (a) Y is selected from the group consisting of H, F, 2-OH, 2,3-Ph, 4-Ph, 3,4-Ph, 4-OCH₃, 4-F, 2-Cl, 2-F, 2,4-(OH)₂, 3-CF₃, 3-Cl, 2-CH₃, 2,6-(OH)₂, 3-N(CH₃), 3,4-OCH₂O, 2,6-diCH₃, 2-COOH, 2-NO₂, 2-OCH₃, 3-NO₂, 2-OCF₃, 4-CH₃, and 4-i-Bu; (b) n is 0, 1, 2, 3, 4, or a vinyl group; (c) m is 0, 1, or 2, a vinyl group, a CHMe group, a CHEt group; a (CH₂)₂O group, a (CH₂)₂C═O group, or a (CH₂OH)₂ group; (d) X is C═O, SO₂, or CH₂; and (e) Z is phenyl, cyclohexyl, or cycloheptyl.

31. The composition of claim 1 wherein the penetration enhancer is selected from the group consisting of compounds of Formula (VII): 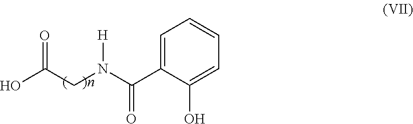
wherein n is 1, 2, 3, 4, 5, 6, 7, 8, 9, 10, or 11, and salts thereof.

32. The composition of claim 31 wherein the penetration enhancer is selected from the group consisting of compounds or salts of Formula (VII) that have n as 7, 8, or 9.

33. The composition of claim 1 wherein the penetration enhancer is sodium N-[8-(2-hydroxybenzoyl)amino]caprylate.

34. The composition of claim 1 wherein the penetration enhancer is selected from the group consisting of phenoxycarboxylic acid compounds of Formula (VIII): 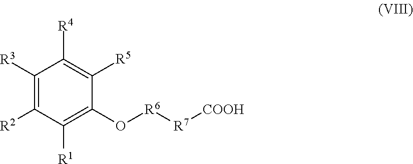
wherein: (i) R¹, R², R³, and R⁴ are each independently hydrogen, hydroxyl, halo, C₁-C₄ alkyl, C₂-C₄ alkenyl, C₁-C₄ alkoxy, —C(O)R⁸, —NO₂, —NR⁹R¹⁰, or —N⁺R⁹R¹⁰R¹¹(R¹²)⁻; (ii) R⁵ is hydrogen, hydroxyl, —NO₂, halo, trifluoromethyl, —NR¹⁴R¹⁵, —N⁺R¹⁴R¹⁵R¹⁶(R¹³)⁻, amide, C₁-C₁₂ alkoxy, C₁-C₁₂ alkyl, C₂-C₁₂ alkenyl, carbamate, carbonate, urea, or —C(O)R¹⁸; (iii) R⁵ is optionally substituted with halo, hydroxyl, sulfhydryl, or carboxyl; (iv) R⁵ is optionally interrupted by O, N, S, or —C(O)—; (v) R⁶ is a C₁-C₁₂ alkylene, C₂-C₁₂ alkenylene, or arylene; (vi) C⁶ is optionally substituted with a C₁-C₄ alkyl, C₂-C₄ alkenyl, C₁-C₄ alkoxy, hydroxyl, sulfhydryl, halo, amino, or —CO₂R⁸; (vii) R⁶ is optionally interrupted by O or N; (viii) R⁷ is a bond or arylene; (ix) R⁷ is optionally substituted with hydroxyl, halogen, —C(O)CH₃, —NR¹⁰R¹¹, or —N⁺R¹⁰R¹¹R¹²(R¹³)⁻; (x) R⁸ is hydrogen, C₁-C₄ alkyl, C₂-C₄ alkenyl, or amino; (xi) R⁹, R¹⁰, R¹¹, and R¹² are each independently hydrogen or C₁-C₁₀ alkyl; (xii) R¹³ is a halide, hydroxide, sulfate, tetrafluoroborate, or phosphate; (xiv) R¹⁴, R¹⁵, and R¹⁶ are each independently hydrogen, C₁-C₁₀ alkyl, C₁-C₁₀ alkyl substituted with carboxyl, C₂-C₁₂ alkenyl, C₂-C₁₂ alkenyl substituted with carboxyl, or C(O)R¹⁷; (xv) R¹⁷ is hydroxyl, C₁-C₁₀ alkyl, or C₂-C₁₂ alkenyl; (xvi) R¹⁸ is hydrogen, C₁-C₆ alkyl, hydroxyl, —NR¹⁴R¹⁵, or —N⁺R¹⁴R¹⁵R¹⁶(R¹³)⁻; with the proviso that: (a) when R¹, R², R³, R⁴, and R⁵ are hydrogen and R⁷ is a bond, then R⁶ is not a C₁-C₆, C₉, or C10 alkyl; (b) when R¹, R², R³, and R⁴ are hydrogen, R⁵ is hydroxyl, and R⁷ is a bond, then R⁶ is not a C₁-C₃ alkyl; (c) when at least one of R¹, R², R³, and R⁴ is not hydrogen, R⁵ is hydroxyl, and R⁷ is a bond, then R⁶ is not a C₁-C₄ alkyl; (d) when R¹, R², and R³ are hydrogen, R⁴ is —OCH₃, R⁵ is C(O)CH₃, and R⁶ is a bond, then R⁷ is not a C₃ alkyl; and (e) when R¹, R², R⁴, and R⁵ are hydrogen, R³ is hydroxyl, and R⁷ is a bond, then R⁶ is not a methyl group.

35. The composition of claim 1 wherein the penetration enhancer is selected from the group consisting of compounds with a cyclic moiety of Formula (IX): 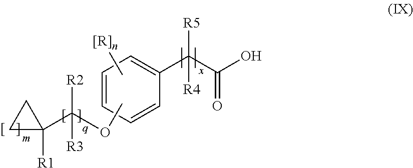
wherein: m is 1, 2, 3, 4, 5, or 6; n is 0, 1, 2, 3, or 4, q and x are independently chosen from 0, 1, 2, 3, 4, 5, 6, 7, 8, 9, or 10; R may be the same or different and is selected from hydrogen, halogen, a substituted or non-substituted alkyl, substituted or non-substituted alkyloxyl, substituted or non-substituted alkenyloxyl, substituted or non-substituted alkynyloxyl and substituted or non-substituted aryloxyl; and R1, R2, R3, R4 and R5 are independently selected from hydrogen, halogen, substituted or non-substituted alkyl, substituted or non-substituted alkenyl, substituted or non-substituted alkynyl, substituted or non-substituted alkyloxyl, substituted or non-substituted aryloxyl, substituted or non-substituted aryl groups, substituted or non-substituted heteroaryl, substituted or non-substituted cycloalkyl, and substituted or non-substituted heterocycloalkyl groups.

36. The composition of claim 1 wherein the penetration enhancer is selected from compounds with an aromatic nucleus of Formula (X): 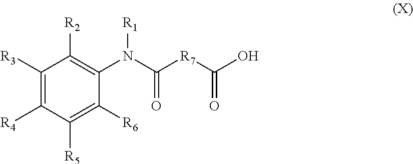
wherein: (i) R₁ is —(CH₂)ₘ—R₈, wherein m is 0 or 1; (ii) R₂, R₃, R₄, R₅, and R₆ are each independently selected from hydrogen, hydroxyl, halo, C₁-C₄ alkyl, C₂-C₄ alkenyl, C₂-C₄ alkynyl, C₁-C₄ alkoxy, and cyano; (iii) R₇ is selected from C₁-C₁₀ alkyl, C₂-C₁₀ alkenyl, and C₂-C₁₀ alkynyl; (iv) R₈ is selected from cyclopentyl, cyclohexyl, and phenyl, wherein, when R₈ is phenyl, m is 1; and (v) R₈ is optionally substituted with C₁-C₄ alkyl, C₁-C₄ alkoxy, halo, hydroxyl, or a combination thereof.

37. The composition of claim 1 wherein the penetration enhancer is selected from the group consisting of: (1) disodium salts of Formula (XI); (2) monohydrates of disodium salts of Formula (XI); and (3) alcohol solvates of disodium salts of Formula (XI), wherein the alcohol is methanol, ethanol, propanol, propylene glycol, or another monohydroxylic or dihydroxylic alcohol: 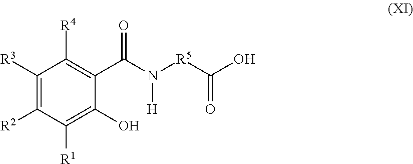
wherein: (i) R¹, R², R³, and R⁴ are each independently hydrogen, hydroxyl, —NR⁶R⁷, halo, C₁-C₄ alkyl, or C₁-C₄ alkoxy; (ii) R⁵ is a substituted or unsubstituted C₂-C₁₆ alkylene, substituted or unsubstituted C₁-C₁₂ alkyl(arylene), or substituted or unsubstituted aryl(C₁-C₁₂ alkylene); and (iii) R⁶ and R⁷ are each independently hydrogen, oxygen, or C₁-C₄ alkyl.

38. The composition of claim 37 wherein the penetration enhancer is selected from the group consisting of N-(5-chlorosalicyloyl)-8-aminocaprylic acid (“5-CNAC”), N-(10-[2-hydroxybenzoyl]amino)decanoic acid (“SNAD”), N-(8-[2-hydroxybenzoyl]amino)caprylic acid (“SNAC”), 8-(N-2-hydroxy-4-methoxybenzoyl)aminocaprylic, and N-(9-(2-hydroxybenzoyl)aminononanoic acid.

39. The composition of claim 1 wherein the penetration enhancer is selected from the group consisting of 8-(N-2-hydroxy-4-methoxybenzoyl)-aminocaprylic acid (“4-MOAC”), N-(8-[2-hydroxybenzoyl]-amino) caprylic acid (“NAC”), N-(8-[2-hydroxybenzoyl]-amino)decanoic acid (“NAD”), N-(8-[2-hydroxy-5-chlorobenzoyl]-amino)octanoic acid (“5-CNAC”), and 4-[(2-hydroxy-4-chlorobenzoyl)amino]butanoate (“4-CNAB”).

40. The composition of claim 1 wherein the penetration enhancer is selected from the group consisting of:
(a) compounds of Formula (XII): 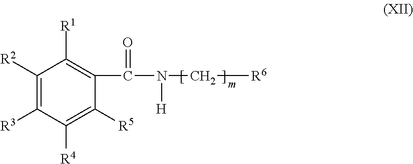
wherein: (i) R¹, R², R³, R⁴, and R⁵ are each independently selected from hydrogen, halo, hydroxyl, —OCH₃, C₁-C₄ alkyl, amino, methylamino, dimethylamino, or nitro; (ii) m is 0, 1, 2, 3, or 4; (iii) R⁶ is phenyl substituted with —O—R⁷—COOH at the ortho, meta, or para position; (iv) R⁶ is optionally substituted with one or more substituents selected from hydrogen, halo, hydroxyl, —OCH₃, C₁-C₄ alkyl, amino, methylamino, dimethylamino, or nitro; and (iv) R⁷ is C₁-C₁₂ alkyl;
(b) compounds of Formula (XIII): 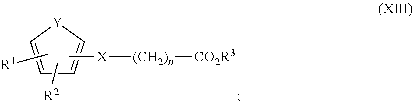
wherein: (i) R¹ and R² are each independently hydrogen, hydroxyl, cyano, C₁-C₆ alkyl, C₁-C₆ alkoxy, CF₃, halo, or NR⁴R^{4′}; (ii) R³ is H or C₁-C₆ alkyl; (iii) X is a 5-membered aromatic heterocycle that is optionally substituted with C₁-C₄ alkyl; wherein the heterocycle contains at least two or three heteroatoms selected from N, S, and O wherein at least one heteroatom is N: (iv) Y is S, CR⁵═N or N═CR⁵; (v) n is 2, 3, 4, 5, 6, or 7; (vi) R⁴ is H, COR⁶, SO₂R⁷, or C₁-C₆ alkyl; (vii) R^{4′} is H or C₁-C₆ alkyl; (viii) R⁵ is H or forms a bond with X; (ix) R⁶ is H or C₁-C₆ alkyl; and (x) R⁷ is H or C₁-C₆ alkyl;
(c) the compound of Formula (XIV) 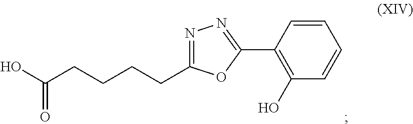 [SMILES: O=C(O)CCCCc1nnc(-c2ccccc2O)o1]
(d) sodium 4-[(4-chloro-2-hydroxybenzoyl)amino]butanoate;
(e) a compound of Formula (XV): 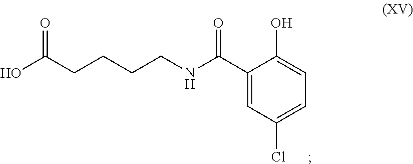 [SMILES: O=C(O)CCCCNC(=O)c1cc(Cl)ccc1O]
(f) a compound that is a polymeric penetration enhancer of Formula (XVa): 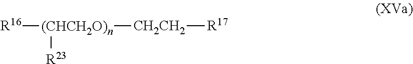
wherein: (i) R¹⁶ is R³-R⁴; (ii) R³
is —NHC(O)NH—, —C(O)NH—, —NHC(O)—, —OOC—, —COO, —NHC(O)O—, —OC(O)NH—, —CH₂NH—, —N HCH₂—, —CH₂NHC(O)O—, —OC(O)NHCH₂—, —CH₂NHCOCH₂O—, —OCH₂C(O)NHCH2-, —NHC(O) CH₂O—, —OCH₂C(O)NH—, —NH—, —O—, or a carbon-carbon bond; R⁴ is Formula (XVIa(1)): 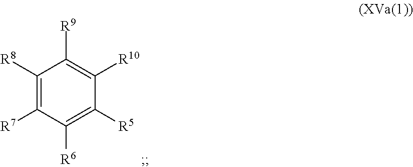
R⁵, R⁶, R⁷, R⁸, and R⁹ are each independently a bond to R³, or hydrogen, chloro, bromo, fluoro, hydroxyl, methyl, methoxy, or —(CH₂)ₘCH₃; R10 is a bond to R³, carboxyl, or —C(O)NHR¹¹R¹²; R¹¹ is a substituted or unsubstituted, linear or branched alkylene having a chain length of 1 to 11 carbon atoms or —R¹³R¹⁴—; R¹² is a bond to R³, carboxyl, amino, hydroxyl, —C(O)—R¹⁵, —COO—R¹⁵, —NHR¹⁵, —OR¹⁵, chloro, or bromo; R¹³ is a substituted or unsubstituted phenylene; R¹⁴ is a substituted or unsubstituted, linear or branched alkylene having a chain length of 1 to 5 carbon atoms; R¹⁵ is a bond to R³; m is 1, 2, 3, or 4: R¹⁷ is hydroxyl or methoxy; R²³ is hydrogen or methyl; and n is an integer from 3 to 200;
(g) a compound of Formula (XVI): 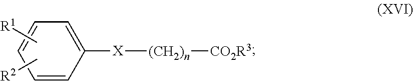
wherein: (i) R¹ and R² are each independently hydrogen, hydroxyl, cyano, C₁-C₆ alkyl, C₁-C₆ alkoxy, CF₃, halo, or NR⁴R^{4′}; (ii) R³ is H or C₁-C₆ alkyl; (iii) R⁴ is H, COR⁵, SO₂R⁶, or C₁-C₆ alkyl: (iv) R^{4′} is H or C₁-C₆ alkyl; (v) R⁵ is H or C₁-C₆ alkyl: (vi) R⁶ is H or C₁-C₆ alkyl: (vii) X is a 5-membered aromatic heterocycle that is optionally substituted with C₁-C₄ alkyl, wherein the heterocycle contains at least two or three heteroatoms selected from N, S, and O, wherein at least one heteroatom is N, and wherein the heterocycle is not 1,3,4-oxadiazole and (ix) n is 2, 3, 4, 5, 6, or 7:
(h) a compound of Formula (XVII): 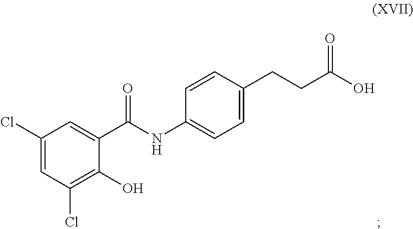 [SMILES: O=C(O)CCc1ccc(NC(=O)c2cc(Cl)cc(Cl)c2O)cc1]
(i) 5-(2-hydroxy-4-chlorobenzoyl) aminovaleric acid; and
(j) a cyanophenoxy carboxylic acid compound of Formula (XVIII): 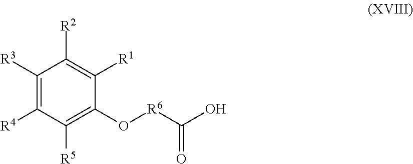
wherein: (i) R¹, R², R³, R⁴, and R⁵ are each independently hydrogen, cyano, hydroxyl, —OCH₃ or halogen, where at least one of R¹, R², R³, R⁴, and R⁵ is cyano; (ii) R⁶ is C₁-C₁₂ linear or branched alkylene, alkenylene, arylene, alkyl(arylene), or aryl(alkylene); with the proviso that where R¹ is cyano, R⁴ is hydrogen or cyano, and R², R³, and R⁵ is not methylene.

41.-49. (canceled)

50. The composition of claim 1 wherein the penetration enhancer is selected from the group consisting of:
(a) a penetration enhancer of Formula (XIX): 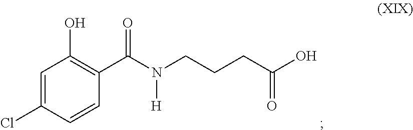 [SMILES: O=C(O)CCCNC(=O)c1ccc(Cl)cc1O]
(b) a penetration enhancer selected from the group consisting of 4-(8-(2-hydroxyphenoxy)octyl)morpholine, 8-(2-hydroxyphenoxy)octyldiethanolamine, 7-(4-2-hydroxyphenoxy)heptylmorpholine, 4-(6-(4-hydroxyphenoxy)hexyl)morpholine, 4-(6-(2-hydroxyphenoxy)hexyl)morpholine, 8-(4-hydroxyphenoxy)octanamine, 6-(2-acetylphenoxy)-1-dimethylaminohexane, 7-(2-hydroxyphenoxy)heptyl-2-isopropylimidazole, 6-(2-hydroxyphenoxy)hexyl-2-methylimidazole, and 5-chloro-4-methyl-2-(8-morpholin-4-yloctyloxy)acetophenone;
(c) a penetration enhancer of Formula (XX): 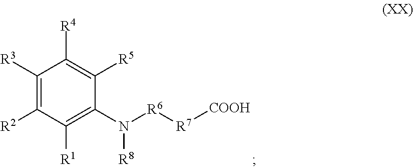
including compounds with the following combinations of substituents: (1) R¹, R², R³, and R⁴ are each hydrogen, R⁵ is carboxyl, R⁶ is (CH₂)₇, R⁷ is a bond, and R⁸ is hydrogen; (2) R¹, R², R³, and R⁴ are each hydrogen, R⁵ is C(O)NH₂, R⁶ is (CH₂)₇, R⁷ is a bond, and R⁸ is hydrogen; (3) R¹, R², R³, and R⁴ are each hydrogen, R⁵ is C(O)CH₃, R⁶ is (CH₂)₇, R⁷ is a bond, and R⁸ is hydrogen; (4) R¹, R², R³, and R⁴ are each hydrogen, R is C(O)NH₂, R⁶ is (CH₂), R⁷ is p-phenyl, and R⁸ is hydrogen; and (5) R¹, R², R³, and R⁴ are each hydrogen, R⁵ is nitro, R⁶ is (CH₂)₇, R⁷ is a bond, and R⁸ is hydrogen;
(d) a diketopiperazine penetration enhancer of Formula (XXI): 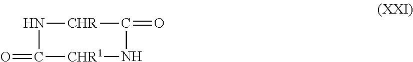
wherein: (i) R and R¹ are C₁-C₂₄ alkyl having a functional group selected from halogen, oxygen, sulfur or nitrogen; (ii) R and R¹ are optionally interrupted with O, N, or S; (iii) R and R¹ are optionally substituted with C₁-C₄ alkyl, C₁-C₄ alkenyl, or CO₂R² or any combination thereof; and (iv) R² is hydrogen, C₁-C₄ alkyl, or C₁-C₄ alkenyl;
(e) a penetration enhancer of Formula (XXII): 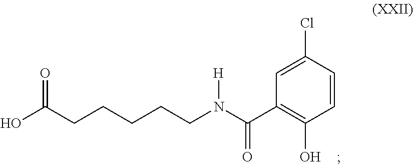 [SMILES: O=C(O)CCCCCNC(=O)c1cc(Cl)ccc1O]
and
(f) a penetration enhancer of Formula (XXIII): 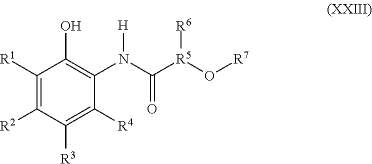
wherein: (i) R¹, R², R³, and R⁴ are each independently hydrogen, hydroxy, halo, C₁-C₄ alkoxy, C₁-C₄ alkyl, C₂-C₄ alkenyl, C₂-C₄ alkynyl, and aryl; (ii) R¹, R², R³, and R⁴ are optionally substituted with halo, hydroxyl, C₁-C₄ alkoxy, or C₁-C₄ alkyl; (iii) R⁵ is C₁-C₄ alkyl; (iv) R⁶ is hydrogen or C₁-C₄ alkyl; (v) R⁷ is hydrogen, C₁-C₄ alkyl, or aryl; and R⁷ is optionally substituted with halogen or hydroxyl.

51.-55. (canceled)

56. The composition of claim 1 wherein the penetration enhancer is selected from the group consisting of:
(a) an amino-substituted carboxylic acid including one or more aromatic moieties therein wherein the aromatic moieties are selected from the group consisting of phenyl, pyrazinyl, pyrimidyl, and chromonyl;
(b) a penetration enhancer of Formula (XXIV): 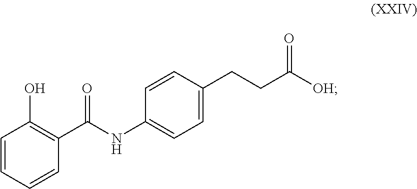 [SMILES: O=C(O)CCc1ccc(NC(=O)c2ccccc2O)cc1]
(c) a penetration enhancer of Formula (XXV): 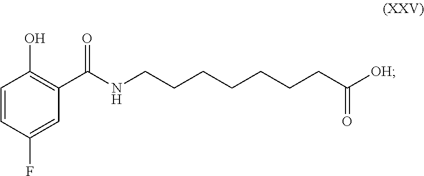 [SMILES: O=C(O)CCCCCCCNC(=O)c1cc(F)ccc1O]
(d) a penetration enhancer of Formula (XXVI): 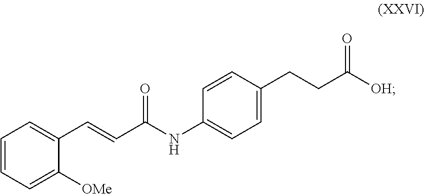 [SMILES: COc1ccccc1/C=C/C(=O)Nc1ccc(CCC(=O)O)cc1]
(e) a penetration enhancer of Formula (XXVII):
2-HO—Ar—CONR⁸—R⁷—COOH (XXVII)
wherein: (i) Ar is a phenyl or naphthyl substituted with at least one of C₁-C₅ alkyl, C₂-C₄ alkenyl, fluoro, chloro, hydroxyl, —SO₂, carboxyl, or —SO₃H; (ii) R⁷ is selected from the group consisting of C₄-C₂₀ alkyl, C₄-C₂₀ alkenyl, phenyl, naphthyl, (C₁-C₁₀ alkyl)phenyl, (C₁-C₁₀ alkenyl)phenyl, C₁-C₁₀ alkyl)naphthyl, (C₁-C₁₀ alkenyl)naphthyl, phenyl(C₁-C₁₀ alkyl), phenyl(C₁-C₁₀ alkenyl), naphthyl(C₁-C₁₀ alkyl), and phenyl(C₁-C₁₀ alkenyl); (iii) R⁷ is optionally substituted with C₁-C₄ alkyl, C₁-C₅ alkenyl, C₁-C₅ alkoxy, hydroxyl, sulfhydryl, and —CO₂R⁹ or any combination thereof; (iv) R⁷ is optionally interrupted by oxygen, nitrogen, sulfur, or any combination thereof; (v) R⁸ is selected from the group consisting of hydrogen, C₁-C₄ alkyl, C₁-C₄ alkenyl, hydroxyl, and C₁-C₄ alkoxy; (vi) R⁸ is optionally substituted with C₁-C₄ alkyl, C₁-C₅ alkenyl, C₁-C₅ alkoxy, hydroxyl, sulfhydryl, and —CO₂R⁹ or any combination thereof; and (vii) R⁹ is hydrogen, C₁-C₄ alkyl, or C₁-C₄ alkenyl, with the proviso that the compounds are not substituted with an amino group in the position α to the acid group; and
(f) a penetration enhancer of Formula (XXVIII): 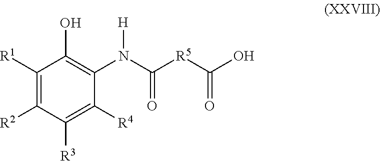
wherein: (i) R¹, R², R³, and R⁴ are independently hydrogen, hydroxyl, halo, C₁-C₄ alkoxy, C₁-C₄ alkyl, C₂-C₄ alkenyl, C₂-C₄ alkynyl, or aryl; (ii) R¹, R², R³, and R⁴ are optionally substituted with halo, hydroxyl, C₁-C₄ alkoxy, or C₁-C₄ alkyl; and (iii) R⁵ is a C₂-C₁₆ branched alkylene, optionally substituted with halogen.

57.-61. (canceled)

62. The composition of claim 1 wherein the penetration enhancer is a penetration enhancer selected from the group consisting of the compounds of Formulas (XXX), (XXXI), (XXXII), (XXXIII), (XXXIV), (XXXV), (XXXVI), (XXXVII), (XXXVIII), (XXXIX), (XL), and (XLI):

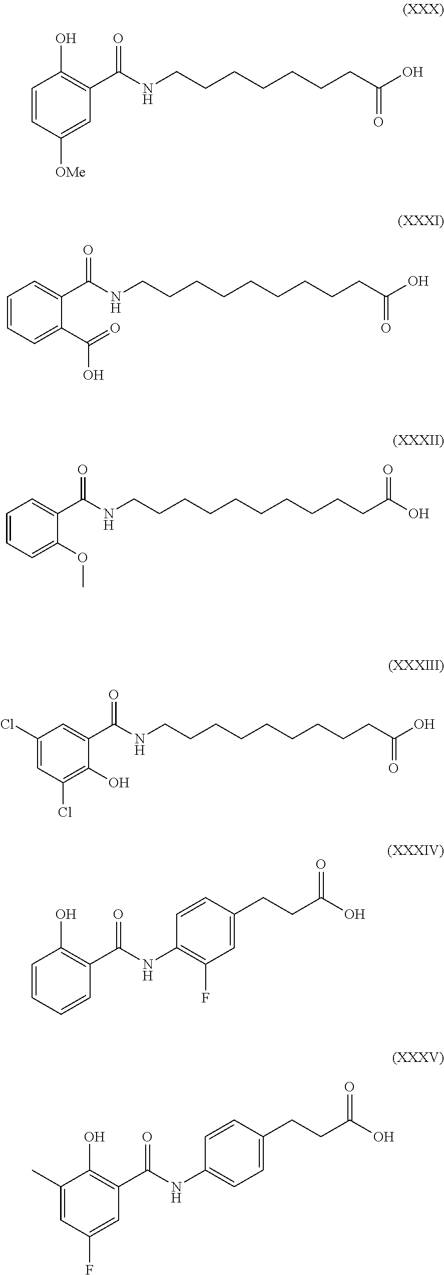 [SMILES: COc1ccc(O)c(C(=O)NCCCCCCCC(=O)O)c1 ; O=C(O)CCCCCCCCCNC(=O)c1ccccc1C(=O)O ; COc1ccccc1C(=O)NCCCCCCCCCCC(=O)O ; O=C(O)CCCCCCCCCNC(=O)c1cc(Cl)cc(Cl)c1O ; O=C(O)CCc1ccc(NC(=O)c2ccccc2O)c(F)c1 ; Cc1cc(F)cc(C(=O)Nc2ccc(CCC(=O)O)cc2)c1O]

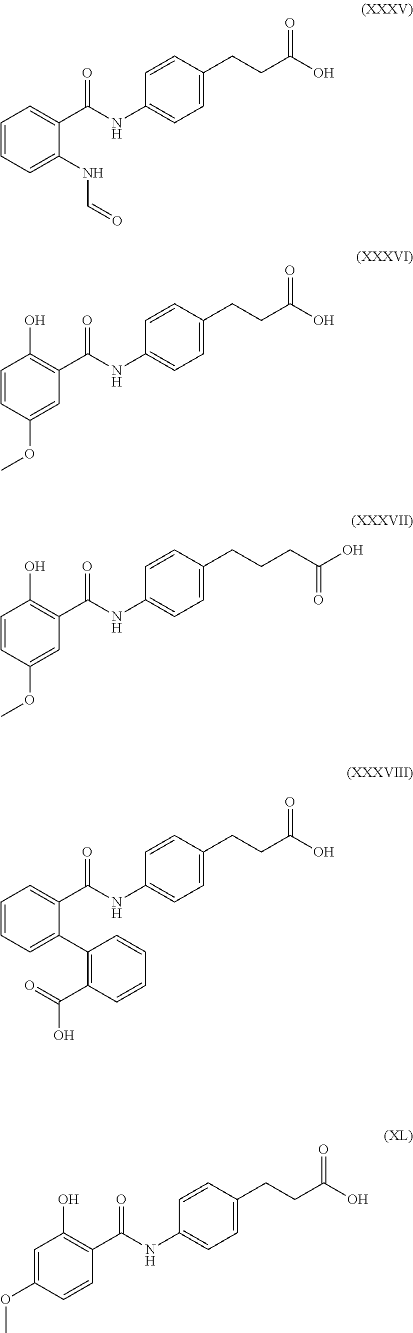 [SMILES: O=CNc1ccccc1C(=O)Nc1ccc(CCC(=O)O)cc1 ; COc1ccc(O)c(C(=O)Nc2ccc(CCC(=O)O)cc2)c1 ; COc1ccc(O)c(C(=O)Nc2ccc(CCCC(=O)O)cc2)c1 ; O=C(O)CCc1ccc(NC(=O)c2ccccc2-c2ccccc2C(=O)O)cc1 ; COc1ccc(C(=O)Nc2ccc(CCC(=O)O)cc2)c(O)c1]

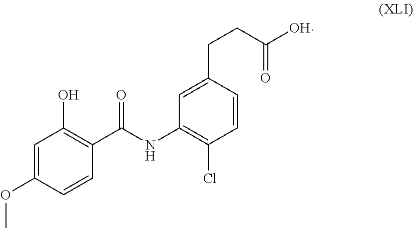 [SMILES: COc1ccc(C(=O)Nc2cc(CCC(=O)O)ccc2Cl)c(O)c1]

63. The composition of claim 1 wherein the penetration enhancer is selected from the group consisting of:
(a) a penetration enhancer of Formula (XLII): 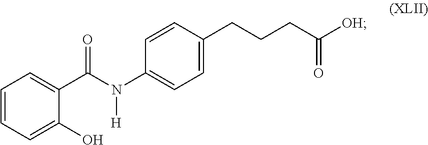 [SMILES: O=C(O)CCCc1ccc(NC(=O)c2ccccc2O)cc1]
b) a compound of Formula (XLIII): 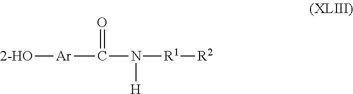
wherein: (i) Ar is phenyl or naphthyl; (ii) Ar is optionally substituted with C₁-C₄ alkyl, C₁-C₄ alkoxy, C₂-C₄ alkenyl, C₂-C₄ alkynyl, aryl, aryloxy, a heterocyclic ring, a C₅-C₇ carbocyclic ring, halo, hydroxyl, sulfhydryl, CO₂R⁶, NR⁷R⁸, or N⁺R⁷R⁸R⁹Y: (iii) (a) R¹ is C₁-C₁₆ alkylene, C₂-C₁₆ alkenylene, C₂-C₁₆ alkynylene, C₆-C₁₆ arylene, (C₁-C₁₆ alkyl)arylene, or aryl(C₁-C₁₆ alkylene); R² is —NR³R⁴, —N⁺R³R⁴, or —N⁺R³R⁴R⁵Y; R³ and R⁴ are each independently hydrogen, oxygen, hydroxyl, substituted or unsubstituted C₁-C₁₆ alkyl, substituted or unsubstituted C₂-C₁₆ alkenyl, substituted or unsubstituted C₂-C₁₆ alkynyl, substituted or unsubstituted aryl, substituted or unsubstituted alkylcarbonyl, substituted or unsubstituted arylcarbonyl, substituted or unsubstituted alkylsulfinyl, substituted or unsubstituted arylsulfinyl, substituted or unsubstituted alkylsulfonyl, substituted or unsubstituted arylsulfonyl, substituted or unsubstituted alkoxycarbonyl, or substituted or unsubstituted aryloxycarbonyl; R⁵ is hydrogen, substituted or unsubstituted C₁-C₁₆ alkyl, substituted or unsubstituted C₂-C₁₆ alkenyl, substituted or unsubstituted C₂-C₁₆ alkynyl, substituted or unsubstituted aryl, substituted or unsubstituted alkylcarbonyl, substituted or unsubstituted arylcarbonyl, substituted or unsubstituted alkylsulfinyl, substituted or unsubstituted arylsulfinyl, substituted or unsubstituted alkylsulfonyl, substituted or unsubstituted arylsulfonyl, substituted or unsubstituted alkoxycarbonyl, or substituted or unsubstituted aryloxycarbonyl; (b) R¹, R², and R⁵ are as above under (a), and R³ and R⁴ are combined to form a 5-, 6-, or 7-membered heterocyclic ring or a aryloxycarbonyl; (b) R¹, R², and R⁵ are as above under (a), and R³ and R⁴ are combined to form a 5-, 6-, or 7-membered heterocyclic ring or a 5-, 6-, or 7-membered heterocyclic ring substituted with C₁-C₆ alkyl, C₁-C₆ alkoxy, aryl, aryloxy, oxo, or carbocyclic ring; or (c) R² and R⁵ are as defined above under (a), and R¹ and R³ are combined to form a 5-, 6-, or 7-membered heterocyclic ring or a 5-, 6-, or 7-membered heterocyclic ring substituted with C₁-C₆ alkyl, C₁-C₆ alkoxy, aryl, aryloxy, oxo, or carbocyclic ring; (iv) R⁴ is hydrogen, oxygen, hydroxyl, substituted or unsubstituted C₁-C₁₆ alkyl, substituted or unsubstituted C₂-C₁₆ alkenyl, substituted or unsubstituted C₂-C₁₆ alkynyl, substituted or unsubstituted aryl, substituted or unsubstituted alkylcarbonyl, substituted or unsubstituted arylcarbonyl, substituted or unsubstituted alkylsulfinyl, substituted or unsubstituted arylsulfinyl, substituted or unsubstituted alkylsulfonyl, substituted or unsubstituted arylsulfonyl, substituted or unsubstituted alkoxycarbonyl, or substituted or unsubstituted aryloxycarbonyl; (v) R⁶ is hydrogen, C₁-C₄ alkyl, C₁-C₄ alkyl substituted with halogen or with hydroxyl, C₂-C₄ alkenyl, or C₂-C₄ alkenyl substituted with halogen or with hydroxyl; (vi) R⁷, R⁸, and R⁹ are each independently hydrogen, oxygen, C₁-C₄ alkyl, C₁-C₄ alkyl substituted with halogen or with hydroxyl, C₂-C₄ alkenyl, or C₂-C₄ alkenyl substituted with halogen or with hydroxyl; and (vii) Y is halogen, hydroxide, sulfate, nitrate, phosphate, alkoxy, perchlorate, tetrafluoroborate, or carboxylate;
(c) a compound of Formula (XLIV): 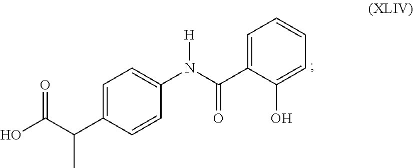 [SMILES: CC(C(=O)O)c1ccc(NC(=O)c2ccccc2O)cc1]
(d) a polymeric delivery agent that comprises a polymer conjugated to modified amino acid or derivative thereof via a linkage group selected from the group consisting of —NHC(O)NH—, —C(O)NH—, —NHC(O)—, —OOC—, —COO—, —NHC(O)O—, —OC(O)NH—, —CH₂NH—, —NHCH₂—, —CH₂NHC(O)O—, —OC(O)NH—, CH₂NCOCH₂O—, —OCH₂C(O)NHCH₂—, —NHC(O)CH₂O—, —OCH₂C(O)NH—, —NH—, —O—, and a carbon-carbon bond, with the proviso that the polymeric delivery agent is not a polypeptide or polyamino acid, wherein the modified amino acids are acylated or sulfonated amino acids, ketones or aldehydes of acylated or sulfonated amino acids, salts thereof, or polyamino acids or polypeptides of any of the foregoing, and the polymer is selected from the group consisting of polyethylene; polyacrylates; polymethacrylates; poly(oxyethylene); poly(propylene); polypropylene glycol; polyethylene glycol (PEG); PEG-maleic anhydride copolymers; and derivatives and combinations thereof;
(e) a compound of Formula (XLV): 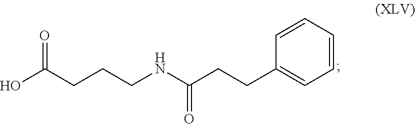 [SMILES: O=C(O)CCCNC(=O)CCc1ccccc1]
(f) a compound of Formula (XLVI): 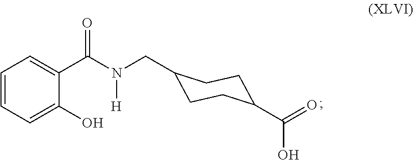 [SMILES: O=C(NCC1CCC(C(=O)O)CC1)c1ccccc1O]
(g) a compound of Formula (XLVII): 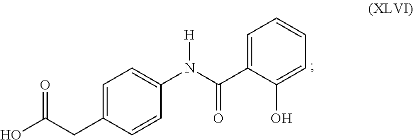 [SMILES: O=C(O)Cc1ccc(NC(=O)c2ccccc2O)cc1]
(h) a compound selected from the group consisting of 6-N-(3,5-dichloro-2-hydroxybenzoyl)aminocaproic acid, 8-(2-aminobenzoylamino)caprylic acid, 8(2-trifluoromethoxy)benzoylaminocaprylic acid, N-(2-hydroxybenzoyl)isonipecotic acid, 4-[4-(2-aminobenzoylamino)phenyl]butyrylhydroxamic acid, 4-(4-(pentafluorobenzoyl)aminophenyl)butyric acid, 4-(4-(3-anisoyl)aminophenyl)butyric acid, 8-(3-anisoyl)aminocaprylic acid, 4-(4-(phenoxyacetyl)aminophenyl)butyric acid, 4-(4-(2-nitrobenzenesulfonyl)aminophenyl)butyric acid, 8-(2-nitrobenzenesulfonyl)aminocaprylic acid, 6-(4-(salicyloyl)aminophenyl)hexanoic acid, 8-(2-methoxybenzoyl)aminocaprylic acid, 2-[4-salicyloylaminophenyl]ethyl methyl sulfone, 1-salicyloyl-2-succinyl-hydrazide, 3-(4-(2,5-dimethoxycinnamoyl)aminophenyl)propionic acid, 4-(4-(2,5-dimethoxycinnamoyl)aminophenyl)butyric acid, 1-salicyloyl-2-glutaryl hydrazide, succinyl-4-aminosalicylic acid, 8-(phenoxyacetylamino)caprylic acid, 8-(2-pyrazinecarbonyl)aminocaprylic acid, 4-(4-(2 pyrazinecarbonyl)aminophenylbutyric acid, 6-(4-(N-2-nitrobenzoyl)aminophenyl)hexanoic acid, 6-(4-(N-2-aminobenzoyl)aminophenyl)hexanoic acid, 4-(4-(2-(3-carbonyl)pyrazinecarboxyl)aminophenyl)butyric acid, 4(2-nitrobenzoyl)aminophenylsuccinic acid, 8-(2-(trifluoromethoxy)benzoyl)aminocaprylic acid, 8-(benzylcarbonylamino)caprylic acid, 8-(phenylcarbonylamino)caprylic acid, 2-[4-(2-methoxybenzoylamino)phenyl]ethyl H₂PO₄, 1-salicyloyl-2-suberyl hydrazide, 4-(4-benzyloxycarbonylaminophenyl)butyric acid, 4-(4-)₂-hydroxynicotinoyl)aminophenyl)butyric acid, 9-salicyloylaminononanic acid, 4-(4-phenyloxycarbonylaminophenyl)butyric acid, 3-(2-methoxybenzoylamino)-1-propanol, 8-(2-hydroxynicotinoyl)aminocaprylic acid, 6-(2-methoxybenzoyl)amino nicotinic acid, salicyloylglycine, 4-(1-(2-pyrimidyl)piperazinoyl)butyric acid, 8-(chromone-3-carbonyl)aminocaprylic acid, 8-(vinylbenzoyl)aminocaprylic acid, 4-(4-(chromone-3-carbonyl)aminophenyl)butyric acid, 8-cinnamoylaminocaprylic acid, 5-(N-salicyloylamino)valeric acid, N-(4-salicyloylamino)-6-caproic acid, 4′-flavonic acid, 11-cinnamoyl aminoundecanoic acid, 4-octanoyl amino-3-hydroxybenzoic acid, (3-phenyl-2,3-dihydroxypropanoyl)-8-aminocaprylic acid, 8-[N-(3-coumarincarbonyl)]aminocaprylic acid, 8-[N-(4-chlorobenzyl)]aminocaprylic acid, 8-[N-(3-fluorobenzyl)]aminocaprylic acid, 8-(N-2,5-dihydroxybenzoyl)aminocaprylic acid, 8-(N-3,5-diacetyloxybenzoyl)aminocaprylic acid, 8-(N-4-hydroxybenzoyl)aminocaprylic acid (dimer), 8-(N-2,4-dihydroxybenzoyl)aminocaprylic acid, 1-(1-(N-2-methoxyanalino)sebacic acid, 10-(N-2-methoxyanilino)sebacic acid, 8-(N-benzoyl)aminocaprylic acid, 2-methoxybenzenaminodecanoic acid, 8-(N-benzoyl)aminocaprylic acid, 8-(N-2-hydroxy-4-methoxybenzoyl)aminocaprylic acid, 8-(N-4-fluorobenzoyl)aminocaprylic acid, 8-(N-3-bromobenzoyl)aminocaprylic acid, 8-(4-(1,2-dihydroxyethyl)benzoyl)aminocaprylic acid, 8-(N-4-bromobenzoyl)aminocaprylic acid, 8-(N-4-iodobenzoyl)aminocaprylic acid, 4-{4-[N-(2-iodobenzoyl)aminophenyl]}butyric acid, 4-{4-[N-(1-hydroxy-2-naphthoyl)aminophenyl]}butyric acid, 4-(4-(2,4-dimethoxybenzoyl)aminophenyl)butyric acid, 4-(o-anisoyl)aminophenylacetic acid, 3-[4-(2,4-dimethoxybenzoyl)aminophenyl]propionic acid, 4-{4-[N-(4-iodobenzoyl)]aminophenyl}butyric acid, 3-[4-(2,3-dimethoxybenzoyl)aminophenyl]propionic acid, 4{4 [N 2 bromobenzoyl)] aminophenyl} butyric acid, 4{4-[N-3[bromobenzoyl) aminophenyl]} butyric acid, 8-(N-3,5-dihydroxybenzoyl)aminocaprylic acid, 8-(N-3,5-dimethoxy 4-hydroxybenzoyl)aminocaprylic acid, 8-(N-2,6-dimethoxybenzoyl)aminocaprylic acid, 4-{4[N-(4 bromobenzoyl)aminophenyl]butyric acid, 8-(2-hydroxy-4-chlorobenzoyl)aminocaprylic acid, 8-(N-2,6-dihydroxybenzoyl)aminocaprylic acid, 8-(N-2-hydroxy-6-methoxybenzoyl)aminocaprylic acid, 8-(5-chloro-o-anisoyl)aminocaprylic acid, 4-(4-(2,3-dimethoxybenzoyl)aminophenyl)butyri c acid, 4-(4-(5 chloro-o-anisoyl)aminophenyl)butyric acid, 4-(4-(4-chloro-o-anisoyl)aminophenyl)butyric acid, 8-(4-chloro-o-anisoyl)aminocaprylic acid, 3-4-(2,5-dimethoxybenzoyl)aminophenyl)propionic acid, 4-{N-[4-(3 iodobenzoyl)aminophenyl]butyric acid, 7-cinnamoylaminoheptanoic acid, 8-N-(3 iodobenzoyl)aminocaprylic acid, 8-N-(4 methoxy-3-nitobenzoyl)aminocaprylic acid, 8-N-(2 methoxy 4 nitobenzoyl)aminocaprylic acid, 4-{N-[4-(2-methoxy-4-nitrobenzoyl)aminophenyl]}butyric acid, 4-(4-(2, 5-dimethoxybenzoyl)aminophenyl)butyric acid, 8-(N-2-hydroxy-5-bromobenzoyl)aminocaprylic acid, 3-indolebutryic acid, 4-(4-(2,6-dimethoxybenzoyl)aminophenylbutyric acid, 4-[4-N-(4 methoxy-3-nitrobenzoyl)aminophenyl]butyric acid, 8-(N-2-hydroxy-5 chlorobenzoyl)aminocaprylic acid, 8-(N-2-hydroxy-5-iodobenzoyl)aminocaprylic acid, 8-(3-hydroxy-2-naphthoyl)aminocaprylic acid, 8-(N-2-hydroxy-2-nitrobenzoyl)aminocaprylic acid, 8-(N-3-methylsalicyloyl)aminocaprylic acid, 8-(N-5-methylsalicyloyl)aminocaprylic acid, 4-[-N-(2 hydroxy-4-bromobenzoyl)aminophenyl]butyric acid, 8-(N-2,3-dihydroxybenzoyl)aminocaprylic acid, 9-(cinnamoylamino)nonanoic acid, 4-(4-(2-chloro-5-nitrobenzoyl)aminophenyl)butyric acid, 4-[N-(2-hydroxy-5-iodobenzoyl)]aminophenylbutyric acid, N-2-nitrophenyl-N′-(8 octanoic acid) urea, 8-[N-(2-acetoxy-3,5-dibromobenzoyl)aminocaprylic acid, 8-N-(2-chloro-6-fluorobenzoyl)aminocaprylic acid, 8-N-(4-hydroxy-3-nitrobenzoyl)caprylic acid, 4-(4-salicyloylaminophenyl)-4-oxobutyric acid, 12-cinnamoyldodecanoic acid, 4-{4-[N-(3-hydroxy-2-naphthoyl)aminophenyl]}butyric acid, 8-(4-chloro-3-nitrobenzoyl)aminocaprylic acid, 8-(2-chloronicotinoyl)aminocaprylic acid, 8-(2-chloro-5-nitrobenzoyl)aminocaprylic acid, 4-(4-phthalimidophenyl)butyric acid, 4-{4-[N-(3-hydroxy-2-napthoyl)aminophenyl]}propanoic acid, 3-(4-(2,6-dimethoxybenzoyl)aminophenyl)propionic acid, 8-(N-2-hydroxy-3,5-diiodobenzoyl)aminocaprylic acid, 8-(N-2-chloro-4-fluorobenzoyl)aminocaprylic acid, 8 (N 1 hydroxy-2-naphthoyl)aminocaprylic acid, 8-(phthalimido)caprylic acid, 10-(4-chloro-2-hydroxyanilino)sebacic acid monoamide, 6-(anisoyl)aminocaproic acid, 4-(4-(4-chloro-3-nitrobenzoyl)aminophenyl)butyric acid, 11-N-(1-hydroxy-2-naphthoyl)aminoundecanoic acid, bis(N-2-carboxylphenyl-N—(N′-8-octanoic acid)ureal)oxalyl diamide, 2-[2-N-(2-chlorobenzoyl)aminoethoxy]ethanol), 2-[2-N-(4 chlorobenzoyl)aminoethoxy]ethanol, 4-(2-methybenzoyl)amino-3-carboxysulfoxide, 4-(2-methoxybenzoyl)amino 3-carboxypropylsulfone, 4-(4-(3-hydroxyphthalimido)phenyl)butyric acid, 2-[2-N-(2 methoxybenzoyl)aminoethoxyl]ethanol, 2-[2-N-(3 chlorobenzoyl)aminoethoxy]ethanol, bis(N-2-carboxyphenyl)-N—(N′-3(4-aminophenyl)propionic acid)ureal)oxalyl diamide, trans 4 (2 aminobenzamidomethyl)cyclohexamecarboxylic acid, 11-N-(3,5-dichloro-2-hydroxybenzoyl)aminoundecanoic acid, 2-[N-(2-bromobenzoyl)aminoethoxy]ethanol, 7-N-(3,5-dichloro-2-hydroxybenzoyl)aminoheptanoic acid, N-[3,5-dichloro-2-hydroxybenzoyl-4(4-aminophenyl)]butyric acid, trans-4-(N salicyloylaminomethyl)cyclohexane carboxylic acid, N-[3,5-dichloro-2-hydroxybenzoyl-3-(4-aminophenyl)]propionic acid, 12-N-(3,5-dichloro-2-hydroxybenzoyl)aminodecanoic acid, N-(2-hydroxy-4-carboxy)-6-heptenamide, N-(2-bromobenzoyl)morpholine, 8-N-cyclohexanoylaminocaprylic acid, 2-[N-(2-iodobenzoyl)aminoethoxy]ethanol, 5-(4-chloro-2-hydroxyanilinocarbonyl)valeric acid, 8-(2-hydroxyphenoxy)-aminocaprylic acid, N-salicyloyl-5-(3-aminophenyl-valeric acid, 4-(4-(2-ethoxylbenzoyl)aminophenyl)butyric acid, 9-[2-(3-hydroxy)pyridylaminocarbonyl]nonanic acid, 7-(2-hydroxyphenoxyacetyl)aminocaprylic acid, 2-[N-2-hydroxybenzoylamino)ethoxy]ethanol. 4-[N-(3,5-chloro-2-hydroxybenzoyl)]aminophenylacetic acid 8-(2-hydroxy-5-chloroanilinocarbonyl)octanoic acid, N-salicyloyl-5-(4-aminophenyl)valeric acid, 9-(2-hydroxy-5-methylanilinocarbonyl)nonanoic acid, 5-(2-hydroxy-5-methylanilinocarbonyl)valeric acid, 8-(pentafluorobenzoyl)aminocaprylic acid, 3-(3-(salicyloyl)aminophenyl)propionic acid, 8-(2-ethoxybenzoyl)aminocaprylic acid, 4-(4-(2-dimethylamino benzoic)aminophenyl)butyric acid, 8-(3-phenoxylpropionylamino)caprylic acid, 4-(salicyloyl)aminophenylethyltetrazole, 4-(4-(N-(2-fluorocinnamoyl))aminophenyl)butyric acid, 4-(4-(N-8-salicyloyl)aminocaprylic)aminophenyl)butyric acid, 8-(p-anisoyl)aminocaprylic acid, 8-(4-hydroxybenzoyl)aminocaprylic acid, 8-(3-hydroxybenzoyl)aminocaprylic acid, 8-(3,4,5-trimethoxybenzoyl)aminocaprylic acid, 8-(N-4-methyl salicyloyl)aminocaprylic acid, N-10-(2-hydroxy-5-nitroanilino)decanoic acid, and 4-(4-(2-chloronicotinoyl)aminophenyl)butyric acid;
(i) a compound of Formula (XLVIII): 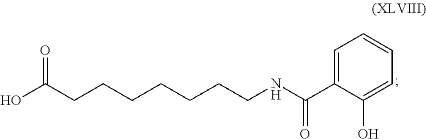 [SMILES: O=C(O)CCCCCCCNC(=O)c1ccccc1O]
and
(k) 8-[(2-hydroxy-4-methoxy-benzoyl) amino]-octanoic acid.

64.-72. (canceled)

73. The composition of claim 1 wherein the penetration enhancer comprises: (i) at least one acylated amino acid; (ii) at least one peptide comprising one acylated amino acid; or (iii) a combination of (i) and (ii), wherein the acylated amino acid is acylated by: (1) a C₃-C₁₀ cycloalkyl acylating agent, the agent being optionally substituted with C₁-C₇ alkyl, C₂-C₇ alkenyl, C₁-C₇ alkoxy, hydroxyl, phenyl, phenoxy, or —CO₂R, wherein R is hydrogen, C₁-C₄ alkyl, or C₂-C₄ alkenyl; or (2) a C₃-C₁₀ cycloalkyl substituted C₁-C₆ alkyl acylating agent, wherein the amino acid is of Formula (XLIX): 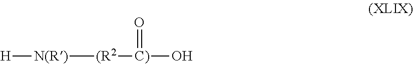
wherein: R¹ is hydrogen, C₁-C₄ alkyl, or C₂-C₄ alkenyl; R² is C₁-C₂₄ alkyl, C₂-C₂₄ alkenyl, C₃-C₁₀ cycloalkyl, C₃-C₁₀ cycloalkenyl, phenyl, naphthyl, (C₁-C₁₀ alkyl) phenyl (C₂-C₁₀ alkenyl) phenyl, (C₁-C₁₀ alkyl) naphthyl (C₂-C₁₀ alkenyl) naphthyl, phenyl (C₁-C₁₀ alkyl), phenyl (C₂-C₁₀ alkenyl), naphthyl (C₁-C₁₀ alkyl) naphthyl (C₂-C₁₀ alkenyl); R² can be optionally substituted with C₁-C₄ alkyl, C₂-C₄ alkenyl, C₁-C₄ alkoxy, hydroxyl, sulfhydryl, —CO₂R³, C₃-C₁₀ cycloalkyl, C₃-C₁₀ cycloalkenyl, heterocycle having 3-10 ring atoms wherein the heteroatom is one or more of N, O. or S, or any combination thereof, aryl, C₁-C₁₀ alkaryl, aryl(C₁-C₁₀ alkyl), or any combination thereof; R² can be optionally interrupted by oxygen, nitrogen, sulfur, or any combination thereof; and R³ is hydrogen, C₁-C₄ alkyl, or C₂-C₄ alkenyl.

74. The composition of claim 1 wherein the penetration enhancer is a modified amino acid prepared by acylation or sulfonation of an amino acid selected from the group consisting of aminobutyric acid, aminocaproic acid, and aminocaprylic acid.

75. The composition of claim 1 wherein the penetration enhancer is selected from the group consisting of:
(a) a compound of Formula (L): 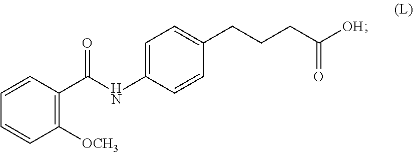 [SMILES: COc1ccccc1C(=O)Nc1ccc(CCCC(=O)O)cc1]
(b) a compound of Formula (LI): 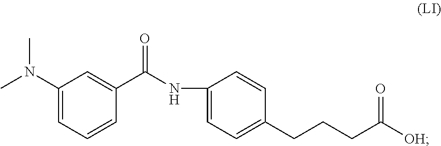 [SMILES: CN(C)c1cccc(C(=O)Nc2ccc(CCCC(=O)O)cc2)c1]
(c) a compound of Formula 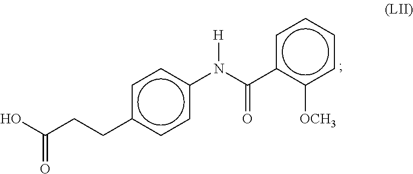
(d) a modified amino acid of either Formula (LIII) or (LIV):
Ar—Y—(R¹)ₙ—OH (LIII);
and 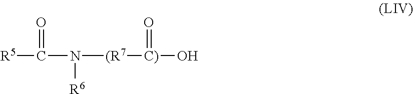
wherein: (i) Ar is an unsubstituted or substituted phenyl or naphthyl; (ii) Y is —C(O)— or —S(Oz)—; (iii) R¹ has the formula —N(R³)—R²—C(O)—; (iv) R² is C₁-C₂₄ alkyl, C₁-C₂₄ alkenyl, phenyl, naphthyl, (C₁-C₁₀ alkyl)phenyl, (C₁-C₁₀ alkenyl)phenyl, (C₁-C₁₀ alkyl)naphthyl, (C₁-C₁₀ alkenyl)naphthyl, phenyl(C₁-C₁ alkyl), phenyl(C₁-C₁₀ alkenyl), naphthyl(C₁-C₁₀ alkyl), or naphthyl(C₁-C₁₀ alkenyl); (v) R² is optionally substituted with C₁-C₄ alkyl, C₁-C₄ alkenyl, C₁-C₄ alkoxy, hydroxyl, sulfhydryl, CO₂R⁴, or any combination thereof; (vi) R⁴ is hydrogen, C₁-C₄ alkyl, or C₁-C₄ alkenyl; (vii) R² is optionally interrupted by oxygen, nitrogen, sulfur, or any combination thereof; (viii) R³ is hydrogen, C₁-C₄ alkyl, or C₁-C₄ alkenyl; (ix) R⁵ is either: (A) C₃-C₁₀ cycloalkyl, optionally substituted with C₁-C₇ alkyl, C₂-C₇ alkenyl, C₁-C₇ alkoxy, hydroxyl, phenyl, phenoxy, or —CO₂R⁸, wherein R⁸ is hydrogen, C₁-C₄ alkyl, or C₂-C₄ alkenyl; or (B) C₁-C₆ alkyl substituted with C₃-C₁₀ cycloalkyl; (x) R⁶ is C₃-C₁₀ cycloalkyl; R⁷ is C₁-C₂₄ alkyl, C₂-C₂₄ alkenyl, C₃-C₁₀ cycloalkyl, phenyl, naphthyl, (C₁-C₁₀ alkyl)phenyl, (C₂-C₁₀ alkenyl)phenyl, (C₁-C₁₀ alkyl)naphthyl, (C₂-C₁₀ alkenyl)naphthyl, phenyl(C₁-C₁₀ alkyl), phenyl(C₂-C₁₀ alkenyl), naphthyl(C₁-C₁₀ alkyl), or naphthyl(C₂-C₁₀ alkenyl); (xi) R⁷ is optionally substituted with C₁-C₄ alkyl, C₂-C₄ alkyl, C₁-C₄ alkoxy, hydroxyl, sulfhydryl, —CO₂R⁹, C₃-C₁₀ cycloalkyl, C₃-C₁₀ cycloalkenyl, a heterocycle having 3-10 ring atoms wherein the heteroatom is one or more of N, O, or S or any combination thereof, aryl, (C₁-C₁₀)alkaryl, aryl(C₁-C₁₀ alkyl), or any combination thereof; (xii) R⁷ is optionally interrupted by oxygen, nitrogen, sulfur, or any combination thereof; and (xiii) R⁹ is hydrogen, C₁-C₄ alkyl, or C₂-C₄ alkenyl;
(e) a compound of Formula (LV): 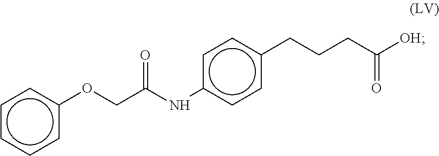
(f) a compound of Formula (LVI): 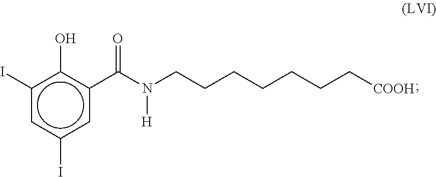
(g) a compound of Formula (LVII): 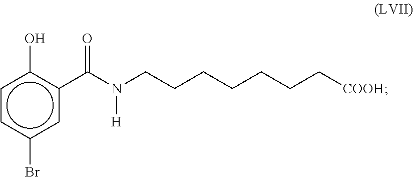
(h) a compound of Formula (LVIII): 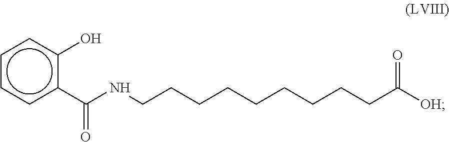
(i) a compound selected from the group consisting of Formulas (LIX), (LX), and (LXI): 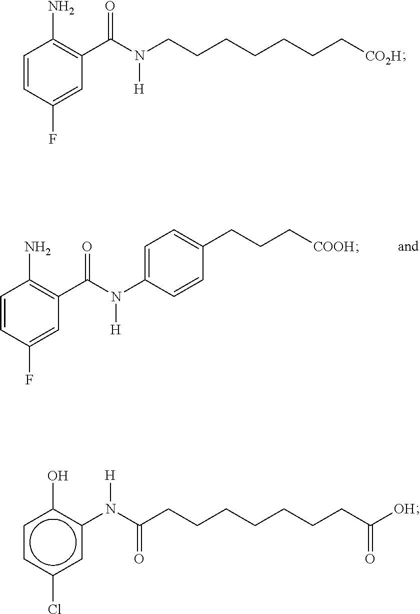
and
(j) a compound of Formula (LXII):

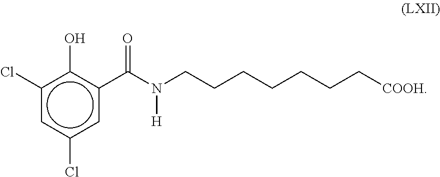

76.-84. (canceled)

85. The composition of claim 1 wherein the penetration enhancer is selected from the group consisting of: (1) (a) at least one acylated aldehyde of an amino acid, (b) at least one acylated ketone of an amino acid, (c) at least one acylated aldehyde of a peptide, (d) at least one acylated ketone of a peptide, (e) any combination of (1)(a), (1)(b), (1)(c), and (1)(d); (2) (a) carboxymethyl-phenylalanylleucine; (b) 2-carboxy-3-phenylpropionylleucine; (c) 2-benzylsuccinic acid; (d) (phenylsulfonamide)phenylbutyric acid; and (e) any combination of (2)(a), (2)(b), (2)(c) and (2)(d); or (3) a combination of (1) and (2).

86. The composition of claim 1 wherein the penetration enhancer is:
(a) a compound of Formula (LXIII): 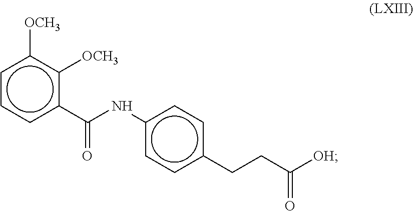
and
(b) a compound of Formula (LXIV):

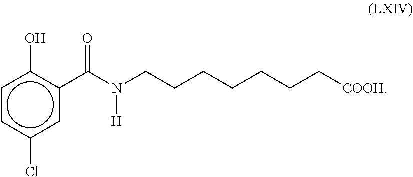

87. (canceled)

88. The composition of claim 1 wherein the penetration enhancer is selected from the group consisting of: (1) (a) at least one acetylated aldehyde of an amino acid; (b) at least one acetylated ketone of an amino acid; (c) at least one acetylated aldehyde of a peptide; (d) at least one acetylated ketone of a peptide; or (e) any combination of (1)(a), (1)(b), (1)(c), and (1)(d); (2) (a) carboxymethyl-phenylalanylleucine; (b) 2-carboxy-3-phenylpropionylleucine; (c) 2-benzylsuccinic acid; (d) an actinonin; (e) a compound having the formula Ar—Y—(R¹)ₙ—OH, wherein: (i) Ar is a substituted or unsubstituted phenyl or naphthyl; (ii) Y is —C(O)— or —SO₂—; (iii) R¹ is —N(R⁴)—R³—C(O)—, wherein: (A) R³ is C₁-C₂₄ alkyl, C₁-C₂₄ alkenyl, phenyl, naphthyl, (C₁-C₁₀ alkyl)phenyl, (C₁-C₁₀ alkyl)naphthyl, (C₁-C₁₀ alkenyl)phenyl, C₁-C₁₀ alkenyl(naphthyl), phenyl(C₁-C₁₀ alkyl), phenyl(C₁-C₁₀ alkenyl), naphthyl(C₁-C₁₀ alkyl), or phenyl(C₁-C₁₀ alkenyl); (B) R³ is optionally substituted with C₁-C₄ alkyl, C₁-C₄ alkenyl, C₁-C₄ alkoxy, hydroxyl, sulfhydryl, —CO₂R⁵, cycloalkyl, cycloalkenyl, heterocyclyl, aryl, alkaryl, heteroaryl, or heteroalkaryl or any combination thereof; (C) R⁵ is hydrogen, C₁-C₄ alkyl, or C₁-C₄ alkenyl; (D) R³ is optionally interrupted by oxygen, nitrogen, sulfur, or any combination thereof; (E) R⁴ is hydrogen, C₁-C₄ alkyl, or C₁-C₄ alkenyl; and (F) n is an integer from 1 to 5; or (f) any combination of (2)(a), (2)(b), (2)(c), (2)(d), and (2)(e); or (3) a combination of (1) and (2).

89. The composition of claim 1 wherein the penetration enhancer is selected from the group consisting of:
(a) an acid or an acid salt wherein the acid is selected from the group consisting of cyclohexanecarboxylic acid, cyclopentanecarboxylic acid, cycloheptanecarboxylic acid, hexanoic acid, 3-cyclohexanepropanoic acid, methylcyclohexanecarboxylic acid, 1,2-cyclohexanedicarboxylic acid, 1,3-cyclohexanedicarboxylic acid, 1,4-cyclohexanedicarboxylic acid, 1-adamantanecarboxylic acid, phenylpropanoic acid, adipic acid, cyclohexanepentanoic acid, cyclohexanebutanoic acid, pentylcyclohexanoic acid, 2-cyclopentanehexanoic acid, cyclohexanebutanoic acid, and (4-methylphenyl) cyclohexane acetic acid;
(b) a compound selected from the group consisting of 4-[(4-chloro-2-hydroxybenzoyl)amino]butanoic acid and 4-[(4-chloro-2-hydroxybenzoyl)amino]butanoate (“4-CNAB”);
(c) a compound of Formula (LXV) or Formula (LXVI): 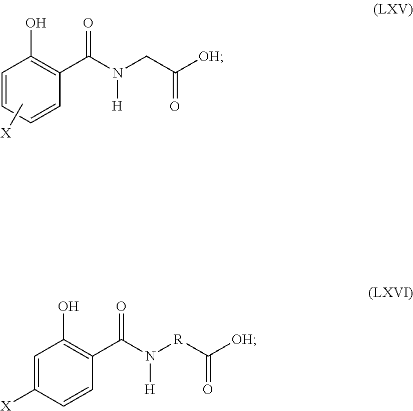
wherein: in Formula (LXIV), X is one or more of hydrogen, halo, hydroxyl, or C₁-C₃ alkoxy; and in Formula (LXV), X is halo and R is substituted or unsubstituted C₁-C₃ alkylene or substituted or unsubstituted C₁-C₃ alkenylene;
(d) a compound selected from the group consisting of 4-(4-methoxyphenyl)butanoic acid, 5-(2-methoxyphenyl)pentanoic acid, 5-(3-fluorophenyl)pentanoic acid, 5-(3-methoxyphenyl)pentanoic acid, 6-(3-fluorophenyl)hexanoic acid, 3-(4-t-butylphenyl)propanoic acid, 3-(4-n-butylphenyl)propanoic acid, 3-(4-n-propylphenyl)propanoic acid, 3-(4-n-propoxyphenyl)propanoic acid, 3-(4-isopropoxyphenyl)propanoic acid, 3-(4-n-butoxyphenyl)propanoic acid, 3-(3-phenoxyphenyl)propanoic acid, 3-(3-ethoxyphenyl)propanoic acid, 3-(3-isopropoxyphenyl)propanoic acid, 3-(3-n-butoxyphenyl)propanoic acid, 3-(3-n-propoxyphenyl)propanoic acid, 3-(3-isobutoxyphenyl)propanoic acid, 3-(4-isobutoxyphenyl)propanoic acid, 4-(4-ethylphenyl)butanoic acid, 4-(4-isopropylphenyl)butanoic acid, and 5-(4-ethylphenyl)pentanoic acid;
(e) a compound selected from the group consisting of a compound of Formula (LXVII), a compound of Formula (LXVIII), and a compound of Formula (LXIX): 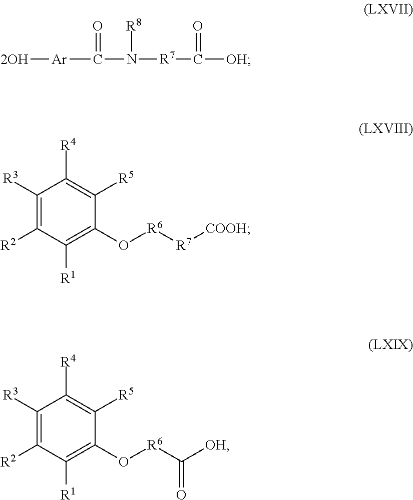
wherein: in Formula (LXVI): (i) Ar is phenyl or naphthyl; (ii) Ar is optionally substituted with one or more of hydroxyl, halo, C₁-C₄ alkyl, C₁-C₄ alkenyl, C₁-C₄ alkoxy, or C₁-C₄ haloalkoxy; (iii) R⁷ is selected from C₄-C₂₀ alkyl, C₄-C₂₀ alkenyl, phenyl, naphthyl, (C₁-C₁₀ alkyl)phenyl, (C₁-C₁₀ alkenyl)phenyl, (C₁-C₁₀ alkyl)napthyl, (C₁-C₁ alkenyl)naphthyl, phenyl(C₁-C₁₀ alkyl), phenyl(C₁-C₁₀ alkenyl), naphthyl(C₁-C₁₀ alkyl), or naphthyl(C₁-C₁₀ alkenyl); (iv) R⁷ is optionally interrupted by oxygen, nitrogen, sulfur, or any combination thereof; (v) R⁷ is optionally substituted with C₁-C₄ alkyl, C₂-C₄ alkenyl, C₁-C₄ alkoxy, C₁-C₄ haloalkoxy, hydroxyl, sulfhydryl, —CO₂R⁹, and combinations thereof; (vi) R⁸ is selected from hydrogen, C₁-C₄ alkyl, C₂-C₄ alkenyl, C₁-C₄ alkoxy, and C₁-C₄ haloalkoxy; and (vii) R⁹ is hydrogen, C₁-C₄ alkyl, or C₂-C₄ alkenyl; in Formula (LXVII): (i) R¹, R², R³, and R⁴ are each independently hydrogen, hydroxy, halo, C₁-C₄ alkyl, C₂-C₄ alkenyl, C₁-C₄ alkoxy, —C(O)R⁸, —NO₂, —NR⁹R¹⁰, and —N⁺R⁹R¹⁰R¹¹(R¹²)⁻; (ii) R⁵ is hydrogen, hydroxyl, nitro, halo, trifluoromethyl, —NR¹⁴R¹⁵, —N⁺R¹⁴R¹⁵R¹⁶(R¹³)⁻, amide, C₁-C₁₂ alkyl, C₂-C₁₂ alkenyl, carbamate, carbonate, urea, or —C(O)R¹⁸; (iii) R⁵ is optionally substituted with halo, hydroxyl, sulfhydryl, or —COOH: (iv) R⁵ is optionally interrupted by oxygen, nitrogen, sulfur, or —C(O)—; (v) R⁶ is a C₁-C₁₂ alkylene, C₁-C₁₂ alkenylene, or arylene; (vi) R⁶ is optionally substituted with C₁-C₄ alkyl, C₂-C₄ alkenyl, C₁-C₄ alkoxy, hydroxyl, sulfhydryl, halo, amino, or —CO₂R⁹; (vii) R⁶ is optionally substituted with C₁-C₄ alkyl, C₂-C₄ alkenyl, C₁-C₄ alkoxy, hydroxyl, sulfhydryl, amino, or —CO₂R⁹; (viii) R⁶ is optionally interrupted by oxygen or nitrogen; (ix) R⁷ is a bond or arylene: (x) R⁷ is optionally substituted with hydroxyl, halogen, —C(O)CH₃, —NR¹⁰R¹¹, —N⁺R¹⁰R¹¹R¹²(R¹³)⁻; (xi) R⁸ is hydrogen, C₁-C₄ alkyl, C₂-C₄ alkenyl, or amino; (xii) R⁹, R¹⁰, R¹¹, and R¹² are each independently hydrogen or C₁-C₁₀ alkyl; (xiii) R¹³ is a halide, hydroxide, sulfate, tetrafluoroborate, or phosphate; (xiv) R¹⁴, R¹⁵, and R¹⁶ are each independently hydrogen, C₁-C₁₀ alkyl, C₁-C₁₀ alkyl substituted with —COOH, C₂-C₁₂ alkenyl, C₂-C₂ alkenyl substituted with —COOH, or —C(O)R¹⁷; (xv) R¹⁷ is hydroxyl, C₁-C₁₀ alkyl, or C₂-C₁₂ alkenyl; and (xvi) R¹⁸ is hydrogen, C₁-C₆ alkyl, hydroxyl, —NR¹⁴R¹⁵, or N⁺R¹⁴R¹⁵R¹⁶(R¹³); and in Formula (XLVIII): (i) R¹, R², R³, R⁴, and R⁵ are independently hydrogen, cyano, hydroxyl, —OCH₃, or halo, provided that at least one of R¹, R², R³, R⁴, and R⁵ is cyano; and (ii) R⁶ is C₁-C₁₂ linear or branched alkylene, alkenylene, arylene, alkyl(arylene), or aryl(alkylene);
(f) a compound selected from the group consisting of a compound of Formula (LXX), a compound of Formula (LXXI), and a compound of Formula (LXXII): 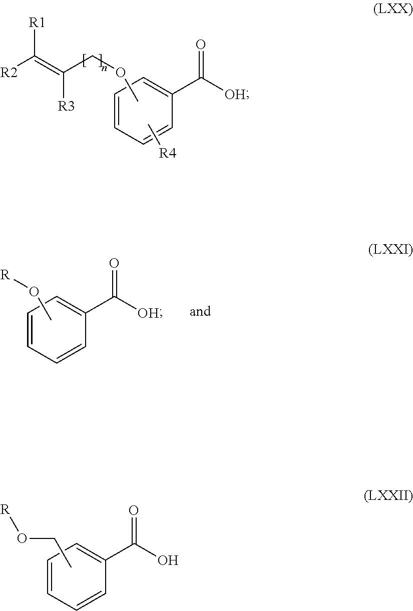
wherein: in Formula (LXX): (i) R1, R2, and R3 are independently hydrogen, methyl, or halo; (ii) R4 is hydrogen, methyl, methoxy, hydroxyl, halo, acetyl, or 2-hydroxy-ethoxy; and (iii) n is 1, 2, 3, or 4; in Formula (LXXI): R is C₁-C₆ straight-chain or branched alkyl; and in Formula (LXXII): R is methyl, ethyl, isopropyl, propyl, butyl, allyl, 1-methylallyl, 2-methylallyl, or butenyl;
(f) a compound with a cyclic moiety of Formula (LXXIII): 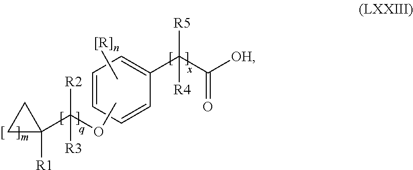
wherein: (i) m is 1, 2, 3, 4, 5, or 6; (ii) n is 0, 1, 2, 3, or 4; (iii) q and x are each independently chosen from 0, 1, 2, 3, 4, 5, 6, 7, 8, 9, or 10; (iv) R in [R]ₙ, (where n may be 0, 1, 2, 3, or 4 as set forth above) may be the same or different (if n is 2, 3, or 4) and is hydrogen, halo, a substituted or non-substituted alkyl, a substituted or non-substituted alkoxy, a substituted or non-substituted alkenyloxy, or a substituted or non-substituted aryloxy; and (v) R1, R2, R3, R4, and R5 are each independently selected from hydrogen, halogen, substituted or non-substituted alkyl, substituted or non-substituted alkenyl, substituted or non-substituted alkynyl, substituted or non-substituted alkoxy, substituted or non-substituted aryloxy, substituted or non-substituted aryl, substituted or non-substituted heteroaryl, substituted or non-substituted cycloalkyl, and substituted or non-substituted heterocycloaryl;
(g) a propylphenoxy ether of Formula (LXXIV): 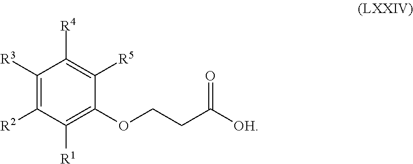
wherein: (i) R¹, R², R³. R⁴, and R⁵ are independently selected from hydrogen, halo, unsubstituted or substituted alkyl, unsubstituted or substituted alkenyl, unsubstituted or substituted alkoxy, unsubstituted or substituted haloalkoxy, hydroxy, —C(O)R⁸, nitro, —NR⁹R¹⁰, —N⁺R⁹R¹⁰R¹¹(R¹²), carbonate, ureido, CX₃, and cyano; (ii) R⁸ is hydrogen, C₁-C₄ alkyl, C₂-C₄ alkenyl, or amino; (iii) R⁹, R¹⁰, R¹¹, and R¹² are each independently hydrogen or C₁-C₁₀ alkyl; and (iv) X is halo;
(h) a dialkyl ether compound of Formula (LXXV): 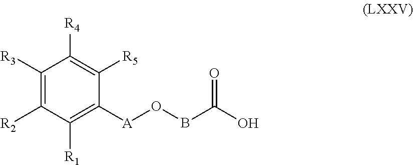
wherein: (i) A is a C₁-C₆ alkylene group that is straight-chain or branched-chain or substituted or unsubstituted; (ii) B is a C₁-C₂ alkylene group that is straight-chain or branched-chain or substituted or unsubstituted; (iii) R₁, R₂, R₃, R₄, and R₅ are each independently hydrogen, halo, unsubstituted or substituted alkyl, unsubstituted or substituted alkenyl, unsubstituted or substituted alkoxy, unsubstituted or substituted haloalkoxy, hydroxy, —C(O)R⁸, nitro, —NR⁹R¹⁰, —N⁺R⁹R¹⁰R¹¹(R¹²), carbonate, ureido, —CX₃, or cyano, optionally interrupted by an O, N, S, or —C(O)— group, wherein A and R₁ may together form a cycloalkyl group; (iii) R⁸ is hydrogen, C₁-C₄ alkyl, C₂-C₄ alkenyl, or amino; (iv) R⁹, R¹⁰, R¹¹, and R¹² are each independently hydrogen or C₁-C₁₀ alkyl; and X is halo; and
(i) an aryl ketone compound selected from the group consisting of 4-oxo-4-phenyl-butyric acid; 10-(4-hydroxy-phenyl)-10-oxodecanoic acid; 10-(2-hydroxy-phenyl)-10-oxo-decanoic acid; 4-(4-methoxy-phenyl)-4-oxo-butyric acid; 5-(4-methoxy-phenyl)-5-oxo-pentanoic acid; 4-(3,5-difluoro-phenyl)-4-oxo-butyric acid; 5-oxo-5-phenyl-pentanoic acid; 4-(2,4-dimethyl-phenyl)-4-oxo-butyric acid; 6-(4-methoxy-3,5-dimethyl-phenyl)-6-oxo-hexanoic acid; 5-(4-isopropyl-phenyl)-5-oxo-pentanoic acid; 4-(2-methoxy-phenyl)-4-oxo-butyric acid; 4-(4-fluoro-phenyl)-4-oxo-butyric acid; 6-(4-methoxy-phenyl)-6-oxo-hexanoic acid; 4-(3,5-dimethyl-phenyl)-4-oxo-butyric acid; 6-(3,4-dimethyl-phenyl)-6-oxo-hexanoic acid; 4-(3,4-dimethyl-phenyl)-4-oxo-butyric acid; 4-oxo-4-(4-phenoxy-phenyl)-butyric acid; 4-(2,5-dimethyl-phenyl)-4-oxo-butyric acid; 8-(3,5-dimethyl-phenyl)-8-oxo-octanoic acid; 6-(2,5-dichloro-phenyl)-6-oxo-hexanoic acid; 4-(2,5-dichloro-phenyl)-4-oxo-butyric acid; 6-(3,5-dimethyl-phenyl)-6-oxo-hexanoic acid; 10-(2,5-dihydroxy-phenyl)-10-oxo-decanoic acid; 8-oxo-8-phenyl-octanoic acid; 6-(2,5-difluoro-phenyl)-6-oxo-hexanoic acid; 7-oxo-7-phenyl-heptanoic acid; 4-(4-ethyl-phenyl)-4-oxo-butyric acid; 4-(2,4-difluoro-phenyl)-4-oxo-butyric acid; 4-(4-butoxy-phenyl)-4-oxo-butyric acid; 4-oxo-4-(4-propyl-phenyl)-butyric acid; 4-oxo-4-(4-pentyl-phenyl)-butyric acid; 4-(4-hexyloxy-phenyl)-4-oxo-butyric acid; 4-(2,5-difluoro-phenyl)-4-oxo-butyric acid; 5-(4-chloro-phenyl)-5-oxo-pentanoic acid; 6-(3,5-difluoro-phenyl)-6-oxo-hexanoic acid; 4-oxo-4-p-tolyl-butyric acid; 6-oxo-6-phenyl-hexanoic acid; 5-oxo-5-(4-phenoxy-phenyl)-pentanoic acid; 5-oxo-5-(3-phenoxy-phenyl)-pentanoic acid; and 7-oxo-7-(3-phenoxy-phenyl)-heptanoic acid.

90.-98. (canceled)

99. The composition of claim 1 wherein the penetration enhancer is a compound selected from the group consisting of:
(a) a compound selected from the group consisting of arachidonic acid, lauric acid, caprylic acid, capric acid, myristic acid, palmitic acid, stearic acid, linoleic acid, linolenic acid, dicaprate, tricaprate, monolein, dilaurin, glyceryl 1-monocaprate, 1-dodecylazacycloheptan-2-one, an acylcarnitine, an acylcholine, or a C₁₋₁₀ alkyl ester, monoglyceride, diglyceride, and a pharmaceutically acceptable salt thereof;
(b) a bile salt selected from the group consisting of cholic acid, dehydrocholic acid, deoxycholic acid, glucholic acid, glycholic acid, glycodeoxycholic acid, taurocholic acid, taurodeoxycholic acid, chenodeoxycholic acid, ursodeoxycholic acid, sodium tauro-24,25-dihydro-fusidate, and sodium glycodihydrofusidate;
(c) polyoxyethylene-9-lauryl ether;
(d) a chelating agent selected from the group consisting of EDTA and citric acid;
(e) a salicylate;
(f) a N-acyl derivative of collagen;
(g) an N-amino acyl derivative of a beta-diketone;
(h) an ionic or nonionic surfactant;
(i) polyoxyethylene-20-cetyl ether;
(j) a perfluorochemical emulsion; and
(k) a compound selected from the group consisting of an unsaturated cyclic urea, a 1-alkyl-alkone, a 1-alkenylazacyclo-alkanone, a glycol, a pyrrole, an azone, and a terpene;
(l) a compound selected from the group consisting of polyvalent aliphatic C₂-C₁₀ alcohols, polyalkylene glycols having C₂-C₄ alkylene groups, nonalkoxylated ethers of polyvalent aliphatic C₂-C₁₀ alcohols and polyalkylene glycols having C₂-C₄ alkylene groups, azones, terpenes, terpenoids, pyrrolidones, and sulfoxides;
(m) a compound selected from the group consisting of polyvalent aliphatic C₂-C₁₀ alcohols, polyalkylene glycols having C₂-C₄ alkylene groups, nonalkoxylated ethers of polyvalent aliphatic C₂-C₁₀ alcohols and polyalkylene glycols having C₂-C₄ alkylene groups, azones, terpenes, terpenoids, pyrrolidones, and sulfoxides;
(n) a nanoparticle or micelle constructed from a polymer selected from the group consisting of dextran, carboxymethyl dextran, chitosan, trimethylchitosan, poly(lactic-co-glycolic acid) (PLGA), polylactic acid (PLA), polyglycolic acid (PGA), polyvinylalcohol (PVA), polyanhydrides, polyacylates, polymethacrylates, polyacylamides, polymethacrylate, dextran, chitosan, cellulose, hypromellose, starch, dendrimers, peptides, proteins, polyethyleneglycols and poly(ethyleneglycol-co-propyleneglycol), and synthetic derivatives thereof;
(o) a synthetic peptide ligand;
(p) a biodegradable polymer that is a copolymer of lactic acid and glycolic acid or enantiomers thereof;
(q) a compound that is selected from the group consisting of membrane translocating full-length peptide sequences, fragments thereof, motifs derived therefrom, derivatives thereof, analogs thereof, and peptidomimetics based on the peptide sequences;
(r) a D-form retro-inverted peptide;
(s) a composition comprising: (1) a penetration enhancer that is: (A) is a solid at room temperature; and (B) is a salt of a medium chain fatty acid having a carbon length of from 8 to 14 carbon atoms in particulate form; and (2) a rate-controlling polymer;
(t) a compound selected from the group consisting of mono-, di-, and triglyceride esters of medium-chain or long-chain fatty acids, esters of fatty acids and glycols and esters of mixed fatty acids and glycols and mixtures thereof, diesters of propylene glycol having from about 7 to about 55 carbon atoms, propylene glycol esters of capric and caprylic acids, and mixtures thereof, having from 19 to 23 carbon atoms; and
(u) a medium chain fatty acid or a medium chain fatty acid derivative having a carbon chain length of from 6 to 20 carbon atoms; with the provisos that (i) where the penetration enhancer is an ester of a medium chain fatty acid, the chain length of from 6 to 20 carbon atoms relates to the chain length of the carboxylate moiety, and (ii) where the enhancer is an ether of a medium chain fatty acid, at least one alkoxy group has a carbon chain length of from 6 to carbon atoms, wherein the penetration enhancer is a solid at room temperature.

100.-108. (canceled)

109. The composition of claim 1 wherein the penetration enhancer is selected from the group consisting of:
(a) a compound of Formula (LXXVII): 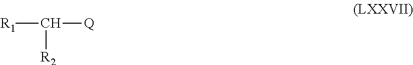
wherein Q is: (1) a partially or completely neutralized —COOH, or (2) a partially or completely neutralized —SO₃H, or (3) a mono- or di-substituted alkyl or alkenyl group having one to about twelve carbon atoms, the substituent(s) thereof being a partially or completely neutralized —COOH or partially or completely neutralized —SO₃H; and R₁ and R₂ are independently: (1) an unsubstituted alkyl or alkenyl group having one to about twelve carbon atoms, or (2) a substituted alkyl or alkenyl group having one to about twelve carbon atoms, the substituent thereof being selected from the group consisting of (i) partially or completely neutralized —COOH, (ii) partially or completely neutralized —SO₃H, (iii) —NH₂, (iv) —CONH₂; and (v) —OH;
(b) a purified synthetic polypeptide ligand comprising a 12-mer L-peptide or homologue thereof;
(c) a peptide including peptide sequences possessing both hydrophobic amino acids and charged amino acids, optionally modified by hydrophobic moieties;
(d) a medium chain fatty acid salt associated with a substantially hydrophobic medium;
(e) a composition comprising: (1) octanoate, sodium decanoate, sodium dodecanoate, and combinations thereof; (2) a hydrophobic medium to produce a suspension, wherein the hydrophobic medium is selected from the group consisting of aliphatic molecules, cyclic molecules, aromatic molecules and combinations thereof; and (3) a lecithin, a bile salt or a non-ionic detergent;
(f) a penetration enhancer including a liquid-forming cationic amphipathic counterion optionally modified by addition of a hydrophobic moiety:
(g) a peptide derived from Escherichia coli optionally modified to increase hydrophobicity:
(h) a calcium phosphate nanoparticle; and
(i) a penetration enhancer comprising a fatty acid, a medium chain glyceride, a surfactant, a steroidal detergent, an acyl carnitine, an alkanoyl choline, an N-acetylated amino acid, esters, salts and derivatives thereof, or any combination thereof.

110-117. (canceled)

118. The composition of claim 1 wherein the penetration enhancer is selected from the group consisting of:
(a) an orthoester derivative of a crown ether of Formula (LXXVIII): 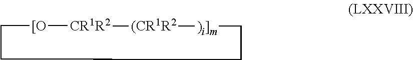
wherein:
(i) m is 4, 5, 6, 7, or 8;
(ii) i is independently for each occurrence, 1 or 2;
(iii) each occurrence of R¹ and R² is independently selected from hydrogen; linear or branched and substituted or unsubstituted C₁-C₁₀ alkyl, alkenyl, or alkynyl; and substituted or unsubstituted aryl with up to 10 ring atoms, or R¹ and R² form an oxo group;
(iv) there is at least one occurrence in the crown ether of R¹, R², and the carbon to which R¹ and R² are bound, the carbon being bound directly to an ether oxygen of Formula (LXXVIII), form together a group of subformula (LXXVIIIa) 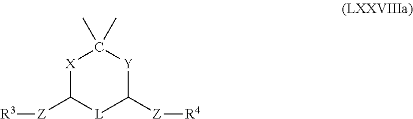
wherein L is a linker that is absent or is selected from a covalent bond and (CR⁵R⁶)ₙ, each occurrence of R⁵ and R⁶ being independently selected from: hydrogen; linear or branched and substituted or unsubstituted C₁-C₁₀ alkyl, alkenyl, or alkynyl; and substituted or unsubstituted aryl with up to 10 ring atoms; n is 1, 2, or 3; X and Y, independently from each other, are selected from O and S; Z, independently for each occurrence, is absent or an electron-withdrawing group; R³ and R⁴, independently for each occurrence, are selected from: hydrogen; linear or branched and substituted or unsubstituted C₁-C₁₀ alkyl, alkenyl, or alkynyl; and substituted or unsubstituted aryl with up to 10 ring atoms; H(OCH₂CH₂)ₖ— H(OCH₂CH₂)ₖO—, wherein k is 1, 2, 3, 4, 5, 6, 7, 8, 9, or 10; and wherein substituents, if present, are selected from hydroxyl, halogens, and O—CH₃;
(b) a crown compound in a nonaqueous hydrophobic vehicle optionally associated with a counterion, wherein the crown compound is selected from the group consisting of: (i) cyclic polyester; (ii) cyclic polyamide; (iii) cyclic polyether; (iv) cyclic polyoxime: (v) polythioester; (vi) polymer of aminoxy acids; (vii) polydisulfide; (viii) cyclic polydioxanones, and (ix) a cyclic compound belonging to more than one of (i) to (ix), where the crown is a cation-binding crown compound capable of forming a charge masking complex with a cation;
(c) a penetration enhancer that has a covalent linkage to a membrane translocator that is a peptide, fatty acid, or bile acid; and
(d) an acyl-L-carnitine.

119.-122. (canceled)

123. The composition of claim 1 wherein the penetration enhancer comprises a compound or composition selected from the group consisting of:
(a) a composition comprising: (i) an anionic agent that is a cholesterol derivative, (ii) a mixture of a negative charge neutralizer and an anionic surface active agent, (iii) non-ionic surface active agents, and (iv) cationic surface active agents;
(b) (4-[(4-chloro, 2-hydroxybenzoyl)amino] butanoic acid;
(c) a compound selected from the group consisting of 3-[4-(cyclopropylmethoxy)phenyl]propanoic acid; 4-(cyclobutylmethoxy)benzoic acid; [4-(cyclobutylmethoxy)-3-methoxyphenyl]acetic acid; 4-(cyclopropylmethoxy)benzoic acid; [4-(cyclopropylmethoxy)phenyl]acetic acid; 2-(cyclobutylmethoxy)benzoic acid; [4-(cyclopentyloxy)-3-methoxyphenyl]acetic acid: [4-(cyclopropylmethoxy)-3-methoxyphenyl]acetic acid; 2-(cyclopropylmethoxy)benzoic acid; 2-(cyclopentyloxy)benzoic acid: 2-(cyclohexylmethoxy)benzoic acid; 3-(cyclopropylmethoxy)benzoic acid; 3-(cyclobutylmethoxy)benzoic acid: 3-(cyclopentyloxy)benzoic acid: 3-(cyclohexylmethoxy)benzoic acid: 4-(cyclopentyloxy)benzoic acid: 4-(cyclopentyloxy)benzoic acid: [4-(cyclobutylmethoxy)phenyl]acetic acid: 3-[4-(cyclobutylmethoxy)phenyl]propanoic acid: [4-(cyclohexylmethoxy)phenyl]acetic acid: 3-[4-(cyclohexylmethoxy)phenyl]propanoic acid: [4-(cyclohexylmethoxy)-3-methoxyphenyl]acetic acid: 3-[2-(cyclopropylmethoxy)phenyl]propanoic acid: [4-(cyclopentyloxy)phenyl]acetic acid; and 3-[4-(cyclopentyloxy)phenyl]propanoic acid:
(d) a disodium salt, an ethanol solvate, or a hydrate of a compound selected from the group consisting of N-(5-chlorosalicyloyl)-8-aminocaprylic acid, N-(10-[2-hydroxybenzoyl]amino)decanoic acid, and sodium N-(8-[2-hydroxybenzoyl]amino)caprylate;
(e) a crystalline form of the disodium salt of N-(5-chlorosalicyloyl)-8-aminocaprylic acid; and
(f) a penetration enhancer of Formula (LXXIX): 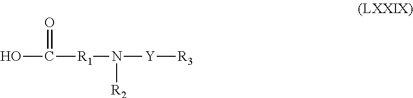
wherein:
(i) Y is carbonyl or SO₂;
(ii) R₁ is C₃-C₂₄ alkyl, C₂-C₂₀ alkenyl, C₂-C₂₀ alkynyl, cycloalkyl, or aromatic;
(iii) R₂ is hydrogen, C₁-C₄ alkyl, or C₂-C₄ alkenyl; and
(iv) R is C₁-C₇ alkyl, C₃-C₁₀ cycloalkyl, aryl, thienyl, pyrrolo, or pyridyl, wherein R₃ is optionally substituted with one or more C₁-C₅ alkyl groups, C₂-C₄ alkenyl groups, halogen, SO₂, COOH, or SO₃H.

124.-128. (canceled)

129. The pharmaceutical composition of claim 1 wherein the penetration enhancer is in the form of a nanoparticle or microparticle having a median particle size of less than about 1000 micrometers and wherein the penetration enhancer is of Formula (LXXX), (LXXXI), (LXXXII), (LXXXIII), or (LXXXIV): 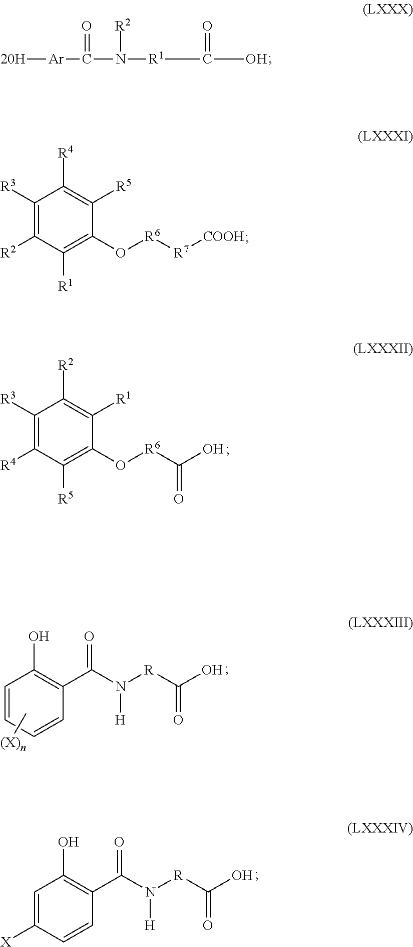
wherein:
(a) in Formula (LXXX):
(i) Ar is phenyl or naphthyl;
(ii) Ar is optionally substituted with one or more of hydroxyl, halogen, C₁-C₄ alkyl, C₁-C₄ alkenyl, C₁-C₄ alkoxy, or C₁-C₄ haloalkoxy;
(iii) R¹ is C₃-C₂₀ alkyl, C₄-C₂₀ alkenyl, phenyl, naphthyl, (C₁-C₁₀ alkyl)phenyl, (C₁-C₁₀ alkenyl)phenyl, (C₁-C₁₀ alkyl)naphthyl, (C₁-C₁₀ alkenyl)naphthyl, phenyl(C₁-C₁₀ alkyl), phenyl(C₁-C₁₀ alkenyl), naphthyl(C₁-C₁₀ alkyl), or naphthyl(C₁-C₁₀ alkenyl);
(iv) R¹ is optionally substituted with C₁-C₄ alkyl, C₂-C₄ alkenyl, C₁-C₄ alkoxy, C₁-C₄ haloalkoxy, hydroxyl, or sulfhydryl or any combination thereof;
(v) R² is hydrogen, C₁-C₄ alkyl, or C₂-C₄ alkenyl; and
(vi) R¹ is optionally interrupted by oxygen, nitrogen, sulfur, or any combination thereof; wherein the term “2-OH—Ar” refers to a phenyl or naphthyl group having a hydroxyl group at the 2-position;
(b) in Formula (LXXXI):
(i) R¹, R², R³, and R⁴ are each independently hydrogen, hydroxyl, halogen, C₁-C₄ alkyl, C₂-C₄ alkenyl, C₁-C₄ alkoxy, —C(O)R⁸, —NO₂, —NR⁹R¹⁰, or —N⁺R⁹R¹⁰R¹¹ (R¹²)⁻;
(ii) R⁵ is hydrogen, hydroxyl, —NO₂, halogen, —CF₃, —NR¹⁴R¹⁵, —N⁺R¹⁴R¹⁵R¹⁶(R¹³)⁻, amide, C₁-C₁₂ alkoxy, C₁-C₁₂ alkyl, C₁-C₁₂ alkenyl, carbamate, carbonate, urea, or —C(O)R¹⁸;
(iii) R⁵ is optionally substituted with halogen, hydroxyl, sulfhydryl, or carboxyl;
(iv) R⁵ is optionally interrupted by O, N, S, or —C(O)—;
(v) R⁶ is a C₁-C₁₂ alkylene, C₁-C₁₂ alkenylene, or arylene;
(vi) R⁶ is optionally substituted with C₁-C₄ alkyl, C₂-C₄ alkenyl, C₁-C₄ alkoxy, hydroxyl, sulfhydryl, halogen, amino, or —CO₂R⁸;
(vii) R⁶ is optionally interrupted by O or N;
(viii) R⁷ is a bond or arylene;
(ix) R⁷ is optionally substituted with hydroxyl, halogen, —C(O)CH₃, —NR¹⁰R¹¹, or —N⁺R¹⁰R¹¹R¹²(R¹³)⁻;
(x) R⁸ is hydrogen, C₁-C₄ alkyl, C₂-C₄ alkenyl, or amino;
(xi) R⁹, R¹⁰, R¹¹, and R¹² are independently hydrogen or C₁-C₁₀ alkyl;
(xii) R¹³ is a halide, hydroxide, sulfate, tetrafluoroborate, or phosphate;
(xiii) R¹⁴, R¹⁵, and R¹⁶ are each independently hydrogen, C₁-C₁₀ alkyl, C₁-C₁₀ alkyl substituted with carboxyl, C₂-C₁₂ alkenyl, C₂-C₁₂ alkenyl substituted with carboxyl, or —C(O)R¹⁷;
(xiv) R¹⁷ is hydroxyl, C₁-C₁₀ alkyl, or C₂-C₁₂ alkenyl; and
(xv) R¹⁸ is hydrogen, C₁-C₆ alkyl, hydroxyl, —NR¹⁴R¹⁵, or —N⁺R¹⁴R¹⁵R¹⁶(R¹³)⁻;
(c) in Formula (LXXXII):
(i) R¹, R², R³, R⁴, and R⁵ are each independently hydrogen, —CN, hydroxyl, —OCH₃, or halogen, wherein at least one of R¹, R², R³, R⁴, and R⁵ is —CN; and
(ii) R⁶ is a C₁-C₁₂ linear or branched alkylene, alkenylene, arylene, alkyl(arylene) or aryl(alkylene);
(d) in Formula (LXXXIII):
(i) each occurrence of X is hydrogen, halogen, hydroxyl, or C₁-C₃ alkoxy;
(ii) R is substituted or unsubstituted C₁-C₃ alkylene or substituted or unsubstituted C₂-C₃ alkenylene; and
(iii) n is 1, 2, 3, or 4; and
(e) in Formula (LXXXIV):
(i) X is halogen; and
(ii) R is substituted or unsubstituted C₁-C₃ alkylene or substituted or unsubstituted C₂-C₃ alkenylene.

130. The pharmaceutical composition of claim 1 wherein the penetration enhancer is selected from the group consisting of:
(a) a compound selected from the group consisting of 3-(3-hexyloxy-2-hydroxy-propoxy)-propane-1,2-diol and 3-[2-hydroxy-3-(2-hydroxy-2-octyloxy-propoxy)-propoxy]-propane-1,2-diol;
(b) a polymorphic form of a penetration enhancer selected from the group consisting of SNAC and sodium 4-CNAB;
(c) a compound selected from the group consisting of 4-(4-methoxyphenyl)butanoic acid, 5-(2-methoxyphenyl)pentanoic acid, 5-(3-fluorophenyl)pentanoic acid, 5-(3-methoxyphenyl)pentanoic acid, 6-(3-fluorophenyl)hexanoic acid, 3-(4-t-butylphenyl)propanoic acid, 3-(4-n-butylphenyl)propanoic acid, 3-(4-n-propylphenyl)propanoic acid, 3-(4-n-propoxyphenyl)propanoic acid, 3-(4-isopropoxyphenyl)propanoic acid, 3-(4-n-butoxyphenyl)propanoic acid, 3-(3-phenoxyphenyl)propanoic acid, 3-(3-ethoxyphenyl)propanoic acid, 3-(3-isopropoxyphenyl)propanoic acid, 3-(3-n-butoxyphenyl)propanoic acid, 3-(3-n-propoxyphenyl)propanoic acid, 3-(3-isobutoxyphenyl)propanoic acid, 3-(4-isobutoxyphenyl)propanoic acid, 4-(4-ethylphenyl)butanoic acid, 4-(4-isopropylphenyl)butanoic acid, and 5-(4-ethylphenyl)pentanoic acid, and pharmaceutically acceptable salts thereof;
(d) a salt of a compound whose non-ionized form is a penetration enhancer, wherein the salt is formed between:
(i) an anion and a positively charged group on an ionized form of a penetration enhancer and wherein the anion is selected from the group consisting of chloride, bromide, iodide, carbonate, nitrate, sulfate, bisulfate, phosphate, monohydrogen phosphate, dihydrogen phosphate, metaphosphate, pyrophosphate, formate, acetate, adipate, butyrate, propionate, succinate, glycolate, gluconate, lactate, malate, tartrate, citrate, ascorbate, glucuronate, maleate, fumarate, pyruvate, aspartate, glutamate, benzoate, anthranilate, mesylate, 4′-hydroxybenzoate, phenylacetate, mandelate, embonate (pamoate), methanesulfonate, ethanesulfonate, ethanedisulfonate, benzenesulfonate, pantothenate, 2-hydroxyethanesulfonate, p-toluenesulfonate, sulfanilate, cyclohexylaminosulfonate, camphorate, camphorsulfonate, digluconate, cyclopentanepropionate, dodecylsulfonate, glucoheptanoate, glycerophosphonate, heptanoate, hexanoate, 2-hydroxyethanesulfonate, nicotinate, isonicotinate, 1-naphthalenesulfonate, 2-naphthalenesulfonate, oxalate, palmoate, pectinate, persulfurate, 2-phenylpropionate, picrate, pivalate, thiocyanate, mesylate, undecanoate, stearate, algenate, β-hydroxybutyrate, salicylate, galactarate, galacturonate, caprylate, isobutyrate, malonate, suberate, sebacate, chlorobenzoate, methylbenzoate, dinitrobenzoate, phthalate, phenylacetate, isethionate, lactobionate, p-aminobenzoate, sulfamate, diethylacetate, pimelate, aminosulfonate, acrylate, γ-hydroxybutyrate, and methoxybenzoate; or
(ii) the salt is formed between an cation and a negatively charged group on an ionized form of a penetration enhancer and wherein the cation is selected from the group consisting of sodium, aluminum, lithium, calcium, magnesium, zinc, ammonium, caffeine, arginine, diethylamine, N-ethylpiperidine, histidine, glucamine, isopropylamine, lysine, morpholine, N-ethylmorpholine, piperazine, piperidine, triethylamine, trimethylamine, ethanolamine, diethanolamine, N-methylglucamine, and tris(hydroxymethyl)aminomethane, and
(e) a compound possessing both at least one hydrophobic group and at least one hydrophilic group, wherein the at least one hydrophobic group is selected from the group consisting of phenyl groups, naphthyl groups, cyclohexyl groups, and long-chain aliphatic groups, and wherein the at least one hydrophilic group is selected from the group consisting of carboxylic acid groups, carboxylic acid ester groups, amide groups, amino groups, and carbonyl groups.

131.-138. (canceled)

139. The composition of claim 1 wherein the pharmaceutical composition comprises the pharmaceutically acceptable carrier, and wherein the pharmaceutically acceptable carrier is selected from the group consisting of an acidifying agent, an aerosol propellant, an air displacement, an alcohol denaturant, an alkalizing agent, an antifoaming agent, an antimicrobial preservative, an antioxidant, a buffering agent, a chelating agent, a coating agent, a colorant, a complexing agent, a desiccant, an emulsifying and/or solubilizing agent, a filtering aid, a flavor or perfume, a glidant and/or anticaking agent, a humectant, a plasticizer, a polymer, a solvent, a sorbent, a carbon dioxide sorbent, a stiffening agent, a suspending and/or viscosity-increasing agent, a sweetening agent, a tablet binder, a tablet and/or capsule diluent, a tablet disintegrant, a tonicity agent, a flavored and/or sweetened vehicle, an oleaginous vehicle, a solid carrier vehicle, a sterile vehicle, a water-repelling agent, and a wetting and/or solubilizing agent.

140. (canceled)

141. The pharmaceutical composition of claim 1 wherein the pharmaceutical composition is in a dosage form selected from the group consisting of a sublingual dosage form, a buccal fast melt dosage form, and a film dosage form.

142. A method of treating lower urinary dysfunctional epithelium (LUDE) or a disease, condition, or syndrome associated with LUDE comprising the step of administering orally: (1) a pharmaceutically effective quantity of sodium pentosan polysulfate; and (2) a quantity of a penetration enhancer effective to improve the bioavailability of sodium pentosan polysulfate to a patient in need of treatment for LUDE or a disease, condition, or syndrome associated with LUDE in order to treat LUDE or a disease, condition, or syndrome associated with LUDE.

143.-300. (canceled)

301. A pharmaceutical composition formulated for treatment or prevention of a disease or condition associated with inflammation comprising:
(a) a therapeutically effective quantity of a pentosan polysulfate salt;
(b) a quantity of a penetration enhancer sufficient to improve the bioavailability of the pentosan polysulfate salt; and
(c) optionally, a pharmaceutically acceptable carrier.

302.-305. (canceled)

306. The pharmaceutical composition of claim 301 wherein the pharmaceutical composition further comprises at least one additional agent that is effective in treating inflammation, wherein the additional therapeutic agent is selected from the group consisting of:
(1) calcitonin selected from the group consisting of salmon calcitonin, eel calcitonin, and human calcitonin;
(2) a calcitonin derivative selected from including (Asu^{1,7})eel calcitonin, variants of calcitonin, fragments of calcitonin including amino acid residues 17-21 of calcitonin, and truncated derivatives of calcitonin lacking amino acid residues 1-9;
(3) a bisphosphonate selected from the group consisting of zoledronic acid, etidronate, clodronate, tiludronate, pamidronate, neridronate, olpadronate, alendronate, ibandronate, minodronate, incadronate, and risedronate;
(4) strontium ranelate;
(5) bone morphogenetic protein 7 (BMP-7), and homologs thereof including one or more conservative amino acid substitutions:
(6) a selective iNOS (inducible nitric oxide synthase) inhibitors including cindunistat; aminoguanidine hydrochloride; 2-amino-5,6-dihydro-6-methyl-4H-1,3-thiazine hydrochloride: AR-C 102222 (5-[(4′-amino-5′,8′-difluorospiro[piperidine-4,2′(1′H)-quinaxolin]-1-yl)carbonyl]-2-pyridinecarbonitrile hydrochloride); BYK 191023 dihydrochloride (2-[2-(4-methoxy-2-pyridinyl)ethyl]-1H-imidazo[4,5-b]pyridine dihydrochloride); (S)-ethylisothiourea hydrobromide; 2-iminopiperidine hydrochloride; (S)-isopropylisothiourea hydrobromide; (S)-methylisothiourea sulfate; N⁶-(1-iminoethyl)-L-lysine hydrochloride; N⁵-(1-iminoethyl)-L-ornithine dihydrochloride; and N-[[3-(Aminomethyl)phenyl]methyl]-ethanimidamide dihydrochloride);
(7) matrix metalloproteinase (MMP) inhibitors, wherein the MMP is selected from the group consisting of aggrecanase, MMP-1, MMP-13, MMP-3, cathepsin K, or another protease that participates in the catabolic process of tissue destruction, including batimastat, marimastat, ilomastat, prinomastat, cipemastat, MMI-166 (N-α-[4-(2-phenyl-2H-tetrazole-5-yl) phenyl sulfonyl]-D-tryptophan), MMI-270 ((2R)—N-hydroxy-2-[(4-methoxyphenyl)sulfonyl-(pyridin-3-ylmethyl)amino]-3-methylbutanamide), ABT-770 ((S)—N-[1-[[4′-trifluoromethoxy-[1,1′-biphenyl]-4-yl]oxy]methyl-2-(4,4-dimethyl-2,5-dioxo-1-imidazolidinyl)ethyl]-N-hydroxyformamide), RS-130830 (4-(((3-(4-chlorophenoxy)phenyl)sulfonyl)methyl)-N-hydroxytetrahydro-2H-pyran-4-carboxamide), CAS Reg. No. 239796-97-5 (1-benzyl-(4-(4-chloroxyphenoxy)phenyl)sulfonyl)-N-hydroxypiperidine-4-carboxamide), solimastat, KB-R-7785, GI-129471, rebimastat, tanomastat, Ro-28-2653, 544678-85-5, pyridine dicarboxamides, 868-68-30-3, CAS Reg. No. 582311-81-7, doxycycline, and metastat;
(8) endogenous inhibitors of metalloproteinases, including TIMP3;
(9) inhibitors of cathepsin K, including odanacatib:
(10) a COX-2 inhibitor selected from the group consisting of rofecoxib, valdecoxib, celecoxib, etoricoxib, lumiracoxib, parecoxib, deracoxib, tiracoxib, meloxicam, nimesulide, (1,1-dimethylheptyl)-6a,7,10,10a-tetrahydro-1-hydroxy-6,6-dimethyl-6H-dibenzo[b,d]pyran carboxylic acid (CT-3), 5,5-dimethyl-3-(2-propoxy)-4-methanesulfonylphenyl)-2(5H)-furanone; carprofen; 2-(acetyloxy)benzoic acid 3-[(nitrooxy)methyl]phenyl ester (NCX4016), P54 (a turmeric derivative); 2,6-bis(1,1-dimethylethyl)[(E)-(2-ethyl-1,1-dioxo isothiazolidinylidene)methyl]phenol (S-2474), 5(R)-thiosulfonamide-3 (2H)-benzofuranone (SVT-2016) and N-[3-(formyl-amino)oxophenoxy-4H benzopyranyl]methanesulfonamide (T-614); and a pharmaceutically acceptable salt thereof;
(11) a mixed COX-1/COX-2 inhibitor;
(12) an inhibitor of TNFα selected from the group consisting of etanercept, aldalimumab, and infliximab:
(13) a non-steroidal anti-inflammatory drug (NSAID) painkiller selected from the group consisting of: an enolic acid selected from the group consisting of piroxicam, tenoxicam and meloxicam; a heteroaryl acetic acid selected from the group consisting of tolmetin, ketorolac, misoprostol, and zomepirac: an indole or indene acetic acid selected from the group consisting of indomethacin, mefenamic acid, sulindac and etodolac: a p-aminophenol derivative selected from the group consisting of phenacetin and acetaminophen; a propionic acid selected from the group consisting of naproxen, flurbiprofen, fenoprofen, oxaprozin, carprofen, ketoprofen and ibuprofen; a sulfonanilide selected from the group consisting of nimesulide; a fenamate selected from the group consisting of mefenamic acid, meclofenamate and flufenamic acid; an alkanone; a pyrazolone selected from the group consisting of phenylbutazone, oxyphenbutazone, antipyrine, aminopyrine, and kebuzone; and a salicylate selected from the group consisting of acetylsalicylic acid (aspirin), salicylate, salsalate, diflunisal, olsalazine, fendosal, sulfasalazine and thiosalicylate;
(14) a bone forming agent selected from the group consisting of an anti-Dkk1 antibody and an activin antagonist;
(15) a bone antiresorbing agent;
(16) a steroid hormone that is an estrogen, a partial estrogen agonist or estrogen-gestagen combination, wherein the hormone is selected from the group consisting of prednisolone, prednisone, methylprednisolone, betamethasone, hydrocortisone, cortisone, triamcinolone, dexamethasone, beclomethasone, budesonide, deoxycortone and fludrocortisone:
(17) a SERM (Selective Estrogen Receptor Modulator) selected from the group consisting of bazedoxifene acetate, ospemifene, raloxifene, arzoxifene, droloxifene, tamoxifen, 4-hydroxy-tamoxifen, 4′-iodotamoxifen, toremifene, (deaminohydroxy)-toremifene, chlomiphene, levormeloxifene, ormeloxifene, chroman derivatives, coumarin derivatives, idoxifene, nafoxidine, miproxifen phosphate (TAT-59), arzoxifene, lasofoxifene, (E)-1-butanamine, 4-(4-(2-chloro-1,2-diphenylethenyl)phenoxy)-N,N-diethyl-dihydrogen citrate (MDL-103323), acolbifene, (EM-652), EM-800, fulvestrant, N-(n-butyl)-11-[3,17β-dihydroxyestra-1,3,5(10)-trien-7α-yl]N-methylundecanamide (ICI 164,384), diethylstilbestrol, genistein, nafoxidine, nitromifene, moxesterol, diphenol hydrochrysene, erythro-MEA, allenolic acid, equilin-3-sulfate, cyclophenyl, chlorotrianisene, ethamoxytriphetol, lasofoxifene, bazedoxifene, genistein, tibolone, ospemifene, tesmilifene, droloxifene, panomifene, zindoxifene, meproxifene and faslodex:
(18) vitamin D or an analog thereof;
(19) parathyroid hormone (PTH), a PTH fragment or a PTH derivative selected from the group consisting of PTH (1-84), PTH (1-34), PTH (1-36), PTH (1-38), PTH (1-31)NH2 and PTS 893;
(20) a PTH releaser selected from the group consisting of 2-chloro-N-[(1R)-1-(3-methoxyphenyl)ethyl]-benzenepropanamine hydrochloride and cinacalcet;
(21) a strontium-containing compound that is an organic strontium salt selected from the group consisting of strontium malonate, strontium succinate, strontium fumarate, strontium ascorbate, strontium aspartate in either L- and/or D-form, strontium glutamate in either L- and/or D-form, strontium pyruvate, strontium tartrate, strontium glutarate, strontium maleate, strontium methanesulfonate, strontium benzenesulfonate, strontium acetyl salicylate, strontium salicylate, strontium citrate, strontium alendronate, strontium risedronate, strontium chlodronate, strontium ethidronate and strontium L-threonate, strontium ibandronate, strontium ibuprofenate, strontium flubiprofenate, strontium ketoprofenate, strontium phorbol 12,13-didecanoate 20-homovanillate, strontium indomethacinate, strontium carprofenate, strontium naproxenate, strontium acetyloxy-benzoate, strontium 2-iminopiperidine, strontium methotrexate, strontium salsalate and strontium sulfasalazinate;
(22) glucosamine:
(23) a disease modifying anti-rheumatic compound (DMARD) selected from the group consisting of doxycycline, chondroitin sulfate, methotrexate, leflunomide, dimethylnitrosamine, azatriopine, hydroxychloroquine, cyclosporine, minocycline, salazopyrine, penicillamine, aurothiomalate (gold salt), cyclophosphamide, azathioprine and pharmacologically active metabolites thereof;
(24) an aromatase inhibitor selected from the group consisting of aminoglutethimide, testolactone, anastrozole, letrozole, exemestane, vorozole, formestane, fadrozole, 4-hydroxyandrostenedione, 1,4,6-androstatrien-3,17-dione, and 4-androstene-3,6,17-trione;
(25) a COX-3 inhibitor selected from the group consisting of acetaminophen, dipyrone, antipyrine, and dimethylaminopyrene:
(26) an opioid selected from the group consisting of fentanyl, morphine,
oxycodone, hydrocodone, methadone, buprenorphine, pentazocine, butorphanol, dezocine, nalbuphine, meperidine, normeperidine, hydromorphone, codeine, levorphanol, tramadol, endorphin, nociceptin, endomorphin, and active metabolites thereof;
(27) an inhibitor/antagonist of IL-1 that is a monoclonal antibody specifically binding IL-1 or a soluble IL-1 receptor derivative;
(28) an inhibitor/antagonist of interleukin-I converting enzyme;
(29) an inhibitor of RANK-ligand selected from the group consisting of OPG and monoclonal antibody 162;
(30) an anabolic growth factor selected from the group consisting of: (i) an anabolic growth factor derived from a bone or cartilage matrix protein selected from the group consisting of segments of or fragments from collagen type I, collagen type II, collagen type IX, collagen type XI, bone sialo protein (BSP), osteonectin, osteopontin, osteocalcin (also known as bone GLA protein), cartilage oligomeric matrix protein (COMP), cartilage intermediate layer protein (CILP) and aggrecan; (ii) human growth hormone (hGH); (iii) glucagon like peptide-2 (GLP-2); and (iv) insulin like growth factor-1 (IGF-1) with or without IGF binding protein 3 (IGFBP-3);
(31) a statin selected from the group consisting of nystatin, pravastatin, fluvostatin, atorvastatin, and cerivastatin and therapeutically active derivatives thereof;
(32) an endothelin-1 antagonist/inhibitor selected from the group consisting of bosentan, sitaxentan, ambrisentan, atrasentan, BQ-123 (2-[(3R,6R,9S,12R,15S)-6-(1H-indol-3-ylmethyl)-9-(2-methylpropyl)-2,5,8,11,14-pentaoxo-12-propan-2-yl-1,4,7,10,13-pentazabicyclo[13.3.0]octadecan-3-yl]acetic acid), zibotentan, macitentan, tenosentan, BQ-788 (N-[(cis-2,6-Dimethyl-1-piperidinyl)carbonyl]-4-methyl-L-leucyl-1-(methoxycarbonyl)-D-tryptophyl-D-norleucine sodium salt), and A192621 ((2R,3R,4S)-4-(1,3-benzodioxol-5-yl)-1-[2-(2,6-diethylanilino)-2-oxoethyl]-2-(4-propoxyphenyl)pyrrolidine-3-carboxylic acid);
(33) an NMDA receptor antagonist selected from the group consisting of R-2-amino-5-phosphonopentanoate, 2-amino-7-phosphonoheptanoic acid, 3-[(R)-2-carboxypiperazin-4-yl]-prop-2-enyl-1-phosphonic acid, selfotel, amantidine, atomoxetine, lanicemine, dextrallorphan, dizocilpine, gacyclidine, memantine, nitromemantine, neramexane, eliprodil, WMS-259 ((2S,4S)-2-[(4S)-2,2-Diphenyl-1,3-dioxolan-4-yl]-4-fluoropiperidine) remacemide, delucemine, aptiganel, rapastinel, NRX-1074 1-aminocyclopropane-1-carboxylic acid, and 5,7-dichlorokynurenic acid;
(34) a calcitonin gene related peptide-α antagonist selected from the group including olcegepant, telcagepant, ubrogepant, and an antibody or fragment thereof specifically binding calcitonin gene related peptide-α;
(35) chondroitin sulfate;
(36) keratin sulfate;
(37) a glycine antagonist selected from the group consisting of bicuculline, brucine, and tutin;
(38) a vanilloid receptor antagonist selected from the group consisting of AMG 517 (N-(4-((6-(4-trifluoromethyl)phenyl)pyrimidin-4-yl)oxy)benzo[d]thiazole-2-yl)acetamide), SB-705498 ((R)-1-(2-bromophenyl)-3-(1-(5-(trifluoromethyl)pyridin-2-yl)pyrrolidin-3-yl)urea), GRC 6211, AZD1386 and NGD 8243;
(39) a N-acetylcholine receptor antagonist selected from the group consisting of hexamethonium, mecamylamine, trimethaphan, atracurium, doxacurium, mivacurium, pancuronium, vecuronium, and 18-methoxycoronaridine;
(40) a neurokinin antagonist selected from the group consisting of RPR-100893 ((2S)-1-[(3 aS,4S,7aS)-4-hydroxy-4-(2-methoxyphenyl-7,7-diphenyl-1,3,3a, 5,6,7a-hexahydroisoindol-2-yl]-2-(2-methoxyphenyl)propan-1-one), CP-99994 ((2S,3S)—N-[(2-methoxyphenyl)methyl]-2-phenyl-3-piperidinamine dihydrochloride), L-733,060 ((2S,3S)-3-{[3,5-bis(trifluoromethyl)benzyl]oxy}-2-phenylpiperidine), aprepitant, fosaprepitant, vofopitant, lanepitant, and TAK-637 (R)-7-(3,5-bis(trifluoromethyl)benzyl)-9-methyl-5-(p-tolyl)8,9,10,11-tetrahydro-7H-[1,4]diazocino[2,1-g][1, 7]naphthyridine-6,13-dione);
(41) a neuroleptic agent selected from the group consisting of benperidol, bromperidol, droperidol, haloperidol, moperone, pipamperone, timiperone, fluspirilene, penfluridol, pimozide, acepromazine, chlorpromazine, cyamemazine, dixyrazine, fluphenazine, levomepromazine, mesoridazine, perazine, pericyazine, perphenazine, pipotiazine, prochlorperazine, promazine, promethazine, prothipendyl, thioproperazine, thioridazine, trifluoperazine, triflupromazine, chlorprothixene, clopenthixol, flupentixol, tiotixene, zuclopenthixol, clotiapine, loxapin, prothipendyl, carpipramine, clocapramine, molindone, mosapramine, sulpiride, sultopiride, veralipride, amisulpride, amoxapine, aripiprazole, asenapine, clozapine, blonanserin, iloperidone, lurasidone, melperone, nemonapride, olanzapine, paliperidone, perosperone, quetiapine, remoxipride, risperidone, sertindole, trimipramine, ziprasidone, and zotepine;
(42) a PAR2 receptor antagonist selected from the group consisting of AC-55541 (N-[[1-(3-bromo-phenyl)-eth-(E)-ylidene-hydrazinocarbonyl]-(4-oxo-3,4-dihydro-phthalazin-1-yl)-methyl]-benzamide) and AC-264613 (2-oxo-4-phenylpyrrolidine-3-carboxylic acid [1-(3-bromo-phenyl)-(E/Z)-ethylidene]-hydrazide; and
(43) a sulfated cyclodextrin.

307.-315. (canceled)
